(19) United States
(12) Patent Application Publication
(10) Pub. No.: US 2026/0000692 A1
(43) Pub. Date: Jan. 1, 2026

(54) Substituted N-(Pyridin-2-yl)Acetamide Derivatives as CDK12/13 Inhibitors

(71) Applicant: Aurigene Oncology Limited, Bangalore (IN)

(72) Inventors: Ramulu Poddutoori, Bengaluru (IN); Susanta Samajdar, Bangalore (IN); Subhendu Mukherjee, Bangalore (IN); Suraj Tatyasaheb Gore, Rahata (IN); Devaraja Thimmasandra Seethappa, Thimmasandra Village (IN); Paul Gillespie, San Ramon, CA (US); Scott Simonovich, Oakland, CA (US)

(73) Assignee: Aurigene Oncology Limited, Bangalore (IN)

(21) Appl. No.: 18/880,985

(22) PCT Filed: Jul. 5, 2023
(86) PCT No.: PCT/IB2023/056952

§ 371 Date: Jan. 3, 2025

(30) Foreign Application Priority Data
Jul. 5, 2022 (IN) 202241038474

(51) Int. Cl. A61K 31/675; A61K 31/4439; A61K 31/444; A61K 31/4545; A61K 31/496; A61K 31/497; A61K 31/4995; A61K 31/506; A61K 31/5377; A61K 31/5386; C07D 213/75; C07D 213/84; C07D 401/14; C07D 403/14; C07D 413/14; C07D 451/06; C07D 487/08; C07D 498/08; C07F 9/6558

(52) U.S. Cl. CPC A61K 31/675; A61K 31/4439; A61K 31/444; A61K 31/4545; A61K 31/496; A61K 31/497; A61K 31/4995; A61K 31/506; A61K 31/5377; A61K 31/5386; C07D 213/75; C07D 213/84; C07D 401/14; C07D 403/14; C07D 413/14; C07D 451/06; C07D 487/08; C07D 498/08; C07F 9/65583

(57) ABSTRACT

The present application provides substituted N-(pyridin-2-yl)acetamide derivatives of formula (I)

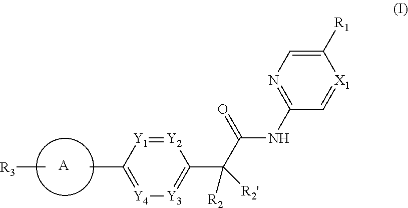

which are therapeutically useful as CDK12/13 inhibitors. These compounds are useful in the treatment and/or prevention of diseases and/or disorders associated with CDK12/13 in a mammal. In addition, the present application provides preparation of the compounds and pharmaceutical compositions comprising at least one of the substituted N-(pyridin-2-yl)acetamide derivatives of formula (I) or a pharmaceutically acceptable salt, an N-oxide or a stereoisomer thereof.

This application claims priority from Indian Application No. 202241038474, filed on 05, July, 2022, which is incorporated herein in its entirety for all purposes.

### TECHNICAL FIELD

This application relates to substituted N-(pyridin-2-yl)acetamide derivatives useful for treatment of cancer and inflammatory diseases associated with CDK12/13. The application also provides pharmaceutically acceptable compositions comprising compounds of formula (I) and methods of using said compositions in the treatment of diseases associated with CDK12/13.

### BACKGROUND

Cyclin dependent kinases (CDKs) are a family of Ser/Thr kinases that integrate various signal transduction pathways and play a role in several key cellular processes. CDK12 and its paralogue CDK13 belong to the class of ‘transcriptional’ CDKs. The CDK12/Cyclin K complex regulates transcriptional elongation, pre-mRNA splicing and alternate splicing. Mutation of CDK12 in serous ovarian carcinoma is associated with decreased expression of DNA damage response (DDR) genes such as BRCA1, FANCI, ATM, ATR or FANCD2 and increased sensitivity to PARP inhibitors. (Cancer Res, 2016, 76(7)1882; Nucleic Acids Research, 2015, Vol. 43, 2575-2589). Hence, maintenance of genomic stability appears to be the key role of this protein.

Transcription of protein-coding genes is controlled by RNA Polymerase II. Phosphorylation of residues in its C-terminal domain (CTD) orchestrate the production of mature mRNA transcripts. Phosphorylation of Ser2, which promotes elongation of RNA Pol II through the gene body, is a key mechanism of CDK12 transcriptional regulation (Genes & Development 2010, 24:2303-2316). As a consequence, CDK12 knockdown has also been associated with downregulation of genes involved in homologous recombination (Genes & Development 2011, 25:2158-2172). The emergence of an increasingly significant role of CDK12 in genomic stability and oncogenesis provides new insight into the function of CDK12 in genome maintenance and oncogenesis.

The frequency and distribution of CDK12 protein expression was assessed by Immunohistochemistry (IHC) in independent cohorts of breast cancer and this was correlated with outcome and genomic status. It was found that 21% of primary unselected breast cancers were CDK12 high, and 10.5% were absent. CDK12 overexpression in breast cancer cells has been demonstrated to regulate splicing of pre-mRNA involved in DDR and tumorigenesis. (Nucleic Acids Res., 2017, Jun. 20; 45(11):6698-6716). Disruption of Cyclin-Dependent Kinase 12 (CDK12) is known to lead to defects in DNA repair and sensitivity to platinum salts and PARP1/2 inhibitors. Interestingly, absence of CDK12 protein was associated with reduced expression of a number of DDR proteins including ATR, Ku70/Ku80, PARP1, DNA-PK, and γH2AX, suggesting a novel mechanism of CDK12-associated DDR dysregulation in breast cancer. This may have important therapeutic implications, particularly for triple-negative breast cancers. (Molecular Cancer Therapeutics (2018), 17(1), 306-315).

As transcription is a highly critical cellular process and is controlled by different transcription regulating kinases, it is desirable to have as selective a compound as possible to overcome unwanted side effects. CDK7 is reported to control transcription initiation by phosphorylation of Ser5 and Ser7 residues of RNA polymerase II while CDK12 is reported to be responsible for elongation of transcription through phosphorylation of Ser2 residue of RNA polymerase II (Nucleic Acids Research, 2015, Vol. 43, No. 5, 2575-2589).

The genetic loss of CDK12, silencing of CDK12 as well as pharmacological inhibition of it result in a selective transcriptional defect for genes involved in cellular response to DNA damage, stress, and heat shock. The genomic instability induced by CDK12 inactivation might provide an opportunity for cancer therapy (Nat Chem Biol. 2021 June; 17(6): 675-683). Similar to CDK7, inhibitors of CDK12/13 may offer an advantage by targeting two processes important to many cancer cells, aberrant transcription and genomic instability (Transcription. 2019; 10(2): 118-136).

There remains a need in the art to find compounds which selectively inhibit CDK12/13 over other CDKs. It is, therefore, an objective of this application to provide compounds useful in the treatment and/or prevention or amelioration of diseases and/or disorders associated with CDK12/13.

### SUMMARY

Provided herein are substituted N-(pyridin-2-yl)acetamide derivatives and pharmaceutical compositions thereof, which are useful as CDK12/13 inhibitors.

In one aspect of the embodiment, it comprises compounds of formula (I):

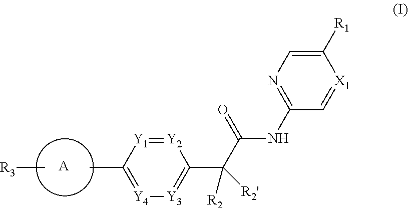

or a pharmaceutically acceptable salt, an N-oxide or a stereoisomer thereof;
wherein,
X₁ is CR₅ or N;
each of Y₁, Y₂, Y₃ and Y₄ is independently CR₆ or N, wherein 0-2 of Y₁, Y₂, Y₃ and Y₄ are N;
ring A is

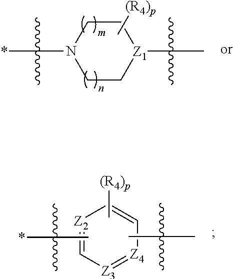

wherein * is the point of attachment with R₃;
‘-----’ is an optional bond;
Z₁ is C or N;
Z₂, Z₃ and Z₄ are each independently C or N;
R₁ is hydrogen, halogen, alkyl, cycloalkyl or alkylthio;
each of R₂ and R₂′ independently is hydrogen or alkyl;
R₃ is —CN or

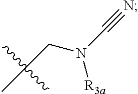

wherein wavy bond indicates the point of attachment
with ring A;
R₃ₐ is hydrogen or alkyl;
R₄ at each occurrence independently is halogen, alkyl, haloalkyl, hydroxyalkyl, —OR₄ₐ, —NR_{4b}R_{4c}, unsubstituted or substituted cycloalkyl, unsubstituted or substituted heterocycloalkyl; wherein the substituent is selected from one or more alkyl, halo, alkoxy, haloalkyl or hydroxy;
alternatively, two R₄ each on different carbon atoms form a bridging (C₁-C₃) alkylene or a bridging bond;
R₄ₐ is alkyl, haloalkyl, alkoxyalkyl, alkylaminoalkyl, unsubstituted or alkyl substituted heterocycloalkyl;
R_{4b} and R_{4c} are each independently hydrogen, alkyl, alkylaminoalkyl or unsubstituted or alkyl substituted heterocycloalkyl;
R₅ is:
i) hydrogen, halogen, hydroxyalkyl, alkyl, —PO(CH₃)₂, —OR₅ₐ or —NR_{5b}R_{5c}; or
ii) unsubstituted or substituted heterocycloalkyl, wherein, the substituents are 1 or 2 substituents independently selected from alkyl and hydroxy;
R₅ₐ is alkyl, unsubstituted or alkyl substituted heterocycloalkylalkyl, unsubstituted or alkyl substituted heterocycloalkyl, —CONR_{5d}R₅ₑ, -alkyl-CONR_{5d}R₅ₑ, cycloalkyl, alkoxyalkyl or alkylaminoalkyl;
R5b and R_{5c} are each independently hydrogen or alkyl;
R_{5d} and R₅ₑ are each independently hydrogen or alkyl;
R₆ at each occurrence, is independently hydrogen, alkyl, alkoxy or halogen;
‘p’ is selected from 0 to 3; and
‘m’ and ‘n’ are each independently selected from 0 to 2.

In yet another aspect, provided herein is a pharmaceutical composition comprising a compound of formula (I) or a pharmaceutically acceptable salt or a stereoisomer thereof and at least one pharmaceutically acceptable carrier or excipient.

In yet another aspect, the present application relates to the preparation of compounds of formula (I).

In yet another aspect of the embodiment, provided herein are substituted N-(pyridin-2-yl)acetamide derivatives of formula (I), which are capable of inhibiting CDK12/13 and therapeutic uses thereof.

In yet another aspect of the embodiment, provided herein are substituted N-(pyridin-2-yl)acetamide derivatives of formula (I), which are used in the treatment of cancer.

In a still further aspect, provided herein is a method of treating a disease and/or disorder or a condition mediated by CDK12/13 in a subject comprising administration of compounds of formula (I) or compositions thereof.

### DETAILED DESCRIPTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present application and not intended to be limiting.

As used herein, the singular form “a”, “an” and “the” include plural references unless indicated otherwise. For example, “a” substituent includes one or more substituents, reference to “an agent” includes a plurality of such agents, and reference to “the cell” includes reference to one or more cells (or to a plurality of cells) and equivalents thereof known to those skilled in the art, and so forth.

When ranges are used herein for physical properties, such as molecular weight, or chemical properties, such as chemical formulae, all combinations and sub-combinations of ranges and specific embodiments therein are intended to be included.

As used herein, the term “optionally substituted” refers to replacement of one or more hydrogen radicals in a given structure with a radical of a specified substituent including, but not limited to: halo, alkyl, alkenyl, alkynyl, aryl, heterocyclyl, thio, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, cycloalkyl, and heteroaryl. It is understood that the substituent may be further substituted.

As used herein, unless otherwise defined the term “alkyl” alone or in combination with other term(s) means saturated aliphatic hydrocarbon chain, including C₁-C₁₀ straight or C₁-C₁₀ branched alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include but are not limited to methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted. A C₁-C₆ straight chained or branched alkyl group is also referred to as a “lower alkyl” group.

As used herein, unless otherwise defined the term “alkylene” alone or in combination with other term(s) means saturated bivalent aliphatic hydrocarbon chain, including C₁-C₁₀ straight or C₁-C₁₀ branched hydrocarbon chains. Preferably, the “alkylene” group refers to C₁-C₃ straight-chain alkylene groups.

As used herein, the term “halo” or “halogen” alone or in combination with other term(s) means fluorine, chlorine, bromine or iodine.

As used herein, the term “haloalkyl” means alkyl substituted with one or more halogen atoms, wherein the alkyl groups are as defined above. The term “halo” is used herein interchangeably with the term “halogen” to mean F, Cl, Br or I. Examples of “haloalkyl” include but are not limited to fluoromethyl, difluoromethyl, chloromethyl, trifluoromethyl, 2,2,2-trifluoroethyl and the like.

As used herein, the term “hydroxy” or “hydroxyl” alone or in combination with other term(s) means —OH.

As used herein, the term “hydroxyalkyl” means alkyl substituted with one or more “hydroxy” or “hydroxyl” group, wherein the alkyl group is as defined above.

As used herein, the term “alkoxy” refers to the group alkyl-O— or —O-alkyl, where alkyl groups are as defined above. Examples of “alkoxy” groups include but are not limited to methoxy, ethoxy, n-propoxy, n-butoxy, t-butoxy and the like. An alkoxy group can be unsubstituted or substituted with one or more suitable groups.

As used herein, the term “alkoxyalkyl” means alkyl substituted with one or more “alkoxy” groups, wherein the alkyl group is as defined above. Examples of “alkoxyalkyl” groups include but are not limited to methoxymethyl, ethoxymethyl, propoxyethyl, butoxypropyl and the like.

As used herein, the term “alkylthio” refers to the group alkyl-S—, where alkyl groups are as defined herein. Examples of “alkylthio” groups include, but are not limited to, methylthio, ethylthio, isopropylthio, and the like. An alkylthio group can be unsubstituted or substituted with one or more suitable groups.

The term “amino” or “amine” refers to a primary amine

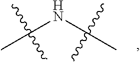 [SMILES: *N*]

wherein ‘N’ is substituted with two substituents other than hydrogen) or tertiary amine

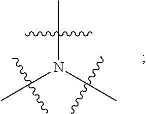 [SMILES: *N(*)*]

wherein ‘N’ is substituted with three substituents other than hydrogen) group.

As used herein, the term “alkylamino” refers to an amino group substituted with one or more “alkyl” group, wherein the alkyl group and amino group is as defined above. Examples of “alkylamino” groups include but are not limited to —NHCH₃, —NHCH₂CH₃, —N(CH₃)₂, —N(CH₃)(CH₂CH₃) and the like.

As used herein, the term “alkylaminoalkyl” means alkyl substituted with one or more “alkylamino” groups, wherein the alkyl group and alkylamino group is as defined above. Examples of “alkylaminoalkyl” groups include but are not limited to —CH₂NH(CH₃), —(CH₂)ₙ—NH(CH₃), —CH₂CH(CH₃)N(CH₃)₂, —(CH₂)n-N(CH₃)₂, —CH₂NHCH₃, —CH₂NHCH₂CH₃, —(CH₂)ₙN(CH₃)₂, —CH₂N(CH₃)(CH₂CH₃) and the like.

As used herein, the term “cyano” refers to a —CN group.

The term “heteroatom” as used herein designates a sulfur, nitrogen or oxygen atom.

As used herein the term “cycloalkyl” alone or in combination with other term(s) means —C₃-C₁₀ saturated or unsaturated non-aromatic cyclic hydrocarbon ring. A cycloalkyl may be a single ring, which typically contains from 3 to 7 carbon ring atoms. Examples of single-ring cycloalkyls include but are not limited to cyclopropyl, cyclobutyl, cyclopentyl, cyclohexyl, cycloheptyl and the like. A cycloalkyl may alternatively be polycyclic or contain more than one ring. Examples of polycyclic cycloalkyls include bridged, fused and spirocyclyls and the like (tetrahydronaphthyl, fluorenyl, indanyl, etc.).

As used herein, the term “aryl” is optionally substituted monocyclic, bicyclic or polycyclic aromatic hydrocarbon ring system of about 6 to 14 carbon atoms. In some embodiments, aryl groups have 6 to 12 ring carbon atoms. In some embodiments, aryl groups have 6 to 10 ring carbon atoms. In some embodiments, aryl groups have 6 ring carbon atoms. In some embodiments, aryl groups have 10 ring carbon atoms. Also included in the definition of aryl are moieties that have one or more cycloalkyl rings fused (i.e., having a bond in common with) to the aryl ring, e.g., tetrahydronaphthyl, fluorenyl, indanyl, and the like. In some embodiments, an aryl group containing a fused cycloalkyl ring can be attached (to the rest of the molecule) through any ring-forming atom including a ring-forming atom of the fused cycloalkyl ring. In some embodiments, an aryl group containing a fused cycloalkyl ring is attached (to the rest of the molecule) through a ring-forming atom of the aromatic ring. Examples of a C₆-C₁₄ aryl group include, but are not limited to phenyl, naphthyl, anthryl, biphenylenyl and acenaphthyl. An aryl group can be unsubstituted or substituted with one or more suitable groups.

As used herein, the term “heterocycloalkyl” refers to a non-aromatic, saturated or partially saturated, bridged bicyclic, spirocyclic, monocyclic or polycyclic ring system of 3 to 15 members, preferably 3 to 10 members, having at least one heteroatom or heterogroup selected from O, N, S, S(O), S(O)₂ or NH with the remaining ring atoms being independently selected from carbon, oxygen, nitrogen, and sulfur. The term “heterocycloalkyl” also refers to the bridged bicyclic ring system having at least one heteroatom or hetero group selected from O, N, S, S(O), S(O)₂ or NH. Examples of “heterocycloalkyl” include, but are not limited to azetidinyl, oxetanyl, imidazolidinyl, pyrrolidinyl, oxazolidinyl, thiazolidinyl, pyrazolidinyl, tetrahydrofuranyl, piperidinyl, piperazinyl, morpholinyl, thiomorpholinyl, 1,4-dioxanyl, dioxidothiomorpholinyl, oxapiperazinyl, oxapiperidinyl, tetrahydrofuryl, tetrahydropyranyl, tetrahydrothiophenyl, dihydropyranyl, indolinyl, aza-bicyclooctanyl, azocinyl, chromanyl, isochromanyl xanthenyl, 2-oxa-6-azaspiro[3.3]heptanyl, 3-oxa-8-azabicyclo[3.2.1]octanyl, hydroxyoxetanyl, tetrahydropyridinyl, 1,2-dimethylpiperidinyl, 2,2-dimethylpiperidinyl, 6,6-dimethylpiperidinyl, 4-cyanopiperazinyl, 3-methylpiperidinyl, N-methyl-2,5-diazabicyclo[2.2.1]heptanyl, 3-azabicyclo[4.1.0]heptanyl, 2-methyl-2-azaspiro[3.3]heptanyl, 8-azabicyclo[3.2.1]octanyl, octahydrocyclopenta[c]pyrrolyl, 1,2,3,3a,4,6a-hexahydrocyclopenta[c]pyrrolyl, 8-methyl-3,8-diazabicyclo[3.2.1]octanyl, 2-azabicyclo[2.2.2]oct-5-enyl, 8-azabicyclo[3.2.1]oct-2-enyl, 8-azabicyclo[3.2.1]octanyl and N-oxides thereof. Attachment of a heterocycloalkyl substituent (to the rest of the molecule) can occur via either a carbon atom or a heteroatom. A heterocycloalkyl group can be optionally further substituted.

As used herein, the term “heteroaryl” refers to an aromatic heterocyclic ring system containing 5 to 20 ring atoms, suitably 5 to 12 ring atoms, which may be a single ring (monocyclic) or multiple rings (bicyclic, tricyclic or polycyclic) fused together or linked covalently. Preferably, “heteroaryl” is a 5- to 6-membered ring. The rings may contain from 1 to 4 heteroatoms independently selected from N, O and S, wherein the N or S atom is optionally oxidized or the N atom is optionally quarternized. Any suitable ring position of the heteroaryl moiety may be covalently linked to the defined chemical structure. Examples of “heteroaryl” include but are not limited to furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl, pyrimidinyl, pyrazinyl, pyridazinyl, benzoxazolyl, benzisoxazolyl, benzothiazolyl, benzofuranyl, benzothienyl, benzotriazinyl, phthalazinyl, thianthrenyl, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carbolinyl, indolizinyl, benzoisothiazolyl, pyrrolopyridyl, furopyridinyl, purinyl, benzothiadiazolyl, benzooxadiazolyl, benzotriazolyl, benzothiadiazolyl, dibenzothienyl, acridinyl and the like. Heteroaryl group may be optionally further substituted.

As used herein, the term “heterocyclyl” or “heterocyclic” alone or in combination with other term(s) includes both “heterocycloalkyl” and “heteroaryl” groups which are as defined above.

As used herein, the term “heterocycloalkylalkyl” refers to an alkyl group substituted with a heterocycloalkyl ring wherein both “alkyl” and “heterocycloalkyl” terms are as defined above.

Certain of the compounds disclosed herein can exist as N-oxides. For example, it is known that the pyrazoles can form N-oxides on treatment with a suitable oxidizing agent. Similarly, it is known that the pyridine ring nitrogen can be oxidized on treatment with a suitable oxidizing agent to form an N-oxide.

As used herein, the term “compound(s)” comprises the compounds disclosed in the present application.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of formula (I) or its pharmaceutically acceptable salt; and a conventional pharmaceutically acceptable carrier.

The pharmaceutical composition(s) of the present application can be administered orally, for example in the form of tablets, coated tablets, pills, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermals, in the form of patches, or in other ways, for example in the form of aerosols or nasal sprays.

The pharmaceutical composition(s) usually contain(s) about 1% to 99%, for example, about 5% to 75%, or from about 10% to about 30% by weight of the compound of formula (I) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the afore mentioned range.

As used herein, the term “treat”, “treating” and “treatment” refer to any treatment of a disease in a mammal, including: (a) Inhibiting the disease, i.e., slowing or arresting the development of clinical symptoms; and/or (b) relieving the disease, i.e., causing the regression of clinical symptoms and/or (c) alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “therapeutically effective amount” refers to that amount of a compound of formula (I) or a pharmaceutically acceptable salt or a stereoisomer thereof; or a composition comprising the compound of formula (I) or a pharmaceutically acceptable salt or a stereoisomer thereof, effective in producing the desired therapeutic response in a particular patient suffering from a diseases or disorder, in particular their use in diseases or disorder associated with cancer. Particularly, the term “therapeutically effective amount” includes the amount of the compound of formula (I) or a pharmaceutically acceptable salt or a stereoisomer thereof, when administered, that induces a positive modification in the disease or disorder to be treated or is sufficient to prevent development of, or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

“Pharmaceutically acceptable salt” refers to a product obtained by reaction of the compound of formula (I) with a suitable acid or a base and that possesses the desired pharmacological activity of the parent compound. Pharmaceutically acceptable salts of the compounds of formula (I) include those derived from suitable inorganic and organic acids and bases. Such salts include: acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentane propionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methane sulfonic acid, ethane sulfonic acid, 1,2-ethane-disulfonic acid, 2-hydroxyethanesulfonic acid, benzene sulfonic acid, 4-chlorobenzenesulfonic acid, 2-naphthalenesulfonic acid, 4-toluenesulfonic acid, camphor sulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxyl naphthoic acid, salicylic acid, stearic acid, muconic acid, and the like. Certain compounds disclosed herein (compounds of formula (I)) can form pharmaceutically acceptable salts with various organic bases such as lysine, arginine, guanidine, diethanolamine or metformin.

As used herein, the term “about” when referring to a number or a numerical range means that the number or numerical range referred to, is an approximation within experimental variability (or within statistical experimental error), and thus the number or numerical range may vary from, for example, between 1% and 15% of the stated number or numerical range.

The term “stereoisomers” refers to any enantiomers, diastereoisomers, or geometrical isomers of the compounds of Formula (I), wherever they are chiral or when they bear one or more double bonds. When the compounds of the Formula (I) and related formulae are chiral, they can exist in racemic or in optically active form. It should be understood that this application encompasses all stereochemical isomeric forms, including diastereomeric, enantiomeric and epimeric forms, (R) and (S) isomers, as well as d-isomers and i-isomers and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of formula (I) may exist as geometric isomers. The present application includes all cis, trans, syn, anti, entgegen (E) and zusammen (Z), (R) and (S) isomers as well as the appropriate mixtures thereof.

It will be apparent to those skilled in the art that various modifications and variations can be made to the compounds, compositions, and methods described herein without departing from the scope or spirit of various embodiments disclosed herein. For instance, features illustrated or described as part of one embodiment can be applied to another embodiment to yield a still further embodiment. Thus, it is intended that the present application includes such modifications and variations and their equivalents. Other objects, features, and aspects of the present application are disclosed in, or are obvious from, the following detailed description. It is to be understood by one of ordinary skill in the art that the present discussion is a description of exemplary embodiments only and is not to be construed as limiting the broader aspects of the present application.

In one aspect of the embodiment, provided herein are substituted N-(pyridin-2-yl)acetamide derivatives of formula (I), which are useful as CDK12/13 inhibitors.

The present application further provides pharmaceutical compositions comprising the said substituted N-(pyridin-2-yl)acetamide compounds of formula (I) and their derivatives as therapeutic agents.

In one embodiment, provided herein are compounds of formula (I),

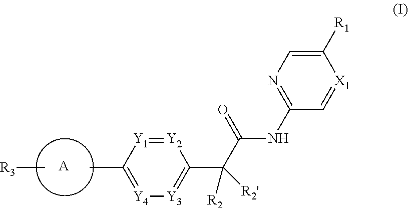

or a pharmaceutically acceptable salt, an N-oxide or a stereoisomer thereof;
wherein,
X₁ is CR₅ or N;
each of Y₁, Y₂, Y₃ and Y₄ is independently C_{R} or N, wherein 0-2 of Y₁, Y₂, Y₃
and Y₄ are N;
ring A is

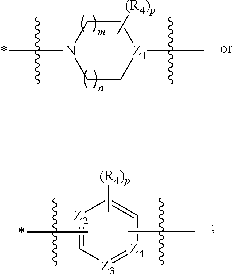

wherein * is the point of attachment with R₃;
‘-----’ is an optional bond;
Z₁ is C or N;
Z₂, Z₃ and Z₄ are each independently C or N;
R₁ is hydrogen, halogen, alkyl, cycloalkyl or alkylthio;
each of R₂ and R₂′ independently is hydrogen or alkyl;
R₃ is —CN or

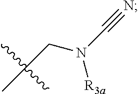

wherein wavy bond indicates the point of attachment with ring A;
R₃ₐ is hydrogen or alkyl;
R₄ at each occurrence independently is halogen, alkyl, haloalkyl, hydroxyalkyl, —OR₄ₐ, —NR_{4b}R_{4c}, unsubstituted or substituted cycloalkyl, unsubstituted or substituted heterocycloalkyl; wherein the substituent is selected from one or more alkyl, halo, alkoxy, haloalkyl or hydroxy;
alternatively, two R₄ each on different carbon atoms form a bridging (C₁-C₃) alkylene or a bridging bond;
R₄ₐ is alkyl, haloalkyl, alkoxyalkyl, alkylaminoalkyl, unsubstituted or alkyl substituted heterocycloalkyl;
R_{4b} and R_{4c} are each independently hydrogen, alkyl, alkylaminoalkyl, unsubstituted or alkyl substituted heterocycloalkyl;
R₅ is:
i) hydrogen, halogen, hydroxyalkyl, alkyl, —PO(CH₃)₂, —OR₅a or —NR_{5b}R_{5c}; or
ii) unsubstituted or substituted heterocycloalkyl, wherein, the substituents are 1 or 2 substituents independently selected from alkyl and hydroxy;
R₅ₐ is alkyl, unsubstituted or alkyl substituted heterocycloalkylalkyl, unsubstituted or alkyl substituted heterocycloalkyl, —CONR_{5d}R₅ₑ, -alkyl-CONR_{5d}R₅ₑ, cycloalkyl, alkoxyalkyl or alkylaminoalkyl;
R_{5b} and R_{5c} are each independently hydrogen or alkyl;
R_{5d} and R₅ₑ are each independently hydrogen or alkyl;
R₆ at each occurrence, is independently hydrogen, alkyl, alkoxy or halogen;
‘p’ is selected from 0 to 3; and
‘m’ and ‘n’ are each independently selected from 0 to 2.

In another aspect of the embodiment, provided herein are compounds of formula (I), wherein, X₁ is CR₅;
each of Y₁, Y₂, Y₃ and Y₄ is independently CR₆ or N; wherein 0-2 of Y₁, Y₂, Y₃ and Y₄ are N;
ring A is

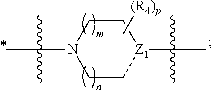

wherein * is the point of attachment with R₃;
‘-----’ is an optional bond;
Z₁ is C or N;
R₁ is hydrogen, halogen, alkyl, cycloalkyl or alkylthio;
each of R₂ and R₂′ independently is hydrogen or alkyl;
R₃ is —CN;
R₄ at each occurrence independently is halogen, alkyl, haloalkyl, hydroxyalkyl, —OR₄ₐ, —NR_{4b}R_{4c}, unsubstituted or substituted cycloalkyl, unsubstituted or substituted heterocycloalkyl; wherein the substituent is selected from one or more alkyl, halo, alkoxy, haloalkyl or hydroxy;
alternatively, two R₄ each on different carbon atoms form a bridging (C₁-C₃) alkylene or a bridging bond;
R₄ₐ is alkyl, haloalkyl, alkoxyalkyl, alkylaminoalkyl, unsubstituted or alkyl substituted heterocycloalkyl;
R_{4b} and R_{4c} are each independently hydrogen, alkyl, alkylaminoalkyl or unsubstituted or alkyl substituted heterocycloalkyl;
R₅ is:
i) hydrogen, halogen, hydroxyalkyl, alkyl, —PO(CH₃)₂, —OR₅a or —NR_{5b}R_{5c}; or
ii) unsubstituted or substituted heterocycloalkyl, wherein, the substituents are 1 or 2 substituents independently selected from alkyl and hydroxy;
R₅ₐ is alkyl, unsubstituted or alkyl substituted heterocycloalkylalkyl, unsubstituted or alkyl substituted heterocycloalkyl, —CONR_{5d}R₅ₑ, -alkyl-CONR_{5d}R₅ₑ, cycloalkyl, alkoxyalkyl or alkylaminoalkyl;
R_{5b} and R_{5c} are each independently hydrogen or alkyl;
R_{5d} and R₅ₑ are each independently hydrogen or alkyl;
R₆ at each occurrence, is independently hydrogen, alkyl, alkoxy or halogen;
‘p’ is selected from 0 to 3; and
‘m’ and ‘n’ are each independently selected from 0 to 2.

In another aspect of the embodiment, provided herein are compounds of formula (I), wherein,
X₁ is CR₅;
each of Y₁, Y₂, Y₃ and Y₄ is independently CR₆ or N; wherein 0-2 of Y₁, Y₂, Y₃ and Y₄ are N;
ring A is

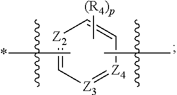

wherein * is the point of attachment with R₃;
Z₂, Z₃ and Z₄ are each independently C or N;
R₁ is hydrogen, halogen, alkyl, cycloalkyl or alkylthio;
each of R₂ and R₂′ independently is hydrogen or alkyl;
R₃ is —CN;
R₄ at each occurrence independently is halogen, alkyl, haloalkyl, hydroxyalkyl, —OR₄ₐ, —NR_{4b}R_{4c}, unsubstituted or substituted cycloalkyl, unsubstituted or substituted heterocycloalkyl; wherein the substituent is selected from one or more alkyl, halo, alkoxy, haloalkyl or hydroxy; alternatively, two R₄ each on different carbon atoms form a bridging (C₁-C₃) alkylene or a bridging bond;
R₄ₐ is alkyl, haloalkyl, alkoxyalkyl, alkylaminoalkyl, unsubstituted or alkyl substituted heterocycloalkyl;
R_{4b} and R_{4c} are each independently hydrogen, alkyl, alkylaminoalkyl or unsubstituted or alkyl substituted heterocycloalkyl;
R₅ is:
i) hydrogen, halogen, hydroxyalkyl, alkyl, —PO(CH₃)₂, —OR₅ₐ or —NR_{5b}R_{5c}; or
ii) unsubstituted or substituted heterocycloalkyl, wherein, the substituents are 1 or 2 substituents independently selected from alkyl and hydroxy;
R₅ₐ is alkyl, unsubstituted or alkyl substituted heterocycloalkylalkyl, unsubstituted or alkyl substituted heterocycloalkyl, —CONR_{5d}R₅ₑ, -alkyl-CONR_{5d}R₅ₑ, cycloalkyl, alkoxyalkyl or alkylaminoalkyl;
R_{5b} and R_{5c} are each independently hydrogen or alkyl;
R_{5d} and R₅ₑ are each independently hydrogen or alkyl;
R₆ at each occurrence, is independently hydrogen, alkyl, alkoxy or halogen;
‘p’ is selected from 0 to 3; and
‘m’ and ‘n’ are each independently selected from 0 to 2.

In yet another aspect of the embodiment, provided herein are compounds of formula (IA),

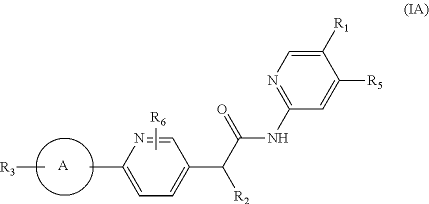

or a pharmaceutically acceptable salt, an N-oxide or a stereoisomer thereof.

In yet another aspect of the embodiment, provided herein are compounds of formula (IB),

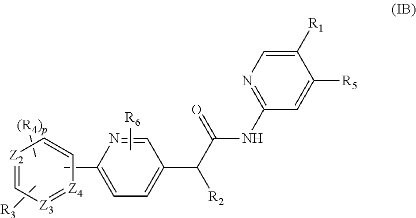

or a pharmaceutically acceptable salt, an N-oxide or a stereoisomer thereof.

In yet another aspect of the embodiment, provided herein are compounds of formula (IC),

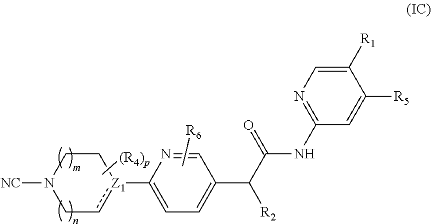

or a pharmaceutically acceptable salt an N-oxide or a stereoisomer thereof.

In yet another aspect of the embodiment, provided herein are compounds of formula (ID),

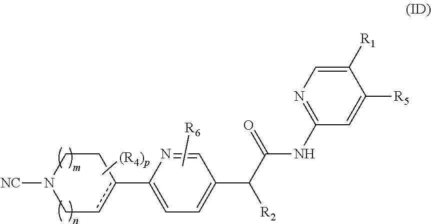

or a pharmaceutically acceptable salt, an N-oxide or a stereoisomer thereof.

In yet another aspect of the embodiment, provided herein are compounds of formula (IE),

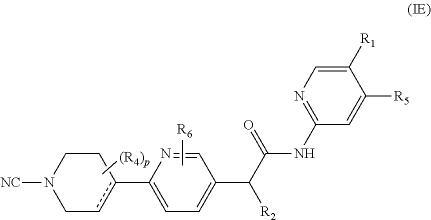

or a pharmaceutically acceptable salt, an N-oxide or a stereoisomer thereof.

In yet another aspect of the embodiment, provided herein are compounds of formula (IF),

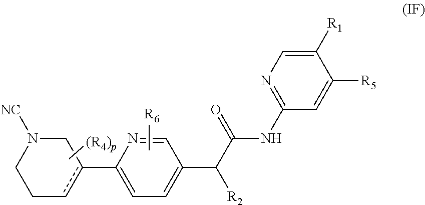

or a pharmaceutically acceptable salt, an N-oxide or a stereoisomer thereof.

In yet another aspect of the embodiment, provided herein are compounds of formula (IG),

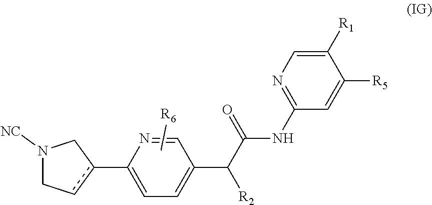

or a pharmaceutically acceptable salt, an N-oxide or a stereoisomer thereof.

In yet another aspect of the embodiment, provided herein are compounds of formula (IH),

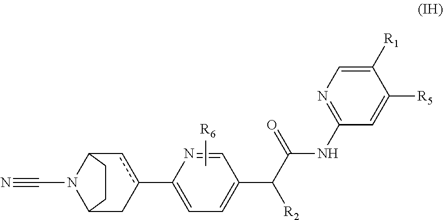

or a pharmaceutically acceptable salt, an N-oxide or a stereoisomer thereof.

In yet another aspect of the embodiment, provided herein are compounds of formula (IJ),

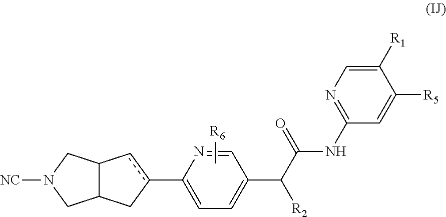

or a pharmaceutically acceptable salt, an N-oxide or a stereoisomer thereof.

In yet another aspect of the embodiment, provided herein are compounds of formula (IK),

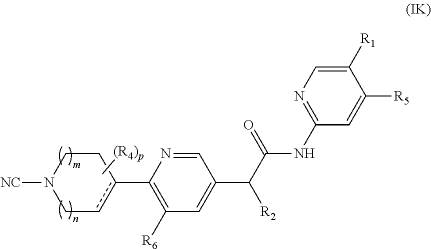

or a pharmaceutically acceptable salt, an N-oxide or a stereoisomer thereof;
wherein,
‘-----’ is an optional bond;
R₁ is hydrogen, halogen, alkyl, cycloalkyl or alkylthio;
R₂ is hydrogen or alkyl;
R₄ at each occurrence independently is halogen or alkyl;
alternatively, two R₄ each on different carbon atoms form a bridging (C₁-C₃) alkylene or a bridging bond;
R₅ is:
i) hydrogen, halogen, hydroxyalkyl, alkyl, —PO(CH₃)₂, —OR₅ₐ or —NR_{5b}R_{5c}; or
ii) substituted or unsubstituted heterocycloalkyl, wherein, the substituents on heterocycloalkyl are 1 or 2 substituents independently selected from alkyl and hydroxy;
R₅ₐ is alkyl, alkyl substituted or unsubstituted heterocycloalkylalkyl, unsubstituted or alkyl unsubstituted heterocycloalkyl, —CONR_{5d}R₅ₑ, -alkyl-CONR_{5d}R₅ₑ, alkoxyalkyl or alkylaminoalkyl;
R_{5b} and R_{5c} are each independently hydrogen or alkyl;
R_{5d} and R₅ₑ are each independently hydrogen or alkyl;
R₆ at each occurrence, is independently hydrogen, alkyl, alkoxy or halogen;
‘p’ is selected from 0 to 3; and
‘m’ and ‘n’ are each independently selected from 0 to 2.

The embodiments below are illustrative of the present application and are not intended to limit the claims to the specific embodiments exemplified.

According to one embodiment, specifically provided are compounds of formula (I), wherein, X₁ is CR₅.

According to one embodiment, specifically provided are compounds of formula (I), wherein, X₁ is N.

According to one embodiment, specifically provided are compounds of formula (I), wherein,

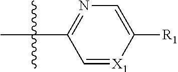

is

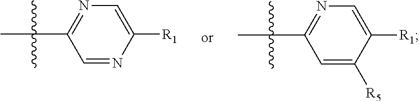

wherein the wavy line indicates the point of attachment to the rest of the molecule in formula (I).

According to one embodiment, specifically provided are compounds of formula (I), wherein, each Y₁, Y₂, Y₃ and Y₄ are CR₆.

According to one embodiment, specifically provided are compounds of formula (I), wherein, Y₁ is N, Y₂ and Y₃ are each CH, Y₄ is CR₆.

According to one embodiment, specifically provided are compounds of formula (I), wherein, Y₁ is N, Y₂, Y₃ and Y₄ are each CH.

According to one embodiment, specifically provided are compounds of formula (I), wherein, Y₁ is N, Y₃ and Y₄ are each CH, Y₂ is CR₆.

According to one embodiment, specifically provided are compounds of formula (I), wherein, each of Y₂, Y₃ and Y₄ is CH, and Y₁ is N.

According to one embodiment, specifically provided are compounds of formula (I), wherein, each of Y₂, Y₃ and Y₄ is CH, Y₁ is N and X₁ is CR₅.

According to one embodiment, specifically provided are compounds of formula (I), wherein, Y₁ is N.

According to one embodiment, specifically provided are compounds of formula (I), wherein, Y₄ is —CR₆.

According to one embodiment, specifically provided are compounds of formula (I), wherein, Y₁ is N and Y₄ is —CR₆.

In yet another embodiment, it provides compounds of formula (I), wherein, ring

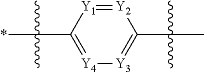

is

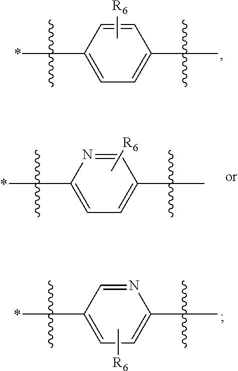

wherein the wavy line with the asterisk symbol indicates the point of attachment to ring A and the wavy line absent of asterisk symbol indicates the point of attachment to the carbon atom having R₂ and R₂′ substituents in formula (I).

In yet another embodiment, it provides compounds of formula (I), wherein, ring

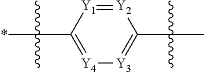

is

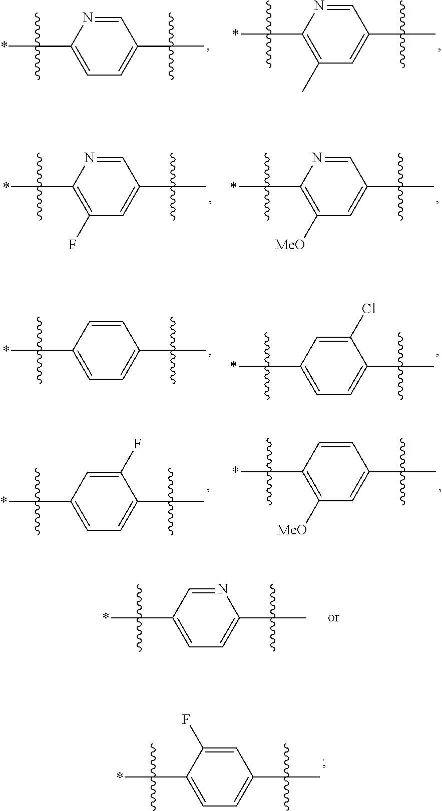

wherein the wavy line with the asterisk symbol indicates the point of attachment to ring A and the wavy line absent of asterisk symbol indicates the point of attachment to the carbon atom having R₂ and R₂′ substituents in formula (I).

In yet another embodiment, it provides compounds of formula (I), wherein in ring

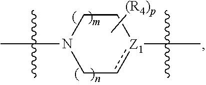

m is 1 and n is 1.

In yet another embodiment, it provides compounds of formula (I), wherein in ring

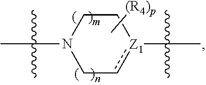

m is 0 and n is 1.

In yet another embodiment, it provides compounds of formula (I), wherein in ring

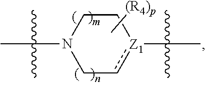

m is 0 and n is 2.

In yet another embodiment, it provides compounds of formula (I), wherein in ring

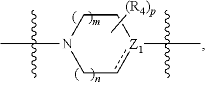

m is 2 and n is 2.

In yet another embodiment, it provides compounds of formula (I), wherein ring

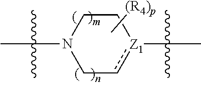

is

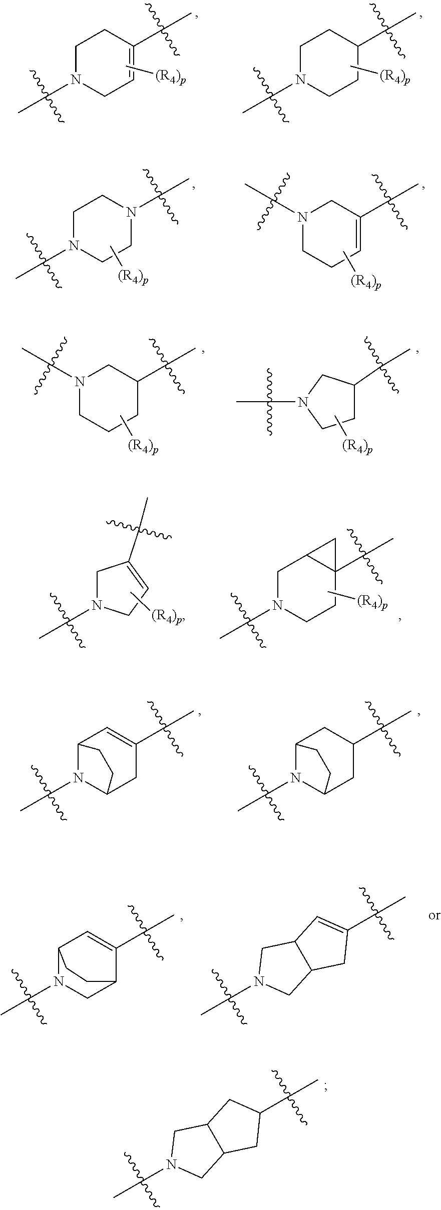

wherein the wavy line next to the nitrogen atom indicates the point of attachment to R₃ and the other wavy line indicates the point of attachment to the rest of molecule in formula (I).

According to the preceding embodiment, two R₄ each on different carbon atoms can form a bridging (C₁-C₃) alkylene or a bridging bond, examples are as shown here:

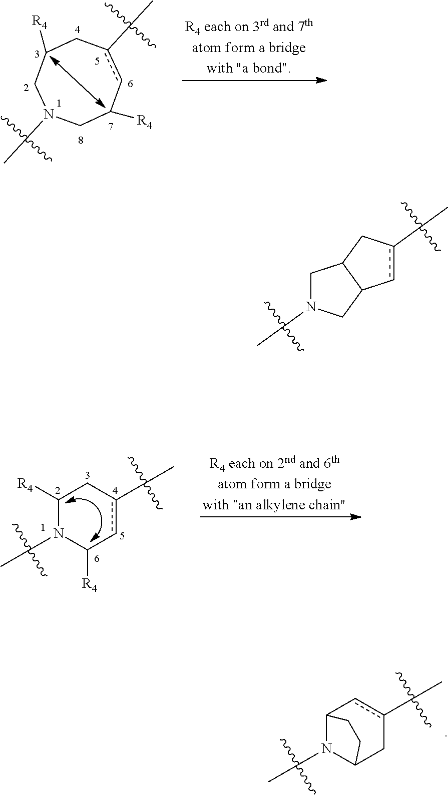

In yet another embodiment, it provides compounds of formula (I), wherein, ring

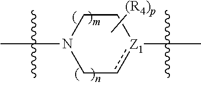

is

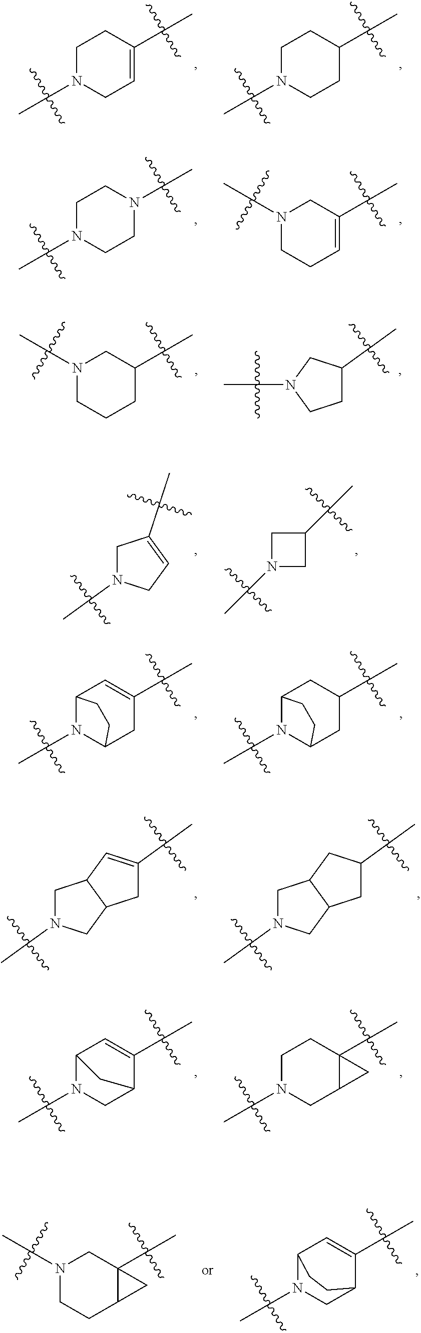

wherein the wavy line next to the nitrogen atom indicates the point of attachment to R₃ and the other wavy line indicates the point of attachment to the rest of molecule in formula (I).

In yet another embodiment, it provides compounds of formula (I), wherein, ring

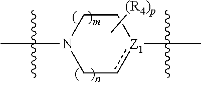

is

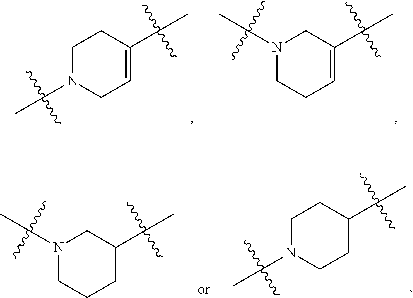 [SMILES: *C1=CCN(*)CC1 ; *C1=CCCN(*)C1 ; *C1CCCN(*)C1 ; *C1CCN(*)CC1]

wherein the wavy line next to the nitrogen atom indicates the point of attachment to R₃ and the other wavy line indicates the point of attachment to rest of the molecule in formula (I).

In yet another embodiment, specifically provided are compounds of formula (I), wherein, R₁ is halogen; the said halogen is chlorine.

In yet another embodiment, specifically provided are compounds of formula (I), wherein, R₁ is hydrogen, Cl, methyl, cyclopropyl or alkylthio such as —SMe.

In yet another embodiment, specifically provided are compounds of formula (I), wherein, R₁ is chloro, fluoro, methyl, cyclopropyl or —SMe.

In yet another embodiment, specifically provided are compounds of formula (I), wherein, each of R₂ and R₂′ is independently hydrogen or alkyl.

In yet another embodiment, specifically provided are compounds of formula (I), wherein, each of R₂ and R₂′ is hydrogen.

In yet another embodiment, specifically provided are compounds of formula (I), wherein, R₂ is alkyl and R₂′ is hydrogen.

In yet another embodiment, specifically provided are compounds of formula (I), wherein, R₂ is methyl and R₂′ is hydrogen.

In yet another embodiment, specifically provided are compounds of formula (I), wherein, R₂ is hydrogen, methyl, ethyl or isopropyl and R₂′ is hydrogen.

In yet another embodiment, specifically provided are compounds of formula (I), wherein, R₃ is —CN.

In yet another embodiment, specifically provided are compounds of formula (I), wherein, R₃ is

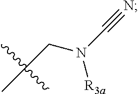

and R₃ₐ is hydrogen, methyl or isopropyl.

According to one embodiment, specifically provided are compounds of formula (I), wherein R₄ at each occurrence independently is halogen, alkyl, haloalkyl, hydroxyalkyl, —OR₄ₐ, —NR_{4b}R_{4c}, unsubstituted or substituted cycloalkyl, unsubstituted or substituted heterocycloalkyl; wherein the substituent is selected from one or more alkyl, halo, alkoxy, haloalkyl and hydroxy.

According to one embodiment, specifically provided are compounds of formula (I), wherein two R₄ each on different carbon atoms can form a bridging (C₁-C₃) alkylene.

According to one embodiment, specifically provided are compounds of formula (I), wherein two R₄ each on different carbon atoms are linked to form a bridging bond.

According to one embodiment, specifically provided are compounds of formula (I), wherein R₅ is hydrogen.

According to one embodiment, specifically provided are compounds of formula (I), wherein R₅ is halogen.

According to one embodiment, specifically provided are compounds of formula (I), wherein R₅ is hydrogen or halogen.

According to yet another embodiment, specifically provided are compounds of formula (I), wherein R₅ is hydrogen, halogen, hydroxyalkyl, alkyl, —PO(CH₃)₂, —OR₅ₐ or —NR_{5b}R_{5c}; wherein R₅ₐ is alkyl, unsubstituted or alkyl substituted heterocycloalkylalkyl, unsubstituted or alkyl substituted heterocycloalkyl, —CONR_{5d}R₅ₑ, -alkyl-CONR_{5d}R₅ₑ, cycloalkyl, alkoxyalkyl or alkylaminoalkyl; R_{5b} and R_{5c} are each independently hydrogen or alkyl; R_{5d} and R₅ₑ are each independently hydrogen or alkyl.

In yet another embodiment, alkylaminoalkyl is dialkylaminoalkyl.

In yet another embodiment, specifically provided are compounds of formula (I), wherein R₅ is unsubstituted or substituted heterocycloalkyl, wherein, the substituents are 1 or 2 substituents independently selected from alkyl and hydroxy.

According to one embodiment, specifically provided are compounds of formula (I), wherein the said R₅ is hydrogen or —OR₅ₐ.

In yet another embodiment, specifically provided are compounds of formula (I), wherein R₅ is:
i) hydrogen, hydroxyalkyl, alkyl or —OR₅a; or
ii) substituted or unsubstituted heterocycloalkyl, wherein, the substituents on heterocycloalkyl are 1 or 2 substituents independently selected from alkyl and hydroxy.

In yet another embodiment, specifically provided are compounds of formula (I), wherein R₅ is hydrogen or —OR₅a, wherein R₅ₐ is alkyl, unsubstituted or alkyl substituted heterocycloalkylalkyl, unsubstituted or alkyl substituted heterocycloalkyl, —CONR_{5d}R₅ₑ, -alkyl-CONR_{5d}R₅ₑ, cycloalkyl, alkoxyalkyl or alkylaminoalkyl, wherein R_{5d} and R₅ₑ are each independently hydrogen or alkyl.

In yet another embodiment, specifically provided are compounds of formula (I), wherein R₅ is hydrogen, hydroxyalkyl, alkyl or —OR₅ₐ.

In yet another embodiment, specifically provided are compounds of formula (I), wherein R₅ is substituted or unsubstituted heterocycloalkyl, wherein, the substituents on heterocycloalkyl are 1 or 2 substituents independently selected from alkyl and hydroxy.

In yet another embodiment, specifically provided are compounds of formula (I), wherein when R₅ is heterocycloalkyl fused with an aryl ring system, then R₅ is attached to the ring carbon atom (at X₁) via the heterocycloalkyl portion of the fused ring system. For example, if R₅ is chromane, then dihydropyranyl ring of chromane is attached to the ring carbon atom (at X₁) and not through the fused phenyl ring of chromane.

According to yet another embodiment, specifically provided are compounds of formula (I), wherein R₅ₐ is

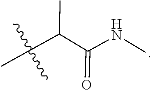 [SMILES: *C(C)C(=O)NC]

According to yet another embodiment, provided are compounds of formula (I), wherein R₅ₐ is heterocycloalkyl wherein the heterocycloalkyl is optionally substituted with alkyl.

In another aspect of an embodiment, provided are compounds of formula (I), wherein R₅ₐ is

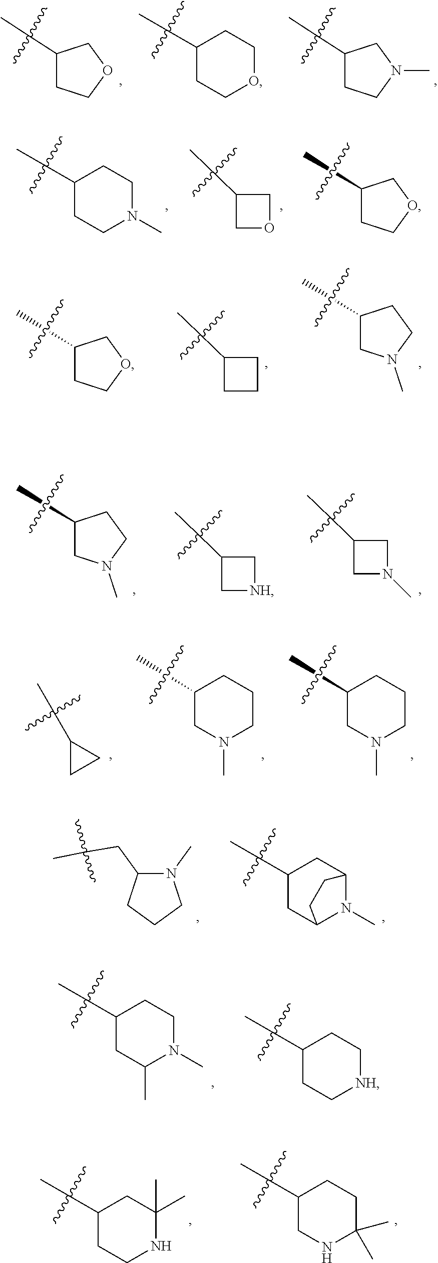

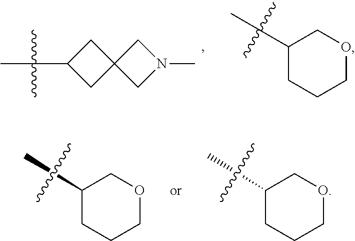

According to another embodiment, provided are compounds of formula (I), wherein R₅ₐ is alkyl, alkoxyalkyl, hydroxyalkyl or cycloalkyl.

In yet another embodiment, specifically provided are compounds of formula (I),

wherein

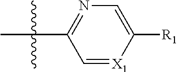

is

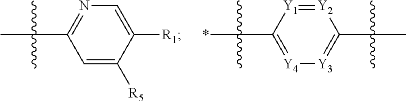

is

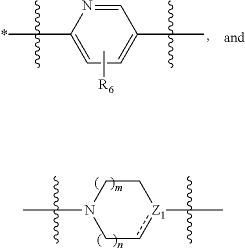

is

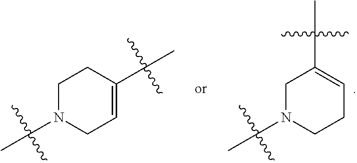 [SMILES: *C1=CCN(*)CC1 ; *C1=CCCN(*)C1]

In yet another embodiment, specifically provided are compounds of formula (I),

wherein

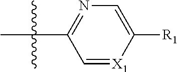

is

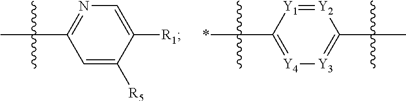

is

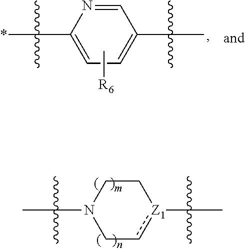

is

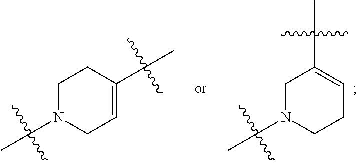 [SMILES: *C1=CCN(*)CC1 ; *C1=CCCN(*)C1]

R₁ is halogen; and
R₃ is —CN.

In yet another embodiment, specifically provided are compounds of formula (I),

wherein

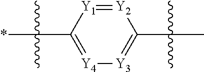

is

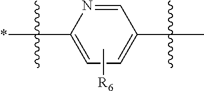

In yet another embodiment, specifically provided are compounds of formula (I),

wherein

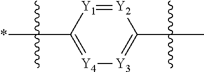

is

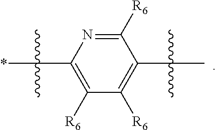

In yet another embodiment, specifically provided are compounds of formula (I),

wherein

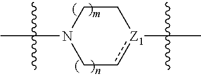

is

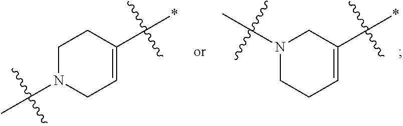

wherein * is the point of attachment with linker L or with ring containing Y₁, Y₂, Y₃ and Y₄.

In yet another embodiment, specifically provided are compounds of formula (I), wherein when Z₁ is C, ‘------’ is a bond.

In yet another embodiment, specifically provided are compounds of formula (I), wherein when Z₁ is C, one of R₄ is on Z₁, R₄ is hydrogen, alkyl or hydroxyl, and ‘-------’ is absent.

In yet another embodiment, specifically provided are compounds of formula (I), wherein when Z₁ is C, and one of R₄ is on Z₁, this R₄ can form a bridge with another R₄ on a different carbon atom of the same ring.

According to the preceding embodiment, the bridge is a (C₁-C₃) alkylene or a bridging bond.

In yet another embodiment, specifically provided are compounds of formula (I), wherein,

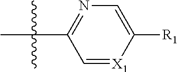

is

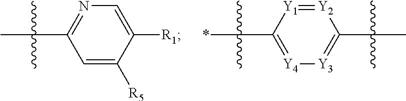

is

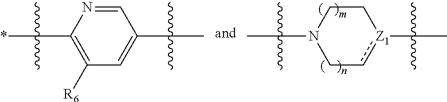

is

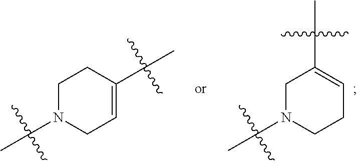 [SMILES: *C1=CCN(*)CC1 ; *C1=CCCN(*)C1]

each of R₂ and R₂′ is independently hydrogen or alkyl;
R₃ is —C≡N; and
R₄ at each occurrence, is independently hydrogen, alkyl, alkoxy, halogen, hydroxy or haloalkyl.

In yet another aspect of the embodiment, provided herein is a compound of formula (I), wherein,

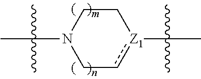

is

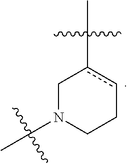

In yet another aspect of the embodiment, provided herein is a compound of formula (I), wherein,

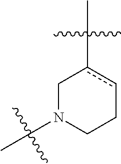

is

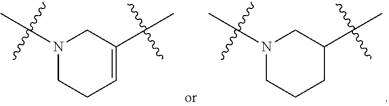 [SMILES: *C1=CCCN(*)C1 ; *C1CCCN(*)C1]

In yet another aspect of the embodiment, provided herein are compounds of formula (I) wherein, ring

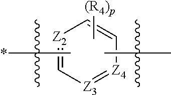

is

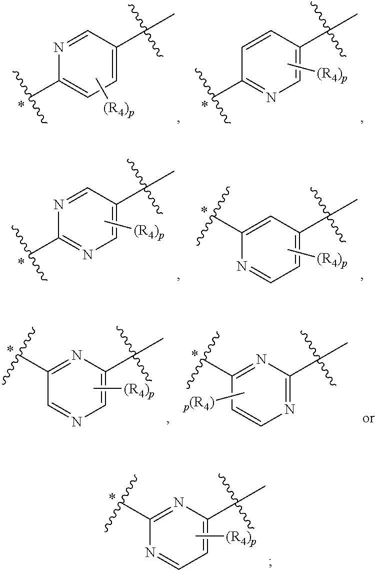

wherein the wavy line with asterisk symbol indicates the point of attachment to R₃ and the wavy line absent of asterisk symbol indicates the point of attachment to the rest of molecule in formula (I).

In yet another aspect of the embodiment, provided herein is a compound of formula (I),

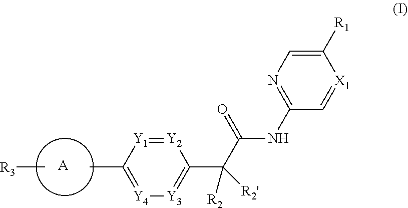

or a pharmaceutically acceptable salt, an N-oxide or a stereoisomer thereof, for use in the treatment of cancer,
   wherein,
X₁ is CR₅ or N;
each of Y₁, Y₂, Y₃ and Y₄ is independently CR₆ or N, wherein 0-2 of Y₁, Y₂, Y₃
and Y₄ are N;
ring A is

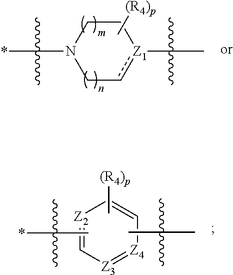

wherein * is the point of attachment with R₃;
‘-----’ is an optional bond;
Z₁ is C or N;
Z₂, Z₃ and Z₄ are each independently C or N;
R₁ is hydrogen, halogen, alkyl, cycloalkyl or alkylthio;
each of R₂ and R₂′ independently is hydrogen or alkyl;
R₃ is —CN or

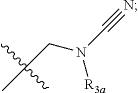

wherein wavy bond indicates the point of attachment with ring A;
R₃ₐ is hydrogen or alkyl;
R₄ at each occurrence independently is halogen, alkyl, haloalkyl, hydroxyalkyl, —OR₄ₐ, —NR_{4b}R_{4c}, unsubstituted or substituted cycloalkyl, unsubstituted or substituted heterocycloalkyl; wherein the substituent is selected from one or more alkyl, halo, alkoxy, haloalkyl or hydroxy;
alternatively, two R₄ each on different carbon atoms form a bridging (C₁-C₃) alkylene or a bridging bond;
R₄ₐ is alkyl, haloalkyl, alkoxyalkyl, alkylaminoalkyl, unsubstituted or alkyl substituted heterocycloalkyl;
R_{4b} and R_{4c} are each independently hydrogen, alkyl, alkylaminoalkyl or unsubstituted or alkyl substituted heterocycloalkyl;
R₅ is:
i) hydrogen, halogen, hydroxyalkyl, alkyl, —PO(CH₃)₂, —OR₅ₐ or —NR_{5b}R_{5c}; or
ii) unsubstituted or substituted heterocycloalkyl, wherein, the substituents are 1 or 2 substituents independently selected from alkyl and hydroxy;
R₅ₐ is alkyl, unsubstituted or alkyl substituted heterocycloalkylalkyl, unsubstituted or alkyl substituted heterocycloalkyl, —CONR_{5d}R₅ₑ, -alkyl-CONR_{5d}R₅ₑ, cycloalkyl, alkoxyalkyl or alkylaminoalkyl;
R_{5b} and R_{5c} are each independently hydrogen or alkyl;
R_{5d} and R₅ₑ are each independently hydrogen or alkyl;
R₆ at each occurrence, is independently hydrogen, alkyl, alkoxy or halogen;
‘p’ is selected from 0 to 3; and
‘m’ and ‘n’ are each independently selected from 0 to 2.

In certain embodiments, the present application provides a compound that is selected from:

| Compound | |
|---|---|
| No. | IUPAC Names |
| 1 | 2-((5-chloro-2-(2-(1′-cyano-1′,2′,3′,6′-tetrahydro-[2,4′-bipyridin]-5-yl)acetamido) |
| | pyridin-4-yl)oxy)-N-methylpropanamide; |
| 2 | 2-((5-chloro-2-(2-(1′-cyano-1′,2′,3′,6′-tetrahydro-[2,4′-bipyridin]-5-yl)acetamido) |
| | pyridin-4-yl)oxy)-N-methylpropanamide (Isomer-1 of Compound 1); |
| 3 | 2-((5-chloro-2-(2-(1′-cyano-1′,2′,3′,6′-tetrahydro-[2,4′-bipyridin]-5-yl)acetamido) |
| | pyridin-4-yl)oxy)-N-methylpropanamide (Isomer-2 of Compound 1); |
| 4 | N-(5-chloropyridin-2-yl)-2-(1′-cyano-1′,2′,3′,6′-tetrahydro-[2,4′-bipyridin]-5-yl) |
| | acetamide; |
| 5 | N-(5-chloropyridin-2-yl)-2-(1′-cyano-1′,2′,5′,6′-tetrahydro-[2,3′-bipyridin]-5-yl) |
| | acetamide; |
| 6 | N-(5-chloropyridin-2-yl)-2-(6-(1-cyano-2,5-dihydro-1H-pyrrol-3-yl)pyridin-3-yl) |
| | acetamide; |
| 7 | N-(5-chloro-4-morpholinopyridin-2-yl)-2-(1′-cyano-1′,2′,3′,6′-tetrahydro-[2,4′- |
| | bipyridin]-5-yl)acetamide; |
| 8 | N-(5-chloro-4-methoxypyridin-2-yl)-2-(1′-cyano-1′,2′,3′,6′-tetrahydro-[2,4′- |
| | bipyridin]-5-yl)acetamide; |
| 9 | N-(5-chloro-4-(4-methylpiperazin-1-yl)pyridin-2-yl)-2-(1′-cyano-1′,2′,3′,6′- |
| | tetrahydro-[2,4′-bipyridin]-5-yl)acetamide; |
| 10 | N-(5-chloro-4-((1-methylpyrrolidin-3-yl)oxy)pyridin-2-yl)-2-(1′-cyano-1′,2′,3′,6′- |
| | tetrahydro-[2,4′-bipyridin]-5-yl)acetamide; |
| 11 | N-(5-chloro-4-((1-methylpiperidin-4-yl)oxy)pyridin-2-yl)-2-(1′-cyano-1′,2′,3′,6′- |
| | tetrahydro-[2,4′-bipyridin]-5-yl)acetamide; |
| 12 | N-(5-chloro-4-((tetrahydrofuran-3-yl)oxy)pyridin-2-yl)-2-(1′-cyano-1′,2′,3′,6′- |
| | tetrahydro-[2,4′-bipyridin]-5-yl)acetamide; |
| 13 | N-(5-chloropyridin-2-yl)-2-(1′-cyano-3-methyl-1′,2′,3′,6′-tetrahydro-[2,4′-bipyridin]- |
| | 5-yl)acetamide; |
| 14 | N-(5-chloro-4-((1-methoxypropan-2-yl)oxy)pyridin-2-yl)-2-(1′-cyano-1′,2′,3′,6′- |
| | tetrahydro-[2,4′-bipyridin]-5-yl)acetamide; |
| 15 | N-(5-chloro-4-(oxetan-3-yloxy)pyridin-2-yl)-2-(1′-cyano-1′,2′,3′,6′-tetrahydro-[2,4′- |
| | bipyridin]-5-yl)acetamide; |
| 16 | N-(5-chloropyridin-2-yl)-2-(6-(1-cyanopyrrolidin-3-yl)pyridin-3-yl)acetamide; |
| 17 | N-(5-chloro-4-(3-hydroxyoxetan-3-yl)pyridin-2-yl)-2-(1′-cyano-1′,2′,3′,6′-tetrahydro- |
| | [2,4′-bipyridin]-5-yl)acetamide; |
| 18 | N-(5-chloropyridin-2-yl)-2-(1′-cyano-3-fluoro-1′,2′,3′,6′-tetrahydro-[2,4′-bipyridin]-5- |
| | yl)acetamide; |
| 19 | N-(5-chloro-4-((tetrahydro-2H-pyran-4-yl)oxy)pyridin-2-yl)-2-(1′-cyano-1′,2′,3′,6′- |
| | tetrahydro-[2,4′-bipyridin]-5-yl)acetamide; |
| 20 | N-(5-chloro-4-ethoxypyridin-2-yl)-2-(1′-cyano-1′,2′,3′,6′-tetrahydro-[2,4′-bipyridin]- |
| | 5-yl)acetamide; |
| 21 | N-(5-chloro-4-isopropoxypyridin-2-yl)-2-(1′-cyano-1′,2′,3′,6′-tetrahydro-[2,4′- |
| | bipyridin]-5-yl)acetamide; |
| 22 | N-(5-chloro-4-(2-methoxyethoxy)pyridin-2-yl)-2-(1′-cyano-1′,2′,3′,6′-tetrahydro- |
| | [2,4′-bipyridin]-5-yl)acetamide; |
| 23 | N-(5-chloro-4-(2-hydroxypropan-2-yl)pyridin-2-yl)-2-(1′-cyano-1′,2′,3′,6′-tetrahydro- |
| | [2,4′-bipyridin]-5-yl)acetamide; |
| 24 | (R)-N-(5-chloro-4-(1-hydroxy-2-methoxyethoxy)pyridin-2-yl)-2-(1′-cyano-1′,2′,3′,6′- |
| | tetrahydro-[2,4′-bipyridin]-5-yl)acetamide; |
| 25 | (S)-N-(5-chloro-4-(1-hydroxy-2-methoxyethoxy)pyridin-2-yl)-2-(1′-cyano-1′,2′,3′,6′- |
| | tetrahydro-[2,4′-bipyridin]-5-yl)acetamide; |
| 26 | (R)-N-(5-chloro-4-((tetrahydrofuran-3-yl)oxy)pyridin-2-yl)-2-(1′-cyano-1′,2′,3′,6′- |
| | tetrahydro-[2,4′-bipyridin]-5-yl)acetamide; |
| 27 | (S)-N-(5-chloro-4-((tetrahydrofuran-3-yl)oxy)pyridin-2-yl)-2-(1′-cyano-1′,2′,3′,6′- |
| | tetrahydro-[2,4′-bipyridin]-5-yl)acetamide; |
| 28 | N-(5-chloro-4-cyclobutoxypyridin-2-yl)-2-(1′-cyano-1′,2′,3′,6′-tetrahydro-[2,4′- |
| | bipyridin]-5-yl)acetamide; |
| 29 | (R)-N-(5-chloro-4-((1-methylpyrrolidin-3-yl)oxy)pyridin-2-yl)-2-(1′-cyano-1′,2′,3′,6′- |
| | tetrahydro-[2,4′-bipyridin]-5-yl)acetamide; |
| 30 | (S)-N-(5-chloro-4-((1-methylpyrrolidin-3-yl)oxy)pyridin-2-yl)-2-(1′-cyano-1′,2′,3′,6′- |
| | tetrahydro-[2,4′-bipyridin]-5-yl)acetamide; |
| 31 | N-(5-chloro-4-((1-(dimethylamino)propan-2-yl)oxy)pyridin-2-yl)-2-(1′-cyano- |
| | 1′,2′,3′,6′-tetrahydro-[2,4′-bipyridin]-5-yl)acetamide; |
| 32 | (R)-N-(5-chloro-4-((1-methylpiperidin-3-yl)oxy)pyridin-2-yl)-2-(1′-cyano-1′,2′,3′,6′- |
| | tetrahydro-[2,4′-bipyridin]-5-yl)acetamide; |
| 33 | (S)-N-(5-chloro-4-((1-methylpiperidin-3-yl)oxy)pyridin-2-yl)-2-(1′-cyano-1′,2′,3′,6′- |
| | tetrahydro-[2,4′-bipyridin]-5-yl)acetamide; |
| 34 | N-(5-chloro-4-methoxypyridin-2-yl)-2-(1′-cyano-1′,2′,5′,6′-tetrahydro-[2,3′- |
| | bipyridin]-5-yl)acetamide; |
| 35 | (S)-N-(5-chloro-4-((1-methoxypropan-2-yl)oxy)pyridin-2-yl)-2-(1′-cyano-1′,2′,5′,6′- |
| | tetrahydro-[2,3′-bipyridin]-5-yl)acetamide; |
| 36 | (R)-N-(5-chloro-4-((1-methoxypropan-2-yl)oxy)pyridin-2-yl)-2-(1′-cyano-1′,2′,5′,6′- |
| | tetrahydro-[2,3′-bipyridin]-5-yl)acetamide; |
| 37 | N-(5-chloro-4-((1-methylpiperidin-4-yl)oxy)pyridin-2-yl)-2-(1′-cyano-1′,2′,5′,6′- |
| | tetrahydro-[2,3′-bipyridin]-5-yl)acetamide; |
| 38 | (R)-N-(5-chloro-4-((1-methylpyrrolidin-3-yl)oxy)pyridin-2-yl)-2-(1′-cyano-1′,2′,5′,6′- |
| | tetrahydro-[2,3′-bipyridin]-5-yl)acetamide; |
| 39 | (S)-N-(5-chloro-4-((1-methylpyrrolidin-3-yl)oxy)pyridin-2-yl)-2-(1′-cyano-1′,2′,5′,6′- |
| | tetrahydro-[2,3′-bipyridin]-5-yl)acetamide; |
| 40 | (R)-N-(5-chloro-4-((tetrahydrofuran-3-yl)oxy)pyridin-2-yl)-2-(1′-cyano-1′,2′,5′,6′- |
| | tetrahydro-[2,3′-bipyridin]-5-yl)acetamide; |
| 41 | (S)-N-(5-chloro-4-((tetrahydrofuran-3-yl)oxy)pyridin-2-yl)-2-(1′-cyano-1′,2′,5′,6′- |
| | tetrahydro-[2,3′-bipyridin]-5-yl)acetamide; |
| 42 | N-(5-chloro-4-((1-(dimethylamino)propan-2-yl)oxy)pyridin-2-yl)-2-(1′-cyano- |
| | 1′,2′,5′,6′-tetrahydro-[2,3′-bipyridin]-5-yl)acetamide; |
| 43 | N-(5-chloro-4-((tetrahydro-2H-pyran-3-yl)oxy)pyridin-2-yl)-2-(1′-cyano-1′,2′,5′,6′- |
| | tetrahydro-[2,3′-bipyridin]-5-yl)acetamide; |
| 44 | N-(5-chloro-4-((tetrahydro-2H-pyran-3-yl)oxy)pyridin-2-yl)-2-(1′-cyano-1′,2′,5′,6′- |
| | tetrahydro-[2,3′-bipyridin]-5-yl)acetamide (Isomer-1 of compound-43); |
| 45 | N-(5-chloro-4-((tetrahydro-2H-pyran-3-yl)oxy)pyridin-2-yl)-2-(1′-cyano-1′,2′,5′,6′- |
| | tetrahydro-[2,3′-bipyridin]-5-yl)acetamide (Isomer-2 of compound-43); |
| 46 | (R)-N-(5-chloro-4-((1-methylpiperidin-3-yl)oxy)pyridin-2-yl)-2-(1′-cyano-1′,2′,5′,6′- |
| | tetrahydro-[2,3′-bipyridin]-5-yl)acetamide; |
| 47 | (S)-N-(5-chloro-4-((1-methylpiperidin-3-yl)oxy)pyridin-2-yl)-2-(1′-cyano-1′,2′,5′,6′- |
| | tetrahydro-[2,3′-bipyridin]-5-yl)acetamide; |
| 48 | N-(5-chloro-4-methoxypyridin-2-yl)-2-(1′-cyano-1′,2′,5′,6′-tetrahydro-[2,3′- |
| | bipyridin]-5-yl)propanamide; |
| 49 | N-(5-chloro-4-methoxypyridin-2-yl)-2-(1′-cyano-1′,2′,5′,6′-tetrahydro-[2,3′- |
| | bipyridin]-5-yl)propenamide (Isomer-1 of compound-48); |
| 50 | N-(5-chloro-4-methoxypyridin-2-yl)-2-(1′-cyano-1′,2′,5′,6′-tetrahydro-[2,3′- |
| | bipyridin]-5-yl)propenamide (Isomer-2 of compound-48); |
| 51 | N-(5-chloro-4-methoxypyridin-2-yl)-2-(1′-cyano-1′,2′,3′,6′-tetrahydro-[2,4′- |
| | bipyridin]-5-yl)propanamide; |
| 52 | N-(5-chloro-4-methoxypyridin-2-yl)-2-(1′-cyano-1′,2′,3′,6′-tetrahydro-[2,4′- |
| | bipyridin]-5-yl)propenamide (Isomer-1 of compound-52); |
| 53 | N-(5-chloro-4-methoxypyridin-2-yl)-2-(1′-cyano-1′,2′,3′,6′-tetrahydro-[2,4′- |
| | bipyridin]-5-yl)propenamide (Isomer-2 of compound-52); |
| 54 | N-(5-chloro-4-((1-methylpyrrolidin-2-yl)methoxy)pyridin-2-yl)-2-(1′-cyano-1′,2′,3′,6′- |
| | tetrahydro-[2,4′-bipyridin]-5-yl)acetamide; |
| 55 | N-(5-chloro-4-((1-methylpyrrolidin-2-yl)methoxy)pyridin-2-yl)-2-(1′-cyano-1′,2′,5′,6′- |
| | tetrahydro-[2,3′-bipyridin]-5-yl)acetamide; |
| 56 | 2-((5-chloro-2-(2-(1′-cyano-1′,2′,5′,6′-tetrahydro-[2,3′-bipyridin]-5-yl)acetamido) |
| | pyridin-4-yl)oxy)-N-methylpropanamide; |
| 57 | 2-((5-chloro-2-(2-(1′-cyano-1′,2′,5′,6′-tetrahydro-[2,3′-bipyridin]-5-yl)acetamido) |
| | pyridin-4-yl)oxy)-N-methylpropanamide (Isomer-1 of compound-56); |
| 58 | 2-((5-chloro-2-(2-(1′-cyano-1′,2′,5′,6′-tetrahydro-[2,3′-bipyridin]-5-yl)acetamido) |
| | pyridin-4-yl)oxy)-N-methylpropanamide (Isomer-2 of compound-56); |
| 59 | N-(5-chloro-4-methoxypyridin-2-yl)-2-(6-(1-cyanopiperidin-4-yl)pyridin-3-yl) |
| | acetamide; |
| 60 | N-(5-chloro-4-methoxypyridin-2-yl)-2-(6-(1-cyano-3-methylpiperidin-4-yl)pyridin- |
| | 3-yl)acetamide; |
| 61 | N-(5-chloro-4-((tetrahydro-2H-pyran-4-yl)oxy)pyridin-2-yl)-2-(1′-cyano-1′,2′,5′,6′- |
| | tetrahydro-[2,3′-bipyridin]-5-yl)acetamide; |
| 62 | N-(5-chloro-4-((8-methyl-8-azabicyclo[3.2.1]octan-3-yl)oxy)pyridin-2-yl)-2-(1′- |
| | cyano-1′,2′,5′,6′-tetrahydro-[2,3′-bipyridin]-5-yl)acetamide; |
| 63 | N-(5-chloro-4-((1,2-dimethylpiperidin-4-yl)oxy)pyridin-2-yl)-2-(1′-cyano-1′,2′,5′,6′- |
| | tetrahydro-[2,3′-bipyridin]-5-yl)acetamide; |
| 64 | N-(5-chloro-4-((1,2-dimethylpiperidin-4-yl)oxy)pyridin-2-yl)-2-(1′-cyano-1′,2′,5′,6′- |
| | tetrahydro-[2,3′-bipyridin]-5-yl)acetamide (Isomer-1 of compound-63); |
| 64A | N-(5-chloro-4-((1,2-dimethylpiperidin-4-yl)oxy)pyridin-2-yl)-2-(1′-cyano-1′,2′,5′,6′- |
| | tetrahydro-[2,3′-bipyridin]-5-yl)acetamide (Isomer-2 of compound-63); |
| 65 | N-(5-chloro-4-methoxypyridin-2-yl)-2-(1′-cyano-2′-methyl-1′,2′,3′,6′-tetrahydro-[2,4′- |
| | bipyridin]-5-yl)acetamide; |
| 66 | N-(5-chloro-4-methoxypyridin-2-yl)-2-(1′-cyano-2′-methyl-1′,2′,3′,6′-tetrahydro-[2,4′- |
| | bipyridin]-5-yl)acetamide (Isomer-1 of compound-65); |
| 67 | N-(5-chloro-4-methoxypyridin-2-yl)-2-(1′-cyano-2′-methyl-1′,2′,3′,6′-tetrahydro-[2,4′- |
| | bipyridin]-5-yl)acetamide (Isomer-2 of compound-65); |
| 68 | N-(5-chloro-4-methoxypyridin-2-yl)-2-(1′-cyano-2′-methyl-1′,2′,3′,6′-tetrahydro-[2,4′- |
| | bipyridin]-5-yl)acetamide; |
| 69 | N-(5-chloro-4-((2-methyl-2-azaspiro[3.3]heptan-6-yl)oxy)pyridin-2-yl)-2-(1′-cyano- |
| | 1′,2′,5′,6′-tetrahydro-[2,3′-bipyridin]-5-yl)acetamide; |
| 70 | N-(5-chloro-4-methoxypyridin-2-yl)-2-(1′-cyano-3′-methyl-1′,2′,3′,6′-tetrahydro-[2,4′- |
| | bipyridin]-5-yl)acetamide; |
| 71 | N-(5-chloro-4-methoxypyridin-2-yl)-2-(1′-cyano-3′-methyl-1′,2′,3′,6′-tetrahydro-[2,4′- |
| | bipyridin]-5-yl)acetamide (Isomer-1 of compound 70); |
| 72 | N-(5-chloro-4-methoxypyridin-2-yl)-2-(1′-cyano-3′-methyl-1′,2′,3′,6′-tetrahydro-[2,4′- |
| | bipyridin]-5-yl)acetamide (Isomer-2 of compound 70); |
| 73 | N-(5-chloro-4-methoxypyridin-2-yl)-2-(6-(8-cyano-8-azabicyclo[3.2.1]oct-2-en-3-yl) |
| | pyridin-3-yl)acetamide; |
| 74 | N-(5-chloro-4-methoxypyridin-2-yl)-2-(6-(8-cyano-8-azabicyclo[3.2.1]octan-3-yl) |
| | pyridin-3-yl)acetamide; |
| 75 | N-(5-chloro-4-methoxypyridin-2-yl)-2-(6-(2-cyano-1,2,3,3a,4,6a-hexahydrocyclopenta[c]pyrrol- |
| | 5-yl)pyridin-3-yl)acetamide; |
| 76 | N-(5-chloro-4-methoxypyridin-2-yl)-2-(6-(2-cyanooctahydrocyclopenta[c]pyrrol-5- |
| | yl)pyridin-3-yl)acetamide; |
| 77 | 5-chloro-2-(2-(1′-cyano-1′,2′,5′,6′-tetrahydro-[2,3′-bipyridin]-5-yl)acetamido)pyridin- |
| | 4-yl dimethylcarbamate; |
| 78 | N-(5-chloro-4-methoxypyridin-2-yl)-2-(6-(1-cyanopiperidin-3-yl)pyridin-3-yl) |
| | acetamide; |
| 79 | N-(5-chloro-4-methoxypyridin-2-yl)-2-(6-(1-cyanopiperidin-3-yl)pyridin-3-yl) |
| | acetamide (Isomer-1 of compound-78); |
| 80 | N-(5-chloro-4-methoxypyridin-2-yl)-2-(6-(1-cyanopiperidin-3-yl)pyridin-3-yl) |
| | acetamide (Isomer-2 of compound-78); |
| 81 | N-(5-chloro-4-methoxypyridin-2-yl)-2-(6-(2-cyano-2-azabicyclo[2.2.2]oct-5-en-5-yl) |
| | pyridin-3-yl)acetamide; |
| 82 | 2-((5-chloro-2-(2-(6-(1-cyanopiperidin-4-yl)pyridin-3-yl)acetamido)pyridin-4-yl) |
| | oxy)-N-methylpropanamide; |
| 83 | 2-((5-chloro-2-(2-(6-(1-cyanopiperidin-4-yl)pyridin-3-yl)acetamido)pyridin-4-yl) |
| | oxy)-N-methylpropanamide (Isomer-1 of compound-82); |
| 84 | 2-((5-chloro-2-(2-(6-(1-cyanopiperidin-4-yl)pyridin-3-yl)acetamido)pyridin-4-yl) |
| | oxy)-N-methylpropanamide (Isomer-2 of compound-82); |
| 85 | 2-((5-chloro-2-(2-(6-(1-cyanopiperidin-3-yl)pyridin-3-yl)acetamido)pyridin-4-yl) |
| | oxy)-N-methylpropanamide; |
| 86 | 2-((5-chloro-2-(2-(6-(1-cyanopiperidin-3-yl)pyridin-3-yl)acetamido)pyridin-4-yl) |
| | oxy)-N-methylpropanamide; |
| 87 | 2-((5-chloro-2-(2-(6-(1-cyanopiperidin-3-yl)pyridin-3-yl)acetamido)pyridin-4-yl) |
| | oxy)-N-methylpropanamide; |
| 88 | 2-((5-chloro-2-(2-(6-(1-cyanopiperidin-3-yl)pyridin-3-yl)acetamido)pyridin-4-yl) |
| | oxy)-N-methylpropanamide; |
| 89 | 2-((5-chloro-2-(2-(6-(1-cyanopiperidin-3-yl)pyridin-3-yl)acetamido)pyridin-4-yl) |
| | oxy)-N-methylpropanamide; |
| 90 | 2-((5-chloro-2-(2-(6-(1-cyanopiperidin-3-yl)pyridin-3-yl)acetamido)pyridin-4-yl) |
| | oxy)-N-methylpropanamide; |
| 91 | 2-((5-chloro-2-(2-(6-(1-cyanopiperidin-3-yl)pyridin-3-yl)acetamido)pyridin-4-yl) |
| | oxy)-N-methylpropanamide; |
| 92 | N-(5-chloro-4-(((R)-tetrahydrofuran-3-yl)oxy)pyridin-2-yl)-2-(6-(1-cyanopiperidin- |
| | 3-yl)pyridin-3-yl)acetamide; |
| 93 | N-(5-chloro-4-(((R)-tetrahydrofuran-3-yl)oxy)pyridin-2-yl)-2-(6-(1-cyanopiperidin- |
| | 3-yl)pyridin-3-yl)acetamide (Isomer-1 of Compound-92); |
| 94 | N-(5-chloro-4-(((R)-tetrahydrofuran-3-yl)oxy)pyridin-2-yl)-2-(6-(1-cyanopiperidin- |
| | 3-yl)pyridin-3-yl)acetamide (Isomer-2 of Compound-92); |
| 95 | N-(5-chloro-4-(((S)-tetrahydrofuran-3-yl)oxy)pyridin-2-yl)-2-(6-(1-cyanopiperidin- |
| | 3-yl)pyridin-3-yl)acetamide; |
| 96 | N-(5-chloro-4-(((S)-tetrahydrofuran-3-yl)oxy)pyridin-2-yl)-2-(6-(1-cyanopiperidin- |
| | 3-yl)pyridin-3-yl)acetamide (Isomer-1 of Compound-95); |
| 97 | N-(5-chloro-4-(((S)-tetrahydrofuran-3-yl)oxy)pyridin-2-yl)-2-(6-(1-cyanopiperidin- |
| | 3-yl)pyridin-3-yl)acetamide (Isomer-2 of Compound-95); |
| 98 | (R)-N-(5-chloro-4-((tetrahydrofuran-3-yl)oxy)pyridin-2-yl)-2-(6-(1-cyanopiperidin- |
| | 4-yl)pyridin-3-yl)acetamide; |
| 99 | (S)-N-(5-chloro-4-((tetrahydrofuran-3-yl)oxy)pyridin-2-yl)-2-(6-(1-cyanopiperidin- |
| | 4-yl)pyridin-3-yl)acetamide; |
| 100 | 5-chloro-2-(2-(1′-cyano-1′,2′,3′,6′-tetrahydro-[2,4′-bipyridin]-5-yl)acetamido)pyridin- |
| | 4-yl dimethylcarbamate; |
| 101 | 5-chloro-2-(2-(6-(1-cyanopiperidin-4-yl)pyridin-3-yl)acetamido)pyridin-4-yl |
| | dimethylcarbamate; |
| 102 | 2-(1′-cyano-1′,2′,5′,6′-tetrahydro-[2,3′-bipyridin]-5-yl)-N-(pyridin-2-yl)acetamide; |
| 103 | 2-(1′-cyano-1′,2′,5′,6′-tetrahydro-[2,3′-bipyridin]-5-yl)-N-(5-fluoropyridin-2-yl) |
| | acetamide; |
| 104 | 2-(1′-cyano-1′,2′,5′,6′-tetrahydro-[2,3′-bipyridin]-5-yl)-N-(5-methylpyridin-2-yl) |
| | acetamide; |
| 105 | 2-(1′-cyano-1′,2′,5′,6′-tetrahydro-[2,3′-bipyridin]-5-yl)-N-(4,5-dichloropyridin-2-yl) |
| | acetamide; |
| 106 | 2-(1′-cyano-1′,2′,5′,6′-tetrahydro-[2,3′-bipyridin]-5-yl)-N-(5-cyclopropylpyridin-2-yl) |
| | acetamide; |
| 107 | 2-(1′-cyano-1′,2′,5′,6′-tetrahydro-[2,3′-bipyridin]-5-yl)-N-(5-(methylthio)pyridin-2-yl) |
| | acetamide; |
| 108 | N-(5-chloropyrazin-2-yl)-2-(1′-cyano-1′,2′,5′,6′-tetrahydro-[2,3′-bipyridin]-5-yl) |
| | acetamide; |
| 109 | N-(5-chloro-4-(dimethylamino)pyridin-2-yl)-2-(1′-cyano-1′,2′,5′,6′-tetrahydro-[2,3′- |
| | bipyridin]-5-yl)acetamide; |
| 110 | N-(5-chloro-4-methoxypyridin-2-yl)-2-(1′-cyano-1′,2′,5′,6′-tetrahydro-[2,3′-bipyridin]- |
| | 5-yl)-2-methylpropanamide; |
| 111 | N-(5-chloro-4-methoxypyridin-2-yl)-2-(1′-cyano-1′,2′,5′,6′-tetrahydro-[3,3′-bipyridin]- |
| | 6-yl)acetamide; |
| 112 | 2-(2-chloro-4-(1-cyano-1,2,5,6-tetrahydropyridin-3-yl)phenyl)-N-(5-chloro-4- |
| | methoxypyridin-2-yl)acetamide; |
| 113 | N-(5-chloro-4-methoxypyridin-2-yl)-2-(4-(1-cyano-1,2,5,6-tetrahydropyridin-3-yl)- |
| | 2-fluorophenyl)acetamide; |
| 114 | N-(5-chloro-4-methoxypyridin-2-yl)-2-(4-(1-cyano-1,2,5,6-tetrahydropyridin-3-yl) |
| | phenyl)acetamide; |
| 115 | N-(5-chloro-4-methoxypyridin-2-yl)-2-(4-(2-cyano-1,2,3,3a,4,6a-hexahydrocyclopenta[c]pyrrol- |
| | 5-yl)phenyl)acetamide; |
| 116 | N-(5-chloro-4-methoxypyridin-2-yl)-2-(4-(1-cyano-1,2,5,6-tetrahydropyridin-3-yl)- |
| | 3-methoxyphenyl)acetamide; |
| 117 | N-(5-chloro-4-methoxypyridin-2-yl)-2-(1′-cyano-3-methoxy-1′,2′,5′,6′-tetrahydro- |
| | [2,3′-bipyridin]-5-yl)acetamide; |
| 118 | (S)-N-(5-chloro-4-((1-methylpyrrolidin-3-yl)oxy)pyridin-2-yl)-2-(6-(1-cyano |
| | piperidin-4-yl)pyridin-3-yl)acetamide; |
| 119 | (R)-N-(5-chloro-4-((1-methylpyrrolidin-3-yl)oxy)pyridin-2-yl)-2-(6-(1-cyano |
| | piperidin-4-yl)pyridin-3-yl)acetamide; |
| 120 | N-(5-chloro-4-methoxypyridin-2-yl)-2-(1′-cyano-3-methoxy-1′,2′,3′,6′-tetrahydro- |
| | [2,4′-bipyridin]-5-yl)acetamide; |
| 121 | N-(5-chloro-4-methoxypyridin-2-yl)-2-(6-(1-cyanopiperidin-4-yl)-5-methoxypyridin- |
| | 3-yl)acetamide; |
| 122 | (R)-N-(5-chloro-4-((tetrahydrofuran-3-yl)oxy)pyridin-2-yl)-2-(1′-cyano-3-methoxy- |
| | 1′,2′,3′,6′-tetrahydro-[2,4′-bipyridin]-5-yl)acetamide; |
| 123 | (R)-N-(5-chloro-4-((tetrahydrofuran-3-yl)oxy)pyridin-2-yl)-2-(6-(1-cyanopiperidin- |
| | 4-yl)-5-methoxypyridin-3-yl)acetamide; |
| 124 | 2-(6-(1-cyanopiperidin-4-yl)pyridin-3-yl)-N-(5-cyclopropyl-4-methoxypyridin-2-yl) |
| | acetamide; |
| 125 | (S)-N-(5-chloro-4-((1-methylpiperidin-3-yl)oxy)pyridin-2-yl)-2-(6-(1-cyano |
| | piperidin-4-yl)pyridin-3-yl)acetamide; |
| 126 | (R)-N-(5-chloro-4-((1-methylpiperidin-3-yl)oxy)pyridin-2-yl)-2-(6-(1-cyano |
| | piperidin-4-yl)pyridin-3-yl)acetamide; |
| 127 | N-(5-chloro-4-methoxypyridin-2-yl)-2-(1′-cyano-1′,2′,3′,6′-tetrahydro-[3,4′- |
| | bipyridin]-6-yl)acetamide; |
| 128 | (S)-2-((5-chloro-2-(2-(6-(1-cyanopiperidin-4-yl)-5-methoxypyridin-3-yl)acetamido) |
| | pyridin-4-yl)oxy)-N-methylpropanamide; |
| 129 | N-(5-chloro-4-((1-methylpiperidin-4-yl)oxy)pyridin-2-yl)-2-(6-(1-cyanopiperidin-4- |
| | yl)pyridin-3-yl)acetamide; |
| 130 | N-(5-chloro-4-methoxypyridin-2-yl)-2-(6′-(cyanamidomethyl)-[2,3′-bipyridin]-5-yl) |
| | acetamide; |
| 131 | N-(5-chloro-4-methoxypyridin-2-yl)-2-(6′-((N-methylcyanamido)methyl)-[2,3′- |
| | bipyridin]-5-yl)acetamide; |
| 132 | N-(5-chloro-4-methoxypyridin-2-yl)-2-(6′-((N-isopropylcyanamido)methyl)-[2,3′- |
| | bipyridin]-5-yl)acetamide; |
| 133 | N-(5-chloro-4-methoxypyridin-2-yl)-2-(6-(3-cyano-3-azabicyclo[4.1.0]heptan-6-yl) |
| | pyridin-3-yl)acetamide; |
| 134 | N-(5-chloro-4-methoxypyridin-2-yl)-2-(6-(4-cyanopiperazin-1-yl)pyridin-3-yl) |
| | acetamide; |
| 135 | N-(5-chloro-4-(dimethylphosphoryl)pyridin-2-yl)-2-(1′-cyano-1′,2′,5′,6′-tetrahydro- |
| | [2,3′-bipyridin]-5-yl)acetamide; |
| 136 | N-(5-chloro-4-(piperidin-4-yloxy)pyridin-2-yl)-2-(1′-cyano-1′,2′,3′,6′-tetrahydro- |
| | [2,4′-bipyridin]-5-yl)acetamide; |
| 137 | N-(4-((2-azaspiro[3.3]heptan-6-yl)oxy)-5-chloropyridin-2-yl)-2-(1′-cyano-1′,2′,3′,6′- |
| | tetrahydro-[2,4′-bipyridin]-5-yl)acetamide; |
| 138 | N-(5-chloro-4-((6,6-dimethylpiperidin-3-yl)oxy)pyridin-2-yl)-2-(1′-cyano-1′,2′,3′,6′- |
| | tetrahydro-[2,4′-bipyridin]-5-yl)acetamide; |
| 139 | N-(5-chloro-4-((2,2-dimethylpiperidin-4-yl)oxy)pyridin-2-yl)-2-(1′-cyano-1′,2′,3′,6′- |
| | tetrahydro-[2,4′-bipyridin]-5-yl)acetamide; |
| 140 | N-(5-chloro-4-methoxypyridin-2-yl)-2-(6′-cyano-[2,3′-bipyridin]-5-yl)acetamide; |
| 141 | N-(5-chloro-4-methoxypyridin-2-yl)-2-(6-(2-cyanopyrimidin-5-yl)pyridin-3-yl) |
| | acetamide; |
| 142 | N-(5-chloro-4-methoxypyridin-2-yl)-2-(2′-cyano-[2,4′-bipyridin]-5-yl)acetamide; |
| 143 | 2-((5-chloro-2-(2-(6′-cyano-[2,3′-bipyridin]-5-yl)acetamido)pyridin-4-yl)oxy)-N- |
| | methylpropanamide; |
| 144 | 2-((5-chloro-2-(2-(2′-cyano-[2,4′-bipyridin]-5-yl)acetamido)pyridin-4-yl)oxy)-N- |
| | methylpropanamide; |
| 145 | (R)-N-(5-chloro-4-((tetrahydrofuran-3-yl)oxy)pyridin-2-yl)-2-(2′-cyano-[2,4′- |
| | bipyridin]-5-yl)acetamide; |
| 146 | (R)-N-(5-chloro-4-((tetrahydrofuran-3-yl)oxy)pyridin-2-yl)-2-(6′-cyano-[2,3′- |
| | bipyridin]-5-yl)acetamide; |
| 147 | (S)-2-((5-chloro-2-(2-(6′-cyano-[2,3′-bipyridin]-5-yl)acetamido)pyridin-4-yl)oxy)-N- |
| | methylpropanamide; |
| 148 | (S)-2-((5-chloro-2-(2-(2′-cyano-[2,4′-bipyridin]-5-yl)acetamido)pyridin-4-yl)oxy)-N- |
| | methylpropanamide; |
| 149 | N-(5-chloro-4-methoxypyridin-2-yl)-2-(6-(4-cyano-3-fluorophenyl)pyridin-3-yl) |
| | acetamide; |
| 150 | (R)-N-(5-chloro-4-((tetrahydrofuran-3-yl)oxy)pyridin-2-yl)-2-(6-(2-cyanopyrimidin- |
| | 5-yl)pyridin-3-yl)acetamide; |
| 151 | (S)-2-((5-chloro-2-(2-(6-(2-cyanopyrimidin-5-yl)pyridin-3-yl)acetamido)pyridin-4- |
| | yl)oxy)-N-methylpropanamide; |
| 152 | N-(5-chloro-4-methoxypyridin-2-yl)-2-(4-(6-cyanopyridin-2-yl)phenyl)acetamide; |
| 153 | N-(5-chloro-4-methoxypyridin-2-yl)-2-(4-(6-cyanopyridin-2-yl)-3-fluorophenyl) |
| | acetamide; |
| 154 | (R)-N-(5-chloro-4-((tetrahydrofuran-3-yl)oxy)pyridin-2-yl)-2-(6′-cyano-[2,2′- |
| | bipyridin]-5-yl)acetamide; |
| 155 | 2-((5-chloro-2-(2-(6′-cyano-[2,2′-bipyridin]-5-yl)acetamido)pyridin-4-yl)oxy)-N- |
| | methylpropanamide; |
| 156 | N-(5-chloro-4-methoxypyridin-2-yl)-2-(6′-cyano-[2,2′-bipyridin]-5-yl)acetamide; |
| 157 | N-(5-chloro-4-methoxypyridin-2-yl)-2-(6-(6-cyanopyrazin-2-yl)pyridin-3-yl) |
| | acetamide; |
| 158 | N-(5-chloro-4-methoxypyridin-2-yl)-2-(6′-cyano-5′-methyl-[2,2′-bipyridin]-5-yl) |
| | acetamide; |
| 159 | N-(5-chloro-4-methoxypyridin-2-yl)-2-(6′-cyano-4′-methyl-[2,2′-bipyridin]-5-yl) |
| | acetamide; |
| 160 | N-(5-chloro-4-methoxypyridin-2-yl)-2-(6′-cyano-3′-methyl-[2,2′-bipyridin]-5-yl) |
| | acetamide; |
| 161 | N-(5-chloro-4-methoxypyridin-2-yl)-2-(6-(4-cyanopyrimidin-2-yl)pyridin-3-yl) |
| | acetamide; |
| 162 | N-(5-chloro-4-methoxypyridin-2-yl)-2-(6-(2-cyanopyrimidin-4-yl)pyridin-3-yl) |
| | acetamide; |
| 163 | N-(5-chloro-4-methoxypyridin-2-yl)-2-(6′-cyano-5′-fluoro-[2,2′-bipyridin]-5-yl) |
| | acetamide; |
| 164 | N-(5-chloro-4-methoxypyridin-2-yl)-2-(6′-cyano-3′-fluoro-[2,2′-bipyridin]-5-yl) |
| | acetamide; |
| 165 | N-(5-chloro-4-methoxypyridin-2-yl)-2-(6′-cyano-4′-methoxy-[2,2′-bipyridin]-5-yl) |
| | acetamide; |
| 166 | N-(5-chloro-4-methoxypyridin-2-yl)-2-(6′-cyano-4′-(trifluoromethoxy)-[2,2′- |
| | bipyridin]-5-yl)acetamide; |
| 167 | N-(5-chloro-4-methoxypyridin-2-yl)-2-(6′-cyano-4′-(trifluoromethyl)-[2,2′-bipyridin]- |
| | 5-yl)acetamide; |
| 168 | N-(5-chloro-4-methoxypyridin-2-yl)-2-(6′-cyano-4′-(difluoromethyl)-[2,2′-bipyridin]- |
| | 5-yl)acetamide; |
| 169 | N-(5-chloro-4-methoxypyridin-2-yl)-2-(6′-cyano-4′-(2-methoxyethoxy)-[2,2′- |
| | bipyridin]-5-yl)acetamide; |
| 170 | N-(5-chloro-4-methoxypyridin-2-yl)-2-(6′-cyano-4′-(2-(dimethylamino)ethoxy)-[2,2′- |
| | bipyridin]-5-yl)acetamide; |
| 171 | N-(5-chloro-4-methoxypyridin-2-yl)-2-(6′-cyano-4′-((1-methylpyrrolidin-3-yl)oxy)- |
| | [2,2′-bipyridin]-5-yl)acetamide; |
| 172 | N-(5-chloro-4-methoxypyridin-2-yl)-2-(6′-cyano-4′-(methyl(1-methylpyrrolidin-3- |
| | yl)amino)-[2,2′-bipyridin]-5-yl)acetamide; |
| 173 | N-(5-chloro-4-methoxypyridin-2-yl)-2-(6′-cyano-4′-((2-(dimethylaminoethyl) |
| | (methyl)amino)-[2,2′-bipyridin]-5-yl)acetamide; |
| 174 | N-(5-chloro-4-methoxypyridin-2-yl)-2-(6′-cyano-4′-(1-hydroxyethyl)-[2,2′- |
| | bipyridin]-5-yl)acetamide; |
| 175 | N-(5-chloro-4-methoxypyridin-2-yl)-2-(6′-cyano-4′-(2-hydroxypropan-2-yl)-[2,2′- |
| | bipyridin]-5-yl)acetamide; |
| 176 | N-(5-chloro-4-methoxypyridin-2-yl)-2-(6′-cyano-4′-((1-methylpiperidin-4-yl)oxy)- |
| | [2,2′-bipyridin]-5-yl)acetamide; |
| 177 | N-(5-chloro-4-methoxypyridin-2-yl)-2-(6′-cyano-4′-(1-methylpiperidin-4-yl)-[2,2′- |
| | bipyridin]-5-yl)acetamide; |
| 178 | N-(5-chloro-4-methoxypyridin-2-yl)-2-(6′-cyano-4′-(1-methylpyrrolidin-3-yl)-[2,2′- |
| | bipyridin]-5-yl)acetamide; |
| 179 | N-(5-chloro-4-methoxypyridin-2-yl)-2-(6′-cyano-4′-(1-hydroxycyclopropyl)-[2,2′- |
| | bipyridin]-5-yl)acetamide; |
| 180 | N-(5-chloro-4-methoxypyridin-2-yl)-2-(6′-cyano-4′-(1-methylpiperidin-3-yl)-[2,2′- |
| | bipyridin]-5-yl)acetamide; |
| 181 | N-(5-chloro-4-methoxypyridin-2-yl)-2-(6′-cyano-4′-(5-methyl-2,5-diazabicyclo |
| | [2.2.1]heptan-2-yl)-[2,2′-bipyridin]-5-yl)acetamide; |
| 182. | N-(5-chloro-4-methoxypyridin-2-yl)-2-(6′-cyano-4′-(3-hydroxyazetidin-1-yl)-[2,2′- |
| | bipyridin]-5-yl)acetamide; |
| 183 | N-(5-chloro-4-methoxypyridin-2-yl)-2-(6′-cyano-4′-(8-methyl-3,8-diazabicyclo |
| | [3.2.1]octan-3-yl)-[2,2′-bipyridin]-5-yl)acetamide; |
| 184 | N-(5-chloro-4-methoxypyridin-2-yl)-2-(6′-cyano-4′-(oxetan-3-yloxy)-[2,2′- |
| | bipyridin]-5-yl)acetamide; |
| 185 | N-(5-chloro-4-methoxypyridin-2-yl)-2-(6′-cyano-4′-((1-methylazetidin-3-yl)oxy)- |
| | [2,2′-bipyridin]-5-yl)acetamide; |
| 186 | N-(5-chloro-4-methoxypyridin-2-yl)-2-(6′-cyano-4′-((tetrahydrofuran-3-yl)oxy)-[2,2′- |
| | bipyridin]-5-yl)acetamide; |
| 187 | N-(5-chloro-4-methoxypyridin-2-yl)-2-(6′-cyano-4′-((2-methyl-2-azaspiro[3.3] |
| | heptan-6-yl)oxy)-[2,2′-bipyridin]-5-yl)acetamide; |
| 188 | N-(5-chloro-4-methoxypyridin-2-yl)-2-(6-(4-cyanophenyl)pyridin-3-yl)acetamide; |
| 189 | N-(5-chloro-4-methoxypyridin-2-yl)-2-(6-(3-cyanophenyl)pyridin-3-yl)acetamide; |
| 190 | (S)-2-((5-chloro-2-(2-(6′-cyano-[2,2′-bipyridin]-5-yl)acetamido)pyridin-4-yl)oxy)-N- |
| | methylpropanamide; |
| 191 | N-(5-chloro-4-methoxypyridin-2-yl)-2-(6′-cyano-4′-(4-methylpiperazin-1-yl)-[2,2′- |
| | bipyridin]-5-yl)acetamide; |
| 192. | N-(5-chloro-4-methoxypyridin-2-yl)-2-(6′-cyano-4′-(4-hydroxy-4-methylpiperidin-1- |
| | yl)-[2,2′-bipyridin]-5-yl)acetamide; |
| 193. | N-(5-chloro-4-methoxypyridin-2-yl)-2-(6′-cyano-4′-(3-methoxyazetidin-1-yl)-[2,2′- |
| | bipyridin]-5-yl)acetamide; |
| 194. | N-(5-chloro-4-methoxypyridin-2-yl)-2-(6′-cyano-4′-(3-hydroxy-3-methylpyrrolidin- |
| | 1-yl)-[2,2′-bipyridin]-5-yl)acetamide; |
| 195. | 2-(4′-((1R,5S)-8-oxa-3-azabicyclo[3.2.1]octan-3-yl)-6′-cyano-[2,2′-bipyridin]-5-yl)- |
| | N-(5-chloro-4-methoxypyridin-2-yl)acetamide; |
| 196. | (S)-N-(5-chloro-4-methoxypyridin-2-yl)-2-(6′-cyano-4′-((tetrahydrofuran-3-yl)oxy)- |
| | [2,2′-bipyridin]-5-yl)acetamide; |
| 197. | (S)-N-(5-chloro-4-methoxypyridin-2-yl)-2-(6′-cyano-4′-((tetrahydrofuran-3-yl)oxy)- |
| | [2,2′-bipyridin]-5-yl)acetamide; |
| 198. | N-(5-chloro-4-methoxypyridin-2-yl)-2-(6′-cyano-4′-((3-fluoro-1-methylpiperidin-4- |
| | yl)oxy)-[2,2′-bipyridin]-5-yl)acetamide; and |
| 199. | N-(5-chloro-4-methoxypyridin-2-yl)-2-(6′-cyano-4′-((1-methylpiperidin-3-yl)oxy)- |
| | [2,2′-bipyridin]-5-yl)acetamide; |

or a pharmaceutically acceptable salt thereof or a stereoisomer thereof.

In certain embodiments, the present application relates to a pharmaceutical composition, comprising at least one compound of formula (I), or a pharmaceutically acceptable salt or a stereoisomer thereof, and at least one pharmaceutically acceptable carrier or excipient.

In certain embodiments, the present application relates to a compound of formula (I) or a pharmaceutically acceptable salt or a stereoisomer thereof, for use as a medicament.

In certain embodiments, the present application provides a compound of formula (I) or a pharmaceutically acceptable salt or a stereoisomer thereof, for use in the treatment of a cancer.

In one of the embodiments, the present application provides a compound of formula (I) or a pharmaceutically acceptable salt or a stereoisomer thereof, for use in the treatment of a cancer wherein the cancer is selected from a carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate and skin, including squamous cell carcinoma; hematopoietic tumors of lymphoid lineage, including leukemia, acute lymphoblastic leukemia, acute lymphocytic leukemia, Hodgkins lymphoma, non-Hodgkins lymphoma, B-cell lymphoma, T-cell lymphoma, hairy cell lymphoma, myeloma, mantle cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoacanthoma, xeroderma pigmentosum, thyroid follicular cancer and Kaposi's sarcoma.

In one of the embodiments, the present application provides a compound of formula (I) or a pharmaceutically acceptable salt or a stereoisomer thereof, for use in the treatment of Myotonic Dystrophy type 1, Myotonic Dystrophy type 2, Fragile X associated tremor/ataxia syndrome, amyotrophic lateral sclerosis (ALS) and frontotemporal dementia, Huntington's Disease like 2, Huntington's Disease, several types of Spinocerebellar Ataxia, Dentatorubral-pallidoluysian atrophy and Spinal and Bulbar Muscular Atrophy.

### Pharmaceutical Compositions

In certain aspects of embodiments, provided herein is a pharmaceutical composition comprising the compound of formula (I) and a pharmaceutically acceptable salt or a stereoisomer thereof, and a pharmaceutically acceptable carrier or excipient.

In certain embodiments, the pharmaceutical composition further comprises at least one agent selected from an anticancer agent, a chemotherapy agent, and an antiproliferative compound.

In certain embodiments, provided herein is a pharmaceutical composition comprising a compound of formula (I), or a pharmaceutically acceptable salt thereof or a stereoisomer thereof as described herein and at least one pharmaceutically acceptable excipient (such as a pharmaceutically acceptable carrier or diluent). Preferably, the pharmaceutical composition comprises a therapeutically effective amount of at least one compound described herein. The compounds described in the present application may be associated with a pharmaceutically acceptable excipient (such as a carrier or a diluent) or be diluted by a carrier or enclosed within a carrier which can be in the form of a capsule, sachet, paper or other container.

The compounds of the present application may be used as single drug or as a pharmaceutical composition in which the compound is mixed with various pharmacologically acceptable materials.

The compounds of the application are typically administered in the form of a pharmaceutical composition. Such compositions can be prepared using procedures well known in the pharmaceutical art and comprise at least one compound of formula (I). The pharmaceutical composition of the present application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents, solvents and the like.

A pharmaceutically acceptable carrier can contain pharmaceutically acceptable agents that act, for example, to stabilize, increase solubility or to increase the absorption of a compound such as a compound of this application. Such pharmaceutically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a pharmaceutically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of this application. Liposomes, for example, which comprise phospholipids or other lipids, are nontoxic, pharmaceutically acceptable and metabolizable carriers that are relatively simple to make and administer.

The pharmaceutical composition can be administered by oral, parenteral or inhalation routes. Examples of the parenteral administration include administration by injection, percutaneous, transmucosal, intranasal and transpulmonary administrations.

Examples of suitable carriers include, but are not limited to, sterile water, salt solutions, alcohols, polyethylene glycols, peanut oil, olive oil, gelatin, lactose, terra alba, sucrose, dextrin, magnesium carbonate, sugar, amylose, magnesium stearate, talc, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present application may be formulated so as to provide desired release profile.

Administration of the compounds disclosed herein, in pure form or in an appropriate pharmaceutical composition, can be carried out using any of the accepted routes of administration of pharmaceutical compositions. The route of administration may be any route which effectively transports the active compound of formula (I) to the appropriate or desired site of action. Suitable routes of administration include, but are not limited to, oral, nasal, buccal, dermal, intradermal, transdermal, parenteral, rectal, subcutaneous, intravenous, intraurethral, intramuscular or topical.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of formula (I), with the carrier and, optionally, one or more accessory ingredients. In general, the formulations are prepared by uniformly and intimately bringing into association a compound of the present application with liquid carriers or finely divided solid carriers or both, and then, if necessary, shaping the product.

Formulations of compounds of formula (I) suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of formula (I) as an active ingredient. Compositions or compounds may also be administered as a bolus, electuary or paste.

Solid oral formulations include, but are not limited to, tablets, capsules (soft or hard gelatin), dragees (containing the active ingredient in powder or pellet form), troches and lozenges.

Solid oral formulations may contain, along with the active compound, lubricants, diluents, binding agents, disintegrating agents, wetting agents, preservatives, and in general, non-toxic, pharmacologically inactive substances used in pharmaceutical compositions.

The formulations may contain, lubricants, for ex., calcium stearate, magnesium stearate, stearic acid, talc, silica or polyethylene glycols; diluents, for ex., cellulose, corn starch, dextrose saccharose, lactose, potato starch, dry starch, sucrose, powdered sugar, mannitol, sorbitol, inositol, calcium carbonate, sodium carbonate, calcium phosphate, dicalcium phosphate and mixtures thereof; binding agents, for ex., Arabic gum, carboxymethylcellulose, gelatin methylcellulose, polyvinyl pyrrolidone or starches; disintegrating agents, for ex., alginic acid, alginates, starch or starch glycolate; wetting agents, for ex., lecithin, laurylsulphates or polysorbates; preservatives, for ex., antioxidants, chelating agents, antimicrobial preservatives, antifungal preservatives, antiprotozoan preservatives, alcohol preservatives.

Excipients such as cocoa butter, suppository waxes, colouring agents, coating agents, sweeteners, flavouring agents and perfuming agents may also be present in the composition.

Liquid formulations include, but are not limited to, syrups, emulsions, suspensions, sterile injectable liquids and solutions.

Topical dosage forms of the compounds include ointments, pastes, creams, lotions, powders, solutions, eye or ear drops, impregnated dressings, and may contain appropriate conventional additives such as preservatives and solvents to assist drug penetration.

The pharmaceutical compositions of compounds of formula (I) may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms, and suitable pharmaceutical excipients can also be well used and adjusted by those skilled in the art. All changes and modifications are envisioned within the scope of the present application.

According to one embodiment, the compounds disclosed herein can also contain unnatural proportions of atomic isotopes at one or more of the atoms that constitute such compounds. For example, the present application also embraces isotopically-labeled variants of the compounds of formula (I) which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of formula (I), and their uses. Exemplary isotopes that can be incorporated in to compounds disclosed herein include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I Isotopically labeled compounds of formula (I) can generally be prepared by following procedures analogous to those disclosed in the schemes and/or in the examples herein below, by substituting an isotopically labeled reagent for a non-isotopically labeled reagent.

### Methods of Treatment

In certain embodiments, provided herein are compounds of formula (I) for use as a medicament.

In certain embodiments, provided herein are compounds of formula (I) or a pharmaceutically acceptable salt, an N-oxide or a stereoisomer thereof, for use as a medicament.

In certain embodiments, provided herein are compounds of formula (I) or a pharmaceutically acceptable salt, an N-oxide or a stereoisomer thereof, for use in the treatment of a cancer, an inflammatory disorder, an auto-inflammatory disorder or an infectious disease.

In certain embodiments, provided herein are uses of the compounds of the present invention in manufacture of a medicament.

In certain embodiments, provided herein is a method of treating cancer or proliferative disorder, comprising administration of a therapeutically effective amount of a compound of formula (I) or a pharmaceutically acceptable salt or a stereoisomer thereof.

In certain embodiments, provided herein are methods for inhibiting growth of tumour cells and/or metastasis by administering a therapeutically effective amount of a compound of formula (I) or a pharmaceutically acceptable salt or a stereoisomer thereof.

In certain embodiments, provided herein are methods for treating cancer or proliferative disorder, by administering a therapeutically effective amount of a compound of formula (I) or a pharmaceutically acceptable salt or a stereoisomer thereof.

According to yet another aspect of an embodiment, the compounds of formula (I) are useful in the treatment of proliferative diseases such as cancer, viral diseases, fungal diseases, neurological/neurodegenerative disorders, autoimmune diseases, inflammation, arthritis, anti-proliferative (e.g., ocular retinopathy), neuronal, alopecia and cardiovascular disease.

According to yet another aspect of an embodiment, provided herein is a compound of formula (I), for use in the treatment of a cancer.

According to yet another aspect of an embodiment, the cancer is selected from a carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate, and skin, including squamous cell carcinoma; hematopoietic tumors of lymphoid lineage, including leukemia, acute lymphoblastic leukemia, acute lymphocytic leukemia, Hodgkin's lymphoma, non-Hodgkin's lymphoma, B-cell lymphoma, T-cell lymphoma, hairy cell lymphoma, myeloma, mantle cell lymphoma, and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoacanthoma, xeroderma pigmentosum, thyroid follicular cancer and Kaposi's sarcoma.

According to yet another aspect of an embodiment, provided herein is a compound of formula (I) for use in the treatment of Myotonic Dystrophy type 1, Myotonic Dystrophy type 2, Fragile X associated tremor/ataxia syndrome, amyotrophic lateral sclerosis (ALS) and frontotemporal dementia, Huntington's disease like 2, Huntington's disease, several types of Spinocerebellar Ataxia, Dentatorubral-pallidoluysian atrophy and Spinal and Bulbar Muscular Atrophy.

In another aspect of an embodiment, provided herein is a method of treating cancer in a subject, comprising administering to the subject a compound of formula (I).

In another aspect of an embodiment, provided herein is a method of inhibiting CDK12/13 in a subject, comprising administering to the subject a compound of formula (I).

In another aspect of an embodiment, provided herein is a method of selectively inhibiting CDK12/13 in a subject, comprising administering to the subject in need thereof a therapeutically effective amount of a compound of formula (I).

In another aspect of an embodiment, provided herein is a pharmaceutical composition for use in treating and/or preventing a disease and/or disorder associated with aberrant activity of CDK12/13.

In another aspect of an embodiment, provided herein is a pharmaceutical composition for use in treating a subject suffering from a disease or condition associated with aberrant activity of CDK12/13.

In another aspect of an embodiment, provided herein is a pharmaceutical composition comprising the compound of formula (I), for use in treating a subject suffering from a disease or condition associated with aberrant activity of CDK12/13.

In another aspect of an embodiment, provided herein is a method of treating a disease and/or disorder or a condition mediated by CDK12/13 in a subject comprising administering to the subject in need thereof a therapeutically effective amount of a compound of formula (I) of the present application.

In another aspect of an embodiment, provided herein is a method of treating a disease and/or disorder or a condition mediated by CDK12/13 in a subject comprising administering a therapeutically effective amount of a compound of formula (I).

According to the foregoing embodiments, provided herein is a method of treating a disease and/or disorder or a condition mediated by CDK12/13 in a subject wherein the CDK12/13 mediated disorder or disease or condition is selected from a cancer, an inflammatory disorder, an auto-inflammatory disorder and an infectious disease.

In another aspect of an embodiment, provided herein is a method of treating a disease and/or disorder or a condition selected from a cancer, an inflammatory disorder, an auto-inflammatory disorder and an infectious disease.

According to the foregoing embodiments, the cancer is selected from a carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate and skin, including squamous cell carcinoma; hematopoietic tumors of lymphoid lineage, including leukemia, acute lymphoblastic leukemia, acute lymphocytic leukemia, Hodgkin's lymphoma, non-Hodgkin's lymphoma, B-cell lymphoma, T-cell lymphoma, hairy cell lymphoma, myeloma, mantle cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoacanthoma, xeroderma pigmentosum, thyroid follicular cancer and Kaposi's sarcoma.

In certain embodiments, the subject is a human or other mammal.

In yet another aspect of an embodiment, the compounds of formula (I) as disclosed in the present application are formulated for pharmaceutical administration.

Yet another aspect of an embodiment provides use of compounds of formula (I) in the treatment and prevention of diseases or disorder associated with the aberrant activity of CDK12/13.

Yet another aspect of an embodiment provides use of compounds of formula (I) in the treatment of a cancer, an inflammatory disorder, an auto-inflammatory disorder or an infectious disease.

Yet another aspect of an embodiment provides the use of a compound of formula (I) or a pharmaceutically acceptable salt thereof, in treating and/or preventing a disease for which the symptoms thereof are treated, improved, diminished and/or prevented by inhibition of CDK12/13.

Yet another aspect of an embodiment provides the use of a compound of formula (I) or a pharmaceutically acceptable salt thereof or a stereoisomer thereof, in the manufacture of a medicament for the treatment of cancer.

According to yet another embodiment, the CDK12/13 mediated disorder and/or disease or condition is a proliferative disease or disorder or condition.

In yet another aspect of an embodiment, the diseases and/or disorder mediated by CDK12/13 is selected from a cancer, an inflammatory disorder, an auto-inflammatory disorder and an infectious disease.

In certain other embodiments, the proliferative disease to be treated or prevented using the compounds of formula (I) will typically be associated with aberrant activity of CDK12/13.

In certain embodiments, CDK12/13 refers to CDK12 or CDK13 or CDK12 and CDK13.

According to yet another aspect of an embodiment, the disorder or condition mediated by CDK12/13 is Myotonic Dystrophy type 1, Myotonic Dystrophy type 2, Fragile X associated tremor/ataxia syndrome, amyotrophic lateral sclerosis (ALS) and frontotemporal dementia, Huntington's disease like 2, Huntington's disease, several types of Spinocerebellar Ataxia, Dentatorubral-pallidoluysian atrophy and Spinal and Bulbar Muscular Atrophy.

According to yet another aspect of an embodiment, provided herein is a method of treating a disorder or condition mediated by CDK12/13 wherein the disorder or condition is Myotonic Dystrophy type 1, Myotonic Dystrophy type 2, Fragile X associated tremor/ataxia syndrome, amyotrophic lateral sclerosis (ALS) and frontotemporal dementia, Huntington's disease like 2, Huntington's disease, several types of Spinocerebellar Ataxia, Dentatorubral-pallidoluysian atrophy and Spinal and Bulbar Muscular Atrophy.

According to yet another aspect of an embodiment, the disease and/or disorder mediated by CDK12/13 is Myotonic dystrophy.

According to yet another aspect of an embodiment, the compounds of formula (I) are useful in the treatment of Myotonic dystrophy.

According to yet another aspect of an embodiment, provided herein is a method of treating Myotonic dystrophy by administering a therapeutically effective amount of a compound of formula (I).

According to yet another aspect of an embodiment, provided herein are compounds of formula (I) in the manufacture of a medicament for treating Myotonic dystrophy.

According to yet another aspect of an embodiment, provided herein is a method further comprising administering to the subject in need thereof one or more chemotherapeutic agents independently selected from anti-proliferative agents, anti-cancer agents, immunosuppressant agents and pain-relieving agents.

According to yet another aspect of an embodiment, the subject is a human or other mammal.

According to yet another aspect of an embodiment, provided herein are compounds of formula (I) or pharmaceutically acceptable salts or stereoisomers thereof, for use as a medicament.

According to yet another aspect of an embodiment, the application provides the use of the compounds of formula (I) in the manufacture of a medicament.

According to yet another aspect of an embodiment, provided herein are compounds of formula (I) or pharmaceutically acceptable salts or stereoisomers thereof, for use in the treatment of cancer.

According to yet another aspect of an embodiment, provided herein are compounds of formula (I) or pharmaceutically acceptable salts or stereoisomers thereof, for use in the treatment of an inflammatory disorder, an auto-inflammatory disorder or an infectious disease.

According to yet another aspect of an embodiment, provided herein are uses of the compounds of formula (I) in the manufacture of a medicament for the treatment of a disease and/or disorder associated with the aberrant activity of CDK12/13.

In yet another aspect of an embodiment, provided herein are uses of the compounds of formula (I) in the manufacture of a medicament for the treatment of cancer.

In yet another embodiment, provided herein are uses of the compounds of formula (I) in the manufacture of a medicament for the treatment of an inflammatory disorder, an auto-inflammatory disorder or an infectious disease.

According to yet another aspect of the embodiment, provided herein are compounds of formula (I) for use as a medicament for treating a subject suffering from a disease and/or disorder associated with aberrant activity of CDK12/13.

Another aspect of the embodiment comprises administering to the subject in need thereof a therapeutically effective amount of a compound of formula (I) along with one or more additional chemotherapeutic agents independently selected from anti-proliferative agents, anti-cancer agents, immunosuppressant agents and pain-relieving agents.

According to yet another aspect of the embodiment, the present application further comprises administering to the subject in need thereof one or more chemotherapeutic agents independently selected from anti-proliferative agents, anti-cancer agents, immunosuppressant agents and pain-relieving agents.

According to yet another aspect of the embodiment, the chemotherapeutic agents are selected from, but not limited to, CPT-11, camptothecin derivatives, taxane, taxane derivatives, encapsulated taxanes, anthracyclin glycosides, for ex., doxorubicin, idarubicin, epirubicin, etoposide, navelbine, vinblastine, carboplatin, cisplatin, estramustine, celecoxib, Sugen SU-5416, Sugen SU-6668, Herceptin, optionally within liposomal formulations thereof.

According to yet another aspect of the embodiment, the anti-cancer agents are selected from, but not limited to, Atezolizumab, Avelumab, Bevacizumab, Cetuximab, ipilimumab, nivolumab, Obinutuzumab, Panitumumab, Pembrolizumab, Pertuzumab, Vinblastine, Vincristine, Zoladex, Abemaciclib, palbociclib, Ribociclib, Kymriah, Letrozole, Avapritinib, Bosutinib, Ceritinib, Crizotinib, Dasatinib, Erlotinib Hydrochloride, Gefitinib, Imatinib Mesylate, Ibrutinib, Sunitinib, and the like.

In certain other embodiments, the chemotherapeutic agent is methotrexate, doxorubicin hydrochloride, chlorambucil, nelarabine, ofatumumab, bosutinib, busulfan, alemtuzumab, daunorubicin hydrochloride, cyclophosphamide, clofarabine, cytarabine, Asparaginase Erwinia Chrysanthemi, fludarabine phosphate, obinutuzumab, ponatinib hydrochloride, ibrutinib, vincristine sulfate liposome, mitoxantrone hydrochloride, mechlorethamine hydrochloride, Pegaspargase, mercaptopurine, Rubidomycin, daunorubicin hydrochloride, omacetaxine mepesuccinate, cytarabine, nilotinib, bendamustine hydrochloride, arsenic trioxide, vincristine sulfate, idelalisib, or a combination thereof.

In certain embodiments, the additional chemotherapeutic agent is an anti-lymphoma agent. In certain embodiments, the additional chemotherapeutic agent is brentuximab vedotin, doxorubicin hydrochloride, nelarabine, tositumomab, bleomycin, dacarbazine, pralatrexate, recombinant interferon alfa-2b, romidepsin, Lomustine, procarbazine hydrochloride, plerixafor, mechlorethamine hydrochloride, lenalidomide, rituximab, bendamustine hydrochloride, vinblastine sulfate, bortezomib, vincristine sulfate, ibritumomab tiuxetan, vorinostat, or a combination thereof.

In certain embodiments, the additional chemotherapeutic agent is Abitrexate, Abraxane, Adriamycin Pfs, Adrucil, Afinitor, Afinitor Disperz, Aldara, Alimta, Aredia, Arimidex, Aromasin, Avastin, Becenum, Bicnu, Blenoxane, Camptosar, Capox, Caprelsa, Carboplatin-Taxol, Carmubris, Casodex, Cerubidlne, Clafen, Cometriq, Cosmegen, Cyfos, Cyramza, Cytosar-U, Cytoxan, Dacogen, Degarelix, Doxil, Doxorubicin Hydrochloride, Efudex, Ellence, Eloxatin, Erbitux, Erivedge, Etopophos, Evacet, Fareston, Faslodex, Femara, Fluoroplex, Cizumab, Folfiri-Cetuximab, Folfirinox, Folfox, Gemcitabine-Oxaliplatin, Gemzar, Gilotrif, Gleevec, Gliadel, Gliadel Wafer, Herceptin, Hycamtin, Ifosfamidum, Inlyta, Keytruda, Kyprolis, Lipodox, Lupron Depot, Megace, Methazolastone, Mitoxantrone Hydrochloride, Mitozytrex, Mozobil, Mustargen, Mutamycin, Mylosar, Navelbine, Nexavar Nolvadex, Paraplatin, Platinol, Proleukin, Stivarga, Tafinlar, Temodar, Thalomid, Toposar, Torisel, Trisenox, Vectibix, Viadur, Vidaza, Zaltrap, Zoladex, Zometa, Zykadia, Zytiga, or a combination thereof.

The method(s) of treatment disclosed herein comprises administering a safe and effective amount of a compound according to formula (I) or a pharmaceutically acceptable salt thereof to a patient (particularly a human) in need thereof.

Compounds of the application are indicated both in the therapeutic and/or prophylactic treatment of the above-mentioned conditions. For the above-mentioned therapeutic uses the dosage administered will, of course, vary with the compound employed, the mode of administration, the treatment desired and the disorder or disease indicated.

### EXPERIMENTAL SECTION

Abbreviations: The following abbreviations refer respectively to the definitions herein:

K₂CO₃ (Potassium carbonate); EtOH (Ethanol); rt (Retention time); RT (Room temperature); DMF (Dimethylformamide); h, hr (hour); NaOH (Sodium hydroxide); THF (tetrahydrofuran); LCMS (Liquid chromatography mass spectroscopy); HCl (Hydrochloric acid); DCM/CH₂Cl₂ (Dichloromethane); TFA (Trifluoroacetic acid); TLC (Thin layer chromatography); DIPEA (Diisopropyl Ethyl amine); Na₂SO₄ (Sodium sulphate); ACN/CH₃CN (Acetonitrile); MeOH (Methanol); (COCl)₂ (Oxalyl chloride); PdCl₂(dppf)-DCM/Pd(dppf)Cl₂·DCM ([1,1′-Bis(diphenylphosphino)ferrocene]dichloropalladium(II)·dichloromethane complex); DMSO-d₆ (Dimethyl sulfoxide-d); Boc₂O (Ditert-butyl dicarbonate); HPLC (High pressure liquid chromatography); NaHCO₃ (Sodium bicarbonate); TEA/Et₃N (triethyl amine), MHz (megahertz); s (singlet); m (multiplet); and d (doublet); KHMDS (Potassium bis(trimethylsilyl)amide); NCS (N-Chlorosuccinimide); n-BuLi (n-Butyllithium); NH₄OH (Ammonium hydroxide); Me-NH₂ (Methyl amine); Ac₂O (Acetic anhydride); KO^{t}Bu/t-BuOK (Potassium tertiary butoxide); NMP (N-Methyl-2-pyrrolidone); T₃P (Propylphosphonic anhydride solution); CNBr (Cyanogen bromide); DMAP (4-Dimethylaminopyridine); EtOAc (Ethyl acetate); Na₂CO₃ (Sodium carbonate); NMR (Nuclear Magnetic Resonance); Int. (Intermediate); min/mins (Minutes); H₂O (Water); K₃PO₄ (tripotassium phosphate); Pd/C (Palladium on carbon); Aq (Aqueous); NH₄Cl (Ammonium Chloride); CDCl₃ (Deuterated chloroform); mL (milliliters); mmol (millimoles); g (grams); LAH (Lithium Aluminum Hydride); LiHMDS (Lithium bis(trimethylsilyl)amide); M (Molar); ° C. (Degree Celsius); m/z (mass to charge ratio); DMSO (dimethyl sulfoxide); MeI (Methyl Iodide); N (Normality); NaOMe (Sodium Methoxide); NiCl₂ (nickel (II) chloride); Pd₂(dba)₃ (Tris(dibenzylideneacetone)dipalladium(0)); mm (millimeter); NBS (N-bromo succinimide); KHF₂ (Potassium bifluoride); Me₂P(O)H (Dimethylphosphine oxide); Pd(PPh₃)₄(Tetrakis(triphenylphosphine)palladium(0)); LiCl (Lithium chloride); KOAc (Potassium acetate); TMSCN (Trimethylsilyl cyanide); Prep. (Preparative); Conc. (Concentrated); MW (Microwave); soln. (solution); bs (broad singlet); dd (doublet of doublets); and psi (Pound per square inch).

### General Modes of Preparation:

The following general guidelines apply to all experimental procedures described here. Unless otherwise stated, experiments are performed under positive pressure of nitrogen, and the temperature described is the external temperature (i.e. oil bath temperature). Reagents and solvents received from vendors are used as such without any further drying or purification. Molarities mentioned here for reagents in solutions are approximate as it was not verified by a prior titration with a standard. All reactions are stirred with a magnetic stir bar. Cooling to minus temperature was done by acetone/dry ice or wet ice/salts. Anhydrous Magnesium sulfate and anhydrous sodium sulfate were used as solvent drying agents after reaction work up and are interchangeable. Removing of solvents under reduced pressure or under vacuum means distilling of solvents in a rotary evaporator.

Compounds of this application may be made by synthetic chemical processes, examples of which are shown herein. It is meant to be understood that the order of the steps in the processes may be varied, that reagents, solvents and reaction conditions may be substituted for those specifically mentioned and that vulnerable moieties may be protected and deprotected, as necessary.

The specifics of the process for preparing compounds of formula (I) are detailed in the experimental section. The reagents and intermediates used herein are either commercially available or prepared in-house.

The present application shall be illustrated by means of some examples, which are not construed to be viewed as limiting the scope of this disclosure.

Unless otherwise stated, work-up includes distribution of the reaction mixture between the organic and aqueous phases, separation of layers and drying the organic layer over anhydrous sodium sulphate, filtration and distillation of the solvent. Purification, unless otherwise mentioned, includes purification by silica gel chromatographic techniques, generally using a mixture of ethyl acetate/petroleum ether or ethyl acetate/hexanes of suitable polarity as the mobile phase.

Analysis of the compounds of the present application unless mentioned, was conducted using general methods well known to a person skilled in the art. Having described this disclosure with reference to certain preferred embodiments, other embodiments will become apparent to one skilled in the art from consideration of the specification. The disclosure is further defined by reference to the following examples, describing in detail the analysis of the compounds of this application.

It will be apparent to those skilled in the art that many modifications, both to materials and methods, may be practiced without departing from the scope of the disclosure. Some of the intermediates were taken to next step based on TLC results, without further characterization, unless otherwise specified. The intermediates used here may be commercially available or synthesized in-house, unless otherwise specified.

### General Schemes

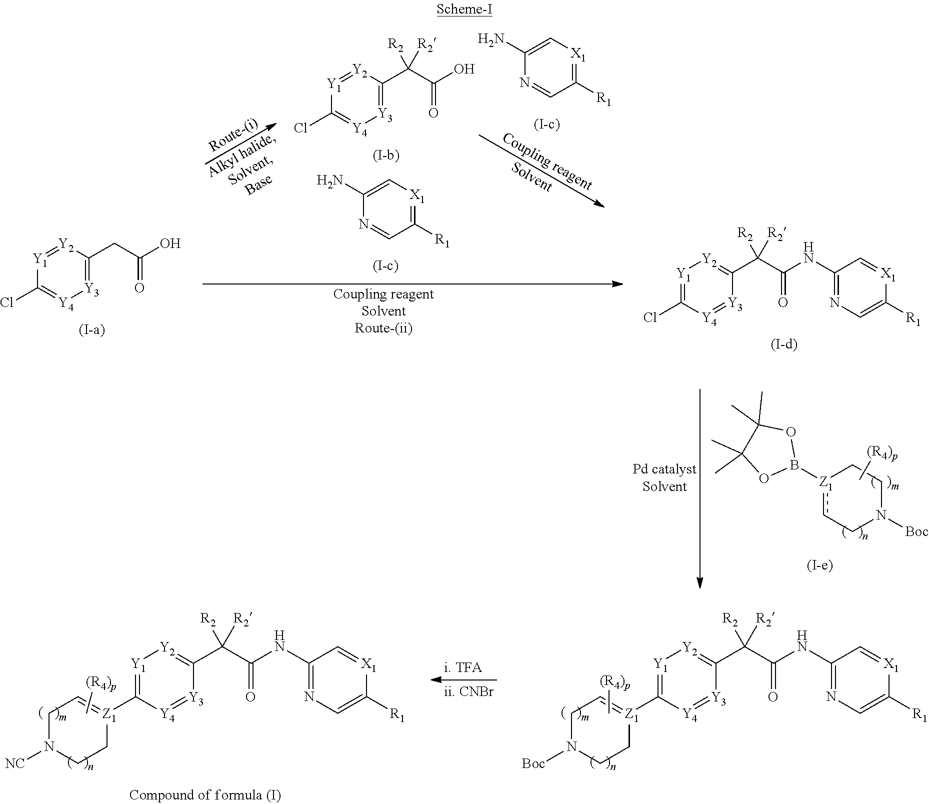

All the intermediates listed in Table-E and the respective final compounds were prepared by this general scheme.

As depicted in the above scheme-I, Intermediate (I-a) was alkylated with an alkyl halide (R₂—X) in presence of a base and solvent (Route-(ii) applicable when R₂=R₂′=H), to afford the carboxylic acid intermediate (I-b), which was further coupled with an amine (I-c) to afford intermediate (I-d). Then, intermediate (I-d) was coupled with intermediate (I-e) under Suzuki coupling conditions to afford intermediate (I-f). The Boc-protected intermediate (I-f) was reacted with trifluoroacetic acid to deprotect the Boc-group and the obtained intermediate was reacted with cyanogen bromide or any other R₃—Br (as exemplified) in presence of a base to afford a compound of formula (I).

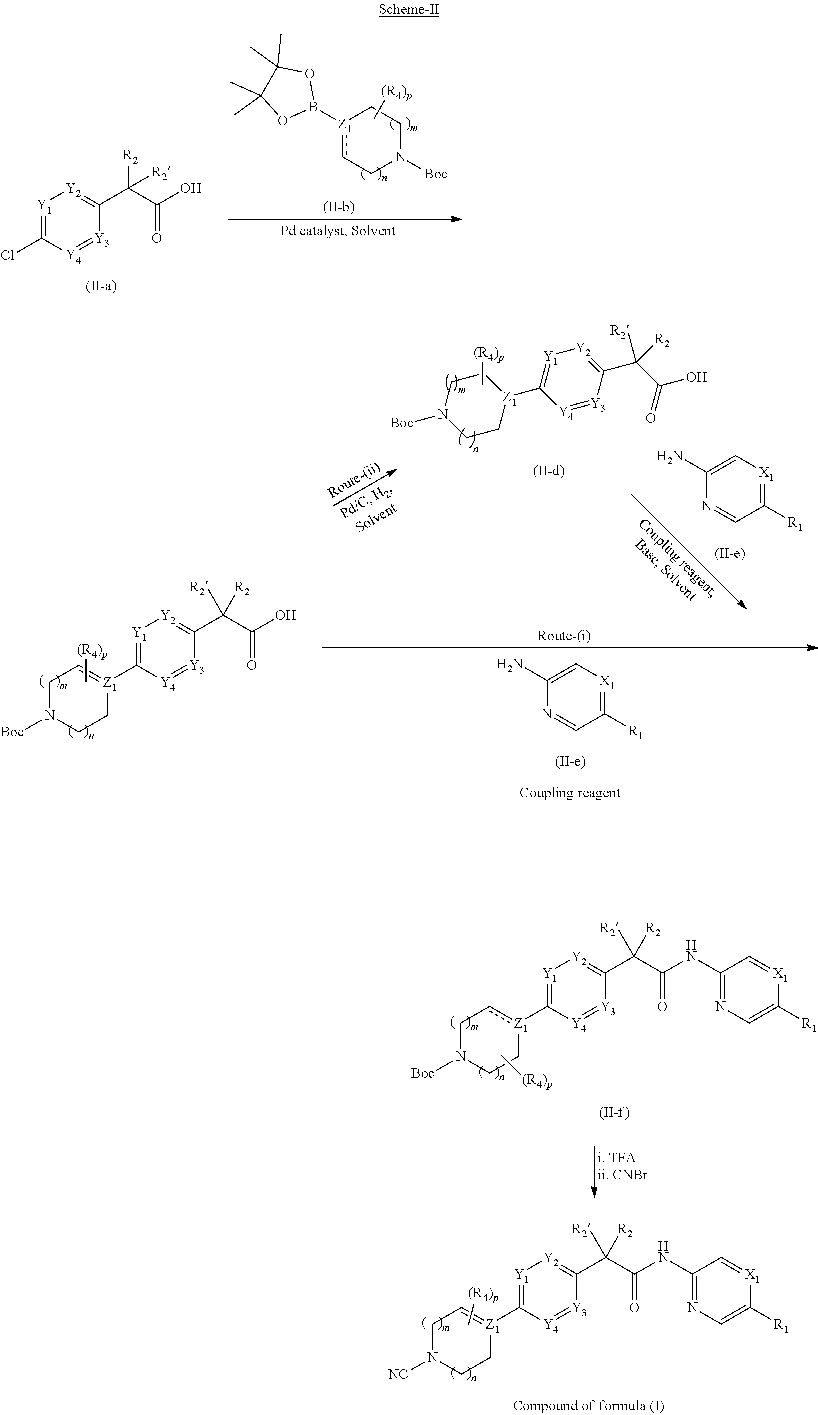

All the intermediates listed in Table-H and the respective final compounds were prepared by this general scheme.

As depicted in the above scheme-II, Intermediate (II-a) (same as intermediate I-b) was coupled with a boronate ester or boronic acid (II-b) in appropriate conditions to afford the carboxylic acid intermediate (II-c). This was further coupled with an amine (II-e) to afford amide intermediate (II-f). Then, (II-f) was reacted with trifluoroacetic acid to deprotect the Boc group to afford an intermediate (free amine or TFA salt) which was further reacted with cyanogen bromide in presence of a base to afford a compound of formula (I). Alternatively, in Route-(ii), intermediate (II-c) was reduced under hydrogen pressure in presence of Pd/C catalyst to afford intermediate (II-d) which was further reacted with an amine (II-e) to afford amide intermediate (II-f). Conversion of intermediate (II-f) to compound of formula (I) is as explained above.

From Scheme-I and Scheme-II, it is apparent to those skilled in the art that, Formula (I-f) and (II-f) are same, only the method of synthesis is different in the order in which experimental steps are carried out. It is also apparent to a person skilled in the art that any intermediate synthesized using Scheme-I can be alternatively synthesized using Scheme-II and vice-versa.

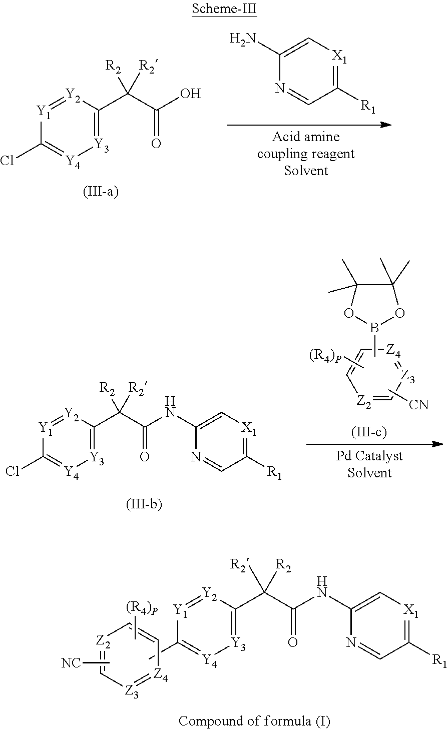

As depicted in the above scheme-III, Intermediate (III-a) (same as intermediate I-b & II-a) was coupled with an amine to afford intermediate (III-b) under acid-amine coupling conditions. This intermediate (III-b) was coupled with a boronate ester or boronic acid (III-c) under appropriate Suzuki coupling conditions in presence of a Pd catalyst and solvent to afford a compound of formula (I).

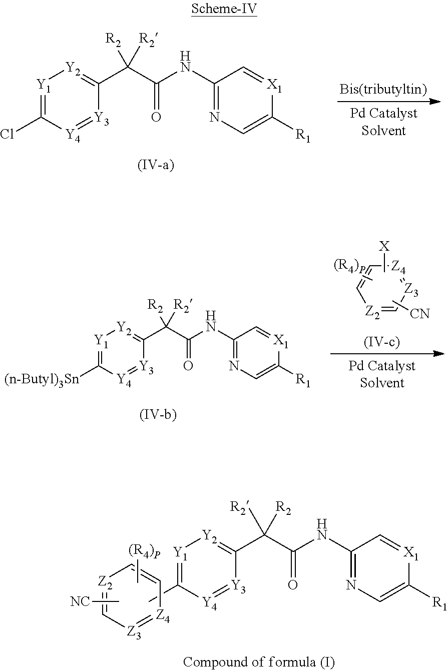

As depicted in the above scheme-IV, Intermediate (IV-a) was stannylated using Bis(tributyltin) to afford intermediate (IV-b) in presence of Pd catalyst and appropriate solvent. This intermediate (IV-b) was coupled with a halo compound (IV-c) under appropriate Stille coupling conditions in presence of a Pd catalyst and solvent to afford a compound of formula (I).

### SYNTHESIS OF INTERMEDIATES

### Intermediate I-1: Synthesis of 5-chloro-N⁴,N⁴-dimethylpyridine-2,4-diamine

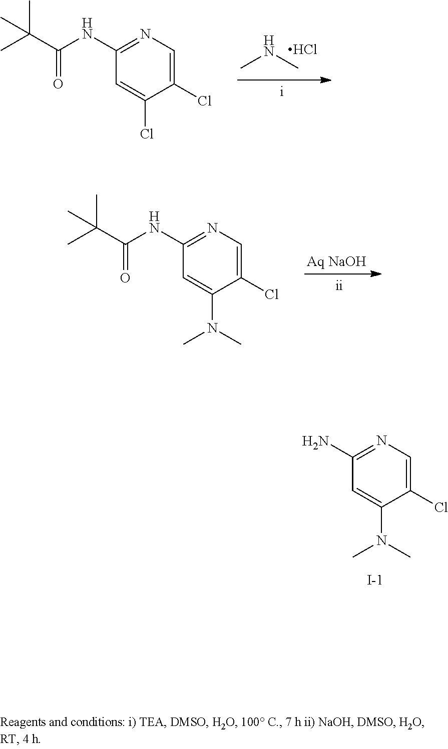

### Step-i: Synthesis of N-(5-chloro-4-(dimethylamino)pyridin-2-yl)pivalamide

To a stirred solution of N-(4,5-dichloropyridin-2-yl)pivalamide (4.1 g, 16.59 mmol) in DMSO (10 mL)/Water (4 mL) was added N,N-dimethylammonium chloride (2.70 g, 33.18 mmol) and TEA (5.10 mL, 36.5 mmol). The reaction mixture was stirred at 100° C. for 7 h. After completion of the reaction, the reaction mixture was quenched with ice water and extracted with EtOAc. The organic layer was dried over sodium sulphate and concentrated. The crude mixture was purified by Combiflash with EtOAc:Hexane as eluent to obtain N-(5-chloro-4-(dimethylamino)pyridin-2-yl)pivalamide (1.7 g, 40%). LCMS m/z: 256.30 (M+H)⁺.

### Step-ii: Synthesis of 5-chloro-N⁴,N⁴-dimethylpyridine-2,4-diamine

To a solution of N-(5-chloro-4-(dimethylamino) pyridin-2-yl)pivalamide (1.7 g, 6.66 mmol) in DMSO (5 mL) was added 2 mL of 10% aqueous NaOH solution at RT, and the resulting mixture was stirred at RT for 4 h. After completion of the reaction, the mixture was quenched with ice water and extracted with EtOAc. The organic layer was dried over sodium sulphate and concentrated. The crude mixture was purified by Combiflash with EtOAc:Hexane as eluent to obtain pure titled product (1.02 g, 90%). LCMS m/z: 172.2 (M+H)⁺.

### Intermediate I-2: Synthesis of 2-(6-chloropyridin-3-yl)propanoic acid

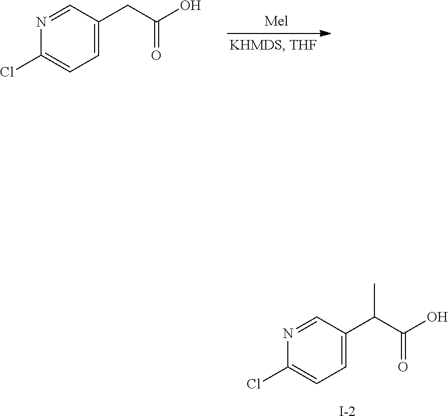

To a solution of 2-(6-chloropyridin-3-yl)acetic acid (8 g, 46.63 mmol) in 100 mL dry THF was added 1M KHMDS (107.25 mL, 107.25 mmol) in THF solution dropwise at −78° C. The mixture was stirred at −78° C. for 1 h. Methyl iodide (3.63 mL, 58.29 mmol) was added dropwise at the same temperature, and the resulting suspension was stirred at RT for 24 h. The reaction mixture was quenched with 2N HCl and extracted with ethyl acetate, dried over sodium sulphate and concentrated. The crude mixture was purified by Combiflash using EtOAc:hexane eluent to obtain the titled compound. ¹HNMR (400 MHz, DMSO-d₆): δ 12.46 (s, 1H), 8.33 (d, 1H), 7.78-7.76 (m, 1H), 7.47 (d, 1H), 3.81-3.76 (m, 1H), 1.38 (d, 3H); LCMS m/z: 186 (M+H)⁺.

### Intermediate I-3: Synthesis of 1,2-dimethylpiperidin-4-ol

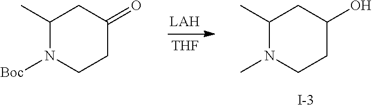

To a stirred solution of tert-butyl 2-methyl-4-oxopiperidine-1-carboxylate (0.50 g, 2.34 mmol) in dry THF (50 mL) was added 2M LAH in THF (4.68 mL, 9.37 mmol) slowly at −78° C. The reaction mixture was slowly warmed to RT and refluxed for 3 h. The reaction mixture was cooled to 0° C., basified using 2M NaOH solution and filtered through a celite bed. The filtrate was diluted with EtOAc and washed with water and brine solution. The organic layer was dried over sodium sulphate and concentrated. The crude product was purified by Combiflash with hexane:EtOAc (70:30) as eluent to give the desired product (0.17 g, 56.13%). LCMS m/z: 130.10 (M+H)⁺.

### Intermediate I-4: Synthesis of 2-methyl-2-azaspiro[3.3]heptan-6-ol

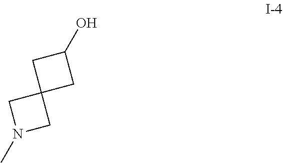

The above Intermediate I-4 was prepared by a procedure similar to the one described in the synthesis of Intermediate I-3 and by using tert-butyl 6-oxo-2-azaspiro[3.3]heptane-2-carboxylate as a reagent. LCMS m/z: 128.60 (M+H)⁺.

### Intermediate I-5: Synthesis of tert-butyl 2-methyl-4-(4,4,5,5-tetramethyl-1,3,2-dioxaborolan-2-yl)-3,6-dihydropyridine-1(2H)-carboxylate

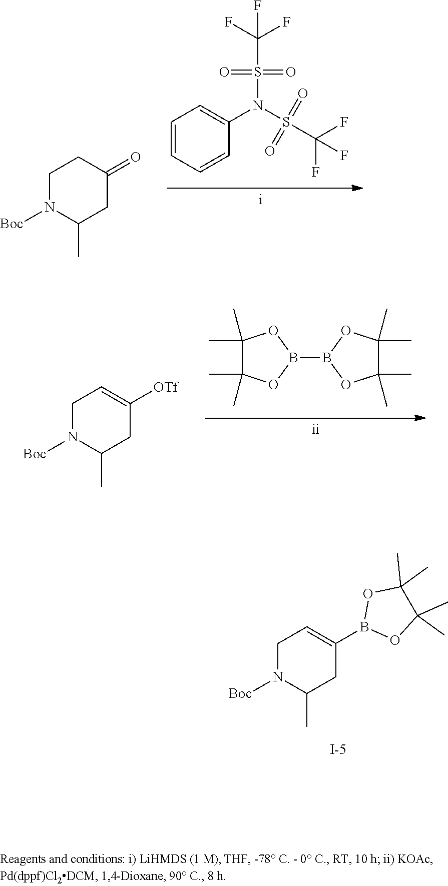

### Step-i: Synthesis of tert-butyl 2-methyl-4-(((trifluoromethyl)sulfonyl)oxy)-3,6-dihydropyridine-1(2H)-carboxylate

To a THF solution (20 mL) of tert-butyl 2-methyl-4-oxopiperidine-1-carboxylate (2 g, 9.37 mmol), a 1.0 M THF solution (11.25 mL, 11.25 mmol) of lithium bis(trimethylsilyl)amide was added at −78° C. and the resultant mixture was stirred for 30 min. Then a THF solution (10 mL) of N-phenylbis(trifluoromethanesulfonimide) (4.02 g, 11.25 mmol)) was added thereto at −78° C., and the resultant mixture was stirred at room temperature for 10 h. The reaction mixture was quenched with saturated NH₄Cl solution and extracted with ethyl acetate (3×100 mL). The combined organic layers were washed twice with water and then once with brine solution and dried over anhydrous sodium sulphate. The organic layer was concentrated under reduced pressure, and the crude product was purified by Combiflash with hexane:EtOAc as eluent to give the titled product (3.1 g, 95.73%). LCMS m/z: 345.90 (M+H)⁺.

### Step-ii: Synthesis of tert-butyl 2-methyl-4-(4,4,5,5-tetramethyl-1,3,2-dioxaborolan-2-yl)-3,6-dihydropyridine-1(2H)-carboxylate

To a degassed solution of tert-butyl 2-methyl-4-(((trifluoromethyl)sulfonyl)oxy)-3,6-dihydropyridine-1(2H)-carboxylate (3.1 g, 8.97 mmol) in 1,4-dioxane (50 mL), KOAc (2.64 g, 26.93 mmol) and 4,4,4′,4′,5,5,5′,5′-Octamethyl-2,2′-bi-1,3,2-dioxaborolane (3.41 g, 13.46 mmol) were added, and the resultant solution was degassed for 10 min. Pd(dppf)Cl₂·DCM (0.328 g, 0.44 mmol) was added, and the mixture was heated at 90° C. for 8 h. The reaction mass was filtered through a celite bed, and the filtrate was evaporated. The crude material was further purified using flash chromatography using ethyl acetate:hexane (20%) mixture as eluent to afford the title compound (2.8 g, 96.49%). LCMS m/z: 224.25 (De-boc mass) (M+H)⁺.

The intermediates listed in Table A were prepared by a procedure similar to the one described in the synthesis of Intermediate I-5 with appropriate variations in reactants/reagents. The characterization data of the intermediates are summarized herein the below table:

TABLE A

| Intermediate | Starting material | | Characterisation |
|---|---|---|---|
| No. | used | Intermediate Structure | Data |
| A-1 | 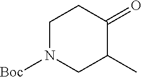 | 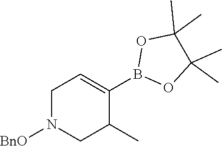 | LCMS m/z: 224.25 (De-boc mass) (M + H)⁺. |
| A-2 | 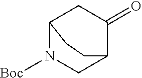 [SMILES: CC(C)(C)OC(=O)N1CC2CCC1CC2=O] | 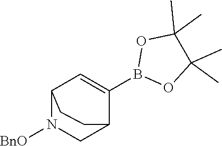 | LCMS m/z: 236.20 (De-boc mass) (M + H)⁺. |
| A-3 | 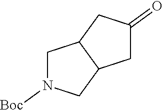 [SMILES: CC(C)(C)OC(=O)N1CC2CC(=O)CC2C1] | 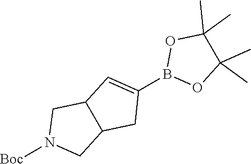 [SMILES: CC(C)(C)OC(=O)N1CC2C=C(B3OC(C)(C)C(C)(C)O3)CC2C1] | LCMS m/z: 336.10 (M + H)⁺. |
| A-4 | 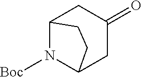 [SMILES: CC(C)(C)OC(=O)N1C2CCC1CC(=O)C2] | 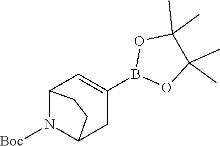 [SMILES: CC(C)(C)OC(=O)N1C2C=C(B3OC(C)(C)C(C)(C)O3)CC1CC2] | LCMS m/z: 335.25 (M + H)⁺. |

### Intermediate I-6: Synthesis of 2-(6-iodo-5-methoxypyridin-3-yl) acetic acid

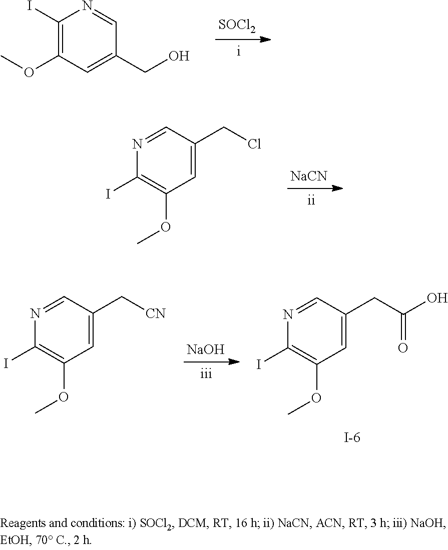

### Step-i: Synthesis of 5-(chloromethyl)-2-iodo-3-methoxypyridine

To a stirred solution of (6-iodo-5-methoxypyridin-3-yl) methanol (4 g, 15.09 mmol) (synthesis carried out as described in reference European Journal of Medicinal Chemistry, 2018, vol. 149, p. 110-121) in DCM (30 mL) was added thionyl chloride (20 ml) at 0° C. for 10 min. After addition, the reaction mixture was stirred at RT for 16 h. The reaction mixture was concentrated, and residue was quenched with cold solution of saturated NaHCO₃ and extracted with DCM. The organic layer and aqueous layer were separated, organic layer was dried over anhydrous sodium sulphate and concentrated under vacuum. The crude compound was purified by silica gel column chromatography by eluting with 0-50% ethyl acetate-hexane to afford the title compound (4.0 g 93.5%). ¹HNMR (DMSO-d₆, 400 MHz): δ 8.02 (s, 1H), 7.43 (s, 1H), 4.78 (s, 2H), 3.93 (s, 3H). LCMS: m/z=284.1 (M+H)⁺.

### Step-ii: Synthesis of 2-(6-iodo-5-methoxypyridin-3-yl) acetonitrile

To a stirred solution of 5-(chloromethyl)-2-iodo-3-methoxypyridine (2.5 g, 8.81 mmol) in acetonitrile (20 mL) was added NaCN (8.1 g, 9.69 mmol) at RT, and the reaction mixture was stirred at RT for 3 h. After the completion of the reaction, the mixture was quenched into ice water and extracted with EtOAc. The organic layer was dried over sodium sulphate and concentrated. The crude product was purified by Combiflash with EtOAc:Hexane as eluent to give the titled product (1.6 g, 66.20%). LCMS m/z: 275.2 (M+H)⁺.

### Step-iii: Synthesis of 2-(6-iodo-5-methoxypyridin-3-yl) acetic acid

To a solution of 2-(6-iodo-5-methoxypyridin-3-yl) acetonitrile (1.6 g, 5.83 mmol) in Ethanol (20 mL) was added NaOH (0.23 g, 5.85 mmol) at RT, and the resulting mixture was stirred at 70° C. for 2 h. After the completion of the reaction, the mixture was quenched with 2N HCl solution and extracted with EtOAc, the organic layer was dried over sodium sulphate and concentrated. The crude product was purified by Combiflash with EtOAc:Hexane as the eluent to obtain pure titled product (1.00 g, 58.45%). LCMS m/z: 294.10 (M+H)⁺.

### Intermediate I-7: Synthesis of 2-(2-amino-5-chloropyridin-4-yl) propan-2-ol

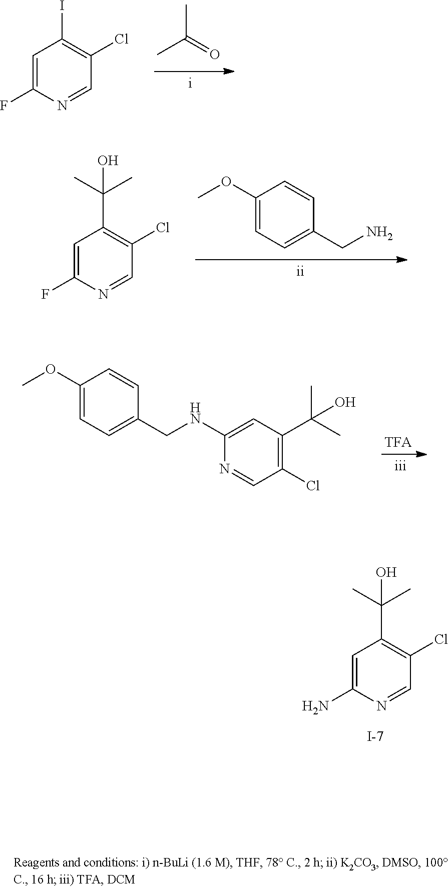

### Step-i: Synthesis of 2-(5-chloro-2-fluoropyridin-4-yl) propan-2-ol

To a stirred solution of 5-chloro-2 fluoro-4-iodopyridine (1.5 g, 5.82 mmol) in dry THF (10 mL), was added n-BuLi (1.6M in THF) (4.7 mL, 7.56 mmol) at −78° C. The reaction mixture was stirred at this temperature for 45 min. Dry Acetone (0.85 mL, 11.64 mmol) in dry THF (3 mL) was added at the same temperature and the reaction mixture was stirred for 2 h. After completion of the reaction, the mixture was quenched with saturated ammonium chloride solution and extracted with DCM. The organic layer was washed with brine, dried over sodium sulphate, and concentrated under vacuum. The crude residue was purified by Combi flash, and the product was eluted at 3% ethyl acetate/hexane as oily liquid (0.35 g, 31%); LCMS m/z: 190 (M+H)⁺.

### Step-ii: Synthesis of 2-(5-chloro-2-((4-methoxybenzyl) amino) pyridin-4-yl) propan-2-ol

To a stirred solution of 2-(5-chloro-2-fluoropyridin-4-yl)propan-2-ol (0.3 g, 1.58 mmol) in DMSO (2 mL) at 0° C. were added K₂CO₃ (0.5 g, 3.16 mmol) and 4-methoxybenzylamine (0.43 g, 3.16 mmol). The mixture was then slowly brought to RT and stirred at 100° C. for 16 h in a sealed tube. After completion of the reaction, the mixture was quenched with ice cold water and extracted with ethyl acetate. The organic layer was washed with brine, dried over sodium sulphate, and concentrated under vacuum. The crude residue was purified by Combi flash, and the product eluted at 30% ethyl acetate/hexane as an off-white solid. (0.2 g, 41.2%); LCMS m/z: 307.1 (M+H)⁺.

### Step-iii: Synthesis of 2-(2-amino-5-chloropyridin-4-yl) propan-2-ol

To a stirred solution of 2-(5-chloro-2-((4-methoxybenzyl) amino) pyridin-4-yl) propan-2-ol (0.2 g, 0.649 mmol) in DCM (1 mL) was added TFA (1 mL). The reaction mixture was stirred at RT for 2 h, subsequently diluted with DCM, and washed with saturated NaHCO₃ and brine solution. The isolated organic layer was dried over sodium sulphate and concentrated under vacuum to yield the desired product which was used without further purification (0.12 g, 98%); LCMS m/z: 187 (M+H)⁺.

### Intermediate I-8: Synthesis of 3-(2-amino-5-chloropyridin-4-yl) oxetan-3-ol

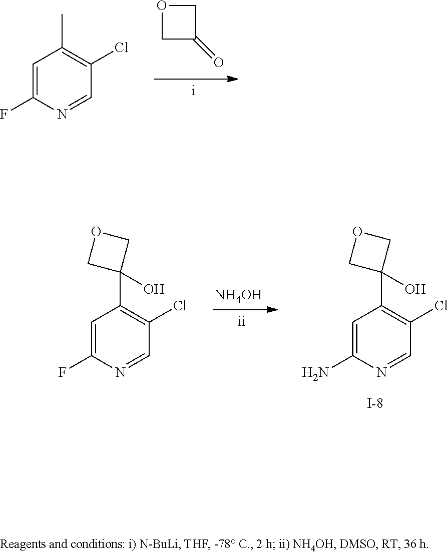

### Step-i: Synthesis of 3-(5-Chloro-2-fluoropyridin-4-yl)oxetan-3-ol

To a stirred solution of 5-chloro-2-fluoro-4-iodopyridine (5 g, 19.42 mmol) in dry THF was added n-BuLi (1.6M in THF) (15.7 mL, 25.24 mmol) at −78° C. The reaction mixture was stirred at this same temperature for 45 min. Dry oxetan-3-one (2.80 g, 38.84 mmol) in dry THF was added at −78° C. and stirred for 2 h. The mixture was quenched with saturated ammonium chloride solution (5 mL), diluted with DCM (150 mL), washed with brine (50 mL), and dried over sodium sulphate to obtain crude product. The residue was purified by Combi flash, and the product was eluted at 30% ethyl acetate/hexane as an oily liquid (0.7 g, 17%). LCMS m/z: 203.95 (M+H)⁺.

### Step-ii: Synthesis of 3-(2-Amino-5-chloropyridin-4-yl)oxetan-3-ol

To a stirred solution of 3-(5-chloro-2-fluoropyridin-4-yl)oxetan-3-ol (0.6 g, 2.95 mmol) in DMSO was added ammonium hydroxide (6 mL). The reaction mixture was stirred at RT for 36 h. The reaction mass was subsequently extracted with ethyl acetate and concentrated to yield pure product (0.2 g, 33%). ¹HNMR (300 MHz, DMSO-d₆): δ 6.43 (s, 1H), 6.34 (s, 1H), 6.16 (s, 2H), 5.52 (s, 1H), 4.92-4.90 (dd, 2H) 4.62-4.60 (dd, 2H).

### Intermediate I-9: Synthesis of 2-((2-amino-5-chloropyridin-4-yl)oxy)-N-methylpropanamide

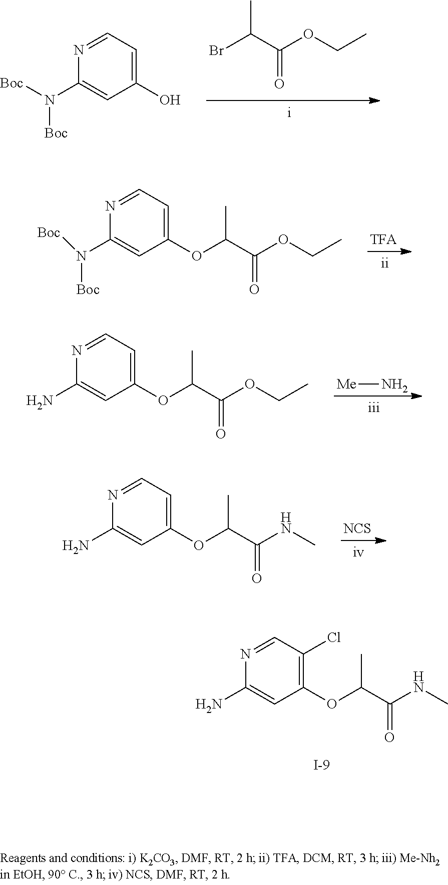

### Step-i: Synthesis of ethyl 2-((2-(bis(tert-butoxycarbonyl)amino)pyridin-4-yl)oxy)propionate

To a stirred solution of tert-butyl (tert-butoxycarbonyl)(4-hydroxypyridin-2-yl)carbamate (2 g, 6.440 mmol) (synthesis carried out as described in reference WO2018/209132 A1) in DMF (10 mL) was added K₂CO₃ (1.34 g, 9.66 mmol) followed by ethyl-2-bromo-propionate (1.4 g, 7.73 mmol) at 0° C. After the addition, the reaction mixture was stirred at RT for 2 h. The mixture was diluted with ethyl acetate (25 mL), washed with water and brine solution, dried over sodium sulphate, and concentrated. The crude product was purified by Combiflash with EtOAc:hexane as an eluent to obtain pure product (2.1 g, 80%). LCMS m/z: 411.15 (M+H)⁺.

### Step-ii: Synthesis of ethyl 2-((2-aminopyridin-4-yl)oxy)propionate

To a stirred solution of ethyl 2-((2-(bis(tert-butoxy carbonyl)amino)pyridin-4-yl)oxy)propionate (2.1 g, 5.11 mmol) in DCM (3.5 mL) was added TFA (3.5 mL). The reaction mixture was then stirred at RT for 3 h. The resulting mixture was diluted with DCM, washed with saturated NaHCO₃ and brine solution, dried over sodium sulphate, and concentrated under vacuum to give the titled product (1.1 g, 94%). LCMS m/z: 211.0 (M+H)⁺.

### Step-iii: Synthesis of 2-((2-aminopyridin-4-yl)oxy)-N-methylpropanamide

To a stirred solution of ethyl 2-((2-aminopyridin-4-yl)oxy)propionate (1.1 g, 5.2 mmol) in ethanol (5 mL) was added methylamine (1M) (10 mL) in a sealed tube. After addition, the mixture was heated at 90° C. for 3 h. The resulting mixture was concentrated to yield the desired product (0.8 g, 78%). LCMS m/z: 196.20 (M+H)⁺.

### Step-iv: Synthesis of 2-((2-amino-5-chloropyridin-4-yl)oxy)-N-methylpropanamide

To a stirred solution of 2-((2-aminopyridin-4-yl)oxy)-N-methylpropanamide (0.8 g, 4.1 mmol) in DMF (5 mL) was added NCS (0.6 g, 4.5 mmol). After the addition, the mixture was stirred at RT for 2 h. The resulting mixture was diluted with EtOAc, washed with 2N NaOH and brine solution, dried over sodium sulphate, and concentrated to obtain the titled product (0.5 g, 53%). LCMS m/z: 230.15 (M+H)⁺.

### Intermediate I-10: Synthesis of 2-amino-5-chloropyridin-4-yl dimethylcarbamate

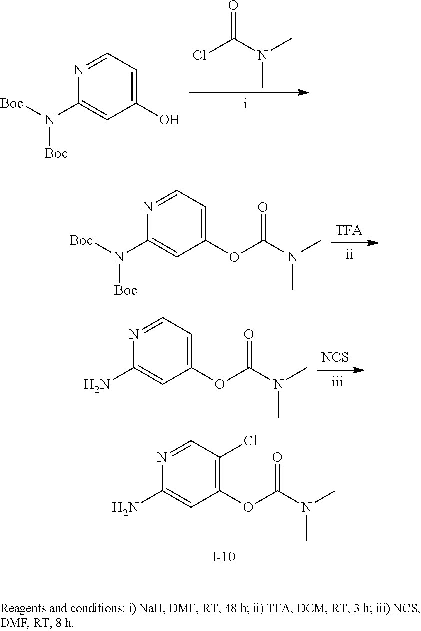

### Step-i: Synthesis of 2-(bis(tert-butoxycarbonyl)amino)pyridin-4-yl dimethylcarbamate

To a stirred solution of tert-butyl (tert-butoxycarbonyl)(4-hydroxypyridin-2-yl)carbamate (2.8 g, 9.022 mmol) (synthesis carried out as described in reference WO2018/209132 A1) in DMF (30 mL) was added sodium hydride (60%, dispersion in Paraffin Liquid) (0.415 g, 18.04 mmol) followed by dimethylcarbamic chloride (1.16 g, 10.82 mmol) at 0° C. After the addition, the reaction mixture was stirred at RT for 48 h. The mixture was diluted with ethyl acetate (50 mL), washed with water and brine solution, dried over sodium sulphate, and concentrated. The crude residue was purified by Combiflash with EtOAc:hexane as eluent to obtain pure product. (2.3 g, 66.84%). LCMS m/z: 382.2 (M+H)⁺.

### Step-ii: Synthesis of 2-aminopyridin-4-yl dimethylcarbamate

To a stirred solution of 2-(bis(tert-butoxycarbonyl)amino)pyridin-4-yl dimethylcarbamate (2.3 g, 6.03 mmol) in DCM (5 mL) was added TFA (2 mL). After the addition, the reaction mixture was stirred at RT for 3 h. The reaction mixture was diluted with DCM, washed with saturated NaHCO₃ and brine solutions, dried over sodium sulphate, and concentrated to obtain the titled product (0.8 g, 73.22%). LCMS m/z: 282.31 (M+H)⁺.

### Step-iii: Synthesis of 2-amino-5-chloropyridin-4-yl dimethylcarbamate

To a stirred solution of 2-aminopyridin-4-yl dimethylcarbamate (0.1 g, 0.55 mmol) in DMF (5 mL) was added NCS (0.088 g, 0.66 mmol). After the addition, the reaction mixture was stirred at RT for 8 h. The reaction mixture was diluted with EtOAc, washed with 2N NaOH and brine solution, dried over sodium sulphate, and concentrated to obtain the titled product (0.1 g, 84.01%). LCMS m/z: 216.41 (M+H)⁺.

### Intermediate I-11: Synthesis of N-(4-Bromo-5-chloropyridin-2-yl)acetamide

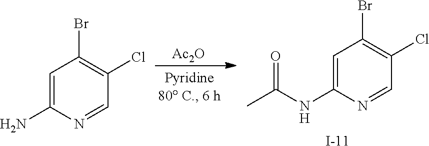

To a stirred solution of 4-bromo-5-chloropyridin-2-amine (30.0 g, 144.16 mmol) in pyridine (150 mL) at 0° C. was added acetic anhydride (22.14 g, 216.92 mmol), and the mixture was slowly warmed to RT and stirred at 80° C. for 6 h. The reaction mixture was quenched with ice water, and the precipitated solid was isolated by filtration and the solid was washed with cold water to yield pure titled product (25.0 g, 69.29%). ¹HNMR (400 MHz, DMSO-d₆) δ 10.85 (s, 1H), 8.48 (s, 2H), 2.10 (s, 3H); LCMS m/z: 250.8 (M+H).

### Intermediate I-12: Synthesis of 5-chloro-4-((1-methylpiperidin-4-yl)oxy)pyridin-2-amine

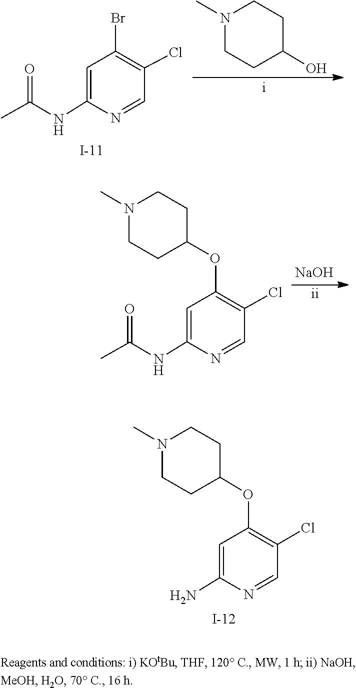

### Step-i: Synthesis of N-(5-chloro-4-((1-methylpiperidin-4-yl)oxy)pyridin-2-yl)acetamide

To a stirred solution of N-(4-bromo-5-chloropyridin-2-yl)acetamide (0.1 g, 4.01 mmol) in dry THF (5 mL) was added 1-methylpiperidin-4-ol (0.55 g, 4.809 mmol) and 1M potassium tert-butoxide in THF (1.08 g, 112.24 mmol). The reaction mixture was then stirred in a microwave reactor at 120° C. for 1 h, after which the mixture was quenched with ice water and extracted with EtOAc. The isolated organic layer was dried over sodium sulphate and concentrated. The crude product was purified by Combiflash with EtOAc:hexane as an eluent to obtain N-(5-chloro-4-((1-methylpiperidin-4-yl)oxy)pyridin-2-yl)acetamide (0.7 g, 61.52%). ¹H NMR (400 MHz, DMSO-d₆): δ 10.6 (bs, 1H), 8.2 (s, 1H), 7.95 (s, 1H), 4.55 (m, 1H) 2.52 (m, 2H), 2.25-2.23 (m, 2H), 2.25-2.0 (m, 3H), 2.10 (s, 3H), 1.98 (m, 2H), 1.76 (m, 2H); LCMS m/z: 284.1 (M+H)⁺.

### Step-ii: Synthesis of 5-chloro-4-((1-methylpiperidin-4-yl)oxy)pyridin-2-amine

To a solution of N-(5-chloro-4-((1-methylpiperidin-4-yl)oxy)pyridin-2-yl)acetamide (0.4 g, 1.409 mmol) in methanol (5 mL) was added NaOH (0.14 g, 40 mmol) at RT. Then, 2 drops of water were added, and the resulting mixture was heated to 70° C. for 16 h. The resulting mixture was cooled to RT, and the methanol was evaporated under vacuum. The crude product was washed with ether to yield a solid, which was dried under vacuum to yield the pure titled product (0.32 g, 93.89%). LCMS m/z: 242.05 (M+H)⁺.

### Intermediate I-13: Synthesis of 5-chloro-4-morpholinopyridin-2-amine

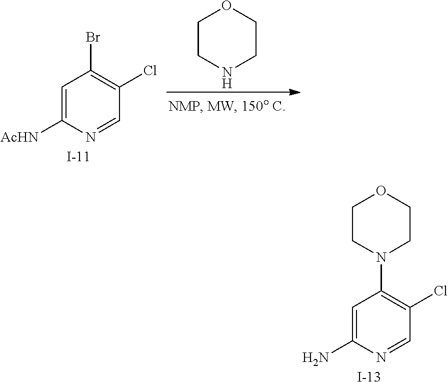

To a stirred solution of N-(4-bromo-5-chloropyridin-2-yl)acetamide (1 g, 4.82 mmol) in NMP (10 mL) was added morpholine (1.26 g, 14.46 mmol), and the resulting mixture was stirred at 150° C. for 1 h under microwave irradiation. After completion of the reaction, the mixture was quenched with ice water and extracted with EtOAc. The isolated organic layer was dried and concentrated. The crude product was purified by Combiflash with EtOAc:hexane as an eluent to obtain pure titled product (0.75 g, 72.83%). LCMS m/z: 214.20 (M+H)⁺.

### Intermediate I-14: Synthesis of 5-chloro-4-(4-methylpiperazin-1-yl)pyridin-2-amine

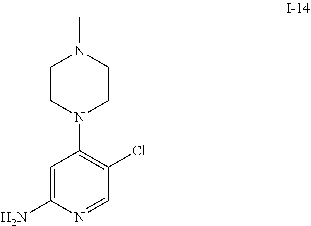

The above Intermediate I-14 was prepared by a procedure similar to the one described in the synthesis of I-13 and by using 1-methylpiperazine as a reagent; LCMS m/z: 227.1 (M+H)⁺.

### Intermediate I-15: Synthesis of 5-chloro-4-((1-methylpyrrolidin-3-yl)oxy)pyridin-2-amine

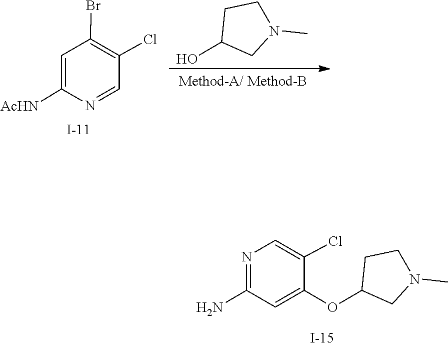

### Methods for Synthesis of Intermediate I-15:

Method-A: To a stirred solution of N-(4-bromo-5-chloropyridin-2-yl)acetamide (0.6 g, 2.4 mmol) in DMSO (3 mL) was added 1-methylpyrrolidin-3-ol (0.48 g, 4.8 mmol) followed by potassium tert-butoxide (LOM in THF) (7.2 mL, 7.2 mmol). Then, the resulting mixture was stirred under microwave irradiation at 140° C. for 1 h. The mixture was then cooled to RT, diluted with ice water and extracted with EtOAc. The organic layer was dried over sodium sulphate and concentrated, and the crude material was purified by Combiflash with an EtOAc:hexane eluent to yield pure titled product (0.18 g, 32%). ¹HNMR (400 MHz, DMSO-d₆) δ 7.74 (s, 1H), 6.04 (s, 1H), 5.99 (s, 2H), 4.83-4.79 (m, 3H), 2.78-2.64 (m, 2H), 2.26 (s, 3H), 1.79-1.76 (m, 2H); LCMS m/z: 228.08 (M+H)⁺.

Method-B: To a stirred solution of N-(4-bromo-5-chloropyridin-2-yl)acetamide (0.6 g, 2.4 mmol) in DMSO (5 mL) was added 1-methylpyrrolidin-3-ol (0.48 g, 4.8 mmol) followed by potassium tert-butoxide (LOM in THF) (7.2 mL, 7.2 mmol). The mixture was subsequently stirred at 100° C. for 16 h in a sealed tube before being cooled to RT, diluted with ice water, and extracted with EtOAc. The organic layer was dried over sodium sulphate and concentrated. The crude material was purified by Combiflash with an EtOAc:hexane eluent to get pure titled product (0.23 g, 55%). ¹HNMR (400 MHz, DMSO-d₆): δ 7.73 (s, 11H), 6.03 (s, 11H), 5.97 (s, 2H), 4.81-4.78 (m, 3H), 2.77-2.62 (m, 2H), 2.24 (s, 3H), 1.78-1.74 (in, 2H); LCMS m/z: 228.08 (M+H)⁺.

The intermediates listed in below Table B were prepared by a procedure similar to the one described in the synthesis of Intermediate 1-15 (Method A or Method B) with appropriate variations in reactants/reagents. The characterization data of the intermediates are summarized herein.

TABLE B

| Intermediate | | | Method | Characterization |
|---|---|---|---|---|
| No. | Reagent used | Intermediate Structure | used | Data |
| B-1 | 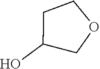 | 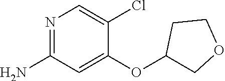 [SMILES: Nc1cc(OC2CCOC2)c(Cl)cn1] | A | LCMS m/z: 215.10 (M + H)⁺ |
| B-2 | 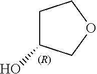 | 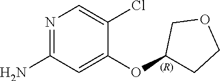 | A | LCMS m/z: 215.10 (M + H)⁺ |
| B-3 | 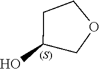 | 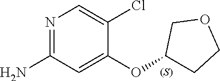 | A | LCMS m/z: 215.10 (M + H)⁺ |
| B-4 | 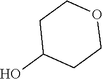 | 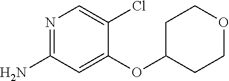 [SMILES: Nc1cc(OC2CCOCC2)c(Cl)cn1] | B | LCMS m/z: 229.10 (M + H)⁺ |
| B-5 | 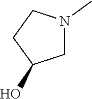 | 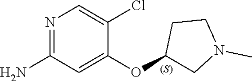 | B | LCMS m/z: 228.50 (M + H)⁺ |
| B-6 | 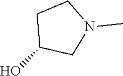 | 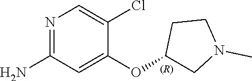 | B | LCMS m/z: 228.50 (M + H)⁺ |
| B-7 | 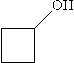 | 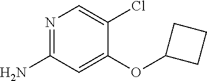 [SMILES: Nc1cc(OC2CCC2)c(Cl)cn1] | B | LCMS m/z: 199.50 (M + H)⁺ |
| B-8 | 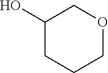 | 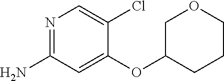 [SMILES: Nc1cc(OC2CCCOC2)c(Cl)cn1] | A | LCMS m/z: 229.00 (M + H)⁺ |
| B-9 | 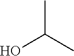 | 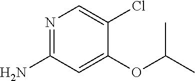 | B | LCMS m/z: 187.00 (M + H)⁺ |
| B-10 | 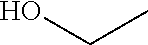 | 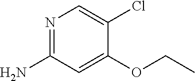 | B | LCMS m/z: 173.10 (M + H)⁺ |
| B-11 | 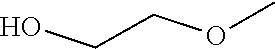 | 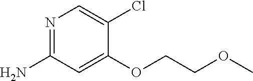 | B | LCMS m/z: 203.40 (M + H)⁺ |
| B-12 | 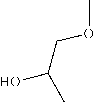 | 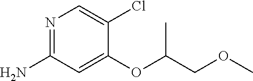 | B | LCMS m/z: 217.30 (M + H)⁺ |
| B-13 | 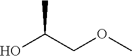 | 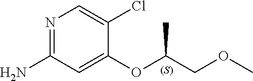 | B | LCMS m/z: 217.30 (M + H)⁺ |
| B-14 | 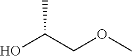 | 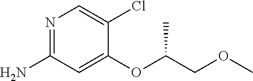 | B | LCMS m/z: 217.30 (M + H)⁺ |
| B-15 | 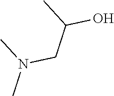 | 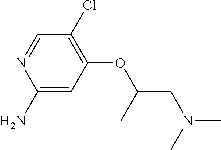 | B | LCMS m/z: 230.50 (M + H)⁺ |
| B-16 | 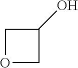 | 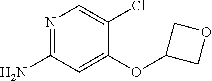 [SMILES: Nc1cc(OC2COC2)c(Cl)cn1] | B | LCMS m/z: 201.40 (M + H)⁺ |
| B-17 | 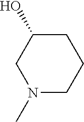 | 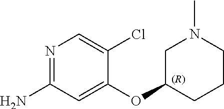 | B | LCMS m/z: 242.40 (M + H)⁺ |
| B-18 | 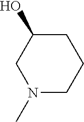 | 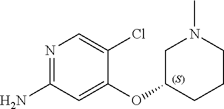 | B | LCMS m/z: 242.40 (M + H)⁺ |
| B-19 | 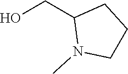 | 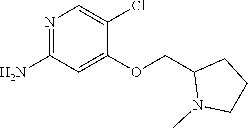 [SMILES: CN1CCCC1COc1cc(N)ncc1Cl] | A | LCMS m/z: 242.50 (M + H)⁺ |
| B-20 | 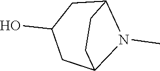 | 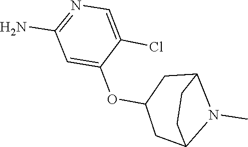 [SMILES: CN1C2CCC1CC(Oc1cc(N)ncc1Cl)C2] | A | LCMS m/z: 268.60 (M + H)⁺ |
| B-21 | I-3 | 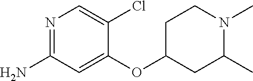 [SMILES: CC1CC(Oc2cc(N)ncc2Cl)CCN1C] | A | LCMS m/z: 256.60 (M + H)⁺ |
| B-22 | I-4 | 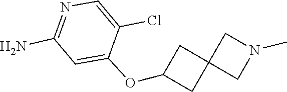 [SMILES: CN1CC2(CC(Oc3cc(N)ncc3Cl)C2)C1] | A | LCMS m/z: 254.70 (M + H)⁺ |
| B-23 | 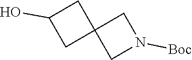 [SMILES: CC(C)(C)OC(=O)N1CC2(CC(O)C2)C1] | 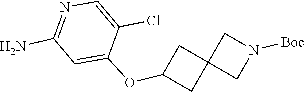 [SMILES: CC(C)(C)OC(=O)N1CC2(CC(Oc3cc(N)ncc3Cl)C2)C1] | A | LCMS m/z: 340.70 (M + H)⁺ |
| B-24 | 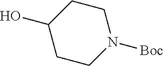 [SMILES: CC(C)(C)OC(=O)N1CCC(O)CC1] | 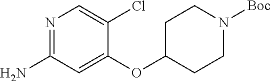 [SMILES: CC(C)(C)OC(=O)N1CCC(Oc2cc(N)ncc2Cl)CC1] | A | LCMS m/z: 328.70 (M + H)⁺ |
| B-25 | 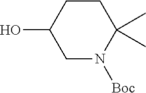 | 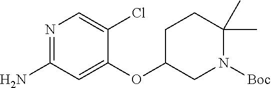 [SMILES: CC(C)(C)OC(=O)N1CC(Oc2cc(N)ncc2Cl)CCC1(C)C] | A | LCMS m/z: 356.70 (M + H)⁺ |
| B-26 | 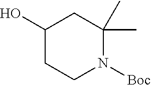 | 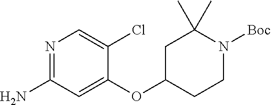 [SMILES: CC(C)(C)OC(=O)N1CCC(Oc2cc(N)ncc2Cl)CC1(C)C] | A | LCMS m/z: 356.70 (M + H)⁺ |

### Intermediate I-16: Synthesis of 5-chloro-4-methoxypyridin-2-amine

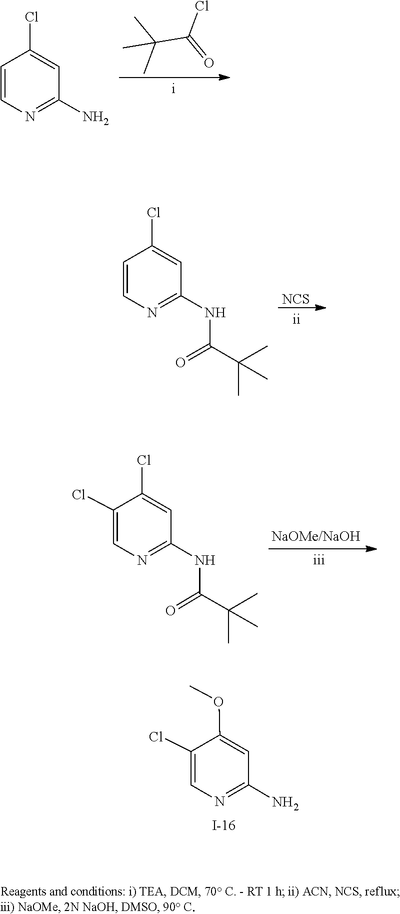

### Step-i: Synthesis of N-(4-chloropyridin-2-yl)pivalamide

To a solution of 4-chloropyridin-2-amine (30 g, 234 mmol) in DCM (200 mL) was added TEA (48.8 mL, 351 mmol) and pivaloyl chloride (37.7 mL, 304 mmol) at 0° C., and the resulting mixture was stirred at room temperature overnight. The reaction solution was partitioned between DCM and water. The combined organic layer was washed with brine, dried over Na₂SO₄, and concentrated in vacuo. The resulting residue was triturated with n-pentane to yield the pure product as a pale-yellow solid (44 g, 88.5%); LCMS m/z: 213.20 (M+H)⁺.

### Step-ii: Synthesis of N-(4,5-dichloropyridin-2-yl)pivalamide

To a stirred solution of N-(4-chloropyridin-2-yl)pivalamide (44 g, 206.9 mmol) in acetonitrile (250 mL) was added N-chlorosuccinimide (55.25 g, 133.56 mmol). The mixture was stirred at reflux overnight. The mixture was cooled, combined with water, and extracted with ethyl acetate. The combined organic layers were dried over sodium sulfate, evaporated under reduced pressure, and purified by gradient column chromatography using ethyl acetate in n-hexane as eluent to afford N-(4,5-dichloropyridin-2-yl)pivalamide as a white solid (41.2 g, 80.6%); LCMS m/z: 247.10 (M+H)⁺.

### Step-iii: Synthesis of 5-chloro-4-methoxypyridin-2-amine

To a solution of N-(4,5-dichloropyridin-2-yl)pivalamide (41 g, 5.2 mmol) in DMSO (35 mL) was added sodium methanolate (25% in Methanol) (53.6 mL, 248.9 mmol). After the addition, the mixture was stirred at 90° C. for 4 h. The vessel was cooled to RT, 10 mL of 2N NaOH were added, and the resulting mixture was stirred at 90° C. for 2 h. Upon quenching with ice cold water, a white solid precipitated. The suspension was filtered, and the solid was washed with n-pentane before being dried under vacuum to yield a free-flowing white solid (20.2 g, 78.69%); LCMS m/z: 159.10 (M+H)⁺.

### Intermediate I-17: Synthesis of tert-butyl ((5-bromopyridin-2-yl)methyl)carbamate

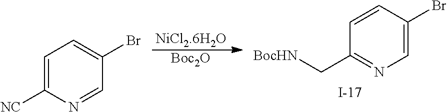

To a solution of 5-bromo-2-cyanopyridine (5.0 g, 27.32 mmol) in methanol (50 mL) at 0° C. were added nickel(II) chloride hexahydrate (649 mg, 27.32 mmol), di-tert-butyl dicarbonate (11.9 g, 54.64 mmol), and sodium borohydride (2.06 g, 54.64 mmol). The reaction mixture was stirred at RT for 18 h. The mixture was concentrated under reduced pressure, and the residue was diluted with ethyl acetate and water. The layers were separated, and the aqueous layer was extracted twice with ethyl acetate. The combined organic extracts were dried over anhydrous sodium sulphate, filtered, and concentrated. The crude compound was purified by silica gel column chromatography by eluting with 10-20% ethyl acetate-hexane to afford the title compound (3.8 g, 49.23%); LCMS: m/z=289 (M+H)⁺.

### Intermediate I-18: Synthesis of tert-butyl ((5-bromopyridin-2-yl)methyl)(methyl)carbamate

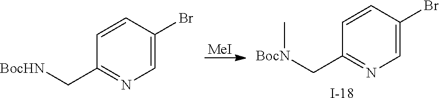

A solution of tert-butyl ((5-bromopyridin-2-yl)methyl)carbamate (1 g, 3.48 mmol) in anhydrous DMF was cooled under N₂ atmosphere to 0° C. and treated with sodium hydride (60% dispersion in oil) (0.13 g, 5.22 mmol). The solution was stirred for 15 min, treated dropwise with methyl iodide (0.74 g, 5.22 mmol), stirred at ambient temperature for 12 h, and quenched cautiously with water. The resulting mixture was partitioned between ethyl acetate and water, dried over Na₂SO₄, and concentrated in vacuo. Chromatography on silica gel provided the desired compound (0.8 g, 76.28%) as a colorless oil; LCMS: m/z=303.20 (M+H)⁺.

### Intermediate I-19: tert-butyl ((5-bromopyridin-2-yl)methyl)(isopropyl)carbamate

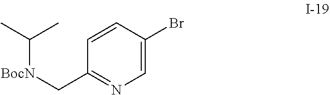 [SMILES: CC(C)N(Cc1ccc(Br)cn1)C(=O)OC(C)(C)C]

The above Intermediate I-19 was prepared by a procedure similar to the one described in the synthesis of Intermediate I-18 using isopropyl iodide as a reagent; LCMS: m/z=330.20 (M+H)⁺.

### Intermediate I-20: Synthesis of tert-butyl ((5-(4,4,5,5-tetramethyl-1,3,2-dioxaborolan-2-yl)pyridin-2-yl)methyl)carbamate

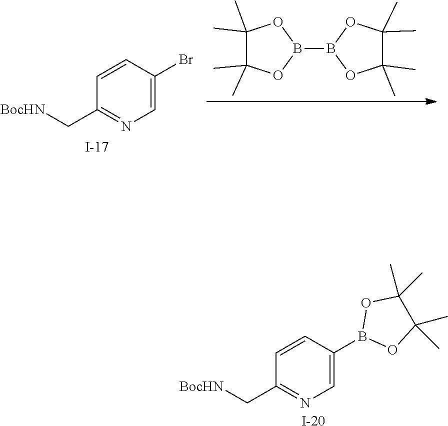

To a stirred solution of tert-butyl ((5-bromopyridin-2-yl)methyl)carbamate (I-17) (3.8 g, 13.23 mmol) in dioxane (50 mL) was added KOAc (2.59 g, 26.46 mmol) and 4,4,4′,4′,5,5,5′,5′-octamethyl-2,2′-bi(1,3,2-dioxaborolane) (4.03 g, 15.88 mmol) at RT, and the resulting mixture was degassed for 30 min. Then [1,1′-Bis(diphenylphosphino) ferrocene]dichloropalladium (II) (1.0 8 g, 1.32 mmol) was added. The mixture was again degassed for 5 min and subsequently stirred at 100° C. for 3 h. The progress of the reaction was monitored by TLC. After completion of the reaction, the mixture was poured into water (100 mL) and filtered through celite. The celite bed was washed with ethyl acetate (100 mL), and the filtrate was separated into two layers. The organic layer was washed with brine solution (200 mL), dried over sodium sulphate, and concentrated under reduced pressure. The crude compound was purified by silica gel column chromatography by eluting with 10-30% ethyl acetate-hexane to afford the title compound (1.8 g, 40.70%). LCMS: m/z=335.20 (M+H)⁺.

The intermediates listed in below Table C were prepared by a procedure similar to the one described in the synthesis of Intermediate I-20. The characterization data of the intermediates are summarized herein the below table.

TABLE C

| Inter- | | | Character- |
|---|---|---|---|
| mediate | SM | | ization |
| No. | used | Intermediate Structure | Data |
| C-1 | I-18 | 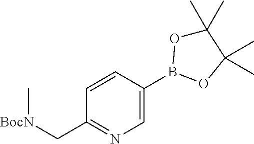 [SMILES: CN(Cc1ccc(B2OC(C)(C)C(C)(C)O2)cn1)C(=O)OC(C)(C)C] | LCMS: m/z = 349.10 (M + H)⁺. |
| C-2 | I-19 | 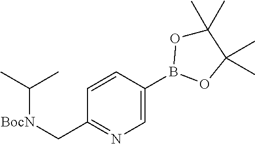 [SMILES: CC(C)N(Cc1ccc(B2OC(C)(C)C(C)(C)O2)cn1)C(=O)OC(C)(C)C] | LCMS: m/z = 377.10 (M + H)⁺. |

### Intermediate I-21: Synthesis of 6-(4,4,5,5-tetramethyl-1,3,2-dioxaborolan-2-yl)picolinonitrile

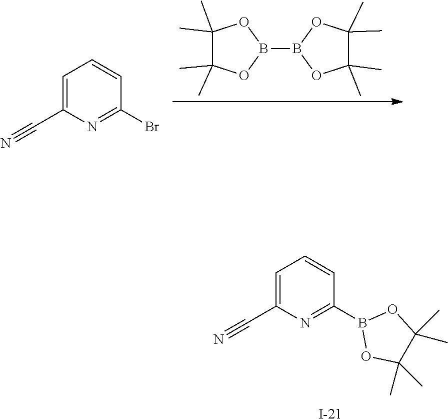

To a solution of 6-bromopicolinonitrile (10 g, 54.62 mmol) in 1,4-dioxane (100 ml) were added bis(pinacolato)diboron (20.81 g, 81.96 mmol) and potassium acetate (10.72 g, 98.14 mmol) at RT. The reaction mixture was degassed for 15 min before addition of Pd₂(dba)₃ (2.5 g, 2.73 mmol) and Tricyclohexylphosphine (1.53 g, 5.46 mmol). The reaction was heated at 90° C. for 2 h. The resulting reaction mixture was cooled to RT and filtered through a celite pad. The filtrate was evaporated and triturated first with diethyl ether, then twice with n-pentane to yield a light-yellow solid. The product was used without further purification (10.1 g, 80.34%); ¹HNMR (DMSO-d₆, 400 MHz): δ 8.04-8.01 (m, 1H), 7.85-7.81 (m, 1H), 7.75-7.72 (m, 1H), 1.42 (s, 12H).

### Intermediate I-22: Synthesis of 4-(4,4,5,5-tetramethyl-1,3,2-dioxaborolan-2-yl)-3,6-dihydropyridine-1(2H)-carbonitrile

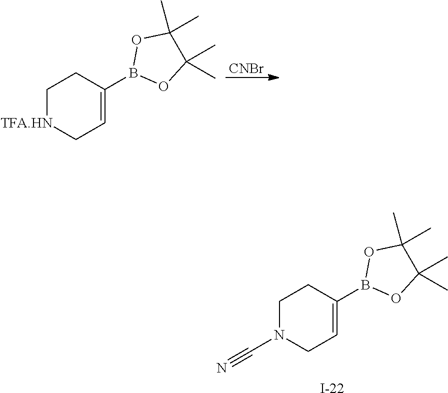

To a solution of the trifluoroacetate salt of 4-(4,4,5,5-tetramethyl-1,3,2-dioxaborolan-2-yl)-1,2,3,6-tetrahydropyridine (2 g, 9.56 mmol) in DCM (5 mL) was added DIPEA (3.70 g, 28.69 mmol) at −10° C. After stirring for 10 min, cyanogen bromide (1.10 g, 10.52 mmol) was added at −10° C., and stirring was continued for 30 min. The reaction was monitored by TLC, and after completion, the mixture was quenched with aq. NaHCO₃ and extracted with DCM. The isolated organic layer was dried and concentrated. The crude product was purified by combiflash chromatography by eluting with 10-30% ethyl acetate-hexane to afford the title compound (1.6 g, 71.46%). LCMS: m/z=235.20 (M+H)⁺.

### Intermediate I-23: Synthesis of N-(5-chloropyridin-2-yl)-2-(6-chloropyridin-3-yl) propanamide

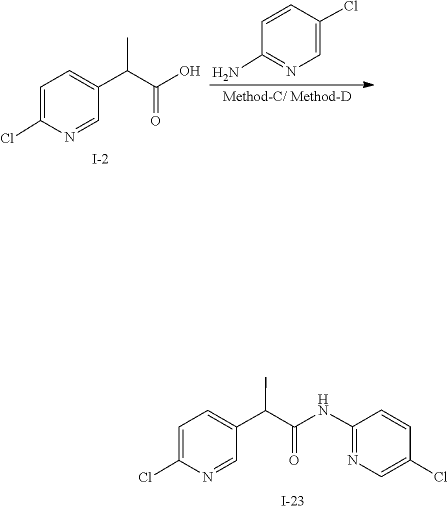

### Methods for Synthesis of Intermediate I-23:

Method C: To a stirred solution of 2-(6-chloropyridin-3-yl) propanoic acid (0.5 g, 2.70 mmol) in DCM (5 mL) at 0° C. was added oxalyl chloride (0.68 g, 5.4 mmol) and a catalytic amount of DMF. The mixture was stirred for 2 h-4 h at RT then evaporated completely under reduced pressure. The residue was re-dissolved in DCM and added slowly to a cooled solution of 5-chloropyridin-2-amine (0.275 g, 2.16 mmol) and DIPEA (1.04 mL, 8.10 mmol) in DCM (10 mL) at 0° C. over 5 min. The mixture was stirred for 16 h at RT and diluted with DCM. The organic layer was washed with saturated NaHCO₃ solution, and brine solution. The organic layer was dried over sodium sulphate, and concentrated. The crude material was purified by Combiflash with EtOAc:hexane as an eluent to obtain pure titled compound (0.42 g, 52%). LCMS m/z: 296.91 (M+H)⁺.

Method D: To a mixture of 2-(6-chloropyridin-3-yl) propanoic acid (1 g, 5.830 mmol) in DMF (10 mL) was added DIPEA (1.5 g, 11.63 mmol) followed by T₃P (50% in EtOAc) (4.26 g, 13.4 mmol) at 0° C. over a period of 5 min. The reaction mass was stirred for 10 min at this same temperature, followed by lot wise addition of 5-chloropyridin-2-amine (0.75 g, 5.83 mmol) over a period of 5 min. After the addition, the reaction mixture was warmed to room temperature and stirred overnight. The reaction was quenched with ice water, and the aqueous layer was extracted with ethyl acetate twice. The combined organic layers were dried over anhydrous sodium sulphate and concentrated under reduced pressure to afford crude compound. This was further purified by using Combiflash with EtOAc:hexane as eluent to yield pure titled compound (0.47 g, 58%). LCMS m/z: 296.91 (M+H)⁺.

The intermediates listed below in Table D were prepared by procedures similar to the one described in the synthesis of Intermediate I-23 (Method-C or Method-D) with appropriate acid and amine reagents with appropriate variations. The characterization data of the intermediates are summarized herein the below table.

TABLE D

| Int- | | |
|---|---|---|
| No. | Acid used | Amine used |
| D-1 | 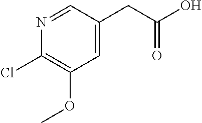 | B-2 |
| D-2 | 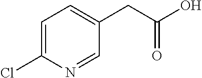 [SMILES: O=C(O)Cc1ccc(Cl)nc1] | I-16 |
| D-3 | 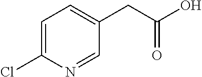 [SMILES: O=C(O)Cc1ccc(Cl)nc1] | 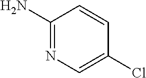 |
| D-4 | 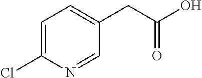 [SMILES: O=C(O)Cc1ccc(Cl)nc1] | B-1 |
| D-5 | 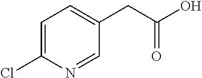 [SMILES: O=C(O)Cc1ccc(Cl)nc1] | B-4 |
| D-6 | 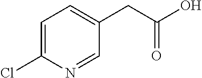 [SMILES: O=C(O)Cc1ccc(Cl)nc1] | I-13 |
| D-7 | 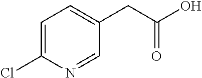 [SMILES: O=C(O)Cc1ccc(Cl)nc1] | I-14 |
| D-8 | 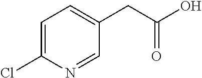 [SMILES: O=C(O)Cc1ccc(Cl)nc1] | I-12 |
| D-9 | 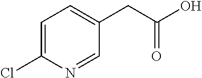 [SMILES: O=C(O)Cc1ccc(Cl)nc1] | I-15 |
| D-10 | 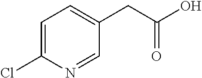 [SMILES: O=C(O)Cc1ccc(Cl)nc1] | B-5 |
| D-11 | 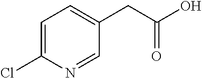 [SMILES: O=C(O)Cc1ccc(Cl)nc1] | B-6 |
| D-12 | 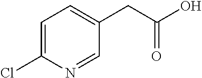 [SMILES: O=C(O)Cc1ccc(Cl)nc1] | B-16 |
| D-13 | 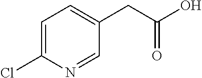 [SMILES: O=C(O)Cc1ccc(Cl)nc1] | B-12 |
| D-14 | 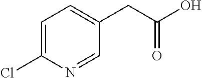 [SMILES: O=C(O)Cc1ccc(Cl)nc1] | B-14 |
| D-15 | 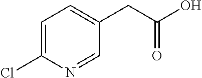 [SMILES: O=C(O)Cc1ccc(Cl)nc1] | B-13 |
| D-16 | 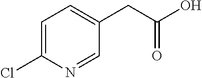 [SMILES: O=C(O)Cc1ccc(Cl)nc1] | B-11 |
| D-17 | 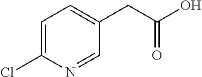 [SMILES: O=C(O)Cc1ccc(Cl)nc1] | B-10 |
| D-18 | 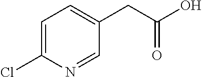 [SMILES: O=C(O)Cc1ccc(Cl)nc1] | B-9 |
| D-19 | 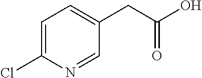 [SMILES: O=C(O)Cc1ccc(Cl)nc1] | B-7 |
| D-20 | 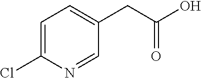 [SMILES: O=C(O)Cc1ccc(Cl)nc1] | I-7 |
| D-21 | 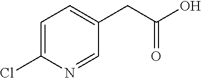 [SMILES: O=C(O)Cc1ccc(Cl)nc1] | I-8 |
| D-22 | 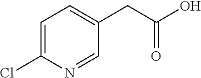 [SMILES: O=C(O)Cc1ccc(Cl)nc1] | I-9 |
| D-23 | 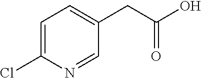 [SMILES: O=C(O)Cc1ccc(Cl)nc1] | B-2 |
| D-24 | 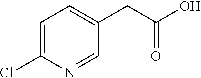 [SMILES: O=C(O)Cc1ccc(Cl)nc1] | B-3 |
| D-25 | 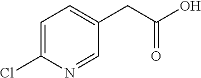 [SMILES: O=C(O)Cc1ccc(Cl)nc1] | B-15 |
| D-26 | 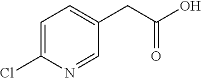 [SMILES: O=C(O)Cc1ccc(Cl)nc1] | B-18 |
| D-27 | 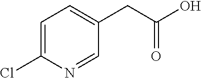 [SMILES: O=C(O)Cc1ccc(Cl)nc1] | B-17 |
| D-28 | 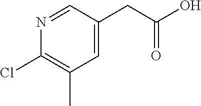 | 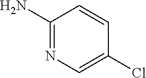 |
| D-29 | 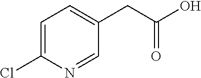 [SMILES: O=C(O)Cc1ccc(Cl)nc1] | B-8 |
| D-30 | 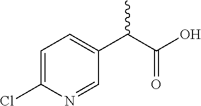 | I-16 |
| D-31 | 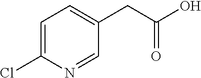 [SMILES: O=C(O)Cc1ccc(Cl)nc1] | B-19 |
| D-32 | 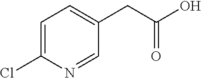 [SMILES: O=C(O)Cc1ccc(Cl)nc1] | B-20 |
| D-33 | 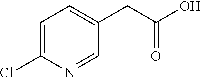 [SMILES: O=C(O)Cc1ccc(Cl)nc1] | B-21 |
| D-34 | 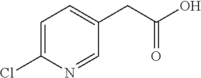 [SMILES: O=C(O)Cc1ccc(Cl)nc1] | B-22 |
| D-35 | 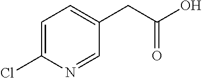 [SMILES: O=C(O)Cc1ccc(Cl)nc1] | I-10 |
| D-36 | 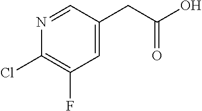 [SMILES: O=C(O)Cc1cnc(Cl)c(F)c1] | 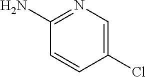 |
| D-37 | 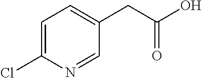 [SMILES: O=C(O)Cc1ccc(Cl)nc1] | 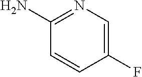 |
| D-38 | 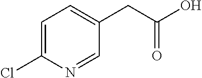 [SMILES: O=C(O)Cc1ccc(Cl)nc1] | 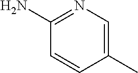 |
| D-39 | 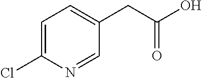 [SMILES: O=C(O)Cc1ccc(Cl)nc1] | 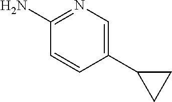 |
| D-40 | 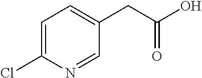 [SMILES: O=C(O)Cc1ccc(Cl)nc1] | 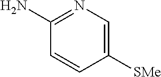 |
| D-41 | 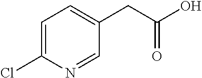 [SMILES: O=C(O)Cc1ccc(Cl)nc1] | 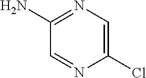 |
| D-42 | 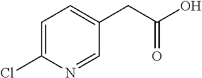 [SMILES: O=C(O)Cc1ccc(Cl)nc1] | I-1 |
| D-43 | 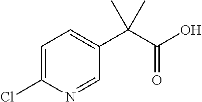 | I-16 |
| D-44 | 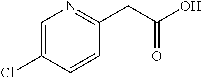 [SMILES: O=C(O)Cc1ccc(Cl)cn1] | I-16 |
| D-45 | 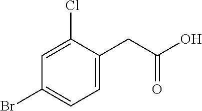 [SMILES: O=C(O)Cc1ccc(Br)cc1Cl] | I-16 |
| D-46 | 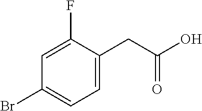 [SMILES: O=C(O)Cc1ccc(Br)cc1F] | I-16 |
| D-47 | 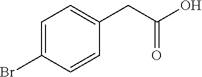 [SMILES: O=C(O)Cc1ccc(Br)cc1] | I-16 |
| D-48 | 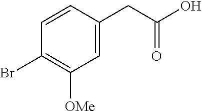 [SMILES: COc1cc(CC(=O)O)ccc1Br] | I-16 |
| D-49 | I-6 | I-16 |
| D-50 | 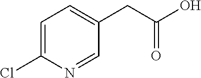 [SMILES: O=C(O)Cc1ccc(Cl)nc1] | B-23 |
| D-51 | 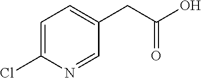 [SMILES: O=C(O)Cc1ccc(Cl)nc1] | B-24 |
| D-52 | 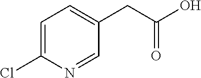 [SMILES: O=C(O)Cc1ccc(Cl)nc1] | B-25 |
| D-53 | 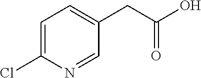 [SMILES: O=C(O)Cc1ccc(Cl)nc1] | B-26 |
| D-54 | 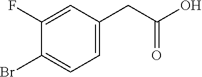 [SMILES: O=C(O)Cc1ccc(Br)c(F)c1] | I-16 |

| | | | Character- |
|---|---|---|---|
| Int- | | Method | ization |
| No. | Intermediate structure | used | Data |
| D-1 | 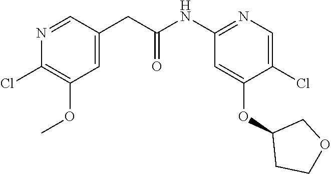 [SMILES: COc1cc(CC(=O)Nc2cc(O[C@@H]3CCOC3)c(Cl)cn2)cnc1Cl] | D | LCMS m/z: 399.00 (M + H) ⁺ |
| D-2 | 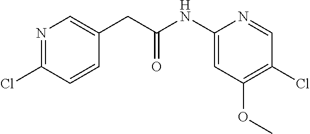 [SMILES: COc1cc(NC(=O)Cc2ccc(Cl)nc2)ncc1Cl] | D | LCMS m/z: 312.20 (M + H) ⁺ |
| D-3 | 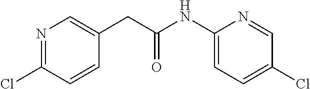 [SMILES: O=C(Cc1ccc(Cl)nc1)Nc1ccc(Cl)cn1] | D | LCMS m/z: 282.10 (M + H) ⁺ |
| D-4 | 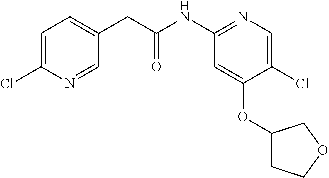 [SMILES: O=C(Cc1ccc(Cl)nc1)Nc1cc(OC2CCOC2)c(Cl)cn1] | D | LCMS m/z: 368.20 (M + H)⁺ |
| D-5 | 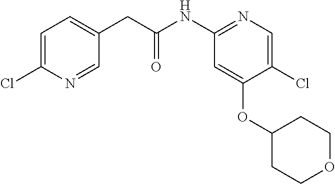 [SMILES: O=C(Cc1ccc(Cl)nc1)Nc1cc(OC2CCOCC2)c(Cl)cn1] | D | LCMS m/z: 382.30 (M + H)⁺ |
| D-6 | 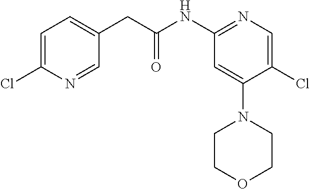 [SMILES: O=C(Cc1ccc(Cl)nc1)Nc1cc(N2CCOCC2)c(Cl)cn1] | D | LCMS m/z: 367.30 (M + H)⁺ |
| D-7 | 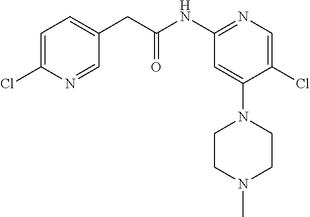 [SMILES: CN1CCN(c2cc(NC(=O)Cc3ccc(Cl)nc3)ncc2Cl)CC1] | D | LCMS m/z: 380.20 (M + H) ⁺ |
| D-8 | 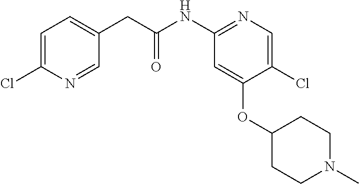 [SMILES: CN1CCC(Oc2cc(NC(=O)Cc3ccc(Cl)nc3)ncc2Cl)CC1] | D | LCMS m/z: 395.20 (M + H) ⁺ |
| D-9 | 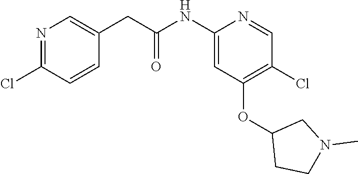 [SMILES: CN1CCC(Oc2cc(NC(=O)Cc3ccc(Cl)nc3)ncc2Cl)C1] | D | LCMS m/z: 381.30 (M + H) ⁺ |
| D-10 | 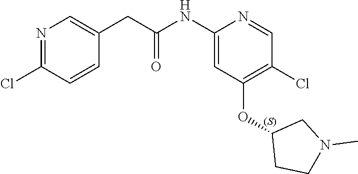 | D | LCMS m/z: 381.30 (M + H) ⁺ |
| D-11 | 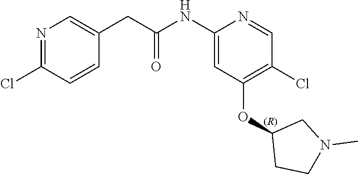 | D | LCMS m/z: 381.30 (M + H) ⁺ |
| D-12 | 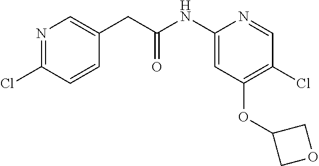 [SMILES: O=C(Cc1ccc(Cl)nc1)Nc1cc(OC2COC2)c(Cl)cn1] | D | LCMS m/z: 354.20 (M + H) ⁺ |
| D-13 | 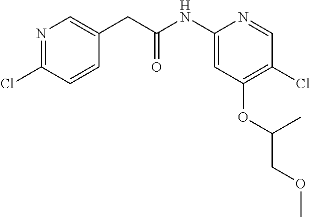 [SMILES: COCC(C)Oc1cc(NC(=O)Cc2ccc(Cl)nc2)ncc1Cl] | D | LCMS m/z: 370.10 (M + H) ⁺ |
| D-14 | 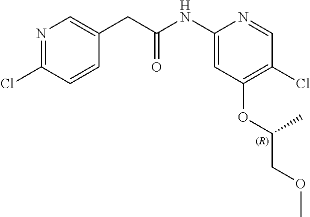 | D | LCMS m/z: 370.10 (M + H) ⁺ |
| D-15 | 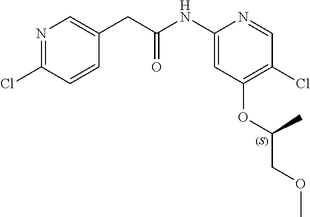 | D | LCMS m/z: 370.10 (M + H) ⁺ |
| D-16 | 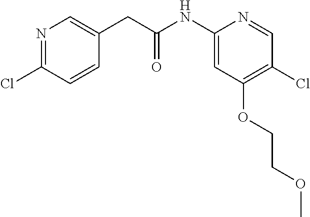 [SMILES: COCCOc1cc(NC(=O)Cc2ccc(Cl)nc2)ncc1Cl] | D | LCMS m/z: 356.20 (M + H) ⁺ |
| D-17 | 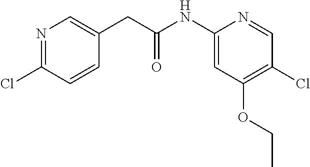 [SMILES: CCOc1cc(NC(=O)Cc2ccc(Cl)nc2)ncc1Cl] | D | LCMS m/z: 326.20 (M + H) ⁺ |
| D-18 | 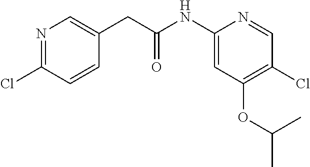 [SMILES: CC(C)Oc1cc(NC(=O)Cc2ccc(Cl)nc2)ncc1Cl] | D | LCMS m/z: 340.30 (M + H) ⁺ |
| D-19 | 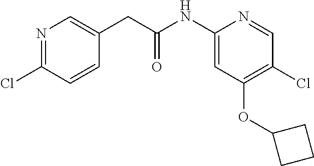 [SMILES: O=C(Cc1ccc(Cl)nc1)Nc1cc(OC2CCC2)c(Cl)cn1] | D | LCMS m/z: 352.30 (M + H) ⁺ |
| D-20 | 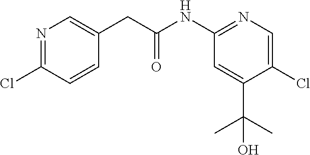 [SMILES: CC(C)(O)c1cc(NC(=O)Cc2ccc(Cl)nc2)ncc1Cl] | D | LCMS m/z: 340.30 (M + H) ⁺ |
| D-21 | 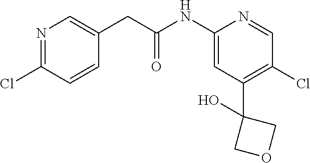 [SMILES: O=C(Cc1ccc(Cl)nc1)Nc1cc(C2(O)COC2)c(Cl)cn1] | D | 354.10 (M + H) ⁺ LCMS m/z: |
| D-22 | 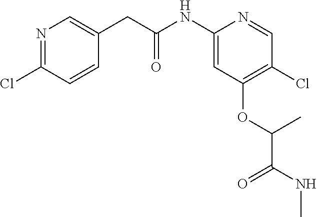 [SMILES: CNC(=O)C(C)Oc1cc(NC(=O)Cc2ccc(Cl)nc2)ncc1Cl] | D | LCMS m/z: 383.30 (M + H) ⁺ |
| D-23 | 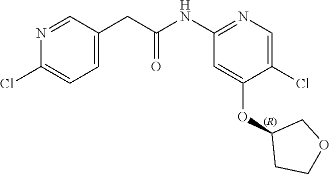 | D | LCMS m/z: 369.10 (M + H)⁺ |
| D-24 | 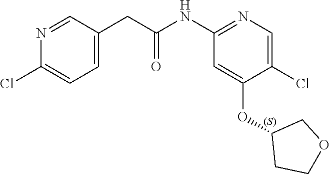 | D | LCMS m/z: 369.10 (M + H)⁺ |
| D-25 | 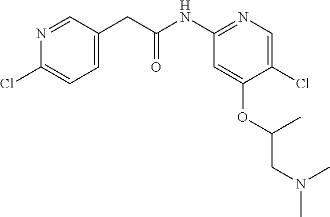 [SMILES: CC(CN(C)C)Oc1cc(NC(=O)Cc2ccc(Cl)nc2)ncc1Cl] | D | LCMS m/z: 384.00 (M + H)⁺ |
| D-26 | 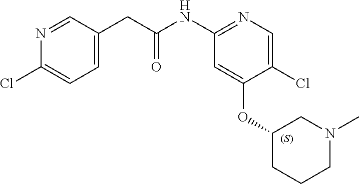 | D | LCMS m/z: 396.10 (M + H)⁺ |
| D-27 | 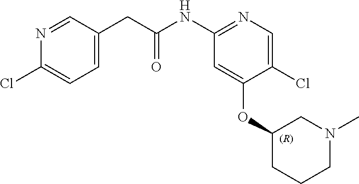 | D | LCMS m/z: 396.10 (M + H)⁺ |
| D-28 | 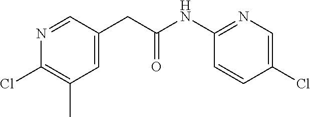 [SMILES: Cc1cc(CC(=O)Nc2ccc(Cl)cn2)cnc1Cl] | C | LCMS m/z: 295.80 (M + H)⁺ |
| D-29 | 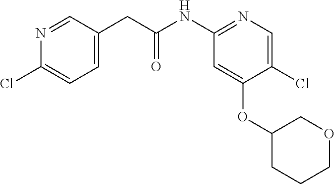 [SMILES: O=C(Cc1ccc(Cl)nc1)Nc1cc(OC2CCCOC2)c(Cl)cn1] | D | LCMS m/z: 383.10 (M + H) ⁺ |
| D-30 | 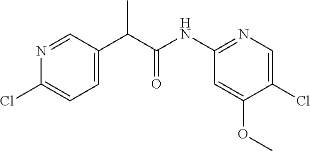 [SMILES: COc1cc(NC(=O)C(C)c2ccc(Cl)nc2)ncc1Cl] | D | LCMS m/z: 326.90 (M + H) ⁺ |
| D-31 | 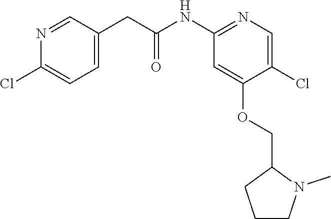 [SMILES: CN1CCCC1COc1cc(NC(=O)Cc2ccc(Cl)nc2)ncc1Cl] | D | LCMS m/z: 396.10 (M + H) ⁺ |
| D-32 | 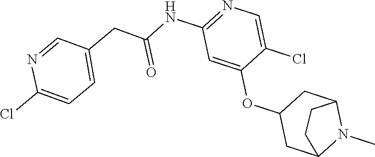 [SMILES: CN1C2CCC1CC(Oc1cc(NC(=O)Cc3ccc(Cl)nc3)ncc1Cl)C2] | D | LCMS m/z: 422.10 (M + H) ⁺ |
| D-33 | 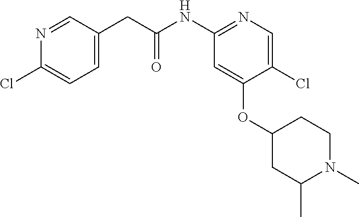 [SMILES: CC1CC(Oc2cc(NC(=O)Cc3ccc(Cl)nc3)ncc2Cl)CCN1C] | D | LCMS m/z: 410.10 (M + H) ⁺ |
| D-34 | 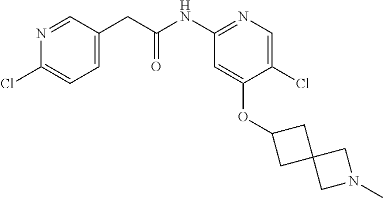 [SMILES: CN1CC2(CC(Oc3cc(NC(=O)Cc4ccc(Cl)nc4)ncc3Cl)C2)C1] | D | LCMS m/z: 408.10 (M + H) ⁺ |
| D-35 | 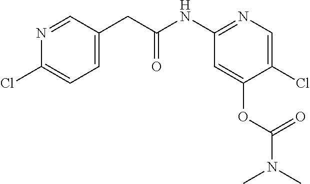 [SMILES: CN(C)C(=O)Oc1cc(NC(=O)Cc2ccc(Cl)nc2)ncc1Cl] | D | LCMS m/z: 370.10 (M + H) ⁺ |
| D-36 | 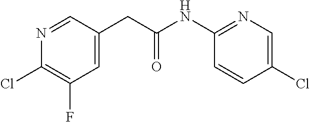 [SMILES: O=C(Cc1cnc(Cl)c(F)c1)Nc1ccc(Cl)cn1] | C | LCMS m/z: 300.80 (M + H) ⁺ |
| D-37 | 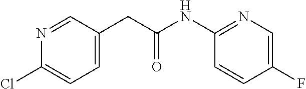 [SMILES: O=C(Cc1ccc(Cl)nc1)Nc1ccc(F)cn1] | D | LCMS m/z: 266.40 (M + H) ⁺ |
| D-38 | 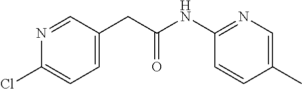 [SMILES: Cc1ccc(NC(=O)Cc2ccc(Cl)nc2)nc1] | D | LCMS m/z: 262.40 (M + H)+ |
| D-39 | 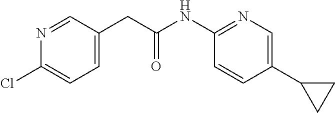 [SMILES: O=C(Cc1ccc(Cl)nc1)Nc1ccc(C2CC2)cn1] | D | LCMS m/z: 288.50 (M + H) ⁺ |
| D-40 | 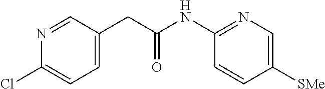 [SMILES: CSc1ccc(NC(=O)Cc2ccc(Cl)nc2)nc1] | D | LCMS m/z: 294.60 (M + H) ⁺ |
| D-41 | 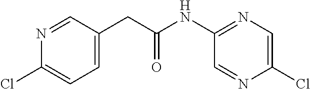 [SMILES: O=C(Cc1ccc(Cl)nc1)Nc1cnc(Cl)cn1] | D | LCMS m/z: 283.90 (M + H) ⁺ |
| D-42 | 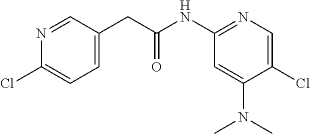 [SMILES: CN(C)c1cc(NC(=O)Cc2ccc(Cl)nc2)ncc1Cl] | D | LCMS m/z: 325.90 (M + H) ⁺ |
| D-43 | 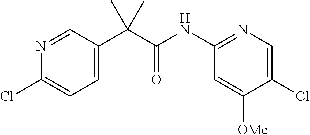 [SMILES: COc1cc(NC(=O)C(C)(C)c2ccc(Cl)nc2)ncc1Cl] | C | LCMS m/z: 341.00 (M + H) ⁺ |
| D-44 | 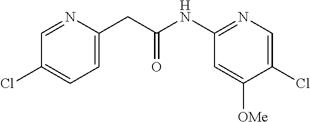 [SMILES: COc1cc(NC(=O)Cc2ccc(Cl)cn2)ncc1Cl] | D | LCMS m/z: 312.20 (M + H) ⁺ |
| D-45 | 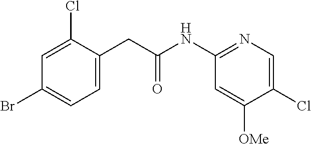 [SMILES: COc1cc(NC(=O)Cc2ccc(Br)cc2Cl)ncc1Cl] | D | LCMS m/z: 391.90 (M + H) ⁺ |
| D-46 | 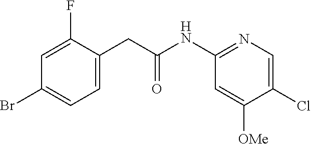 [SMILES: COc1cc(NC(=O)Cc2ccc(Br)cc2F)ncc1Cl] | D | LCMS m/z: 375.00 (M + 2H) ⁺ |
| D-47 | 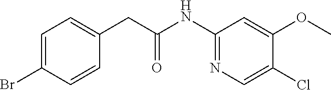 [SMILES: COc1cc(NC(=O)Cc2ccc(Br)cc2)ncc1Cl] | D | LCMS m/z: 357.00 (M + 2H) ⁺ |
| D-48 | 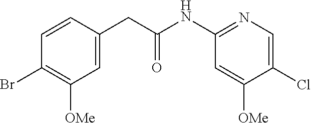 [SMILES: COc1cc(NC(=O)Cc2ccc(Br)c(OC)c2)ncc1Cl] | D | LCMS m/z: 386.40 (M + 2H) ⁺ |
| D-49 | 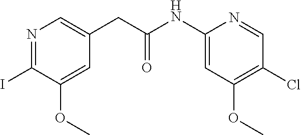 | D | LCMS m/z: 444.00 (M + H) ⁺ |
| D-50 | 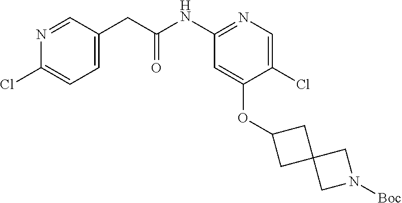 [SMILES: CC(C)(C)OC(=O)N1CC2(CC(Oc3cc(NC(=O)Cc4ccc(Cl)nc4)ncc3Cl)C2)C1] | D | LCMS m/z: 494.60 (M + H) ⁺ |
| D-51 | 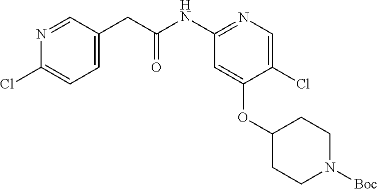 [SMILES: CC(C)(C)OC(=O)N1CCC(Oc2cc(NC(=O)Cc3ccc(Cl)nc3)ncc2Cl)CC1] | D | LCMS m/z: 481.90 (M + H) ⁺ |
| D-52 | 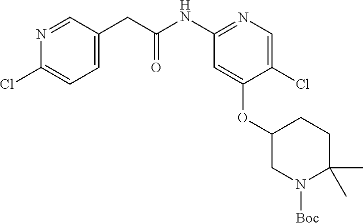 [SMILES: CC(C)(C)OC(=O)N1CC(Oc2cc(NC(=O)Cc3ccc(Cl)nc3)ncc2Cl)CCC1(C)C] | D | LCMS m/z: 510.20 (M + H) ⁺ |
| D-53 | 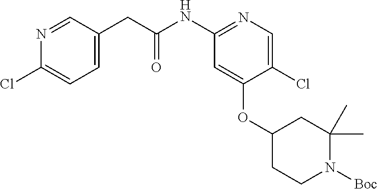 [SMILES: CC(C)(C)OC(=O)N1CCC(Oc2cc(NC(=O)Cc3ccc(Cl)nc3)ncc2Cl)CC1(C)C] | D | LCMS m/z: 510.20 (M + H) ⁺ |
| D-54 | 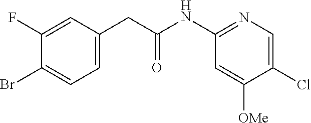 [SMILES: COc1cc(NC(=O)Cc2ccc(Br)c(F)c2)ncc1Cl] | D | LCMS m/z: 375.00 (M + 2H) ⁺ |

### Intermediate 1-24: Synthesis of tert-butyl 5-(2-((5-chloropyridin-2-yl)amino)-2-oxoethyl)-5′,6′-dihydro-[2,3′-bipyridine]-11′(2′H)-carboxylate

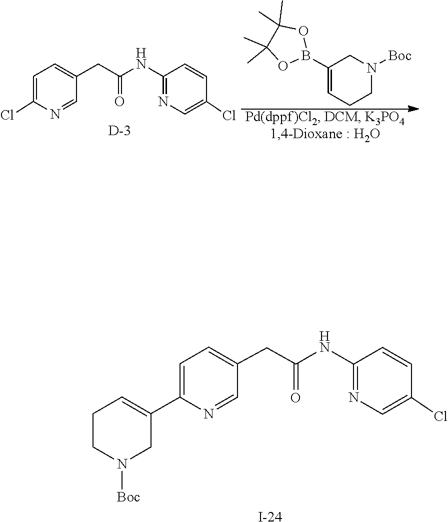

To a degassed solution of N-(5-chloropyridin-2-yl)-2-(6-chloropyridin-3-yl)acetamide (D3) (0.5 g, 1.770 mmol) in 1,4-dioxane (14 mL), K₃PO₄ (0.75 g, 3.45 mmol) dissolved in water (2.0 M solution) and tert-butyl 5-(4,4,5,5-tetramethyl-1,3,2-dioxaborolan-2-yl)-3,6-dihydropyridine-1(2H)-carboxylate (0.55 g, 1.77 mmol) were added, and the resultant solution was degassed for 10 min. Pd(dppf)Cl₂·DCM (0.13 g, 10 mmol) was added, and the mixture was heated at 90° C. for 5 h. The reaction mass was filtered through a celite bed, and the organic layer was separated from the filtrate. The organics were washed with brine and dried over anhydrous sodium sulphate to yield crude title compound, which was further purified using flash chromatography with an ethyl acetate:hexane (1:1) mixture as eluent to afford title compound (0.5 g, 65.86%), LCMS: m/z=429 (M+H)⁺; HPLC: 94.27%, rt: 5.57 min.

The intermediates listed in below Table E were prepared by reacting an amide intermediate with an appropriate boronate ester with a procedure similar to the one described in the synthesis of Intermediate 1-24 with appropriate variations. The characterization data of the intermediates are summarized herein the below table:

TABLE E

| Int. no. | Amide used | Boronate ester used |
|---|---|---|
| E-1 | D-53 | I-22 |
| E-2 | D-2 | 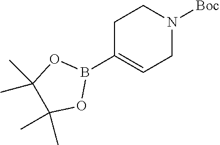 [SMILES: CC(C)(C)OC(=O)N1CC=C(B2OC(C)(C)C(C)(C)O2)CC1] |
| E-3 | D-3 | 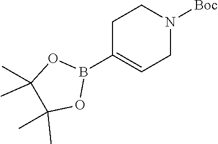 [SMILES: CC(C)(C)OC(=O)N1CC=C(B2OC(C)(C)C(C)(C)O2)CC1] |
| E-4 | D-3 | 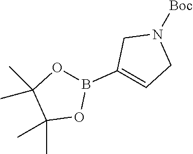 [SMILES: CC(C)(C)OC(=O)N1CC=C(B2OC(C)(C)C(C)(C)O2)C1] |
| E-5 | D-4 | 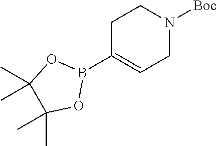 [SMILES: CC(C)(C)OC(=O)N1CC=C(B2OC(C)(C)C(C)(C)O2)CC1] |
| E-6 | D-3 | 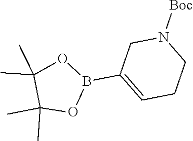 [SMILES: CC(C)(C)OC(=O)N1CCC=C(B2OC(C)(C)C(C)(C)O2)C1] |
| E-7 | D-6 | 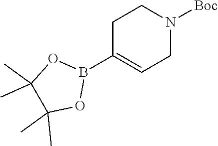 [SMILES: CC(C)(C)OC(=O)N1CC=C(B2OC(C)(C)C(C)(C)O2)CC1] |
| E-8 | D-7 | 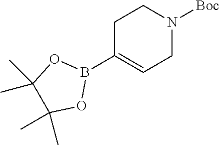 [SMILES: CC(C)(C)OC(=O)N1CC=C(B2OC(C)(C)C(C)(C)O2)CC1] |
| E-9 | D-5 | 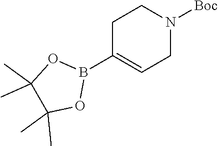 [SMILES: CC(C)(C)OC(=O)N1CC=C(B2OC(C)(C)C(C)(C)O2)CC1] |
| E-10 | D-8 | 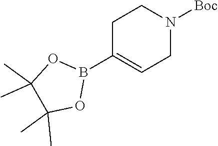 [SMILES: CC(C)(C)OC(=O)N1CC=C(B2OC(C)(C)C(C)(C)O2)CC1] |
| E-11 | D-9 | 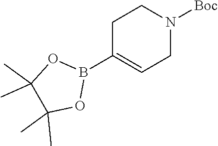 [SMILES: CC(C)(C)OC(=O)N1CC=C(B2OC(C)(C)C(C)(C)O2)CC1] |
| E-12 | D-10 | 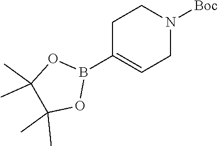 [SMILES: CC(C)(C)OC(=O)N1CC=C(B2OC(C)(C)C(C)(C)O2)CC1] |
| E-13 | D-11 | 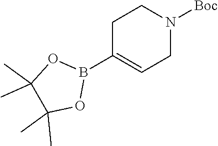 [SMILES: CC(C)(C)OC(=O)N1CC=C(B2OC(C)(C)C(C)(C)O2)CC1] |
| E-14 | D-12 | 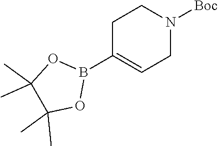 [SMILES: CC(C)(C)OC(=O)N1CC=C(B2OC(C)(C)C(C)(C)O2)CC1] |
| E-15 | D-13 | 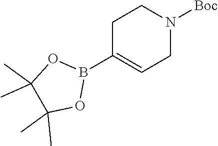 [SMILES: CC(C)(C)OC(=O)N1CC=C(B2OC(C)(C)C(C)(C)O2)CC1] |
| E-16 | D-14 | 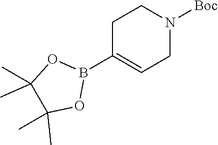 [SMILES: CC(C)(C)OC(=O)N1CC=C(B2OC(C)(C)C(C)(C)O2)CC1] |
| E-17 | D-15 | 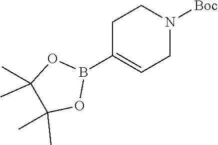 [SMILES: CC(C)(C)OC(=O)N1CC=C(B2OC(C)(C)C(C)(C)O2)CC1] |
| E-18 | D-16 | 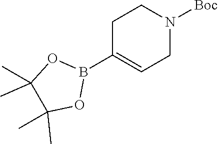 [SMILES: CC(C)(C)OC(=O)N1CC=C(B2OC(C)(C)C(C)(C)O2)CC1] |
| E-19 | D-17 | 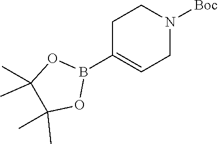 [SMILES: CC(C)(C)OC(=O)N1CC=C(B2OC(C)(C)C(C)(C)O2)CC1] |
| E-20 | D-18 | 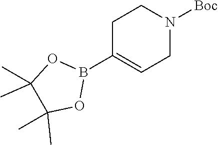 [SMILES: CC(C)(C)OC(=O)N1CC=C(B2OC(C)(C)C(C)(C)O2)CC1] |
| E-21 | D-19 | 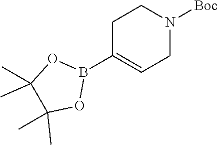 [SMILES: CC(C)(C)OC(=O)N1CC=C(B2OC(C)(C)C(C)(C)O2)CC1] |
| E-22 | D-20 | 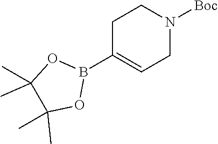 [SMILES: CC(C)(C)OC(=O)N1CC=C(B2OC(C)(C)C(C)(C)O2)CC1] |
| E-23 | D-21 | 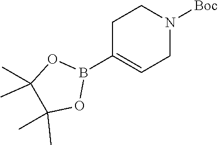 [SMILES: CC(C)(C)OC(=O)N1CC=C(B2OC(C)(C)C(C)(C)O2)CC1] |
| E-24 | D-22 | 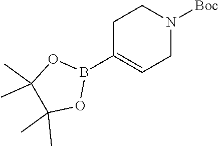 [SMILES: CC(C)(C)OC(=O)N1CC=C(B2OC(C)(C)C(C)(C)O2)CC1] |
| E-25 | D-23 | 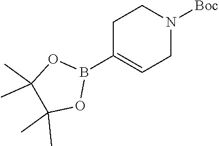 [SMILES: CC(C)(C)OC(=O)N1CC=C(B2OC(C)(C)C(C)(C)O2)CC1] |
| E-26 | D-24 | 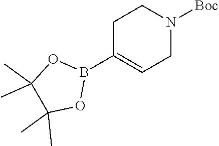 [SMILES: CC(C)(C)OC(=O)N1CC=C(B2OC(C)(C)C(C)(C)O2)CC1] |
| E-27 | D-25 | 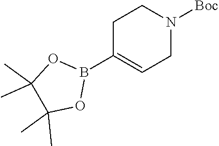 [SMILES: CC(C)(C)OC(=O)N1CC=C(B2OC(C)(C)C(C)(C)O2)CC1] |
| E-28 | D-26 | 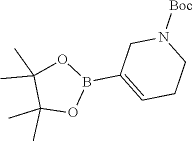 [SMILES: CC(C)(C)OC(=O)N1CCC=C(B2OC(C)(C)C(C)(C)O2)C1] |
| E-29 | D-27 | 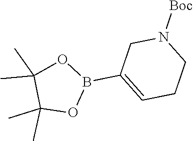 [SMILES: CC(C)(C)OC(=O)N1CCC=C(B2OC(C)(C)C(C)(C)O2)C1] |
| E-30 | D-27 | 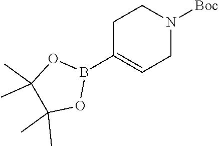 [SMILES: CC(C)(C)OC(=O)N1CC=C(B2OC(C)(C)C(C)(C)O2)CC1] |
| E-31 | D-26 | 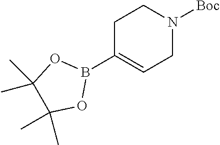 [SMILES: CC(C)(C)OC(=O)N1CC=C(B2OC(C)(C)C(C)(C)O2)CC1] |
| E-32 | D-2 | 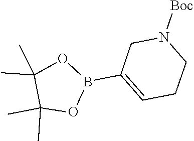 [SMILES: CC(C)(C)OC(=O)N1CCC=C(B2OC(C)(C)C(C)(C)O2)C1] |
| E-33 | D-8 | 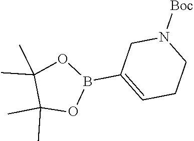 [SMILES: CC(C)(C)OC(=O)N1CCC=C(B2OC(C)(C)C(C)(C)O2)C1] |
| E-34 | D-15 | 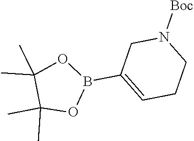 [SMILES: CC(C)(C)OC(=O)N1CCC=C(B2OC(C)(C)C(C)(C)O2)C1] |
| E-35 | D-14 | 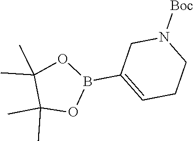 [SMILES: CC(C)(C)OC(=O)N1CCC=C(B2OC(C)(C)C(C)(C)O2)C1] |
| E-36 | D-11 | 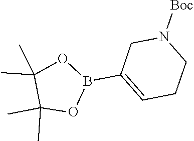 [SMILES: CC(C)(C)OC(=O)N1CCC=C(B2OC(C)(C)C(C)(C)O2)C1] |
| E-37 | D-10 | 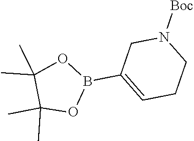 [SMILES: CC(C)(C)OC(=O)N1CCC=C(B2OC(C)(C)C(C)(C)O2)C1] |
| E-38 | D-23 | 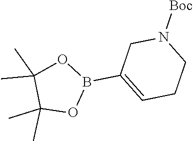 [SMILES: CC(C)(C)OC(=O)N1CCC=C(B2OC(C)(C)C(C)(C)O2)C1] |
| E-39 | D-24 | 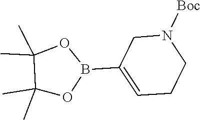 [SMILES: CC(C)(C)OC(=O)N1CCC=C(B2OC(C)(C)C(C)(C)O2)C1] |
| E-40 | D-25 | 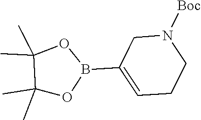 [SMILES: CC(C)(C)OC(=O)N1CCC=C(B2OC(C)(C)C(C)(C)O2)C1] |
| E-41 | D-29 | 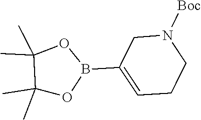 [SMILES: CC(C)(C)OC(=O)N1CCC=C(B2OC(C)(C)C(C)(C)O2)C1] |
| E-42 | D-30 | 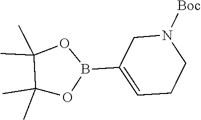 [SMILES: CC(C)(C)OC(=O)N1CCC=C(B2OC(C)(C)C(C)(C)O2)C1] |
| E-43 | D-30 | 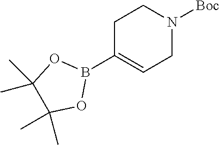 [SMILES: CC(C)(C)OC(=O)N1CC=C(B2OC(C)(C)C(C)(C)O2)CC1] |
| E-44 | D-31 | 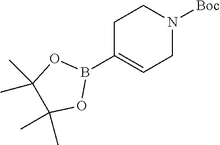 [SMILES: CC(C)(C)OC(=O)N1CC=C(B2OC(C)(C)C(C)(C)O2)CC1] |
| E-45 | D-31 | 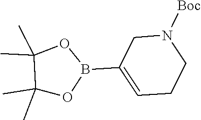 [SMILES: CC(C)(C)OC(=O)N1CCC=C(B2OC(C)(C)C(C)(C)O2)C1] |
| E-46 | D-22 | 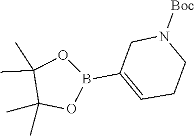 [SMILES: CC(C)(C)OC(=O)N1CCC=C(B2OC(C)(C)C(C)(C)O2)C1] |
| E-47 | D-5 | 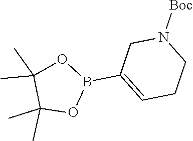 [SMILES: CC(C)(C)OC(=O)N1CCC=C(B2OC(C)(C)C(C)(C)O2)C1] |
| E-48 | D-32 | 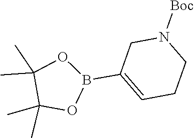 [SMILES: CC(C)(C)OC(=O)N1CCC=C(B2OC(C)(C)C(C)(C)O2)C1] |
| E-49 | D-33 | 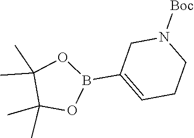 [SMILES: CC(C)(C)OC(=O)N1CCC=C(B2OC(C)(C)C(C)(C)O2)C1] |
| E-50 | D-2 | I-5 |
| E-51 | D-34 | 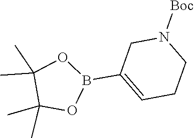 [SMILES: CC(C)(C)OC(=O)N1CCC=C(B2OC(C)(C)C(C)(C)O2)C1] |
| E-52 | D-2 | A-1 |
| E-53 | D-2 | A-3 |
| E-54 | D-35 | 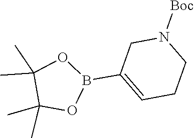 [SMILES: CC(C)(C)OC(=O)N1CCC=C(B2OC(C)(C)C(C)(C)O2)C1] |
| E-55 | D-35 | 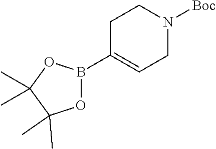 [SMILES: CC(C)(C)OC(=O)N1CC=C(B2OC(C)(C)C(C)(C)O2)CC1] |
| E-56 | D-22 | 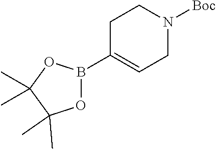 [SMILES: CC(C)(C)OC(=O)N1CC=C(B2OC(C)(C)C(C)(C)O2)CC1] |
| E-57 | D-36 | 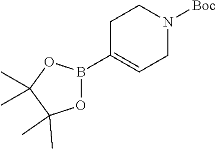 [SMILES: CC(C)(C)OC(=O)N1CC=C(B2OC(C)(C)C(C)(C)O2)CC1] |
| E-58 | D-2 | A-2 |
| E-59 | 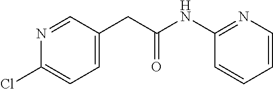 [SMILES: O=C(Cc1ccc(Cl)nc1)Nc1ccccn1] | 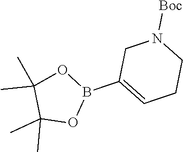 [SMILES: CC(C)(C)OC(=O)N1CCC=C(B2OC(C)(C)C(C)(C)O2)C1] |
| E-60 | D-37 | 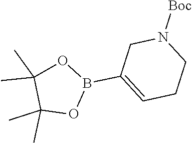 [SMILES: CC(C)(C)OC(=O)N1CCC=C(B2OC(C)(C)C(C)(C)O2)C1] |
| E-61 | D-38 | 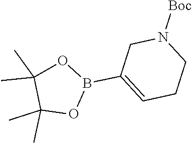 [SMILES: CC(C)(C)OC(=O)N1CCC=C(B2OC(C)(C)C(C)(C)O2)C1] |
| E-62 | D-39 | 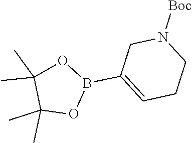 [SMILES: CC(C)(C)OC(=O)N1CCC=C(B2OC(C)(C)C(C)(C)O2)C1] |
| E-63 | D-40 | 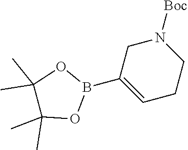 [SMILES: CC(C)(C)OC(=O)N1CCC=C(B2OC(C)(C)C(C)(C)O2)C1] |
| E-64 | D-41 | 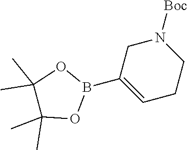 [SMILES: CC(C)(C)OC(=O)N1CCC=C(B2OC(C)(C)C(C)(C)O2)C1] |
| E-65 | D-42 | 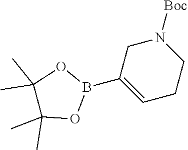 [SMILES: CC(C)(C)OC(=O)N1CCC=C(B2OC(C)(C)C(C)(C)O2)C1] |
| E-66 | D-43 | 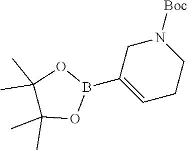 [SMILES: CC(C)(C)OC(=O)N1CCC=C(B2OC(C)(C)C(C)(C)O2)C1] |
| E-67 | D-44 | 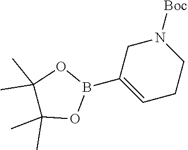 [SMILES: CC(C)(C)OC(=O)N1CCC=C(B2OC(C)(C)C(C)(C)O2)C1] |
| E-68 | D-45 | 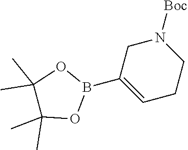 [SMILES: CC(C)(C)OC(=O)N1CCC=C(B2OC(C)(C)C(C)(C)O2)C1] |
| E-69 | D-46 | 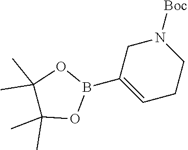 [SMILES: CC(C)(C)OC(=O)N1CCC=C(B2OC(C)(C)C(C)(C)O2)C1] |
| E-70 | D-47 | 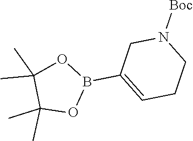 [SMILES: CC(C)(C)OC(=O)N1CCC=C(B2OC(C)(C)C(C)(C)O2)C1] |
| E-71 | D-47 | 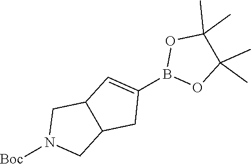 [SMILES: CC(C)(C)OC(=O)N1CC2C=C(B3OC(C)(C)C(C)(C)O3)CC2C1] |
| E-72 | D-48 | 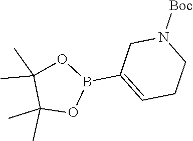 [SMILES: CC(C)(C)OC(=O)N1CCC=C(B2OC(C)(C)C(C)(C)O2)C1] |
| E-73 | D-49 | 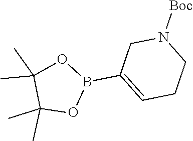 [SMILES: CC(C)(C)OC(=O)N1CCC=C(B2OC(C)(C)C(C)(C)O2)C1] |
| E-74 | D-49 | 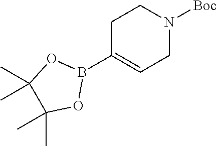 [SMILES: CC(C)(C)OC(=O)N1CC=C(B2OC(C)(C)C(C)(C)O2)CC1] |
| E-75 | D-1 | 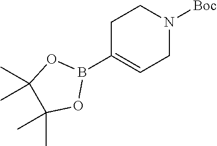 [SMILES: CC(C)(C)OC(=O)N1CC=C(B2OC(C)(C)C(C)(C)O2)CC1] |
| E-76 | D-44 | 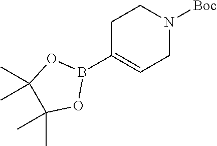 [SMILES: CC(C)(C)OC(=O)N1CC=C(B2OC(C)(C)C(C)(C)O2)CC1] |
| E-77 | D-2 | I-20 |
| E-78 | D-2 | C-1 |
| E-79 | D-2 | C-2 |
| E-80 | D-50 | I-22 |
| E-81 | D-51 | I-22 |
| E-82 | D-52 | I-22 |

| | | Characterization |
|---|---|---|
| Int. no. | Intermediate structure | Data |
| E-1 | 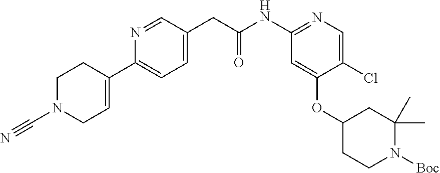 [SMILES: CC(C)(C)OC(=O)N1CCC(Oc2cc(NC(=O)Cc3ccc(C4=CCN(C#N)CC4)nc3)ncc2Cl)CC1(C)C] | LCMS m/z: 581.80 (M + H) ⁺ |
| E-2 | 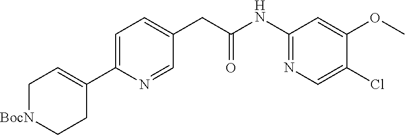 [SMILES: COc1cc(NC(=O)Cc2ccc(C3=CCN(C(=O)OC(C)(C)C)CC3)nc2)ncc1Cl] | LCMS m/z: 459.80 (M + H) |
| E-3 | 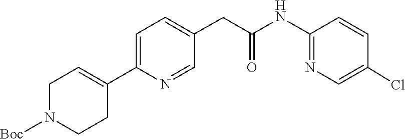 [SMILES: CC(C)(C)OC(=O)N1CC=C(c2ccc(CC(=O)Nc3ccc(Cl)cn3)cn2)CC1] | LCMS m/z: 429.00 (M + H) ⁺ |
| E-4 | 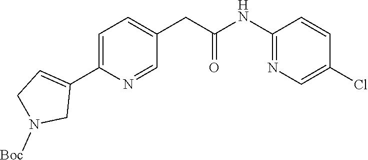 [SMILES: CC(C)(C)OC(=O)N1CC=C(c2ccc(CC(=O)Nc3ccc(Cl)cn3)cn2)C1] | LCMS m/z: 415.90 (M + H) ⁺ |
| E-5 | 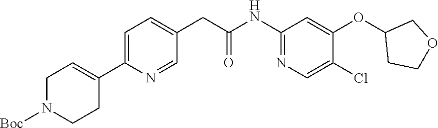 [SMILES: CC(C)(C)OC(=O)N1CC=C(c2ccc(CC(=O)Nc3cc(OC4CCOC4)c(Cl)cn3)cn2)CC1] | LCMS m/z: 515.10 (M + H) ⁺ |
| E-6 | 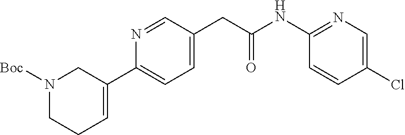 [SMILES: CC(C)(C)OC(=O)N1CCC=C(c2ccc(CC(=O)Nc3ccc(Cl)cn3)cn2)C1] | LCMS m/z: 429.10 (M + H) ⁺ |
| E-7 | 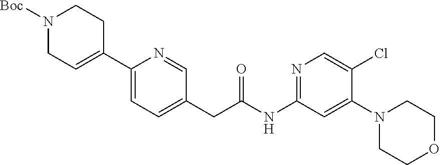 [SMILES: CC(C)(C)OC(=O)N1CC=C(c2ccc(CC(=O)Nc3cc(N4CCOCC4)c(Cl)cn3)cn2)CC1] | LCMS m/z: 514.10 (M + H)⁺ |
| E-8 | 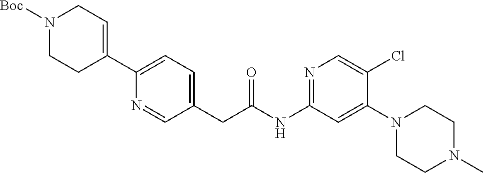 [SMILES: CN1CCN(c2cc(NC(=O)Cc3ccc(C4=CCN(C(=O)OC(C)(C)C)CC4)nc3)ncc2Cl)CC1] | LCMS m/z: 527.10 (M + H)⁺ |
| E-9 | 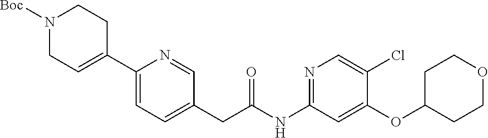 [SMILES: CC(C)(C)OC(=O)N1CC=C(c2ccc(CC(=O)Nc3cc(OC4CCOCC4)c(Cl)cn3)cn2)CC1] | LCMS m/z: 529.10 (M + H)⁺ |
| E-10 | 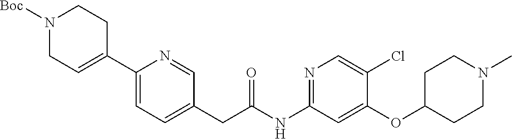 [SMILES: CN1CCC(Oc2cc(NC(=O)Cc3ccc(C4=CCN(C(=O)OC(C)(C)C)CC4)nc3)ncc2Cl)CC1] | LCMS m/z: 542.10 (M + H)⁺ |
| E-11 | 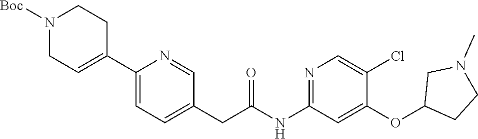 [SMILES: CN1CCC(Oc2cc(NC(=O)Cc3ccc(C4=CCN(C(=O)OC(C)(C)C)CC4)nc3)ncc2Cl)C1] | LCMS m/z: 528.20 (M + H) ⁺ |
| E-12 | 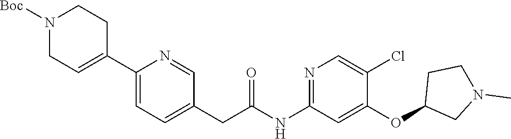 [SMILES: CN1CC[C@H](Oc2cc(NC(=O)Cc3ccc(C4=CCN(C(=O)OC(C)(C)C)CC4)nc3)ncc2Cl)C1] | LCMS m/z: 528.20 (M + H) ⁺ |
| E-13 | 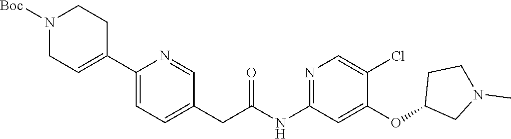 [SMILES: CN1CC[C@@H](Oc2cc(NC(=O)Cc3ccc(C4=CCN(C(=O)OC(C)(C)C)CC4)nc3)ncc2Cl)C1] | LCMS m/z: 528.20 (M + H) ⁺ |
| E-14 | 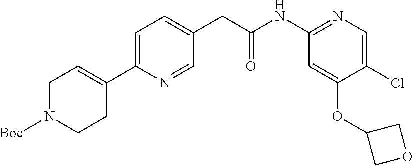 [SMILES: CC(C)(C)OC(=O)N1CC=C(c2ccc(CC(=O)Nc3cc(OC4COC4)c(Cl)cn3)cn2)CC1] | LCMS m/z: 501.10 (M + H) ⁺ |
| E-15 | 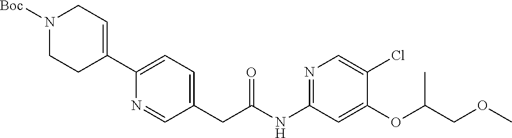 [SMILES: COCC(C)Oc1cc(NC(=O)Cc2ccc(C3=CCN(C(=O)OC(C)(C)C)CC3)nc2)ncc1Cl] | LCMS m/z: 517.30 (M + H) ⁺ |
| E-16 | 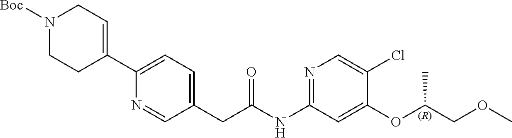 | LCMS m/z: 517.30 (M + H) ⁺ |
| E-17 | 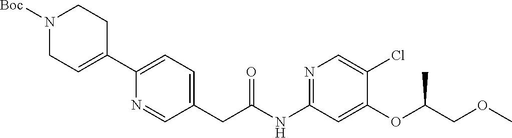 [SMILES: COC[C@H](C)Oc1cc(NC(=O)Cc2ccc(C3=CCN(C(=O)OC(C)(C)C)CC3)nc2)ncc1Cl] | LCMS m/z: 517.30 (M + H) ⁺ |
| E-18 | 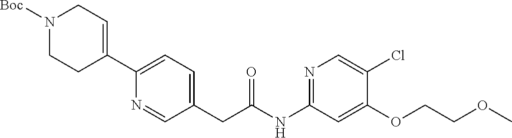 [SMILES: COCCOc1cc(NC(=O)Cc2ccc(C3=CCN(C(=O)OC(C)(C)C)CC3)nc2)ncc1Cl] | LCMS m/z: 503.20 (M + H)⁺. |
| E-19 | 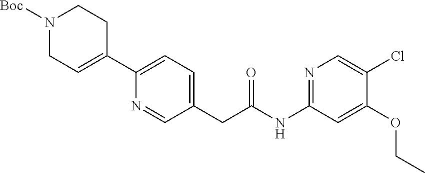 [SMILES: CCOc1cc(NC(=O)Cc2ccc(C3=CCN(C(=O)OC(C)(C)C)CC3)nc2)ncc1Cl] | LCMS m/z: 473.20 (M + H)⁺. |
| E-20 | 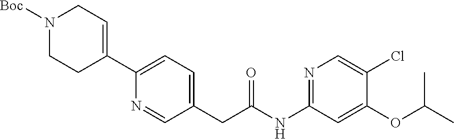 [SMILES: CC(C)Oc1cc(NC(=O)Cc2ccc(C3=CCN(C(=O)OC(C)(C)C)CC3)nc2)ncc1Cl] | LCMS m/z: 487.40 (M + H)⁺. |
| E-21 | 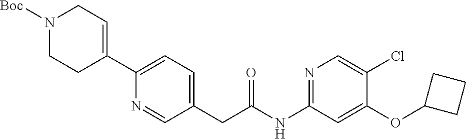 [SMILES: CC(C)(C)OC(=O)N1CC=C(c2ccc(CC(=O)Nc3cc(OC4CCC4)c(Cl)cn3)cn2)CC1] | LCMS m/z: 499.20 (M + H)⁺. |
| E-22 | 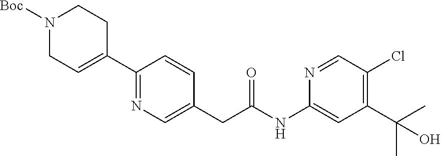 [SMILES: CC(C)(C)OC(=O)N1CC=C(c2ccc(CC(=O)Nc3cc(C(C)(C)O)c(Cl)cn3)cn2)CC1] | LCMS m/z: 487.00 (M + H)⁺ |
| E-23 | 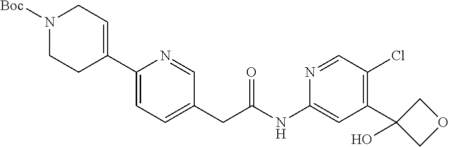 [SMILES: CC(C)(C)OC(=O)N1CC=C(c2ccc(CC(=O)Nc3cc(C4(O)COC4)c(Cl)cn3)cn2)CC1] | LCMS m/z: 501.30 (M + H) ⁺ |
| E-24 | 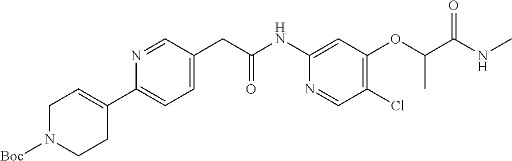 [SMILES: CNC(=O)C(C)Oc1cc(NC(=O)Cc2ccc(C3=CCN(C(=O)OC(C)(C)C)CC3)nc2)ncc1Cl] | LCMS m/z: 530.80 (M + H) ⁺ |
| E-25 | 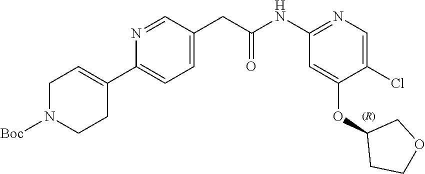 | LCMS m/z: 515.70 (M + H) ⁺ |
| E-26 | 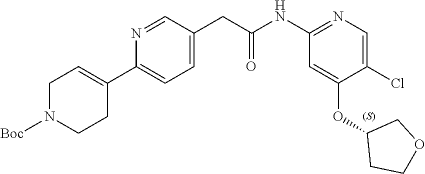 | LCMS m/z: 515.70 (M + H) ⁺ |
| E-27 | 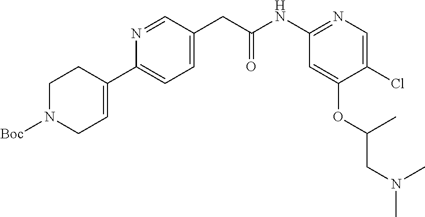 [SMILES: CC(CN(C)C)Oc1cc(NC(=O)Cc2ccc(C3=CCN(C(=O)OC(C)(C)C)CC3)nc2)ncc1Cl] | LCMS m/z: 530.80 (M + H) |
| E-28 | 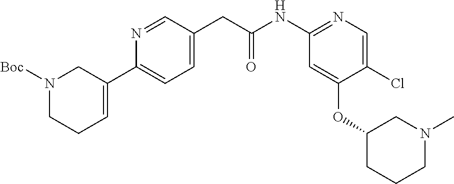 [SMILES: CN1CCC[C@H](Oc2cc(NC(=O)Cc3ccc(C4=CCCN(C(=O)OC(C)(C)C)C4)nc3)ncc2Cl)C1] | LCMS m/z: 542.80 (M + H) |
| E-29 | 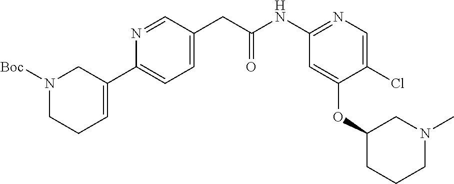 [SMILES: CN1CCC[C@@H](Oc2cc(NC(=O)Cc3ccc(C4=CCCN(C(=O)OC(C)(C)C)C4)nc3)ncc2Cl)C1] | LCMS m/z: 542.80 (M + H) |
| E-30 | 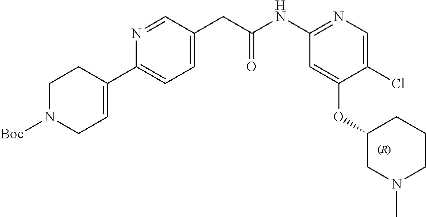 | LCMS m/z: 542.00 (M + H) ⁺ |
| E-31 | 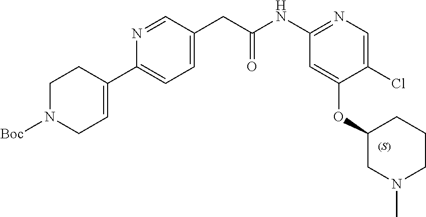 | LCMS m/z: 542.00 (M + H) ⁺ |
| E-32 | 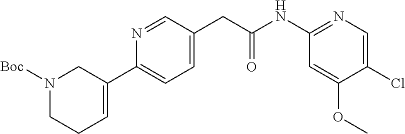 [SMILES: COc1cc(NC(=O)Cc2ccc(C3=CCCN(C(=O)OC(C)(C)C)C3)nc2)ncc1Cl] | LCMS m/z: 459.10 (M + H) ⁺ |
| E-33 | 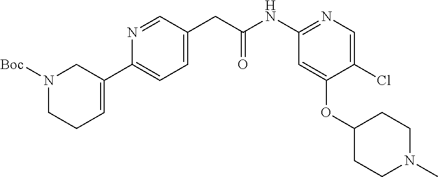 [SMILES: CN1CCC(Oc2cc(NC(=O)Cc3ccc(C4=CCCN(C(=O)OC(C)(C)C)C4)nc3)ncc2Cl)CC1] | LCMS m/z: 542.00 (M + H) ⁺ |
| E-34 | 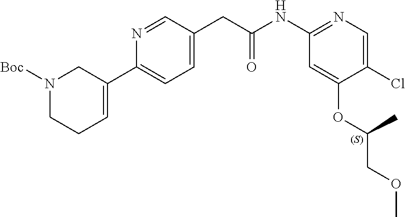 | LCMS m/z: 517.80 (M + H) ⁺ |
| E-35 | 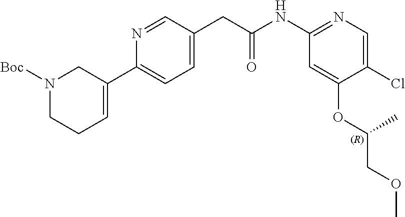 | LCMS m/z: 517.80 (M + H) ⁺ |
| E-36 | 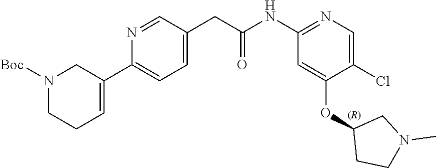 | LCMS m/z: 528.80 (M + H) ⁺ |
| E-37 | 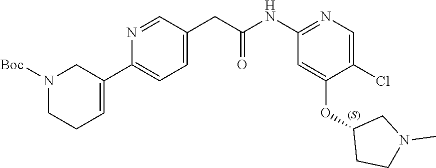 | LCMS m/z: 528.80 (M + H) ⁺ |
| E-38 | 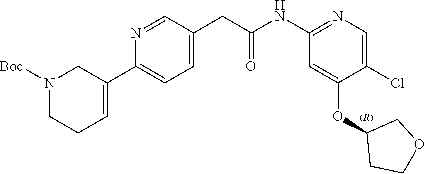 | LCMS m/z: 516.10 (M + H) ⁺ |
| E-39 | 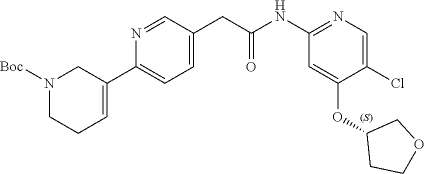 | LCMS m/z: 516.00 (M + H) ⁺ |
| E-40 | 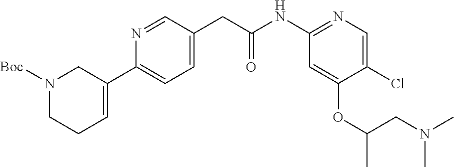 [SMILES: CC(CN(C)C)Oc1cc(NC(=O)Cc2ccc(C3=CCCN(C(=O)OC(C)(C)C)C3)nc2)ncc1Cl] | LCMS m/z: 530.90 (M + H) ⁺ |
| E-41 | 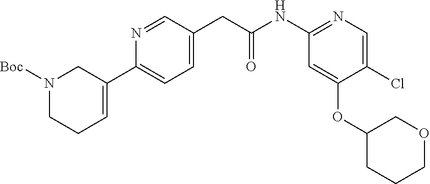 [SMILES: CC(C)(C)OC(=O)N1CCC=C(c2ccc(CC(=O)Nc3cc(OC4CCCOC4)c(Cl)cn3)cn2)C1] | LCMS m/z: 529.80 (M + H) ⁺ |
| E-42 | 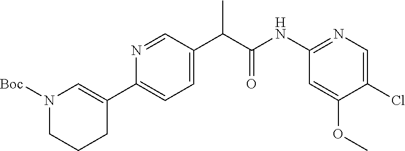 [SMILES: COc1cc(NC(=O)C(C)c2ccc(C3=CN(C(=O)OC(C)(C)C)CCC3)nc2)ncc1Cl] | LCMS m/z: 473.70 (M + H) ⁺ |
| E-43 | 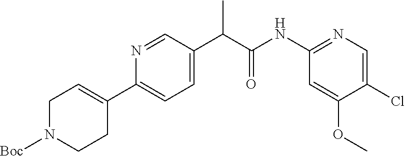 [SMILES: COc1cc(NC(=O)C(C)c2ccc(C3=CCN(C(=O)OC(C)(C)C)CC3)nc2)ncc1Cl] | LCMS m/z: 473.70 (M + H) ⁺ |
| E-44 | 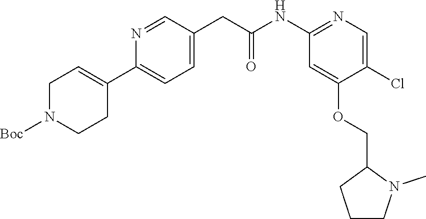 [SMILES: CN1CCCC1COc1cc(NC(=O)Cc2ccc(C3=CCN(C(=O)OC(C)(C)C)CC3)nc2)ncc1Cl] | LCMS m/z: 542.80 (M + H) ⁺ |
| E-45 | 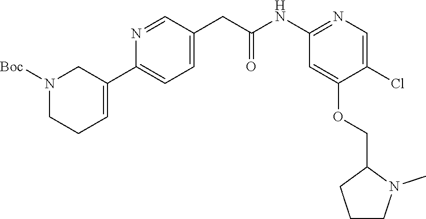 [SMILES: CN1CCCC1COc1cc(NC(=O)Cc2ccc(C3=CCCN(C(=O)OC(C)(C)C)C3)nc2)ncc1Cl] | LCMS m/z: 542.80 (M + H) ⁺ |
| E-46 | 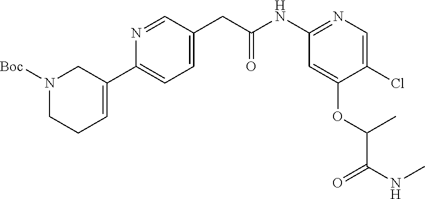 [SMILES: CNC(=O)C(C)Oc1cc(NC(=O)Cc2ccc(C3=CCCN(C(=O)OC(C)(C)C)C3)nc2)ncc1Cl] | LCMS m/z: 530.80 (M + H) ⁺ |
| E-47 | 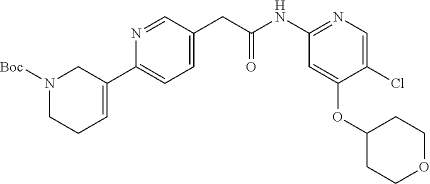 [SMILES: CC(C)(C)OC(=O)N1CCC=C(c2ccc(CC(=O)Nc3cc(OC4CCOCC4)c(Cl)cn3)cn2)C1] | LCMS m/z: 529.80 (M + H) ⁺ |
| E-48 | 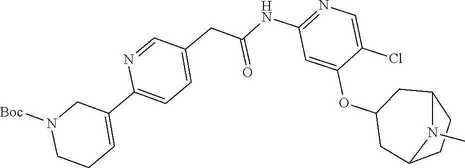 [SMILES: CN1C2CCC1CC(Oc1cc(NC(=O)Cc3ccc(C4=CCCN(C(=O)OC(C)(C)C)C4)nc3)ncc1Cl)C2] | LCMS m/z: 568.90 (M + H) ⁺ |
| E-49 | 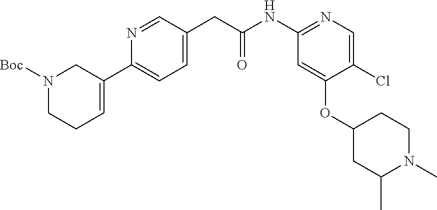 [SMILES: CC1CC(Oc2cc(NC(=O)Cc3ccc(C4=CCCN(C(=O)OC(C)(C)C)C4)nc3)ncc2Cl)CCN1C] | LCMS m/z: 556.90 (M + H) ⁺ |
| E-50 | 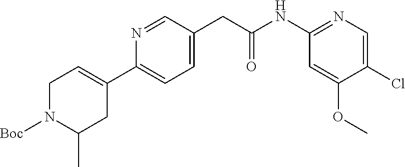 [SMILES: COc1cc(NC(=O)Cc2ccc(C3=CCN(C(=O)OC(C)(C)C)C(C)C3)nc2)ncc1Cl] | LCMS m/z: 473.70 (M + H) ⁺ |
| E-51 | 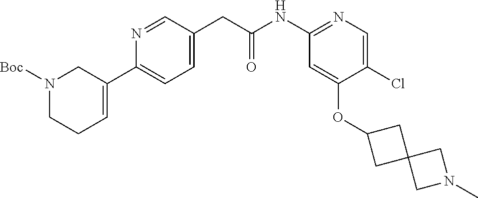 [SMILES: CN1CC2(CC(Oc3cc(NC(=O)Cc4ccc(C5=CCCN(C(=O)OC(C)(C)C)C5)nc4)ncc3Cl)C2)C1] | LCMS m/z: 554.90 (M + H) ⁺ |
| E-52 | 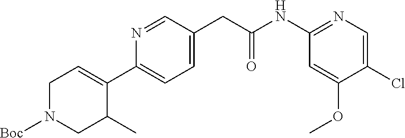 [SMILES: COc1cc(NC(=O)Cc2ccc(C3=CCN(C(=O)OC(C)(C)C)CC3C)nc2)ncc1Cl] | LCMS m/z: 473.80 (M + H) ⁺ |
| E-53 | 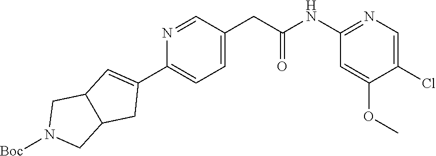 [SMILES: COc1cc(NC(=O)Cc2ccc(C3=CC4CN(C(=O)OC(C)(C)C)CC4C3)nc2)ncc1Cl] | LCMS m/z: 485.80 (M + H) ⁺ |
| E-54 | 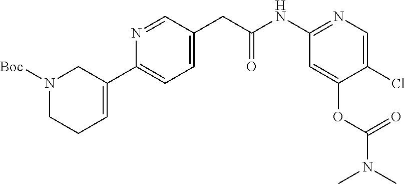 [SMILES: CN(C)C(=O)Oc1cc(NC(=O)Cc2ccc(C3=CCCN(C(=O)OC(C)(C)C)C3)nc2)ncc1Cl] | LCMS m/z: 516.80 (M + H) ⁺ |
| E-55 | 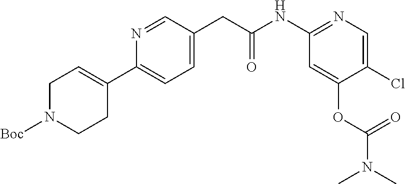 [SMILES: CN(C)C(=O)Oc1cc(NC(=O)Cc2ccc(C3=CCN(C(=O)OC(C)(C)C)CC3)nc2)ncc1Cl] | LCMS m/z: 516.80 (M + H) ⁺ |
| E-56 | 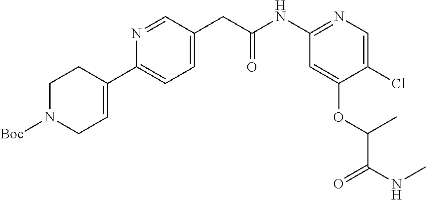 [SMILES: CNC(=O)C(C)Oc1cc(NC(=O)Cc2ccc(C3=CCN(C(=O)OC(C)(C)C)CC3)nc2)ncc1Cl] | LCMS m/z: 530.80 (M + H) ⁺ |
| E-57 | 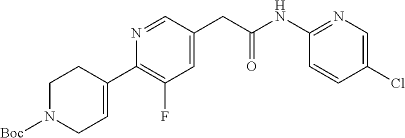 [SMILES: CC(C)(C)OC(=O)N1CC=C(c2ncc(CC(=O)Nc3ccc(Cl)cn3)cc2F)CC1] | LCMS m/z: 447.80 (M + H) ⁺ |
| E-58 | 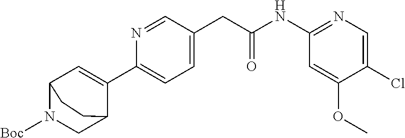 [SMILES: COc1cc(NC(=O)Cc2ccc(C3=CC4CCC3CN4C(=O)OC(C)(C)C)nc2)ncc1Cl] | LCMS m/z: 485.60 (M + H) ⁺ |
| E-59 | 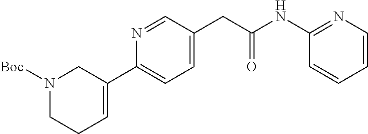 [SMILES: CC(C)(C)OC(=O)N1CCC=C(c2ccc(CC(=O)Nc3ccccn3)cn2)C1] | LCMS m/z: 395.20 (M + H) ⁺ |
| E-60 | 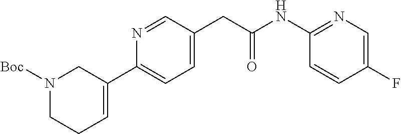 [SMILES: CC(C)(C)OC(=O)N1CCC=C(c2ccc(CC(=O)Nc3ccc(F)cn3)cn2)C1] | LCMS m/z: 413.20 (M + H) ⁺ |
| E-61 | 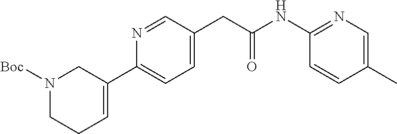 [SMILES: Cc1ccc(NC(=O)Cc2ccc(C3=CCCN(C(=O)OC(C)(C)C)C3)nc2)nc1] | LCMS m/z: 409.20 (M + H) ⁺ |
| E-62 | 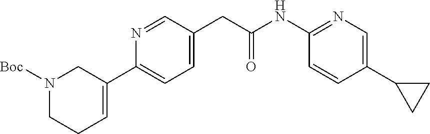 [SMILES: CC(C)(C)OC(=O)N1CCC=C(c2ccc(CC(=O)Nc3ccc(C4CC4)cn3)cn2)C1] | LCMS m/z: 435.20 (M + H) ⁺ |
| E-63 | 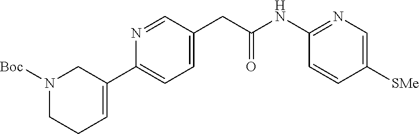 [SMILES: CSc1ccc(NC(=O)Cc2ccc(C3=CCCN(C(=O)OC(C)(C)C)C3)nc2)nc1] | LCMS m/z: 441.20 (M + H) ⁺ |
| E-64 | 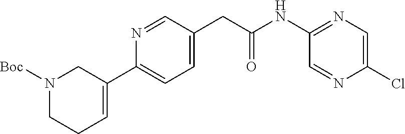 [SMILES: CC(C)(C)OC(=O)N1CCC=C(c2ccc(CC(=O)Nc3cnc(Cl)cn3)cn2)C1] | LCMS m/z: 430.80 (M + H) ⁺ |
| E-65 | 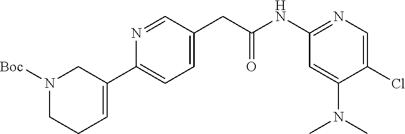 [SMILES: CN(C)c1cc(NC(=O)Cc2ccc(C3=CCCN(C(=O)OC(C)(C)C)C3)nc2)ncc1Cl] | LCMS m/z: 472.80 (M + H) ⁺ |
| E-66 | 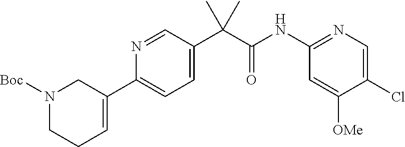 [SMILES: COc1cc(NC(=O)C(C)(C)c2ccc(C3=CCCN(C(=O)OC(C)(C)C)C3)nc2)ncc1Cl] | LCMS m/z: 488.80 (M + H) ⁺ |
| E-67 | 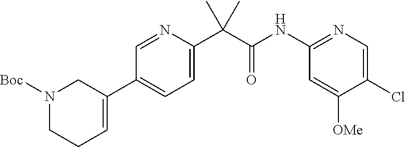 | LCMS m/z: 459.80 (M + H) ⁺ |
| E-68 | 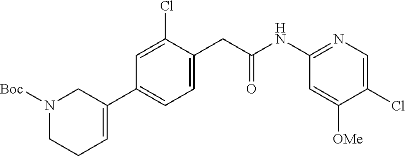 [SMILES: COc1cc(NC(=O)Cc2ccc(C3=CCCN(C(=O)OC(C)(C)C)C3)cc2Cl)ncc1Cl] | LCMS m/z: 493.10 (M + H) ⁺ |
| E-69 | 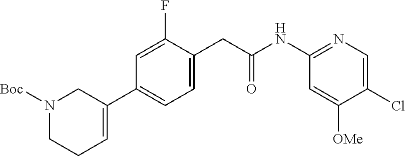 [SMILES: COc1cc(NC(=O)Cc2ccc(C3=CCCN(C(=O)OC(C)(C)C)C3)cc2F)ncc1Cl] | LCMS m/z: 476.80 (M + H) ⁺ |
| E-70 | 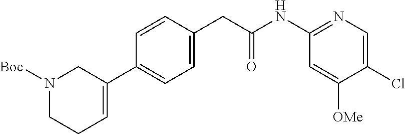 [SMILES: COc1cc(NC(=O)Cc2ccc(C3=CCCN(C(=O)OC(C)(C)C)C3)cc2)ncc1Cl] | LCMS m/z: 458.70 (M + H) ⁺ |
| E-71 | 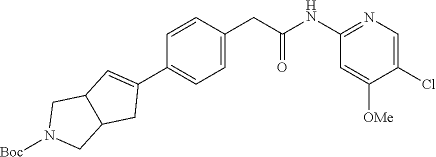 [SMILES: COc1cc(NC(=O)Cc2ccc(C3=CC4CN(C(=O)OC(C)(C)C)CC4C3)cc2)ncc1Cl] | LCMS m/z: 484.90 (M + H) ⁺ |
| E-72 | 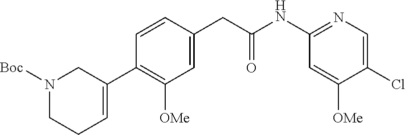 [SMILES: COc1cc(NC(=O)Cc2ccc(C3=CCCN(C(=O)OC(C)(C)C)C3)c(OC)c2)ncc1Cl] | LCMS m/z: 488.90 (M + H) ⁺ |
| E-73 | 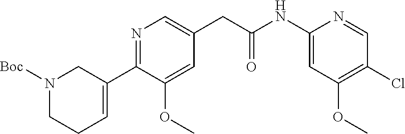 [SMILES: COc1cc(NC(=O)Cc2cnc(C3=CCCN(C(=O)OC(C)(C)C)C3)c(OC)c2)ncc1Cl] | LCMS m/z: 488.70 (M + H) ⁺ |
| E-74 | 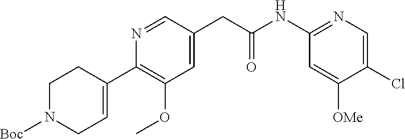 [SMILES: COc1cc(NC(=O)Cc2cnc(C3=CCN(C(=O)OC(C)(C)C)CC3)c(OC)c2)ncc1Cl] | LCMS m/z: 488.70 (M + H) ⁺ |
| E-75 | 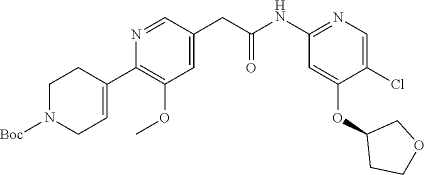 [SMILES: COc1cc(CC(=O)Nc2cc(O[C@@H]3CCOC3)c(Cl)cn2)cnc1C1=CCN(C(=O)OC(C)(C)C)CC1] | LCMS m/z: 545.70 (M + H) ⁺ |
| E-76 | 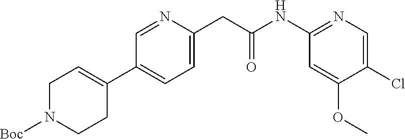 [SMILES: COc1cc(NC(=O)Cc2ccc(C3=CCN(C(=O)OC(C)(C)C)CC3)cn2)ncc1Cl] | LCMS m/z: 459.80 (M + H) ⁺ |
| E-77 | 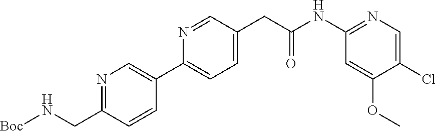 [SMILES: COc1cc(NC(=O)Cc2ccc(-c3ccc(CNC(=O)OC(C)(C)C)nc3)nc2)ncc1Cl] | LCMS m/z: 484.80 (M + H) ⁺ |
| E-78 | 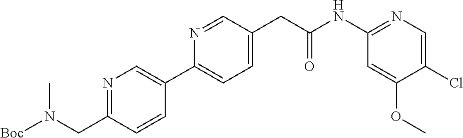 [SMILES: COc1cc(NC(=O)Cc2ccc(-c3ccc(CN(C)C(=O)OC(C)(C)C)nc3)nc2)ncc1Cl] | LCMS m/z: 498.60 (M + H) ⁺ |
| E-79 | 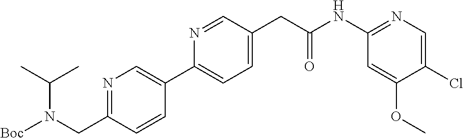 [SMILES: COc1cc(NC(=O)Cc2ccc(-c3ccc(CN(C(=O)OC(C)(C)C)C(C)C)nc3)nc2)ncc1Cl] | LCMS m/z: 527.60 (M + H) ⁺ |
| E-80 | 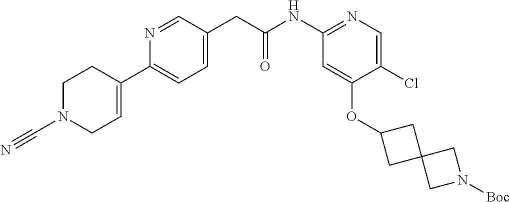 [SMILES: CC(C)(C)OC(=O)N1CC2(CC(Oc3cc(NC(=O)Cc4ccc(C5=CCN(C#N)CC5)nc4)ncc3Cl)C2)C1] | LCMS m/z: 565.80 (M + H) ⁺ |
| E-81 | 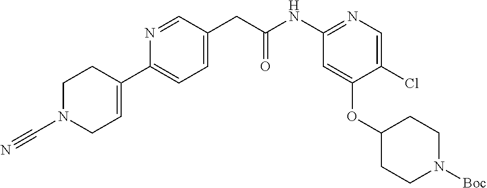 [SMILES: CC(C)(C)OC(=O)N1CCC(Oc2cc(NC(=O)Cc3ccc(C4=CCN(C#N)CC4)nc3)ncc2Cl)CC1] | LCMS m/z: 553.80 (M + H) ⁺ |
| E-82 | 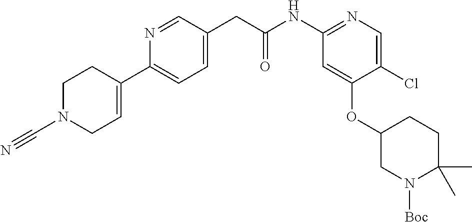 [SMILES: CC(C)(C)OC(=O)N1CC(Oc2cc(NC(=O)Cc3ccc(C4=CCN(C#N)CC4)nc3)ncc2Cl)CCC1(C)C] | LCMS m/z: 581.80 (M + H) ⁺ |

### Intermediate I-25: Synthesis of 2-(1′-(tert-butoxycarbonyl)-1′,2′,5′,6′-tetrahydro-[2,3′-bipyridin]-5-yl)acetic acid

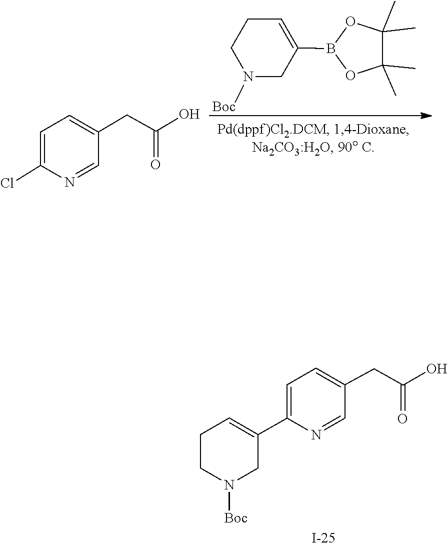

To a degassed solution of 2-(6-chloropyridin-3-yl)acetic acid (1 g, 5.82 mmol) in 1,4-dioxane (14 mL), were added tert-butyl-5-(4,4,5,5-tetramethyl-1,3,2-dioxaborolan-2-yl)-3,6-dihydropyridine-1(2H)-carboxylate (2.16 g, 7.00 mmol) and Na₂CO₃ (1.24 g, 11.66 mmol) in water (6 mL). The resultant solution was degassed for 10 min. Pd(dppf)Cl₂·DCM (0.43 g, 0.58 mmol) was added, and the mixture was heated at 90° C. for 2 h. The reaction mass was concentrated and diluted with water and ethylacetate. The reaction mixture was filtered through a celite bed, separated the organic and aqueous layer. The aqueous layer was acidified with citric acid and extracted with ethylacetate washed with brine and dried over anhydrous sodium sulphate to yield crude title compound. This material was further purified using flash chromatography using an ethyl acetate:hexane (1:1) mixture as eluent to afford title compound (1.5 g, 81.08%); ¹HNMR (DMSO-d₆, 400 MHz): δ 12.60 (bs, 1H), 8.40 (s, 1H), 7.65 (d, 1H), 7.48 (d, 1H), 6.74 (m, 1H), 4.31 (s, 2H), 3.66 (s, 2H), 3.48-3.33 (m, 2H), 2.82-2.80 (m, 2H), 1.42 (s, 9H); LCMS: m/z=319.2 (M+H)⁺.

The intermediates listed in below Table F were prepared by reacting an acid intermediate with a respective boronate ester with a procedure similar to the one described in synthesis of intermediate I-25 with appropriate variations in catalyst and base. The characterization data of the intermediates are summarized herein the below table.

TABLE F

| Intermediate | | Boronate |
|---|---|---|
| No | Acid used | ester used |
| F-1 | 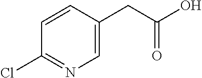 [SMILES: O=C(O)Cc1ccc(Cl)nc1] | 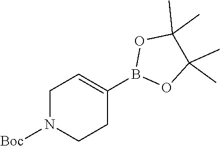 [SMILES: CC(C)(C)OC(=O)N1CC=C(B2OC(C)(C)C(C)(C)O2)CC1] |
| F-2 | 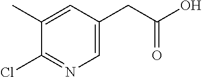 | 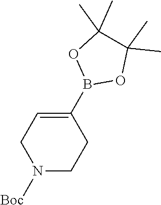 [SMILES: CC(C)(C)OC(=O)N1CC=C(B2OC(C)(C)C(C)(C)O2)CC1] |
| F-3 | 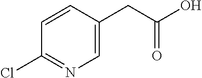 [SMILES: O=C(O)Cc1ccc(Cl)nc1] | 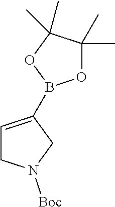 [SMILES: CC(C)(C)OC(=O)N1CC=C(B2OC(C)(C)C(C)(C)O2)C1] |
| F-4 | 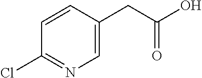 [SMILES: O=C(O)Cc1ccc(Cl)nc1] | A-1 |
| F-5 | 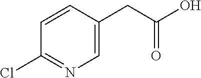 [SMILES: O=C(O)Cc1ccc(Cl)nc1] | A-4 |
| F-6 | 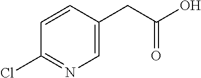 [SMILES: O=C(O)Cc1ccc(Cl)nc1] | A-3 |
| F-7 | 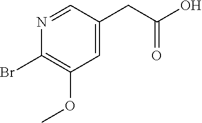 | 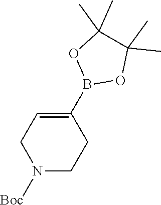 [SMILES: CC(C)(C)OC(=O)N1CC=C(B2OC(C)(C)C(C)(C)O2)CC1] |

| Intermediate | Intermediate | Characterization |
|---|---|---|
| No | structure | Data |
| F-1 | 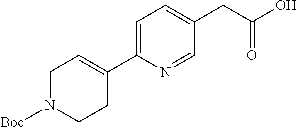 [SMILES: CC(C)(C)OC(=O)N1CC=C(c2ccc(CC(=O)O)cn2)CC1] | LCMS m/z: 319.20 (M + H)⁺ |
| F-2 | 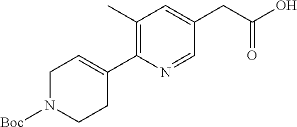 [SMILES: Cc1cc(CC(=O)O)cnc1C1=CCN(C(=O)OC(C)(C)C)CC1] | LCMS m/z: 333.12 (M + H)⁺ |
| F-3 | 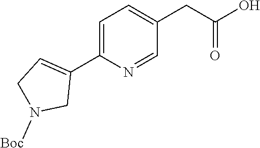 [SMILES: CC(C)(C)OC(=O)N1CC=C(c2ccc(CC(=O)O)cn2)C1] | LCMS m/z: 305.25 (M + H)⁺ |
| F-4 | 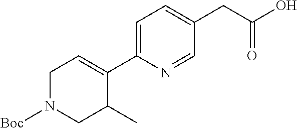 [SMILES: CC1CN(C(=O)OC(C)(C)C)CC=C1c1ccc(CC(=O)O)cn1] | LCMS m/z: 333.20 (M + H) ⁺ |
| F-5 | 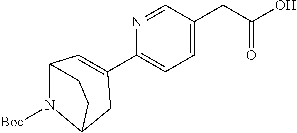 [SMILES: CC(C)(C)OC(=O)N1C2C=C(c3ccc(CC(=O)O)cn3)CC1CC2] | LCMS m/z: 345.20 (M + H) ⁺ |
| F-6 | 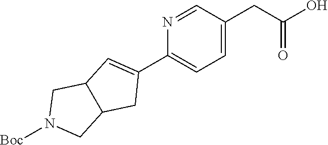 [SMILES: CC(C)(C)OC(=O)N1CC2C=C(c3ccc(CC(=O)O)cn3)CC2C1] | LCMS m/z: 345.20 (M + H) ⁺ |
| F-7 | 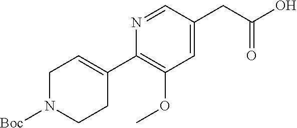 [SMILES: COc1cc(CC(=O)O)cnc1C1=CCN(C(=O)OC(C)(C)C)CC1] | LCMS m/z: 349.20 (M + H) ⁺ |

### Intermediate 1-26: Synthesis of 2-(6-(i-(tert-butoxycarbonyl) piperidin-3-yl) pyridin-3-yl) acetic acid

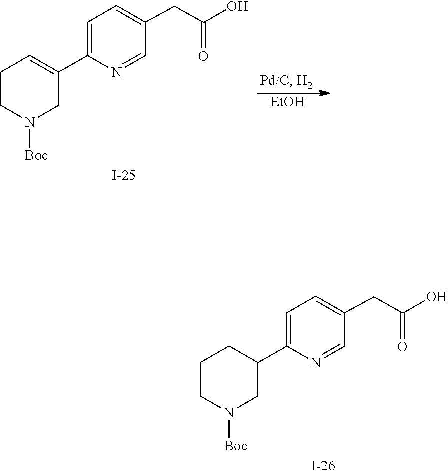

To a solution of 2-(1′-(tert-butoxycarbonyl)-1′,2′,5′,6′-tetrahydro-[2,3′-bipyridin]-5-yl)acetic acid (1.6 g, 5.03 mmol) in EtOH (20 mL) in a 500 mL parr shaker vessel was added Pd/C (0.7 g 10%). The mixture was shaken under 50 psi hydrogen atmosphere for 5 h. After completion of the reaction, the mixture was filtered through a celite pad, and the filtrate was concentrated to yield the product (1.5 g, 99.1%, LCMS: m/z=321.2 (M+H)⁺.

The intermediates listed in below Table G were prepared by a procedure similar to the one described in Intermediate I-26 with appropriate variations in reactants/reagents. The characterization data of the intermediates are summarized herein the below table.

TABLE G

| Intermediate | | | Characterization |
|---|---|---|---|
| No. | Reactant used | Intermediate structure | Data |
| G-1 | F-1 | 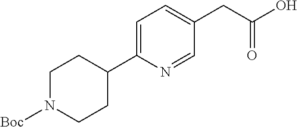 [SMILES: CC(C)(C)OC(=O)N1CCC(c2ccc(CC(=O)O)cn2)CC1] | LCMS m/z: 321.10 (M + H)⁺ |
| G-2 | F-3 | 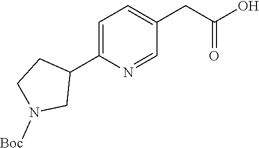 [SMILES: CC(C)(C)OC(=O)N1CCC(c2ccc(CC(=O)O)cn2)C1] | LCMS m/z: 307.10 (M + H)⁺ |
| G-3 | F-4 | 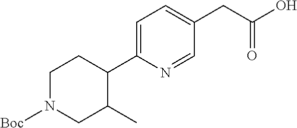 [SMILES: CC1CN(C(=O)OC(C)(C)C)CCC1c1ccc(CC(=O)O)cn1] | LCMS m/z: 335.20 (M + H) ⁺ |
| G-4 | F-5 | 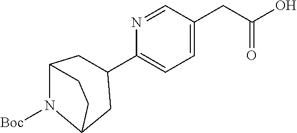 [SMILES: CC(C)(C)OC(=O)N1C2CCC1CC(c1ccc(CC(=O)O)cn1)C2] | LCMS m/z: 347.30 (M + H) ⁺ |
| G-5 | F-6 | 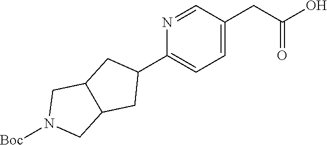 [SMILES: CC(C)(C)OC(=O)N1CC2CC(c3ccc(CC(=O)O)cn3)CC2C1] | LCMS m/z: 347.30 (M + H) ⁺ |
| G-6 | F-7 | 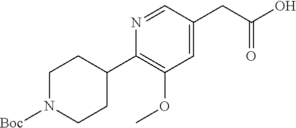 [SMILES: COc1cc(CC(=O)O)cnc1C1CCN(C(=O)OC(C)(C)C)CC1] | LCMS m/z: 351.30 (M + H) ⁺ |

### Intermediate 1-27: Synthesis of tert-butyl 5-(2-((4-bromo-5-chloropyridin-2-yl) amino)-2-oxoethyl)-5′,6′-dihydro-[2,3′-bipyridine]-1′(2′H)-carboxylate

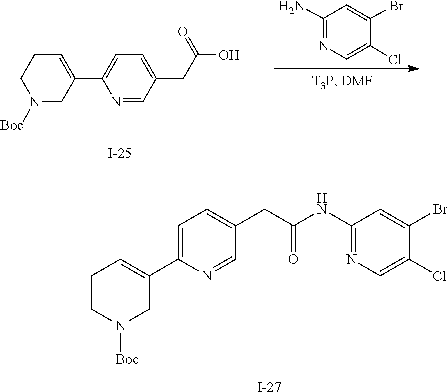

To a mixture of 2-(1′-(tert-butoxycarbonyl)-1′,2′,5′,6′-tetrahydro-[2,3′-bipyridin]-5-yl)acetic acid (2 g, 6.28 mmol) in DMF (10 mL) were added pyridine (0.99 g, 12.56 mmol) and a solution of T₃P (50% in EtOAc) (4.59 g, 14.44 mmol) at 0° C. over a period of 10 mi. The reaction mass was stirred for 10 min at the same temperature, followed by lot-wise addition of 4-bromo-5-chloropyridin-2-amine (1.69 g, 8.15 mmol) over a period of 5 min. After addition, the reaction mixture was warmed to room temperature and stirred overnight. The reaction mass was quenched with ice water, extracted with ethyl acetate and the organic layer was separated from the aqueous layer. The aqueous layer was further extracted with ethyl acetate, and the combined organic layers were dried over anhydrous sodium sulphate and concentrated under reduced pressure to afford crude product. This material was further purified by Combiflash using an ethyl acetate:hexane (20-30%) mixture as mobile phase to afford pure title compound (2.1 g, 65.85%). ¹HNMR (DMSO-d₆, 400 MHz): δ 8.57 (s, 1H), 8.15 (s, 1H), 8.21 (s, 1H), 7.90 (s, 1H), 7.67 (d, 1H), 7.41 (d, 1H), 6.62 (s, 1H), 4.14 (s, 2H), 3.74 (s, 2H), 3.64-3.63 (m, 2H), 2.65 (i, 2H), 1.48 (s, 9H); LCMS: m/z=509.10 (M+H)⁺.

The intermediates listed in below Table H were prepared by reacting a carboxylic acid with an appropriate amine using a procedure similar to the one described in the synthesis of Intermediate I-27 with appropriate variations. The characterization data of the intermediates are summarized herein the below table.

TABLE H

| | Carboxylic | | | |
|---|---|---|---|---|
| Int. | Acid | | | Characterization |
| No. | used | Amine used | Intermediate structure | data |
| H-1 | I-25 | 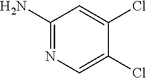 | 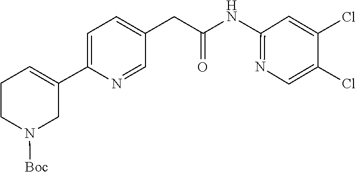 [SMILES: CC(C)(C)OC(=O)N1CCC=C(c2ccc(CC(=O)Nc3cc(Cl)c(Cl)cn3)cn2)C1] | LCMS m/z: 463.10 (M + H) ⁺ |
| H-2 | F-3 | 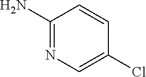 | 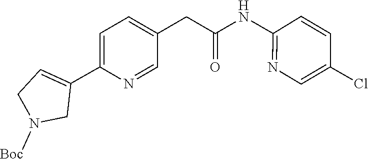 [SMILES: CC(C)(C)OC(=O)N1CC=C(c2ccc(CC(=O)Nc3ccc(Cl)cn3)cn2)C1] | LCMS m/z: 415.20 (M + H)⁺ |
| H-3 | G-2 | 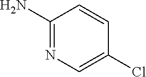 | 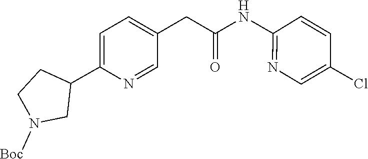 [SMILES: CC(C)(C)OC(=O)N1CCC(c2ccc(CC(=O)Nc3ccc(Cl)cn3)cn2)C1] | LCMS m/z: 417.80 (M + H)⁺ |
| H-4 | F-2 | 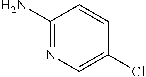 | 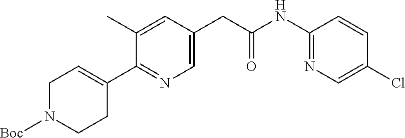 [SMILES: Cc1cc(CC(=O)Nc2ccc(Cl)cn2)cnc1C1=CCN(C(=O)OC(C)(C)C)CC1] | LCMS m/z: 443.80 (M + H)⁺ |
| H-5 | G-1 | I-16 | 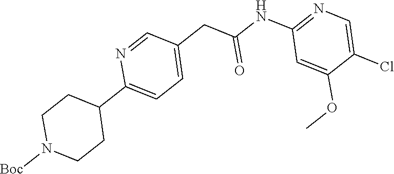 [SMILES: COc1cc(NC(=O)Cc2ccc(C3CCN(C(=O)OC(C)(C)C)CC3)nc2)ncc1Cl] | LCMS m/z: 461.80 (M + H) ⁺ |
| H-6 | G-3 | I-16 | 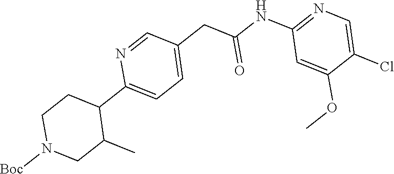 [SMILES: COc1cc(NC(=O)Cc2ccc(C3CCN(C(=O)OC(C)(C)C)CC3C)nc2)ncc1Cl] | LCMS m/z: 475.80 (M + H) ⁺ |
| H-7 | I-26 | I-16 | 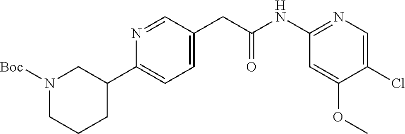 [SMILES: COc1cc(NC(=O)Cc2ccc(C3CCCN(C(=O)OC(C)(C)C)C3)nc2)ncc1Cl] | LCMS m/z: 461.80 (M + H) ⁺ |
| H-8 | G-1 | I-9 | 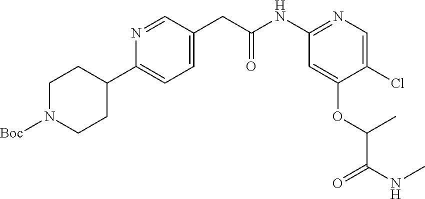 [SMILES: CNC(=O)C(C)Oc1cc(NC(=O)Cc2ccc(C3CCN(C(=O)OC(C)(C)C)CC3)nc2)ncc1Cl] | LCMS m/z: 532.83 (M + H) ⁺ |
| H-9 | G-1 | B-2 | 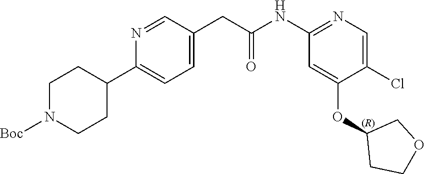 | LCMS m/z: 517.90 (M + H) ⁺ |
| H-10 | G-1 | B-3 | 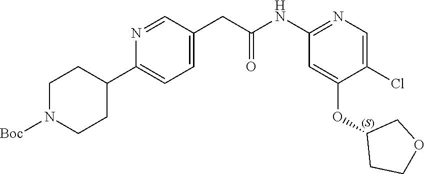 | LCMS m/z: 517.90 (M + H) ⁺ |
| H-11 | G-1 | I-10 | 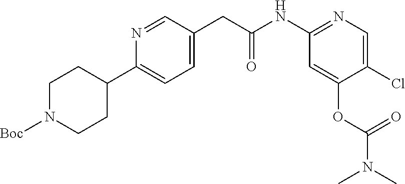 [SMILES: CN(C)C(=O)Oc1cc(NC(=O)Cc2ccc(C3CCN(C(=O)OC(C)(C)C)CC3)nc2)ncc1Cl] | LCMS m/z: 518.90 (M + H) ⁺ |
| H-12 | G-4 | I-16 | 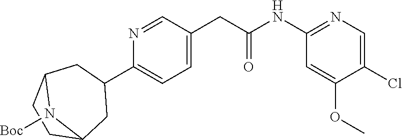 [SMILES: COc1cc(NC(=O)Cc2ccc(C3CC4CCC(C3)N4C(=O)OC(C)(C)C)nc2)ncc1Cl] | LCMS m/z: 487.90 (M + H) ⁺ |
| H-13 | G-5 | I-16 | 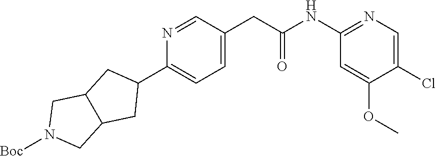 [SMILES: COc1cc(NC(=O)Cc2ccc(C3CC4CN(C(=O)OC(C)(C)C)CC4C3)nc2)ncc1Cl] | LCMS m/z: 487.90 (M + H) ⁺ |
| H-14 | I-26 | I-9 | 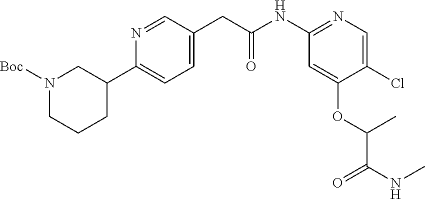 [SMILES: CNC(=O)C(C)Oc1cc(NC(=O)Cc2ccc(C3CCCN(C(=O)OC(C)(C)C)C3)nc2)ncc1Cl] | LCMS m/z: 532.83 (M + H) ⁺ |
| H-15 | I-26 | B-2 | 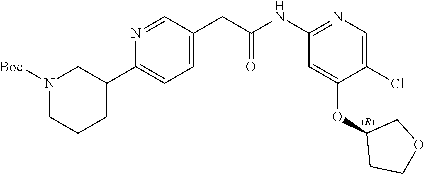 | LCMS m/z: 517.80 (M + H) ⁺ |
| H-16 | I-26 | B-3 | 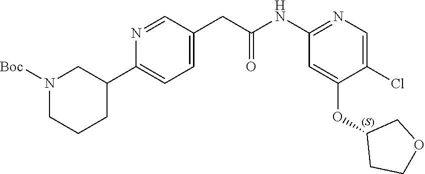 | LCMS m/z: 517.80 (M + H) ⁺ |
| H-17 | G-1 | B-5 | 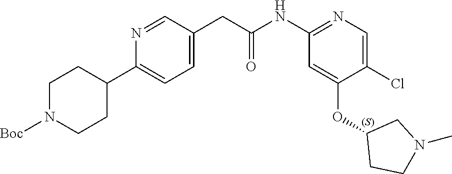 | LCMS m/z: 530.80 (M + H) ⁺ |
| H-18 | G-1 | B-6 | 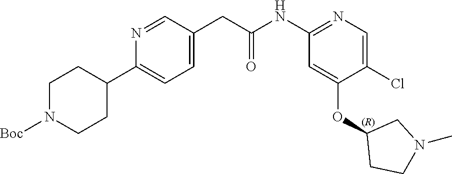 | LCMS m/z: 530.80 (M + H) ⁺ |
| H-19 | G-1 | 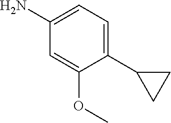 | 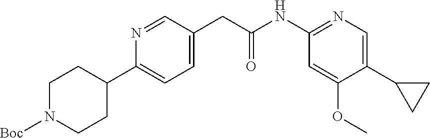 [SMILES: COc1cc(NC(=O)Cc2ccc(C3CCN(C(=O)OC(C)(C)C)CC3)nc2)ncc1C1CC1] | LCMS m/z: 467.40 (M + H) ⁺ |
| H-20 | G-6 | I-16 | 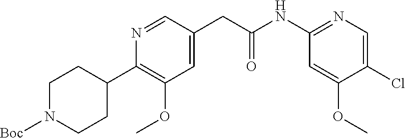 [SMILES: COc1cc(NC(=O)Cc2cnc(C3CCN(C(=O)OC(C)(C)C)CC3)c(OC)c2)ncc1Cl] | LCMS m/z: 491.70 (M + H) ⁺ |
| H-21 | G-6 | B-2 | 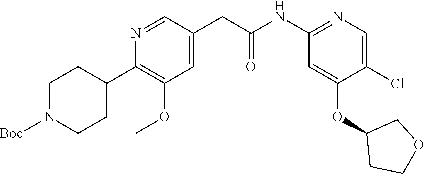 [SMILES: COc1cc(CC(=O)Nc2cc(O[C@@H]3CCOC3)c(Cl)cn2)cnc1C1CCN(C(=O)OC(C)(C)C)CC1] | LCMS m/z: 447.70 (M + H) ⁺ |
| H-22 | G-1 | B-18 | 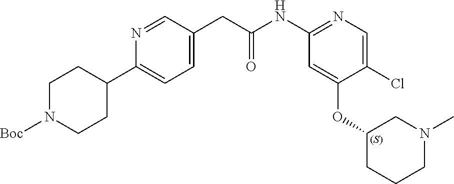 | LCMS m/z: 545.70 (M + H) ⁺ |
| H-23 | G-1 | B-17 | 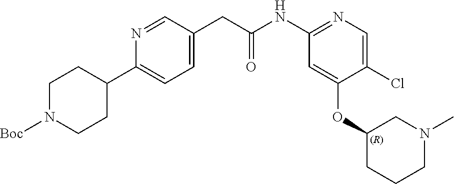 | LCMS m/z: 545.70 (M + H) ⁺ |
| H-24 | G-1 | I-12 | 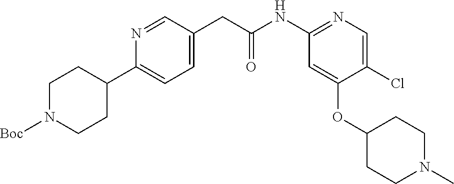 [SMILES: CN1CCC(Oc2cc(NC(=O)Cc3ccc(C4CCN(C(=O)OC(C)(C)C)CC4)nc3)ncc2Cl)CC1] | LCMS m/z: 545.70 (M + H) ⁺ |
| H-25 | F-5 | I-16 | 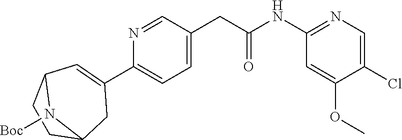 [SMILES: COc1cc(NC(=O)Cc2ccc(C3=CC4CCC(C3)N4C(=O)OC(C)(C)C)nc2)ncc1Cl] | LCMS m/z: 485.70 (M + H) ⁺ |
| H-26 | G-6 | I-9 | 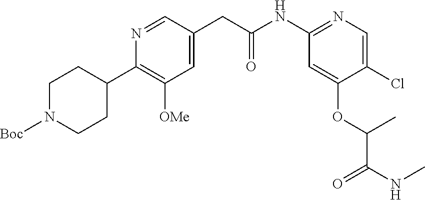 [SMILES: CNC(=O)C(C)Oc1cc(NC(=O)Cc2cnc(C3CCN(C(=O)OC(C)(C)C)CC3)c(OC)c2)ncc1Cl] | LCMS m/z: 562.80 (M + H) ⁺ |

### Intermediate I-28: Synthesis of 2-Bromo-4-methoxy-6-cyano-pyridine

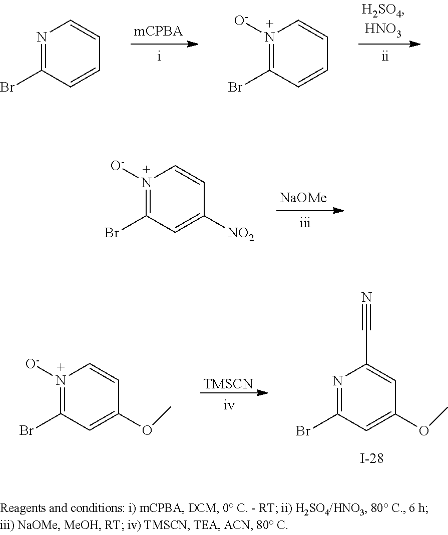

### Step-i: Synthesis of 2-Bromopyridine-N-oxide

To a stirred solution of 2-bromopyridine (10 g, 63.28 mmol) in DCM (50 mL) was added mCPBA (16.38 g, 94.93 mmol) at 0° C. for 10 min. After the addition mixture was stirred at RT overnight. The reaction mixture was diluted with saturated NaHCO₃ and separated the organic layer and aqueous layer. The organic layer was dried over anhydrous sodium sulphate and concentrated under vacuum. The crude compound was purified by silica gel column chromatography by eluting with 0-50% ethyl acetate-hexane to afford the title compound (8.3 g 75.37%), LCMS m/z: 174 (M+H)⁺.

### Step-ii: Synthesis of 2-Bromo-4-nitro-pyridine-N-oxide

2-Bromopyridine-N-oxide (5 g, 28.73 mmol) was added to H₂SO₄ (30 mL) at 0° C. and reaction mixture was heated at 80° C., Nitric acid (14 mL) was added dropwise to the mixture at same temperature for 5 min. After addition reaction mixture was stirred at 80° C. for 6 h, then mixture was cooled to RT, diluted with ice water, adjusted pH to 8 using 2N NaOH solution, the yellow solid obtained was filtered and dried (2.6 g 41.3%), LCMS: m/z=219 (M+H)⁺.

### Step-iii: Synthesis of 2-Bromo-4-methoxy-pyridine-N-oxide

To a stirred solution of 2-Bromo-4-nitro-pyridine-N-oxide (1 g, 4.56 mmol) in MeOH (10 mL) was added NaOMe (0.25 g, 4.56 mmol) at 0° C. and mixture was stirred at RT overnight. After completion of the reaction, the mixture was quenched with ice cold water and extracted with ethyl acetate. The organic layer was washed with brine, dried over sodium sulphate, and concentrated under vacuum. The crude residue was purified by Combi flash, and the product eluted at 0-5% DCM-MeOH as an off-white solid (0.7 g 75.16%). LCMS: m/z=204 (M+H)⁺.

### Step-iv: Synthesis of 2-Bromo-4-methoxy-6-cyanopyridine

To a stirred solution of 2-Bromo-4-methoxy-pyridine-N-oxide (2.0 g, 9.803 mmol) in acetonitrile (10 mL) was added TEA (2.97 g, 29.4 mmol) followed N, N-dimethylcarbomyl chloride (2.10 g, 19.6 mmol) and TMSCN (1.94 g, 19.6 mmol) at 0° C. The reaction mixture was heated at 80° C. overnight. After the completion of the reaction, the mixture was quenched into ice water and extracted with EtOAc. The organic layer was dried over sodium sulphate and concentrated. The crude product was purified by Combiflash with 0-5% DCM-MeOH as eluent to give the titled product (0.9 g 43.1%), ¹HNMR (CDCl3, 400 MHz): δ 7.19 (s, 2H), 3.92 (s, 3H).

### Intermediate I-29: Synthesis of 6-bromo-4-(2-methoxyethoxy) picolinonitrile

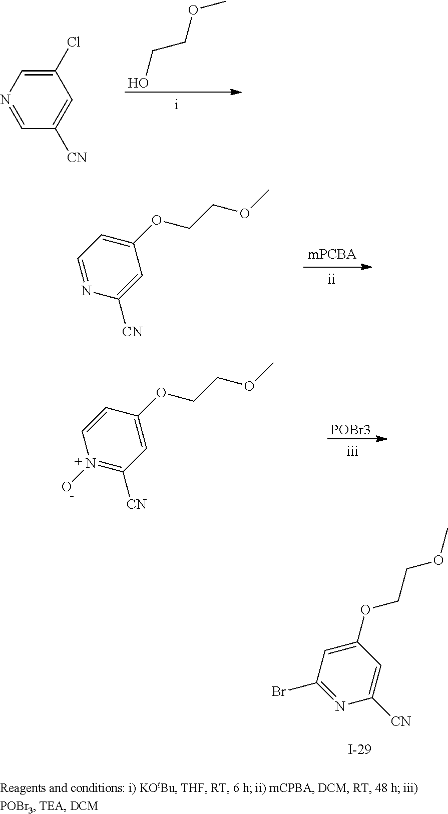

### Step-i: Synthesis of 4-(2-methoxyethoxy) picolinonitrile

To a stirred solution of 2-methoxyethan-1-ol (0.65 g, 8.66 mmol) in dry THF (5 mL) was added potassium tert-butoxide (0.81 g, 7.21 mmol) and reaction mixture stirred at 0° C. for 10 min. 4-chloropicolinonitrile (1.0 g, 7.21 mmol) was added to the mixture and stirred for 6 h at RT. After the completion of the reaction, mixture was quenched into ice water and extracted with EtOAc, organic layer was dried over sodium sulphate and concentrated. Crude was purified by Combi flash with EtOAc:hexane as eluent to get 4-(2-methoxyethoxy) picolinonitrile (1.0 g, 77.76%). LCMS: m/z=179.1 (M+H)⁺.

### Step-ii: Synthesis of 2-cyano-4-(2-methoxyethoxy) pyridine 1-oxide

To a stirred solution of 4-(2-methoxyethoxy) picolinonitrile (2 g, 11.2 mmol) in DCM (15 mL) was added mCPBA (1.93, 11.22 mmol) at 0° C. for 10 min. After addition stirred the reaction mixture at RT for 48 h. The mixture was diluted with saturated NaHCO₃. Separated the organic layer and aqueous layer. The organic layer was dried over anhydrous sodium sulphate and concentrated under vacuum. The crude compound was purified by silica gel column chromatography by eluting with 0-50% ethyl acetate-hexane to afford the title compound (1.5 g, 68.82%),LCMS: m/z=195.1 (M+H)⁺.

### Step-iii: Synthesis of 6-bromo-4-(2-methoxyethoxy) picolinonitrile

To a stirred solution of 2-cyano-4-(2-methoxyethoxy) pyridine 1-oxide (1.2 g, 6.17 mmol) in dry DCM (15 mL) was added TEA (1.25 g, 12.36 mmol), then reaction mixture stirred in at 0° C. for 10 min, added POBr₃ (3.5 g, 12.36 mmol). After the completion of the reaction, mixture was quenched with saturated NaHCO₃, extracted with DCM. The organic layer was dried over sodium sulphate and concentrated. Crude was purified by Combi flash with EtOAc:hexane as eluent to get 4-(2-methoxyethoxy) picolinonitrile (0.5 g, 31.47%). LCMS: m/z=259 (M+H)⁺. ¹HNMR(CDCl3, 400 MHz): δ 7.21 (s, 1H), 7.05 (s, 1H), 4.22 (t, 2H), 3.77-3.75 (m, 2H), 3.43 (s, 3H).

### Intermediate I-30: Synthesis of Synthesis of 6-chloro-4-fluoropicolinonitrile

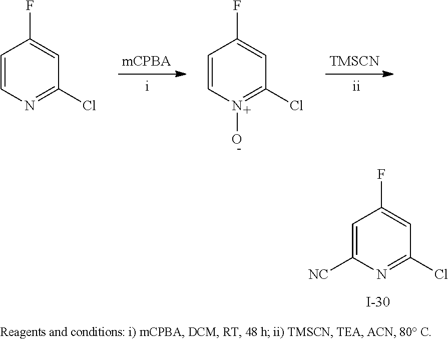

### Step-i: Synthesis of 2-chloro-4-fluoro-Pyridine-N-oxide

To a stirred solution of 2-chloro-4-fluoro-Pyridine (10 g, 76.028 mmol) in DCM (50 mL) was added mCPBA (26.24 g, 152.05 mmol) at 0° C. for 10 min. After addition stirred the reaction mixture at RT for 48 h. The reaction mixture was diluted with saturated NaHCO₃, separated the organic layer and aqueous layer. The organic layer was dried over anhydrous sodium sulphate and concentrated under vacuum. The crude compound was purified by silica gel column chromatography by eluting with 0-50% ethyl acetate-hexane to afford the title compound (9.5 g 84.70%), LCMS: m/z=148.1 (M+H)⁺.

### Step-ii: Synthesis of 6-chloro-4-fluoropicolinonitrile

To a stirred solution of 2-Bromo-4-methoxy-Pyridine-N-oxide (9.0 g, 61.00 mmol) in acetonitrile (60 mL) was added TEA (12.34 g, 122.01 mmol), TMSCN (18.15 g, 183.01 mmol) at 0° C. The reaction mixture was heated at 80° C. overnight. The reaction mixture was diluted with ice water, extracted with EtOAc, The organic layer was dried over anhydrous sodium sulphate and concentrated under vacuum. The crude compound was purified by silica gel column chromatography by eluting with 0-50% EtOAc/Hexane to afford the title compound (1 g 10.47%), LCMS: m/z=157.1 (M+H)⁺.

### Intermediate I-31: Synthesis of 6-chloro-4-((1-methylpiperidin-4-yl) oxy) picolinonitrile

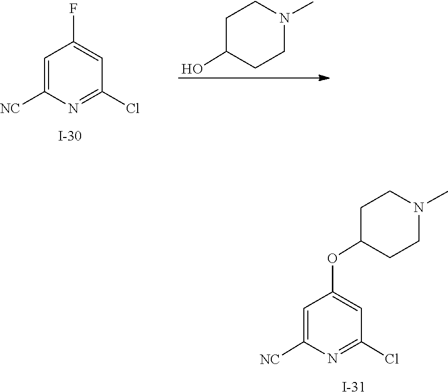

To a stirred solution of 1-methylpiperidin-4-ol (0.26 g, 2.29 mmol) in DMF (3 mL) was added NaH (60%, dispersion in Paraffin Liquid) (0.07 g, 2.87 mmol) at 0° C. followed by 6-chloro-4-fluoropicolinonitrile (0.3 g, 1.915 mmol). The reaction mixture was stirred at 0° C. for 1 h. The reaction mixture was diluted with ice water, extracted with EtOAc, the organic layer was dried over anhydrous sodium sulphate and concentrated under vacuum. The crude compound was purified by combiflash by eluting with 0-5% DCM-MeOH to afford the title compound (0.23 g, 47.69%), LCMS: m/z=152.1 (M+H)⁺.

The compounds listed in below Table P were prepared by reacting 6-chloro-4-fluoropicolinonitrile with respective reagent and suitable base with a procedure similar to the one described in the synthesis of Intermediate 1-31 with appropriate variations. The characterization data of the intermediates are summarized herein the below table.

TABLE P

| Intermediate | Reagent | Base | | Characterization |
|---|---|---|---|---|
| No. | used | used | Intermediate Structure | data |
| P-1 | 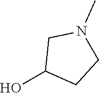 | NaH | 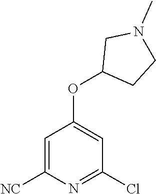 [SMILES: CN1CCC(Oc2cc(Cl)nc(C#N)c2)C1] | LCMS m/z: 238.10 (M + H)⁺ |
| P-2 | 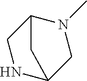 | DIPEA | 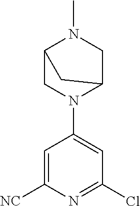 [SMILES: CN1CC2CC1CN2c1cc(Cl)nc(C#N)c1] | LCMS m/z: 249.10 (M + H)⁺ |
| P-3 | 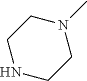 | DIPEA | 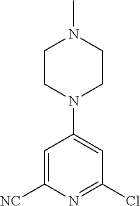 [SMILES: CN1CCN(c2cc(Cl)nc(C#N)c2)CC1] | LCMS m/z: 237.01 (M + H)⁺ |
| P-4 | 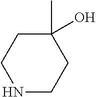 | DIPEA | 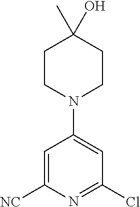 [SMILES: CC1(O)CCN(c2cc(Cl)nc(C#N)c2)CC1] | LCMS m/z: 252.3 (M + H) ⁺ |
| P-5 | 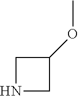 | DIPEA | 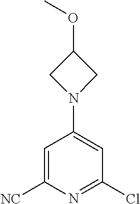 | LCMS m/z: 224.10 (M + H) ⁺ |
| P-6 | 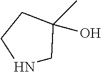 | DIPEA | 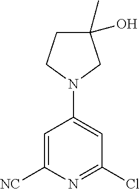 [SMILES: CC1(O)CCN(c2cc(Cl)nc(C#N)c2)C1] | LCMS m/z: 238.3 (M + H) ⁺ |
| P-7 | 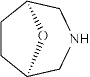 | DIPEA | 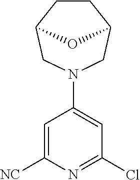 [SMILES: N#Cc1cc(N2C[C@H]3CC[C@@H](C2)O3)cc(Cl)n1] | LCMS m/z: 250.6 (M + H) ⁺ |
| P-8 | 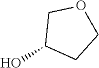 | DIPEA | 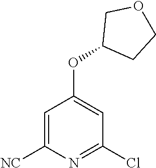 [SMILES: N#Cc1cc(O[C@H]2CCOC2)cc(Cl)n1] | LCMS m/z: 225.5 (M + H) ⁺ |
| P-9 | 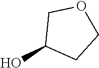 | DIPEA | 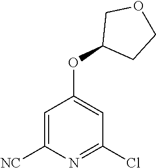 [SMILES: N#Cc1cc(O[C@@H]2CCOC2)cc(Cl)n1] | LCMS m/z: 225.6 (M + H) ⁺ |
| P-10 | 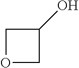 | DIPEA | 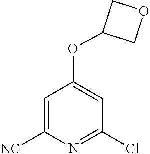 [SMILES: N#Cc1cc(OC2COC2)cc(Cl)n1] | LCMS m/z: 211.5 (M + H) ⁺ |
| P-11 | 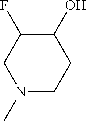 | DIPEA | 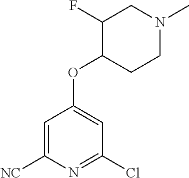 [SMILES: CN1CCC(Oc2cc(Cl)nc(C#N)c2)C(F)C1] | LCMS m/z: 270.6 (M + H) ⁺ |
| P-12 | 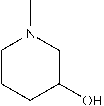 | DIPEA | 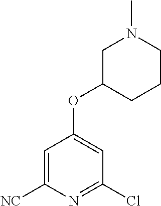 [SMILES: CN1CCCC(Oc2cc(Cl)nc(C#N)c2)C1] | LCMS m/z: 252.6 (M + H) ⁺ |
| P-13 | 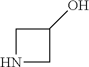 | DIPEA | 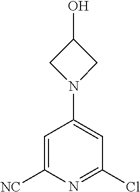 [SMILES: N#Cc1cc(N2CC(O)C2)cc(Cl)n1] | LCMS m/z: 210.5 (M + H) ⁺ |
| P-14 | 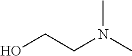 | DIPEA | 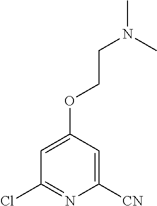 | LCMS m/z: 226.6 (M + H) ⁺ |
| P-15 | 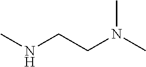 | DIPEA | 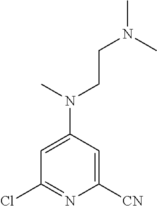 [SMILES: CN(C)CCN(C)c1cc(Cl)nc(C#N)c1] | LCMS m/z: 239.5 (M + H) ⁺ |

### EXAMPLES

### Example 1: Synthesis of 2-((5-chloro-2-(2-(1′-cyano-1′,2′,3′,6′-tetrahydro-[2,4′-bipyridin]-5-yl)acetamido)pyridin-4-yl)oxy)-N-methylpropanamide (Compound 1)

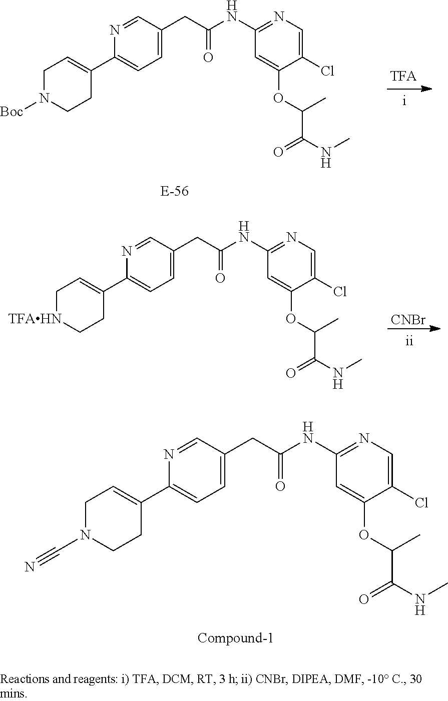

### Step-i: Synthesis of Trifluoroacetate salt of 2-((5-chloro-2-(2-(1′,2′,3′,6′-tetrahydro-[2,4′-bipyridin]-5-yl)acetamido)pyridin-4-yl)oxy)-N-methylpropanamide

To a stirred solution of tert-butyl 5-(2-((5-chloro-4-((i-(methylamino)-i-oxopropan-2-yl)oxy)pyridin-2-yl)amino)-2-oxoethyl)-3′,6′-dihydro-[2,4′-bipyridine]-1′(2′H)-carboxylate (0.2 g, 0.38 mmol) in DCM (5 mL) was added TFA (0.27 mL, 3.51 mmol) at 0° C. Then the reaction was stirred for 3 h at RT. Solvent was evaporated under vacuum, and the residue was washed with diethyl ether and subsequently dried under vacuum to yield the trifluoroacetate salt of 2-((5-chloro-2-(2-(1′,2′,3′,6′-tetrahydro-[2,4′-bipyridin]-5-yl)acetamido)pyridin-4-yl)oxy)-N-methylpropanamide. (0.16 g, 94%); LCMS: 430.20 (M+H)⁺.

### Step-ii: Synthesis of 2-((5-chloro-2-(2-(1′-cyano-1′,2′,3′,6′-tetrahydro-[2,4′-bipyridin]-5-yl)acetamido)pyridin-4-yl)oxy)-N-methylpropanamide

To a solution of the trifluoroacetate salt of 2-((5-chloro-2-(2-(1′,2′,3′,6′-tetrahydro-[2,4′-bipyridin]-5-yl)acetamido)pyridin-4-yl)oxy)-N-methylpropanamide (0.15 g, 0.35 mmol) in DMF (5 mL) was added DIPEA (0.12 mL, 0.70 mmol) at −10° C. The mixture was stirred for 10 min and then cyanogen bromide (0.04 g, 0.35 mmol) was added at −10° C. Stirring was continued for 30 min. The reaction was monitored by TLC, and, after completion, was quenched by aq. NaHCO₃ and extracted with DCM. The organic layer was dried over anhydrous sodium sulphate and concentrated. The crude product was further purified by Combiflash using a DCM:MeOH (0-5%) mixture as mobile phase to afford pure titled compound (0.070 g, 44%). ¹HNMR (DMSO-d₆, 400 MHz): δ 10.95 (s, 1H), 8.48 (s, 1H), 8.23 (s, 1H), 7.79-7.75 (m, 2H), 7.49-7.45 (m, 1H), 6.64-6.60 (m, 1H), 4.74-4.72 (m, 1H), 3.99-3.98 (m, 2H), 3.88 (s, 2H), 3.48-3.45 (m, 2H), 2.61 (s, 2H), 2.56-2.55 (m, 3H), 2.45 (s, 1H), 1.44-1.42 (m, 3H); LCMS m/z: 455.20 (M+H)⁺; HPLC: 99.10%, rt: 6.20 min.

Racemic tert-butyl 5-(2-((5-chloro-4-((1-(methylamino)-1-oxopropan-2-yl)oxy)pyridin-2-yl)amino)-2-oxoethyl)-3′,6′-dihydro-[2,4′-bipyridine]-1′(2′H)-carboxylate was separated using a chiral preparative HPLC column (Method: Column: Regis reflect, Cellulose-C, 21.1 mm×250 mm, 5 micron), Elution:isocratic (45:55), A=Hexane, B=MeOH, Flow: 20 mL/min) to afford the pure Isomer-1 and Isomer-2.

Isomer-1 (Compound 2): ¹HNMR (DMSO-d₆, 400 MHz): δ 10.95 (s, 1H), 8.48 (s, 1H), 8.23 (s, 1H), 7.79-7.75 (m, 2H), 7.49-7.45 (m, 1H), 6.64-6.60 (m, 1H), 4.74-4.72 (m, 1H), 3.99-3.98 (m, 2H), 3.88 (s, 2H), 3.48-3.45 (m, 2H), 2.61 (s, 2H), 2.56-2.55 (m, 3H), 2.45 (s, 1H), 1.44-1.42 (m, 3H); LCMS m/z: 455.20 (M+H)⁺; HPLC: 99.10%, rt: 6.20 min.

Isomer-2 (Compound 3): ¹HNMR (DMSO-d₆, 400 MHz): δ 10.95 (s, 1H), 8.48 (s, 1H), 8.23 (s, 1H), 7.79-7.75 (m, 2H), 7.49-7.45 (m, 1H), 6.64-6.60 (m, 1H), 4.74-4.72 (m, 1H), 3.99-3.98 (m, 2H), 3.88 (s, 2H), 3.48-3.45 (m, 2H), 2.61 (s, 2H), 2.56-2.55 (m, 3H), 2.45 (s, 1H), 1.44-1.42 (m, 3H); LCMS m/z: 455.20 (M+H)⁺; HPLC: 99.10%, rt: 6.20 min.

The compounds listed in below Table I were prepared by reacting an appropriate Boc-protected amine intermediate as listed in the below table with TFA followed by cyanogen bromide using a procedure similar to the one described in Example 1 with appropriate variations known to a person skilled in the art. The E-intermediates and H-intermediates cited in below table were prepared as per either Scheme-I or Scheme-II as appropriately referred in the respective tables. The characterization data of the compounds are summarized herein the below table.

TABLE I

| Compd. | Int. | | |
|---|---|---|---|
| No | No. | Compound structure | Characterization Data |
| 4 | E-3 | 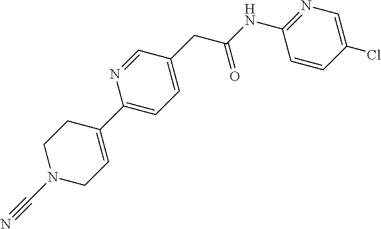 [SMILES: N#CN1CC=C(c2ccc(CC(=O)Nc3ccc(Cl)cn3)cn2)CC1] | ¹HNMR (DMSO-d₆, 400 MHz): δ 10.98 (s, 1H), 8.48 (s, 1H), 8.38 (s, 1H), 8.08-8.06 (m, 1H), 7.89-7.87 (m, 1H), 7.74-7.72 (m, 1H), 7.55- 7.53 (m, 1H) 6.64 (s, 1H), 3.98 (s, 2H), 3.78 (s, 2H), 3.44-3.41 (m, 2H), 2.66-2.65 (m, 2H); LCMS m/z: 354.10 (M + H)⁺; HPLC: 98.94%, rt: 3.89 min. |
| 5 | E-6 | 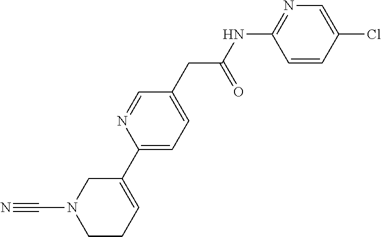 [SMILES: N#CN1CCC=C(c2ccc(CC(=O)Nc3ccc(Cl)cn3)cn2)C1] | ¹HNMR (DMSO-d₆, 400 MHz): δ 10.96 (s, 1H), 8.46 (s, 1H), 8.38 (s, 1H), 8.78-8.05-8.01 (m, 1H), 7.90- 7.87 (m, 1H), 7.74-7.71 (m, 1H), 7.71-7.60 (m, 1H), 6.79 (s, 1H), 4.22- 4.21 (m, 2H), 3.77 (s, 2H), 3.36-3.34 (m, 2H), 2.41-2.40 (m, 2H); LCMS m/z: 354.10 (M + H)⁺; HPLC: 98.21%, rt: 4.71 min. |
| 6 | E-4 | 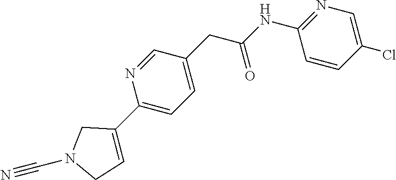 [SMILES: N#CN1CC=C(c2ccc(CC(=O)Nc3ccc(Cl)cn3)cn2)C1] | ¹HNMR (DMSO-d₆, 400 MHz): δ 10.92 (s, 1H), 8.48 (s, 1H), 8.38 (s, 1H), 8.08-8.05 (m, 1H), 7.90 (d, 1H), 7.77 (d, 1H), 7.65-7.62 (m, 1H), 6.63-6.62 (m, 1H), 4.68-4.64 (m, 2H), 4.48-4.47 (m, 2H), 3.81 (s, 2H); LCMS m/z: 340.10 (M + H)⁺; HPLC: 98.01%, rt: 4.63 min. |
| 7 | E-7 | 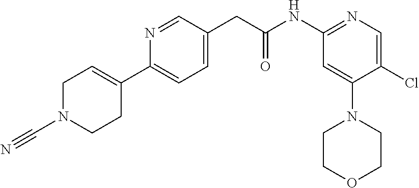 [SMILES: N#CN1CC=C(c2ccc(CC(=O)Nc3cc(N4CCOCC4)c(Cl)cn3)cn2)CC1] | ¹HNMR (DMSO-d₆, 400 MHz): δ 10.86 (s, 1H), 8.47 (d, 1H), 8.18 (s, 1H), 7.8 (s, 1H), 7.66 (d, 1H), 7.56 (d, 1H), 6.76- (s, 1H), 3.97 (d, 2H), 3.77 (s, 2H), 3.72-3.69 (m, 4H), 3.43-3.41 (m, 2H), 3.10-3.27 (m, 4H), 2.65 (s, 2H); LCMS m/z: 439.10 (M + H)⁺; HPLC: 97.33%, rt: 5.83 min. |
| 8 | E-2 | 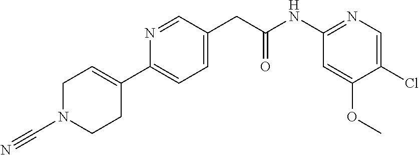 [SMILES: COc1cc(NC(=O)Cc2ccc(C3=CCN(C#N)CC3)nc2)ncc1Cl] | ¹HNMR (DMSO-d₆, 400 MHz): δ 10.95 (s, 1H), 8.48 (s, 1H), 8.23 (s, 1H), 7.91 (s, 1H), 7.73 (d, 1H), 7.55 (d, 1H), 6.64 (s, 1H), 3.99 (s, 2H), 3.88 (s, 3H), 3.78 (s, 2H), 3.44-3.41 (m, 2H), 2.66-2.61 (m, 2H); LCMS m/z: 384.80 (M + H)⁺; HPLC: 98.40%, rt: 4.09 min. |
| 9 | E-8 | 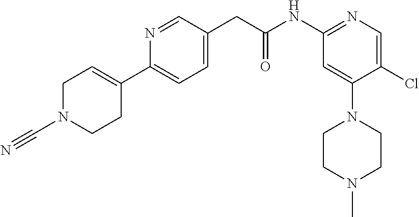 [SMILES: CN1CCN(c2cc(NC(=O)Cc3ccc(C4=CCN(C#N)CC4)nc3)ncc2Cl)CC1] | ¹HNMR (DMSO-d₆, 400 MHz): δ 10.81 (s, 1H), 8.45 (s, 1H), 8.13 (s, 1H), 7.99 (s, 1H), 7.74-7.72 (m, 1H), 7.54-7.51 (m, 1H), 6.62 (s, 1H), 3.97 (s, 2H), 3.74 (s, 2H), 3.41-3.39 (m, 2H), 3.10-3.08 (m, 4H), 2.65-2.64 (m, 2H), 2.47-2.43 (m, 4H), 2.19 (s, 3H); LCMS m/z: 452.20 (M + H)⁺; HPLC: 95.56%, rt: 5.15 min. |
| 10 | E-11 | 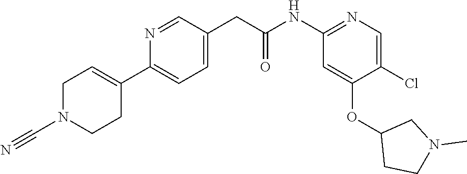 [SMILES: CN1CCC(Oc2cc(NC(=O)Cc3ccc(C4=CCN(C#N)CC4)nc3)ncc2Cl)C1] | ¹HNMR (DMSO-d₆, 400 MHz): δ 10.93 (s, 1H), 8.47 (s, 1H), 8.22 (s, 1H), 7.84(s, 1H), 7.72-7.70 (m, 1H), 7.55 (d, 1H), 6.64 (bs, 1H), 4.91-4.88 (m, 1H), 3.99 (d, 2H), 3.78 (s, 2H), 3.42-3.40 (m, 2H), 2.67-2.72 (m, 6H), 2.28-2.33 (m, 2H), 2.24 (s, 3H); LCMS m/z: 453.10 (M + H)⁺; HPLC: 98.1%, rt: 6.60 min. |
| 11 | E-10 | 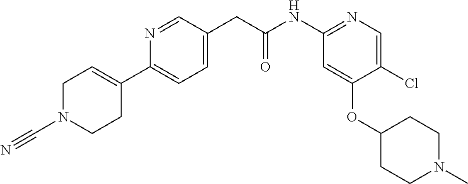 [SMILES: CN1CCC(Oc2cc(NC(=O)Cc3ccc(C4=CCN(C#N)CC4)nc3)ncc2Cl)CC1] | ¹HNMR (DMSO-d₆, 400 MHz): δ 10.82 (s, 1H), 8.47 (s, 1H), 8.23 (s, 1H), 7.90 (s, 1H), 7.72-7.69 (m, 1H), 7.55 (d, 1H), 6.64-6.61 (m, 1H), 4.50-4.48 (m, 1H), 3.98 (d, 2H), 3.78 (s, 2H), 3.43-3.40 (m, 3H), 2.67-2.63 (m, 3H), 2.32-2.30 (m, 2H), 2.21 (s, 3H), 1.92-1.89 (m, 2H), 1.70-1.67 (m, 2H); LCMS m/z: 467.10 (M + H)⁺; HPLC: 97.3%, rt: 2.83 min. |
| 12 | E-5 | 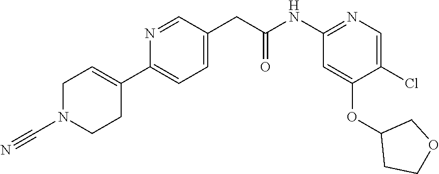 [SMILES: N#CN1CC=C(c2ccc(CC(=O)Nc3cc(OC4CCOC4)c(Cl)cn3)cn2)CC1] | ¹HNMR (CDCl₃, 400 MHz): δ 10.96 (s, 1H), 8.48-8.47 (s, 1H), 8.25 (s, 1H), 7.88 (s, 1H), 7.74- 7.72 (d, 1H), 7.56-7.54 (d, 1H), 6.64-6.61 (m, 1H), 5.11-5.08 (m, 1H), 3.99 (d, 2H), 3.87-3.72 (m, 6H), 3.44-3.41 (m, 2H), 2.66 (s, 2H), 2.28-2.23 (m, 1H), 2.00-1.96 (m, 1H); LCMS m/z: 440.00 (M + H)⁺; HPLC 97.38%, rt: 3.48 min. |
| 13 | H-4 | 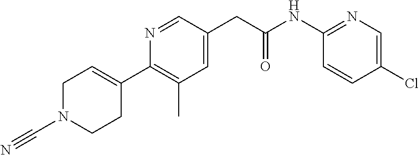 [SMILES: Cc1cc(CC(=O)Nc2ccc(Cl)cn2)cnc1C1=CCN(C#N)CC1] | ¹HNMR (DMSO-d₆, 400 MHz): δ 10.97 (s, 1H), 8.38 (s, 1H), 8.31 (s, 1H), 8.08-8.06 (m, 1H), 7.90-7.87 (m, 1H), 7.57 (s, 1H), 5.81 (bs, 1H), 3.93-3.92 (m, 2H), 3.73 (m, 2H), 3.44-3.42 (m, 2H), 2.50 (s, 2H), 2.31 (s, 3H); LCMS m/z: 368.10 (M + H)⁺; HPLC: 98.53%, rt: 6.40 min. |
| 14 | E-15 | 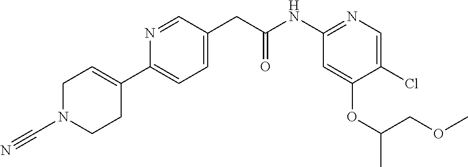 [SMILES: COCC(C)Oc1cc(NC(=O)Cc2ccc(C3=CCN(C#N)CC3)nc2)ncc1Cl] | ¹HNMR (DMSO-d₆, 400 MHz): δ 10.92 (s, 1H), 8.48 (s, 1H), 8.23 (s, 1H), 7.93 (s, 1H), 7.74-7.72 (m, 1H), 7.56-7.54 (m, 1H), 6.64 (s, 1H), 4.71-4.67 (m, 1H), 3.99 (s, 2H), 3.78 (s, 2H), 3.55-3.43 (m, 2H), 3.53-3.52 (m, 2H), 3.28 (s, 3H), 2.66-2.62 (m, 2H), 1.26-1.24 (m, 3H); LCMS m/z: 442.10 (M + H)⁺; HPLC: 96.14%, rt: 6.49 min. |
| 15 | E-14 | 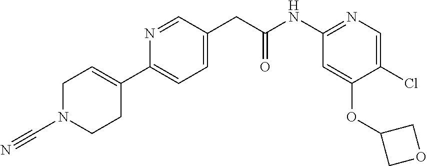 [SMILES: N#CN1CC=C(c2ccc(CC(=O)Nc3cc(OC4COC4)c(Cl)cn3)cn2)CC1] | ¹HNMR (DMSO-d₆, 400 MHz): δ 10.99 (s, 1H), 8.46 (s, 1H), 8.30 (s, 1H), 7.73-7.71 (d, 1H), 7.56-7.51 (m, 2H), 6.64 (s, 1H), 5.43-5.39 (m, 1H), 4.91-4.88 (m, 2H), 4.60- 4.57 (m, 2H), 3.99-3.98 (m, 2H), 3.78 (s, 2H), 3.45-3.43 (m, 2H), 2.66-2.62 (m, 2H); LCMS m/z: 426.10 (M + H)⁺; HPLC: 97.92%, rt: 7.48 min. |
| 16 | H-3 | 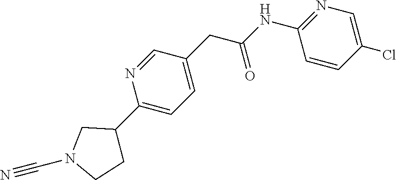 [SMILES: N#CN1CCC(c2ccc(CC(=O)Nc3ccc(Cl)cn3)cn2)C1] | ¹HNMR (DMSO-d₆, 400 MHz): δ 10.98 (s, 1H), 8.46 (s, 1H), 8.38 (s, 1H), 8.08 (d, 1H), 7.91 (d, 1H), 7.70 (d, 1H), 7.33-7.31 (d, 1H), 3.76 (s, 2H), 3.74-3.72 (m, 1H), 3.54-3.33 (m, 4H), 2.26-2.21 (m, 1H), 2.09-2.04 (m, 1H); LCMS m/z: 342.00 (M + H)⁺; HPLC: 98.04%, rt: 4.63 min. |
| 17 | E-23 | 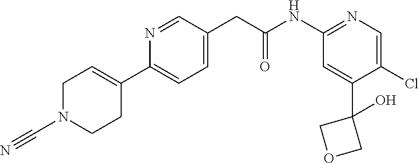 [SMILES: N#CN1CC=C(c2ccc(CC(=O)Nc3cc(C4(O)COC4)c(Cl)cn3)cn2)CC1] | ¹HNMR (DMSO-d₆, 400 MHz): δ 11.06 (s, 1H), 8.48 (s, 1H), 8.37 (s, 1H), 8.11 (s, 1H)7.74-7.72 (dd, 1H), 7.56-7.54 (d, 1H), 6.64 (bs, 1H), 6.57 (s, 1H), 4.97-4.96 (d, 2H), 4.68-4.67 (d, 2H), 3.99-3.98 (m, 2H), 3.79 (s, 2H) 3.44-3.43 (t, 2H), 2.67-2.66( m, 2H); LCMS m/z: 426.40 (M + H)⁺; HPLC: 97.52%, rt: 6.68 min. |
| 18 | E-57 | 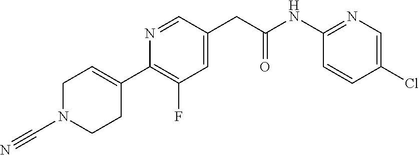 [SMILES: N#CN1CC=C(c2ncc(CC(=O)Nc3ccc(Cl)cn3)cc2F)CC1] | ¹HNMR (DMSO-d₆, 400 MHz): δ 11.00 (s, 1H), 8.39-8.37 (m, 2H), 8.08-8.06 (m, 1H), 7.91-7.81 (m, 1H), 7.71-7.67 (m, 1H), 6.41 (bs, 1H), 4.01 (s, 2H), 3.85 (s, 2H), 3.45-3.33 (m, 2H), 2.68 (bs, 2H); LCMS m/z: 372.20 (M + H)⁺; HPLC: 99.24%, rt: 6.02 min. |
| 19 | E-9 | 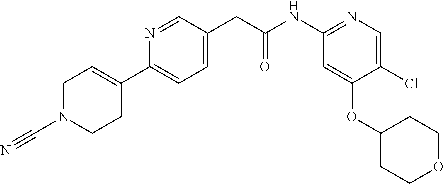 [SMILES: N#CN1CC=C(c2ccc(CC(=O)Nc3cc(OC4CCOCC4)c(Cl)cn3)cn2)CC1] | ¹HNMR (DMSO-d₆, 400 MHz): δ 10.93 (s, 1H), 8.46 (s, 1H), 8.25 (s, 1H), 7.92 (s, 1H), 7.72 (d, 1H), 7.55 (d, 1H), 6.64-6.61 (m, 1H), 4.74-4.70 (m, 1H), 3.99 ( d, 2H), 3.83-3.79 (m, 2H), 3.78 (s, 2H), 3.53-3.47 (m, 2H), 3.44-3.41 (m, 2H), 2.67-2.66 (m, 2H), 2.00-1.97 (m, 2H), 1.68-1.60 (m, 2H); LCMS m/z: 454.00 (M + H)⁺; HPLC: 99.55%, rt: 7.02 min. |
| 20 | E-19 | 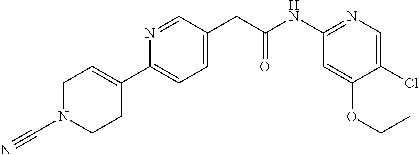 [SMILES: CCOc1cc(NC(=O)Cc2ccc(C3=CCN(C#N)CC3)nc2)ncc1Cl] | ¹HNMR (DMSO-d₆, 400 MHz): δ 10.92 (s, 1H), 8.47 (s, 1H), 8.22 (s, 1H), 7.88 (s, 1H), 7.74-7.71 (m, 1H), 7.55 (d, 1H), 6.64 (s, 1H), 4.17-4.11 (m, 2H), 3.98-3.95 (m, 2H), 3.77 (s, 2H), 3.44-3.43 (m, 2H), 2.69-2.60 (m, 2H), 1.37-1.33 (m, 3H); LCMS m/z: 398.10 (M + H)⁺; HPLC: 99.18%, rt: 8.79 min. |
| 21 | E-20 | 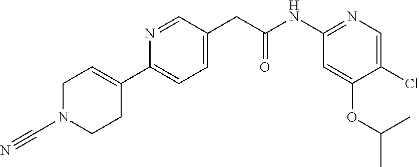 [SMILES: CC(C)Oc1cc(NC(=O)Cc2ccc(C3=CCN(C#N)CC3)nc2)ncc1Cl] | ¹HNMR (DMSO-d₆, 400 MHz): δ 10.91 (s, 1H), 8.47 (s, 1H), 8.22 (s, 1H), 7.89 (s, 1H), 7.74-7.71 (m, 1H), 7.54 (d, 1H), 6.64 (s, 1H), 4.71-4.65 (m, 1H), 3.98-3.95 (m, 2H), 3.78 (s, 2H), 3.44-3.41 (m, 2H), 2.66-2.60 (m, 2H), 1.32-1.17 (m, 6H); LCMS m/z: 412.10 (M + H)⁺; HPLC: 99.64%, rt: 9.63 min. |
| 22 | E-18 | 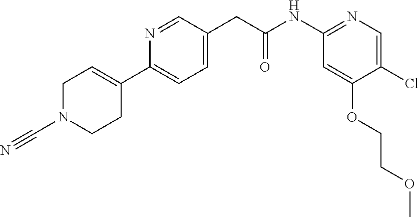 [SMILES: COCCOc1cc(NC(=O)Cc2ccc(C3=CCN(C#N)CC3)nc2)ncc1Cl] | ¹HNMR (DMSO-d₆, 400 MHz): δ 10.94 (s, 1H), 8.47 (s, 1H), 8.23 (s, 1H), 7.89 (s, 1H), 7.72 (d, 1H), 7.55 (d, 1H), 6.64 (s, 1H), 4.20- 4.19 (m, 2H), 3.98-3.95 (m, 2H), 3.78 (s, 2H), 3.69-3.68 (m, 2H), 3.44-3.41 (m, 2H), 3.33 (s, 3H), 2.66-2.60 (m, 2H); LCMS m/z: 428.10 (M + H)⁺; HPLC: 99.52%, rt: 7.68 min. |
| 23 | E-22 | 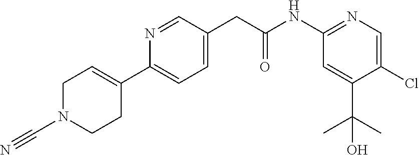 [SMILES: CC(C)(O)c1cc(NC(=O)Cc2ccc(C3=CCN(C#N)CC3)nc2)ncc1Cl] | ¹HNMR (DMSO-d₆, 400 MHz): δ 10.87 (s, 1H), 8.55 (s, 1H), 8.48 (s, 1H), 7.75-7.72 (dd, 1H), 7.56-7.54 (d, 1H), 6.64 (bs, 1H), 5.46 (s, 2H), 3.98 (s, 2H), 3.77 (s, 2H), 3.44- 3.43 (t, 2H), 2.66(s, 2H), 1.53 (s, 6H); LCMS m/z: 412.40 (M + H)⁺; HPLC: 98.12%, rt: 5.61 min. |
| 24 | E-17 | 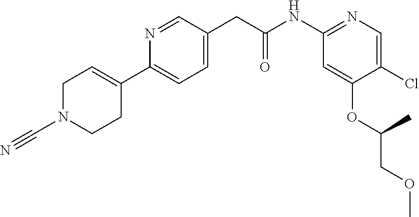 [SMILES: COC[C@H](C)Oc1cc(NC(=O)Cc2ccc(C3=CCN(C#N)CC3)nc2)ncc1Cl] | ¹HNMR (DMSO-d₆, 400 MHz): δ 8.51-8.50 (s, 1H), 8.07 (s, 1H), 7.89 (s, 1H), 7.66 (d, 1H), 7.39 (d, 1H), 7.26 (s, 1H), 6.55-6.61 (m, 1H), 4.59-4.55 (m, 1H), 4.01 (d, 2H), 3.72 (s, 2H), 3.49-3.46 (m, 2H), 2.78-2.77 (m, 2H), 2.65-2.59 (m, 1H), 2.41-2.33 (m, 1H), 2.29 (s, 3H), 2.01-1.92 (m, 3H); LCMS m/z: 442.10 (M − H)⁻; HPLC: 99.47%, rt: 5.02 min. |
| 25 | E-16 | 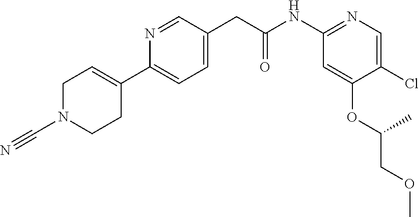 [SMILES: COC[C@@H](C)Oc1cc(NC(=O)Cc2ccc(C3=CCN(C#N)CC3)nc2)ncc1Cl] | ¹HNMR (DMSO-d₆, 400 MHz): δ 10.91 (s, 1H), 8.47 (s, 1H), 8.23 (s, 1H), 7.92 (s, 1H), 7.72 (d, 1H), 7.55 (d, 1H), 6.64-6.62 (m, 1H), 4.69-4.68 (m, 1H), 3.98-3.96 (m, 2H), 3.78 (s, 2H), 3.52-3.51 (m, 2H), 3.44-3.41 (m, 2H), 3.32 (s, 3H), 2.69-2.62 (m, 2H), 1.25 (d, 3H); LCMS m/z: 442.15 (M + H)⁻; HPLC: 99.00%, rt: 4.56 min. |
| 26 | E-25 | 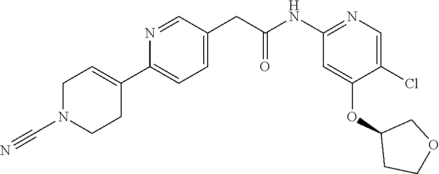 [SMILES: N#CN1CC=C(c2ccc(CC(=O)Nc3cc(O[C@@H]4CCOC4)c(Cl)cn3)cn2)CC1] | ¹HNMR (DMSO-d₆, 400 MHz): δ 10.96 (s, 1H), 8.47 (s, 1H), 8.24 (s, 1H), 7.88 (s, 1H), 7.73 (d, 1H), 7.55 (d, 1H), 6.65-6.63 (s, 1H), 5.76-5.74 (m, 1H), 3.99 (d, 2H), 3.86 (s, 2H), 3.85-3.84 (m, 1H), 3.80-3.79 (m, 2H), 3.76-3.75 (m, 1H), 3.74-3.72 (m, 2H), 3.43-3.39 (m, 2H), 2.26-2.23 (m, 1H), 1.99- 1.96 (m, 1H); LCMS m/z: 440.10 (M + H)⁺; HPLC: 97.65%, rt: 5.08 |
| | | | min. |
| 27 | E-26 | 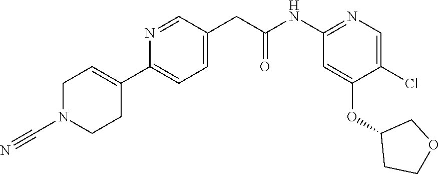 [SMILES: N#CN1CC=C(c2ccc(CC(=O)Nc3cc(O[C@H]4CCOC4)c(Cl)cn3)cn2)CC1] | ¹HNMR (DMSO-d₆, 400 MHz): δ 10.96 (s, 1H), 8.47 (s, 1H), 8.24 (s, 1H), 7.88 (s, 1H), 7.73 (d, 1H), 7.55 (d, 1H), 6.65-6.63 (s, 1H), 5.12-5.10 (s, 1H), 3.99 (s, 2H), 3.89-3.86 (m, 2H), 3.85-3.72 (m, 4H), 3.44-3.41 (m, 2H), 2.67 (s, 2H), 2.32-2.21 (m, 1H), 2.01-01.95 (m, 1H); LCMS m/z: 440.20 (M + H)⁺; HPLC: 97.94%, rt: 3.18 min. |
| 28 | E-21 | 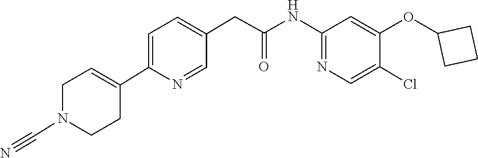 [SMILES: N#CN1CC=C(c2ccc(CC(=O)Nc3cc(OC4CCC4)c(Cl)cn3)cn2)CC1] | ¹HNMR (DMSO-d₆, 400 MHz): δ 10.90 (s, 1H), 8.47 (d, 1H), 8.22 (s, 1H), 7.79 (s, 1H), 7.72 (d, 1H), 7.54 (d, 1H), 6.64-6.62 (m, 1H), 4.79-4.75 (m, 1H), 3.98 (d, 2H), 3.78-3.74 (m, 2H), 3.44-3.41 (m, 2H), 2.67-2.66 (m, 2H), 2.42-2.40 (m, 2H), 2.12-2.07 (m, 2H), 1.81- 1.68 (m, 2H); LCMS m/z: 424.00 (M + H)⁺; HPLC: 99.34%, rt: 3.71 |
| | | | min. |
| 29 | E-12 | 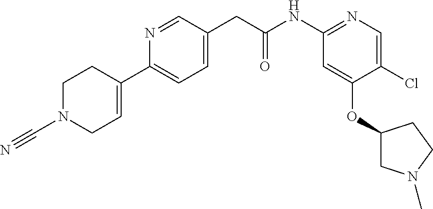 [SMILES: CN1CC[C@H](Oc2cc(NC(=O)Cc3ccc(C4=CCN(C#N)CC4)nc3)ncc2Cl)C1] | ¹HNMR (DMSO-d₆, 400 MHz): δ 10.93 (s, 1H), 8.47 (s, 1H), 8.22 (s, 1H), 7.84 (s, 1H), 7.71 (d, 1H), 7.56 (d, 1H), 6.64 (s, 1H), 4.92- 4.90 (m, 1H), 3.98 (s, 2H), 3.78 (s, 2H), 3.44-3.41 (m, 2H), 3.44-3.41 (m, 2H), 2.70-2.66 (m, 4H), 2.32- 2.21 (m, 2H), 2.01-01.95 (s, 3H); LCMS m/z: 453.20 (M + H)⁺; HPLC: 97.21%, rt: 4.84 min. |
| 30 | E-13 | 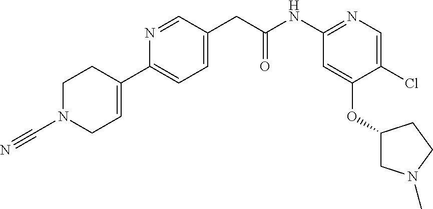 [SMILES: CN1CC[C@@H](Oc2cc(NC(=O)Cc3ccc(C4=CCN(C#N)CC4)nc3)ncc2Cl)C1] | ¹HNMR (DMSO-d₆, 400 MHz): 10.94 (s, 1H), 8.47-8.47 (m, 1H), 8.23 (s, 1H), 7.84 (s, 1H), 7.72 (d, 1H), 7.55 (d, 1H), 6.64-6.60 (m, 1H), 4.94-4.93 (m, 1H), 3.98 (d, 2H), 3.78 (s, 2H), 3.44-3.43 (m, 2H), 2.73 (d, 3H), 2.66 (s, 2H), 2.35-2.32 (m, 2H), 2.30-2.27 (m, 3H), 1.95-1.75 (m, 1H); LCMS m/z: 453.10 (M + H)⁺; HPLC: 99%, rt: 4.83 min. |
| 31 | E-27 | 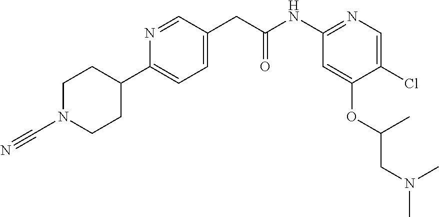 [SMILES: CC(CN(C)C)Oc1cc(NC(=O)Cc2ccc(C3CCN(C#N)CC3)nc2)ncc1Cl] | ¹HNMR (DMSO-d₆, 400 MHz): δ 10.92 (s, 1H), 8.47 (s, 1H), 8.22 (s, 1H), 7.94 (s, 1H), 7.72 (d, 1H), 7.55 (d, 1H), 6.64 (s, 1H), 4.66- 4.65 (m, 1H), 3.98 (s, 2H), 3.78 (s, 2H), 3.44-3.43 (m, 2H), 2.66-2.64 (m, 3H), 2.57-5.54 (m, 1H), 2.22 (s, 6H), 1.34 (d, 3H); LCMS m/z: 455.20 (M + H)⁺; HPLC: 98.27%, rt: 4.82 min. |
| 32 | E-30 | 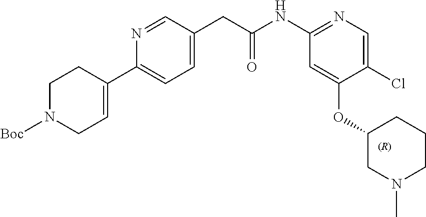 | ¹HNMR (DMSO-d6, 400 MHz): δ 10.94 (s, 1H), 8.49 (s, 1H), 8.28 (s, 1H), 7.93 (s, 1H), 7.74 (d, 1H), 7.56 (d, 1H), 6.65 (s, 1H), 4.71(s, 1H), 4.00 (s, 2H), 3.80 (s, 2H), 3.36-3.33 (m, 1H), 3.46-3.43 (m, 2H), 3.16-3.15 (m, 1H), 2.90-2.88 (m, 1H), 2.67 (s, 3H), 1.86 (s, 2H), 1.65 (s, 2H), 1.28-1.25 (m, 3H); LCMS m/z: 467.20 (M + H)⁺; HPLC: 96.77%, rt: 4.92 min. |
| 33 | E-31 | 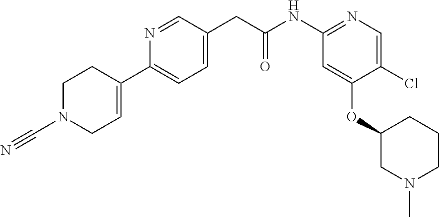 [SMILES: CN1CCC[C@H](Oc2cc(NC(=O)Cc3ccc(C4=CCN(C#N)CC4)nc3)ncc2Cl)C1] | ¹HNMR (DMSO-d₆, 400 MHz): δ 10.92 (s, 1H), 8.47 (s, 1H), 8.23 (s, 1H), 7.91 (s, 1H), 7.74-7.72(m, 1H), 7.55 (d, 1H), 6.64 (s, 1H), 4.50-4.48 (m, 1H), 3.98 (d, 2H), 3.78 (s, 2H), 3.44-3.41 (m, 2H), 2.67-2.66 (m, 2H), 2.65-2.35 (m, 2H), 2.15 (s, 3H), 2.063 (m, 2H), 1.92-1.75 (m, 2H), 1.70-1.67 (m, 2H); LCMS m/z: 467.10 (M + H)⁺; HPLC: 92.84%, rt: 5.28 min. |
| 34 | E-32 | 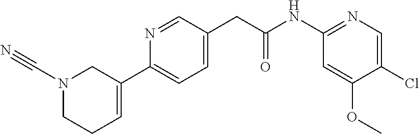 [SMILES: COc1cc(NC(=O)Cc2ccc(C3=CCCN(C#N)C3)nc2)ncc1Cl] | ¹HNMR (DMSO-d₆, 400 MHz): δ 10.96 (s, 1H), 8.46 (s, 1H), 8.24 (s, 1H), 7.91 (s, 1H), 7.74 (d, 1H), 7.63 (d, 1H), 6.80-6.79 (m, 1H), 4.22 (s, 2H), 3.89 (s, 3H), 3.79 (s, 2H), 3.70-3.68 (m, 2H), 2.66-2.64 (m, 2H); LCMS m/z: 384.10 (M + H)⁺; HPLC: 96.51%, rt: 7.02 min. |
| 35 | E-34 | 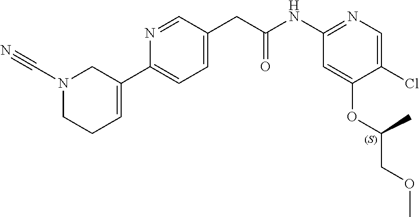 | ¹HNMR (DMSO-d₆, 400 MHz): δ 10.92 (s, 1H), 8.48 (d, 1H), 8.24 (s, 1H), 7.93 (s, 1H), 7.75-7.74 (m, 1H), 7.63-7.61 (m, 1H), 6.80-6.78 (m, 1H), 4.71-4.67 (m, 1H), 4.22 (d, 2H), 3.78 (s, 2H), 3.56-3.50 (m, 2H), 3.37-3.35 (m, 2H), 3.29 (s, 3H), 2.43-2.42 (m, 2H), 1.26 (d, 3H); LCMS m/z: 442.10 (M + H)⁺; HPLC: 99.45%, rt: 5.22 min. |
| 36 | E-35 | 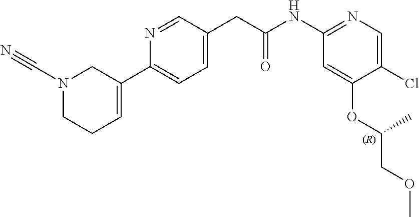 | ¹HNMR (DMSO-d₆, 400 MHz): δ 10.92 (s, 1H), 8.44 (d, 1H), 8.23 (s, 1H), 7.92 (s, 1H), 7.73-7.71 (m, 1H), 7.62-7.60 (m, 1H), 6.79-6.78 (m, 1H), 4.70-4.66 (m, 1H), 4.21 (d, 2H), 3.77 (s, 2H), 3.55-3.50 (m, 2H), 3.37-3.35 (m, 2H), 3.28 (s, 3H), 2.43-2.42 (m, 2H), 1.26 (d, 3H); LCMS m/z: 442.10 (M + H)⁺; HPLC: 99.45%, rt: 5.22 min. |
| 37 | E-33 | 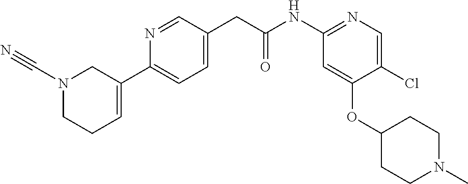 [SMILES: CN1CCC(Oc2cc(NC(=O)Cc3ccc(C4=CCCN(C#N)C4)nc3)ncc2Cl)CC1] | ¹HNMR (DMSO-d₆, 400 MHz): δ 10.91 (s, 1H), 8.44 (s, 1H), 8.23 (s, 1H), 7.89 (s, 1H), 7.71 (d, 1H), 7.61 (d, 1H), 6.79 (s, 1H), 4.50 (s, 1H), 4.21 (s, 2H), 3.77 (s, 2H), 3.36-3.33 (m, 2H), 2.50 (s, 2H), 2.42 (s, 2H), 2.23-2.19 (m, 2H), 2.15-2.08 (m, 3H), 1.95-1.91 (m, 2H), 1.72-1.68 (m, 2H); LCMS m/z: 467.20 (M + H)⁺; HPLC: 96.12%, rt: 4.80 min. |
| 38 | E-36 | 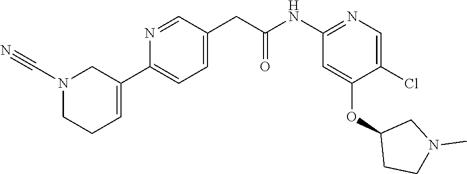 [SMILES: CN1CC[C@@H](Oc2cc(NC(=O)Cc3ccc(C4=CCCN(C#N)C4)nc3)ncc2Cl)C1] | ¹HNMR (DMSO-d₆, 400 MHz): δ 10.93 (s, 1H), 8.44 (d, 1H), 8.22 (s, 1H), 7.83 (s, 1H), 7.74-7.72 (m, 1H), 7.63-7.61 (m, 1H), 6.80-6.78 (m, 1H), 4.97-4.96 (m, 1H), 4.22 (s, 2H), 3.79 (s, 2H), 3.36 (d, 2H), 2.73-2.69 (m, 3H), 2.42-2.41 (m, 3H), 2.33-2.27 (s, 4H), 1.64-1.62 (m, 1H); LCMS m/z: 453.10 (M + H)⁺; HPLC: 96.59%, rt: 4.76 min. |
| 39 | E-37 | 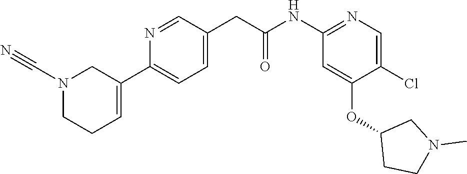 [SMILES: CN1CC[C@H](Oc2cc(NC(=O)Cc3ccc(C4=CCCN(C#N)C4)nc3)ncc2Cl)C1] | ¹HNMR (DMSO-d₆, 400 MHz): δ 10.93 (s, 1H), 8.46 (s, 1H), 8.23 (s, 1H), 7.85 (s, 1H), 7.74-7.72 (m, 1H), 7.63-7.61 (m, 1H), 6.80-6.78 (m, 1H), 4.97-4.96 (m, 1H), 4.22 (s, 2H), 3.79 (s, 2H), 3.36 (d, 2H), 2.73-2.69 (m, 3H), 2.42-2.41 (m, 3H), 2.33-2.27 (s, 4H), 1.64-1.62 (m, 1H); LCMS m/z: 453.10 (M + H)⁺; HPLC: 96.59%, rt: 4.76 min. |
| 40 | E-38 | 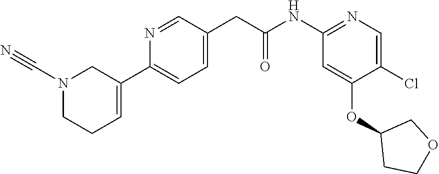 [SMILES: N#CN1CCC=C(c2ccc(CC(=O)Nc3cc(O[C@@H]4CCOC4)c(Cl)cn3)cn2)C1] | ¹HNMR (DMSO-d₆, 400 MHz): δ 10.97 (s, 1H), 8.46 (s, 1H), 8.26 (s, 1H), 7.89 (s, 1H), 7.41 (d, 1H), 7.62 (d, 1H), 6.80 (s, 1H), 5.12 (s, 1H), 4.22 (s, 2H), 3.87-3.73 (m, 6H), 3.37-3.34 (m, 2H), 2.43 (s, 2H), 2.28-2.24 (m, 1H), 2.01- 1.97 (m, 1H); LCMS m/z: 440.10 (M + H)⁺; HPLC: 98.41%, rt: 3.73 min. |
| 41 | E-39 | 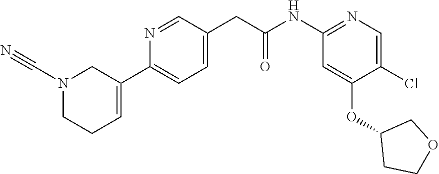 [SMILES: N#CN1CCC=C(c2ccc(CC(=O)Nc3cc(O[C@H]4CCOC4)c(Cl)cn3)cn2)C1] | ¹HNMR (DMSO-d₆, 400 MHz): δ 10.97 (s, 1H), 8.46 (s, 1H), 8.26 (s, 1H), 7.89 (s, 1H), 7.41 (d, 1H), 7.62 (d, 1H), 6.80 (s, 1H), 5.12 (s, 1H), 4.22 (s, 2H), 3.87-3.73 (m, 6H), 3.37-3.34 (m, 2H), 2.43 (s, 2H), 2.28-2.24 (m, 1H), 2.01- 1.97 (m, 1H); LCMS m/z: 440.10 (M + H)⁺; HPLC: 98.49%, rt: 3.73 min. |
| 42 | E-40 | 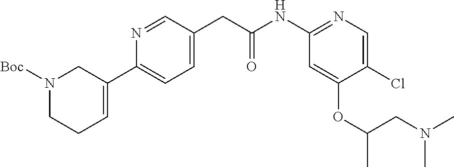 | ¹HNMR (DMSO-d₆, 400 MHz): δ 10.91 (s, 1H), 8.44 (s, 1H), 8.22 (s, 1H), 7.93 (s, 1H), 7.71 (d, 1H), 7.62-7.60 (m, 1H), 6.79-6.78 (m, 1H), 4.65-4.62 (m, 1H), 4.21 (s, 2H), 3.77 (s, 2H), 3.36-3.34 (m, 2H), 2.53-2.52 (m, 2H), 2.44-2.42 (m, 2H), 2.18 (s, 6H), 1.27 (d, 3H); LCMS m/z: 455.10 (M + H)⁺; HPLC: 98.27%, rt: 5.07 min. |
| 43 | E-41 | 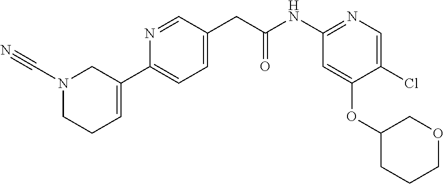 [SMILES: N#CN1CCC=C(c2ccc(CC(=O)Nc3cc(OC4CCCOC4)c(Cl)cn3)cn2)C1] | ¹HNMR (DMSO-d₆, 400 MHz): δ 10.93 (s, 1H), 8.44 (s, 1H), 8.24 (s, 1H), 7.88 (s, 1H), 7.73-7.71 (m, 1H), 7.61 (d, 1H), 6.79-6.77 (m, 1H), 4.49-4.47 (m, 1H), 4.21 (s, 2H), 3.80-3.77 (m, 3H), 3.58-3.56 (m, 3H), 3.38-3.36 (m, 1H), 2.42- 2.41 (m, 2H), 2.01-1.98 (m, 1H), 1.79-1.77 (m, 2H), 1.51-1.50 (m, 2H); LCMS m/z: 454.10 (M + H)⁺; HPLC: 98.33%, rt: 6.36 min. |
| 44 | Chiral | Isomer-1 of compound-43 | ¹HNMR (DMSO-d₆, 400 MHz): δ |
| | separation | | 10.93 (s, 1H), 8.44 (s, 1H), 8.24 (s, |
| | of Cpd-43 | | 1H), 7.88 (s, 1H), 7.73-7.71 (m, |
| | | | 1H), 7.61 (d, 1H), 6.79-6.77 (m, |
| | | | 1H), 4.49-4.47 (m, 1H), 4.21 ( s, |
| | | | 2H), 3.80-3.77 (m, 3H), 3.58-3.54 |
| | | | (m, 3H), 3.39-3.36 (m, 1H), 2.41- |
| | | | 2.32 (m, 2H), 2.01-1.98 (m, 1H), |
| | | | 1.79-1.77 (m, 2H), 1.51-1.50 (m, |
| | | | 2H); LCMS m/z: 454.10 (M + H)⁺; |
| | | | HPLC: 99.53%, rt: 5.18 min, |
| | | | Chiral HPLC: 97.63%, rt: 26.16 |
| | | | min. |
| 45 | Chiral | Isomer-2 of compound-43 | ¹HNMR (DMSO-d₆, 400 MHz): δ |
| | separation | | 10.93 (s, 1H), 8.44 (s, 1H), 8.24 (s, |
| | of Cpd-43 | | 1H), 7.88 (s, 1H), 7.73-7.71 (m, |
| | | | 1H), 7.61 (d, 1H), 6.79-6.77 (m, |
| | | | 1H), 4.49-4.47 (m, 1H), 4.21 (s, |
| | | | 2H), 3.80-3.77 (m, 3H), 3.58-3.54 |
| | | | (m, 3H), 3.39-3.36 (m, 1H), 3.41- |
| | | | 3.32 (m, 2H), 2.01-1.98 (m, 1H), |
| | | | 1.79-1.77 (m, 2H), 1.59-1.46 (m, |
| | | | 2H); LCMS m/z: 454.10 (M + H)⁺; |
| | | | HPLC: 99.43%, rt: 5.18 min, |
| | | | Chiral HPLC: 92.17%, rt: 29.64 |
| | | | min. |
| 46 | E-29 | 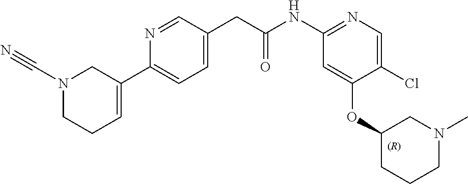 | ¹HNMR (DMSO-d₆, 400 MHz): δ 10.92 (s, 1H), 8.47 (s, 1H), 8.23 (s, 1H), 7.91 (s, 1H), 7.74-7.72(m, 1H), 7.55 (d, 1H), 6.64 (s, 1H), 4.50-4.48 (m, 1H), 4.20 (d, 2H), 3.76 (s, 2H), 3.44-3.41 (m, 2H), 2.79-2.76 (s, 1H), 2.67-2.66 (m, 2H), 2.15 (s, 3H), 2.063 (m, 2H), 1.95-1.72 (m, 3H), 1.50-1.4 (m, 2H); LCMS m/z: 467.10 (M + H)⁺; HPLC: 92.84%, rt: 5.28 min. |
| 47 | E-28 | 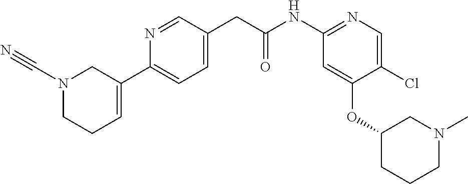 [SMILES: CN1CCC[C@H](Oc2cc(NC(=O)Cc3ccc(C4=CCCN(C#N)C4)nc3)ncc2Cl)C1] | ¹HNMR (DMSO-d₆, 400 MHz): δ 10.94 (s, 1H), 8.49 (s, 1H), 8.28 (s, 1H), 7.93 (s, 1H), 7.74 (d, 1H), 7.56 (d, 1H), 6.65 (s, 1H), 4.71(s, 1H), 4.00 (s, 2H), 3.80 (s, 2H), 3.36-3.33 (m, 1H), 3.46-3.43 (m, 2H), 3.16-3.15 (m, 1H), 2.90-2.88 (m, 1H), 2.67 (s, 3H), 1.86 (s, 2H), 1.65 (s, 2H), 1.28-1.25 (m, 3H); LCMS m/z: 467.20 (M + H)⁺; HPLC: 96.77%, rt: 4.92 min. |
| 48 | E-42 | 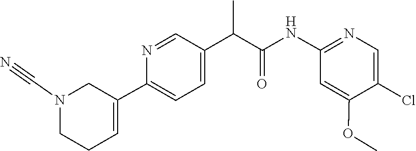 [SMILES: COc1cc(NC(=O)C(C)c2ccc(C3=CCCN(C#N)C3)nc2)ncc1Cl] | ¹HNMR (DMSO-d₆, 400 MHz): δ 10.92 (s, 1H), 8.53 (d, 1H), 8.20 (s, 1H), 7.93 (s, 1H), 7.79-7.78 (m, 1H), 7.63-7.61 (m, 1H), 6.79-6.77 (m, 1H), 4.20 (s, 2H), 4.07-4.03 (m, 1H), 3.90 (s, 3H), 3.35-3.32 (m, 2H), 2.41-2.38 (m, 2H), 1.44 (d, 3H); LCMS m/z: 398.10 (M + H)⁺; HPLC: 99.22%, rt: 5.32 min. |
| 49 | Chiral | Isomer-1 of compound-48 | ¹HNMR (DMSO-d₆, 400 MHz): δ |
| | separation | | 10.91 (s, 1H), 8.53 (d, 1H), 8.20 (s, |
| | of Cpd-48 | | 1H), 7.93 (s, 1H), 7.79-7.78 (m, |
| | | | 1H), 7.63-7.61 (m, 1H), 6.79-6.77 |
| | | | (m, 1H), 4.20 (s, 2H), 4.08-4.03 |
| | | | (m, 1H), 3.90 (s, 3H), 3.35-3.32 |
| | | | (m, 2H), 2.41-2.39 (m, 2H), 1.44 |
| | | | (d, 3H); LCMS m/z: 398.10 |
| | | | (M + H)⁺; HPLC: 99.22%, rt: 5.32 |
| | | | min, Chiral HPLC: 98.08%, rt: |
| | | | 7.11 min. |
| 50 | Chiral | Isomer-2 of compound-48 | ¹HNMR (DMSO-d₆, 400 MHz): δ |
| | separation | | 10.91 (s, 1H), 8.53 (d, 1H), 8.20 (s, |
| | of Cpd-48 | | 1H), 7.93 (s, 1H), 7.79-7.78 (m, |
| | | | 1H), 7.63-7.61 (m, 1H), 6.79-6.77 |
| | | | (m, 1H), 4.20 (s, 2H), 4.07-4.05 |
| | | | (m, 1H), 3.90 (s, 3H), 3.35-3.33 |
| | | | (m, 2H), 2.40-2.38 (m, 2H), 1.44 |
| | | | (d, 3H); LCMS m/z: 398.00 |
| | | | (M + H)⁺; HPLC: 99.55%, rt: 6.62 |
| | | | min, Chiral HPLC: 98.41%, rt: |
| | | | 9.42 min. |
| 51 | E-43 | 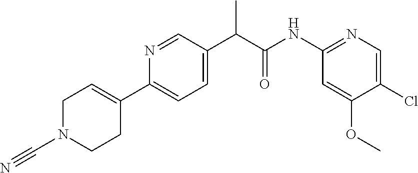 [SMILES: COc1cc(NC(=O)C(C)c2ccc(C3=CCN(C#N)CC3)nc2)ncc1Cl] | ¹HNMR (DMSO-d₆, 400 MHz): δ 10.91 (s, 1H), 8.55 (s, 1H), 8.20 (s, 1H), 7.93 (s, 1H), 7.78-7.76 (m, 1H), 7.56-7.54 (m, 1H), 6.62-6.60 (m, 1H), 4.07-4.05 (m, 1H), 3.97 (s, 2H), 3.89 (s, 3H), 3.43-3.41 (m, 2H), 2.65-2.63 (m, 2H), 1.44 (d, 3H); LCMS m/z: 398.00 (M + H)⁺; HPLC: 98.96%, rt: 7.10 min |
| 52 | Chiral | Isomer-1 of compound-51 | ¹HNMR (DMSO-d₆, 400 MHz): δ |
| | separation | | 10.91 (s, 1H), 8.55 (s, 1H), 8.20 (s, |
| | of Cpd-51 | | 1H), 7.93 (s, 1H), 7.78-7.76 (m, |
| | | | 1H), 7.56-7.54 (m, 1H), 6.62-6.60 |
| | | | (m, 1H), 4.07-4.05 (m, 1H), 3.97 |
| | | | (s, 2H), 3.89 (s, 3H), 3.43-3.41 (m, |
| | | | 2H), 2.65-2.63 (m, 2H), 1.44 (d, |
| | | | 3H); LCMS m/z: 398.10 (M + H)⁺; |
| | | | HPLC: 95.18%, rt: 6.70 min. |
| 53 | Chiral | Isomer-2 of compound-51 | ¹HNMR (DMSO-d₆, 400 MHz): δ |
| | separation | | 10.91 (s, 1H), 8.55 (s, 1H), 8.19 (s, |
| | of Cpd-51 | | 1H), 7.93 (s, 1H), 7.78-7.76 (m, |
| | | | 1H), 7.56-7.54 (m, 1H), 6.62-6.60 |
| | | | (m, 1H), 4.07-4.05 (m, 1H), 3.97 |
| | | | (s, 2H), 3.90 (s, 3H), 3.41-3.40 (m, |
| | | | 2H), 2.65-2.63 (m, 2H), 1.44 (d, |
| | | | 3H); LCMS m/z: 398.10 (M + H)⁺; |
| | | | HPLC: 95.36%, rt: 6.67 min. |
| 54 | E-44 | 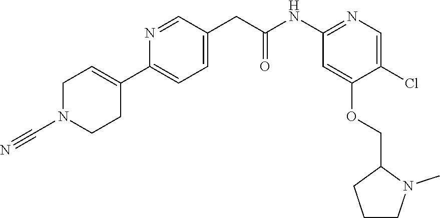 [SMILES: CN1CCCC1COc1cc(NC(=O)Cc2ccc(C3=CCN(C#N)CC3)nc2)ncc1Cl] | ¹HNMR (DMSO-d₆, 400 MHz): δ 10.94 (s, 1H), 8.47 (d, 1H), 8.23 (s, 1H), 7.89 (s, 1H), 7.74-7.72 (m, 1H), 7.56-7.54 (m, 1H), 6.64-6.60 (m, 1H), 4.02-3.98 (m, 4H), 3.78 (s, 2H), 3.44-3.43 (m, 2H), 3.12- 2.98 (m, 2H), 2.66 (s, 3H), 2.42- 2.40 (m, 2H), 2.12-1.96 (m, 1H), 1.71-1.62 (m, 4H); LCMS m/z: 467.10 (M + H)⁺; HPLC: 97.06%, rt: 5.23 min. |
| 55 | E-45 | 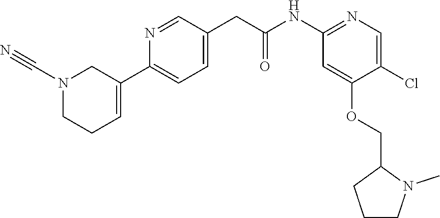 [SMILES: CN1CCCC1COc1cc(NC(=O)Cc2ccc(C3=CCCN(C#N)C3)nc2)ncc1Cl] | ¹HNMR (DMSO-d₆, 400 MHz): δ 10.93 (s, 1H), 8.44 (d, 1H), 8.23 (s, 1H), 7.89 (s, 1H), 7.74-7.72 (m, 1H), 7.62-7.60 (m, 1H), 6.91-6.88 (m, 1H), 4.23-4.21 (m, 2H), 4.12- 3.98 (s, 2H), 3.77 (s, 2H), 3.36- 3.34 (m, 2H), 3.12-2.98 (m, 2H), 2.42-2.40 (m, 4H), 2.35-2.32 (m, 1H), 2.05-1.98 (m, 2H), 1.53-1.50 (m, 3H); LCMS m/z: 467.10 (M + H)⁺; HPLC: 99.22%, rt: 5.65 min. |
| 56 | E-46 | 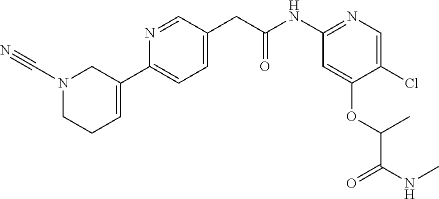 [SMILES: CNC(=O)C(C)Oc1cc(NC(=O)Cc2ccc(C3=CCCN(C#N)C3)nc2)ncc1Cl] | ¹HNMR (DMSO-d₆, 400 MHz): δ 10.89 (s, 1H), 8.44 (s, 1H), 8.24 (s, 1H), 8.14-8.13 (m, 1H), 7.76-7.70 (m, 2H), 7.62-7.59 (m, 1H), 6.78- 6.77(m, 1H), 4.78-4.76 (m, 1H), 4.21 (s, 2H), 3.76 (s, 2H), 3.38- 3.36 (m, 2H), 2.58-2.56 (m, 2H), 2.41-2.40 (m, 3H), 1.44 (d, 3H); LCMS m/z: 455.10 (M + H)⁺; HPLC: 98.71%, rt: 6.47 min. |
| 57 | Chiral | Isomer-1 of compound-56 | ¹HNMR (DMSO-d₆, 400 MHz): δ |
| | separation | | 10.89 (s, 1H), 8.44 (s, 1H), 8.24 (s, |
| | of Cpd-56 | | 1H), 8.14-8.13 (m, 1H), 7.76-7.70 |
| | | | (m, 2H), 7.62-7.59 (m, 1H), 6.78- |
| | | | 6.77(m, 1H), 4.78-4.76 (m, 1H), |
| | | | 4.21 (s, 2H), 3.76 (s, 2H), 3.38- |
| | | | 3.36 (m, 2H), 2.58-2.56 (m, 2H), |
| | | | 2.41-2.40 (m, 3H), 1.45 (d, 3H); |
| | | | LCMS m/z: 455.10 (M + H)⁺; |
| | | | HPLC: 99.43%, rt: 6.35 min, |
| | | | Chiral HPLC: 98.68%, rt: 11.22 |
| | | | min. |
| 58 | Chiral | Isomer-2 of compound-56 | ¹HNMR (DMSO-d₆, 400 MHz): δ |
| | separation | | 10.89 (s, 1H), 8.44 (s, 1H), 8.24 (s, |
| | of Cpd-56 | | 1H), 8.14-8.13 (m, 1H), 7.76-7.70 |
| | | | (m, 2H), 7.62-7.59 (m, 1H), 6.78- |
| | | | 6.77(m, 1H), 4.77-4.75 (m, 1H), |
| | | | 4.21 (s, 2H), 3.76 (s, 2H), 3.38- |
| | | | 3.36 (m, 2H), 2.58-2.56 (m, 2H), |
| | | | 2.41-2.40 (m, 3H), 1.45 (d, 3H); |
| | | | LCMS m/z: 455.10 (M + H)⁺; |
| | | | HPLC: 99.43%, rt: 6.35 min, |
| | | | Chiral HPLC: 98.66%, rt: 14.40 |
| | | | min. |
| 59 | H-5 | 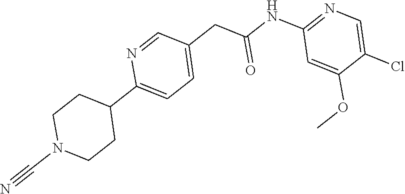 [SMILES: COc1cc(NC(=O)Cc2ccc(C3CCN(C#N)CC3)nc2)ncc1Cl] | ¹HNMR (DMSO-d₆, 400 MHz): δ 10.93 (s, 1H), 8.44 (d, 1H), 8.22 (s, 1H), 7.91 (s, 1H), 7.69-7.66 (m, 1H), 7.26-7.24 (m, 1H), 3.80 (s, 3H), 3.75 (s, 2H), 3.47-3.44 (m, 2H), 3.20-3.13 (m, 2H), 2.84-2.78 (m, 1H), 1.90-1.84 (m, 4H); LCMS m/z: 386.70 (M + H)⁺; HPLC: 97.84%, rt: 6.39 min. |
| 60 | H-6 | 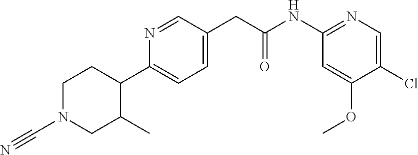 [SMILES: COc1cc(NC(=O)Cc2ccc(C3CCN(C#N)CC3C)nc2)ncc1Cl] | ¹HNMR (DMSO-d₆, 400 MHz): δ 10.93 (s, 1H), 8.44 (d, 1H), 8.22 (s, 1H), 7.91 (s, 1H), 7.65-7.63 (m, 1H), 7.22-7.20 (d, 1H), 3.88 (s, 3H), 3.75 (s, 2H), 3.61-3.53 (m, 1H), 3.29-3.28 (m, 1H), 3.15-3.13 (m, 1H), 2.98-2.90 (m, 2H), 2.28- 2.25 (m, 1H), 2.15-2.10 (m, 1H), 1.85-1.80 (m, 1H), 0.7 (d, 3H); LCMS m/z: 400.10 (M + H)⁺; |
| | | | HPLC: 96.82%, rt: 5.18 min. |
| 61 | E-47 | 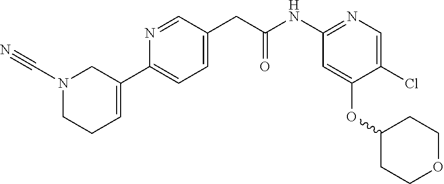 [SMILES: N#CN1CCC=C(c2ccc(CC(=O)Nc3cc(OC4CCOCC4)c(Cl)cn3)cn2)C1] | ¹HNMR (DMSO-d₆, 400 MHz): δ 10.92 (s, 1H), 8.44 (s, 1H), 8.25 (s, 1H), 7.92 (s, 1H), 7.72 (d, 1H), 7.62-7.60 (m, 1H), 6.82-6.80 (m, 1H), 4.74-4.70 (m, 1H), 4.21 (s, 2H), 3.80-3.77 (m, 3H), 3.52-3.50 (m, 2H), 3.36-3.35 (m, 3H), 2.42- 2.41 (m, 2H), 2.05-1.97 (m, 2H), 1.68-1.60 (m, 2H); LCMS m/z: 454.10 (M + H)⁺; HPLC: 98.03%, rt: 4.88 min. |
| 62 | E-48 | 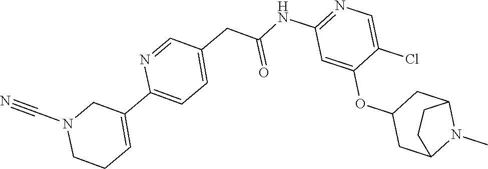 [SMILES: CN1C2CCC1CC(Oc1cc(NC(=O)Cc3ccc(C4=CCCN(C#N)C4)nc3)ncc1Cl)C2] | ¹HNMR (DMSO-d₆, 400 MHz): δ 10.91 (s, 1H), 8.45 (s, 1H), 8.20 (s, 1H), 7.99 (s, 1H), 7.73 (d, 1H), 7.63-7.61 (m, 1H), 6.81-6.80 (m, 1H), 4.68-4.64 (m, 1H), 4.21 (d, 2H), 3.78 (s, 2H), 3.36-3.35 (m, 2H), 3.16-3.14 (m, 2H), 2.42-2.41 (m, 4H), 2.20 (s, 3H), 1.94-1.93 (m, 2H), 1.68-1.66 (m, 2H), 1.58- 1.56 (m, 2H); LCMS m/z: 493.20 (M + H)⁺; HPLC: 98.43%, rt: 5.42 min. |
| 63 | E-49 | 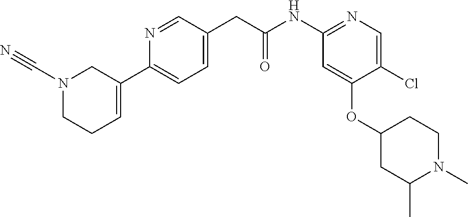 [SMILES: CC1CC(Oc2cc(NC(=O)Cc3ccc(C4=CCCN(C#N)C4)nc3)ncc2Cl)CCN1C] | -NA- |
| 64 | Chiral | Isomer-1 of compound-63 | ¹HNMR (DMSO-d₆, 400 MHz): δ |
| | separation | | 10.90 (s, 1H), 8.44 (s, 1H), 8.23 (s, |
| | of Cpd-63 | | 1H), 7.86 (s, 1H), 7.73-7.71 (m, |
| | | | 1H), 7.63-7.60 (m, 1H), 6.79-6.78 |
| | | | (m, 1H), 4.74-4.72 (m, 1H), 4.21 |
| | | | (s, 2H), 3.76 (s, 2H), 3.38-3.36 (m, |
| | | | 2H), 2.83-2.81 (m, 1H), 2.56-2.54 |
| | | | (m, 2H), 2.42-2.40 (m, 1H), 2.32- |
| | | | 2.25 (m, 1H), 2.15 (s, 3H), 1.80- |
| | | | 1.78 (m, 3H), 1.55-1.50 (m, 1H), |
| | | | 0.97- 0.87 (m, 3H); LCMS m/z: |
| | | | 481.20 (M + H)⁺; HPLC: 98.34%, |
| | | | rt: 5.80 min. |
| 64A | Chiral | Isomer-2 of compound-63 | ¹HNMR (DMSO-d₆, 400 MHz): δ |
| | separation | | 10.90 (s, 1H), 8.44 (s, 1H), 8.23 (s, |
| | of Cpd-63 | | 1H), 7.86 (s, 1H), 7.73-7.71 (m, |
| | | | 1H), 7.63-7.60 (m, 1H), 6.79-6.78 |
| | | | (m, 1H), 4.74-4.72 (m, 1H), 4.21 |
| | | | (s, 2H), 3.76 ( s, 2H), 3.38-3.36 (m, |
| | | | 2H), 2.56-2.54 (m, 3H), 2.42-2.40 |
| | | | (m, 1H), 2.32-2.25 (m, 1H), 2.15 |
| | | | (s, 3H), 1.80-1.78 (m, 3H), 1.55- |
| | | | 1.50 (m, 1H), 0.97- 0.87 (m, 3H); |
| | | | LCMS m/z: 481.10 (M + H)⁺; |
| | | | HPLC: 96.86%, rt: 5.74 min. |
| 65 | E-50 | 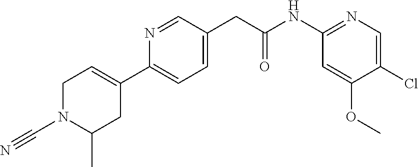 [SMILES: COc1cc(NC(=O)Cc2ccc(C3=CCN(C#N)C(C)C3)nc2)ncc1Cl] | ¹HNMR (DMSO-d₆, 400 MHz): δ 10.94 (s, 1H), 8.47 (s, 1H), 8.23 (s, 1H), 7.90 (s, 1H), 7.74-7.71 (m, 1H), 7.55-7.53 (d, 1H), 6.59-6.58 (m, 1H), 4.18-4.05 (m, 1H), 3.88 (d, 3H), 3.78 (s, 2H), 3.60-3.58 (m, 2H), 2.86-2.80 (m, 1H), 2.70-2.64 (m, 1H), 1.35 (d, 3H); LCMS m/z: 398.10 (M + H)⁺; HPLC: 98.41%, rt: 7.03 min. |
| 66 | Chiral | Isomer-1 of compound-65 | ¹HNMR (DMSO-d₆, 400 MHz): δ |
| | separation | | 10.94 (s, 1H), 8.48 (s, 1H), 8.23 (s, |
| | of Cpd-65 | | 1H), 7.90 (s, 1H), 7.75-7.71 (m, |
| | | | 1H), 7.56-7.53 (d, 1H), 6.59-6.58 |
| | | | (m, 1H), 4.18-4.05 (m, 1H), 3.88 |
| | | | (d, 3H), 3.79 (s, 2H), 3.58-3.50 (m, |
| | | | 2H), 2.45-2.42 (m, 1H), 2.70-2.63 |
| | | | (m, 1H), 1.35 (d, 3H); LCMS m/z: |
| | | | 398.10 (M + H)⁺; HPLC: 96.10%, |
| | | | rt: 5.18 min, Chiral HPLC: |
| | | | 99.01%, rt: 10.90 min. |
| 67 | Chiral | Isomer-2 of compound-65 | ¹HNMR (DMSO-d₆, 400 MHz): δ |
| | separation | | 10.94 (s, 1H), 8.48 (s, 1H), 8.23 (s, |
| | of Cpd-65 | | 1H), 7.91 (s, 1H), 7.74-7.71 (m, |
| | | | 1H), 7.56-7.53 (d, 1H), 6.59-6.58 |
| | | | (m, 1H), 4.18-4.05 (m, 1H), 3.88 |
| | | | (d, 3H), 3.79 (s, 2H), 3.60-3.58 (m, |
| | | | 2H), 2.86-2.80 (m, 1H), 2.70-2.63 |
| | | | (m, 1H), 1.35 (d, 3H); LCMS m/z: |
| | | | 398.10 (M + H)⁺; HPLC: 99.19%, |
| | | | rt: 6.47 min, Chiral HPLC: |
| | | | 99.01%, rt: 13.35 min. |
| 68 | Chiral separation of Cpd-65 | 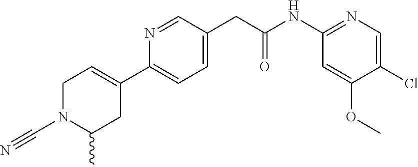 [SMILES: COc1cc(NC(=O)Cc2ccc(C3=CCN(C#N)C(C)C3)nc2)ncc1Cl] | ¹HNMR (DMSO-d₆, 400 MHz): δ 10.94 (s, 1H), 8.47 (s, 1H), 8.22 (s, 1H), 7.90 (s, 1H), 7.74-7.71 (m, 1H), 7.56-7.53 (d, 1H), 6.65-6.60 (m, 1H), 4.18-4.03 (m, 1H), 3.87 (d, 3H), 3.78 (s, 2H), 3.60-3.58 (m, 2H), 2.86-2.80 (m, 1H), 2.70-2.63 (m, 1H), 1.35 (d, 3H); LCMS m/z: 398.10 (M + H)⁺; HPLC: 99.41%, rt: 4.40 min, Chiral HPLC: 94.85%, rt: 14.45 min. |
| | | Isomer-3 of compound-65 | |
| 69 | E-51 | 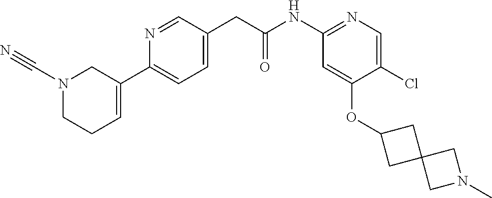 [SMILES: CN1CC2(CC(Oc3cc(NC(=O)Cc4ccc(C5=CCCN(C#N)C5)nc4)ncc3Cl)C2)C1] | ¹HNMR (DMSO-d₆, 400 MHz): δ 10.92 (s, 1H), 8.44 (s, 1H), 8.22 (s, 1H), 7.74-7.71 (m, 2H), 7.63-7.61 (m, 1H), 6.79-6.78 (m, 1H), 4.70- 4.68 (m, 2H), 4.22 (s, 2H), 3.78 (s, 2H), 3.36-3.35 (m, 2H), 3.15 (s, 3H), 3.08 (s, 3H), 2.42-2.41 (m, 2H), 2.22-1.14 (d, 4H); LCMS m/z: 479.20 (M + H)⁺; HPLC: 98.41%, rt: 13.84 min. |
| 70 | E-52 | 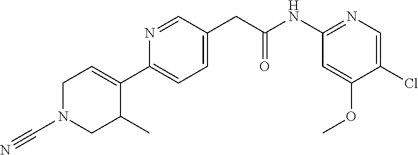 [SMILES: COc1cc(NC(=O)Cc2ccc(C3=CCN(C#N)CC3C)nc2)ncc1Cl] | ¹HNMR (DMSO-d₆, 400MHz): δ 10.94 (s, 1H), 8.48 (d, 1H), 8.22 (s, 1H), 7.91 (s, 1H), 7.74-7.71 (m, 1H), 7.57-7.55 (d, 1H), 6.47-6.45 (m, 1H), 4.00 (d, 2H), 3.88 (s, 3H), 3.78 (s, 2H), 3.37-3.36 (m, 1H), 3.29-3.28 (m, 2H), 1.11-1.10 (d, 3H); LCMS m/z: 398.10 (M + H)⁺; HPLC: 97.41%, rt: 6.02 min. |
| 71 | Chiral | Isomer-1 of compound 70 | ¹HNMR (DMSO-d₆, 400 MHz): δ |
| | separation | | 10.94 (s, 1H), 8.48 (d, 1H), 8.22 (s, |
| | of Cpd-70 | | 1H), 7.91 (s, 1H), 7.74-7.71 (m, |
| | | | 1H), 7.57-7.55 (d, 1H), 6.47-6.45 |
| | | | (m, 1H), 4.00 (d, 2H), 3.88 (s, 3H), |
| | | | 3.78 (s, 2H), 3.37-3.36 (m, 1H), |
| | | | 3.29-3.28 (m, 2H), 1.12-1.10 (d, |
| | | | 3H); LCMS m/z: 398.10 (M + H)⁺; |
| | | | HPLC: 96.55%, rt: 6.05min, Chiral |
| | | | HPLC: 96.01%, rt: 10.34 min. |
| 72 | Chiral | Isomer-2 of compound-70 | ¹HNMR (DMSO-d₆, 400 MHz): δ |
| | separation | | 10.94 (s, 1H), 8.48 (d, 1H), 8.22 (s, |
| | of Cpd-70 | | 1H), 7.91 (s, 1H), 7.74-7.71 (m, |
| | | | 1H), 7.57-7.55 (d, 1H), 6.47-6.45 |
| | | | (m, 1H), 4.00 (d, 2H), 3.88 (s, 3H), |
| | | | 3.78 (s, 2H), 3.37-3.36 (m, 1H), |
| | | | 3.29-3.28 (m, 2H), 1.12-1.10 (d, |
| | | | 3H); LCMS m/z: 398.10 (M + H)⁺; |
| | | | HPLC: 93.68%, rt: 6.02min, Chiral |
| | | | HPLC: 97.21%, rt: 11.76 min. |
| 73 | H-25 | 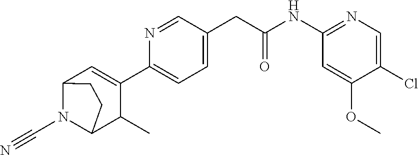 | ¹HNMR (DMSO-d₆, 400 MHz): δ 10.93 (s, 1H), 8.48 (s, 1H), 8.22 (s, 1H), 7.90 (s, 1H), 7.73 (d, 1H), 7.55-7.53 (m, 1H), 6.95-6.94 (m, 1H), 4.33-4.26 (m, 1H), 3.88 (s, 3H), 3.78 (m, 2H), 3.10-3.01 (m, 1H), 2.25-2.15 (m, 2H), 2.10-1.96 (m, 2H), 1.75-1.61 (m, 2H); LCMS m/z: 410.10 (M + H)⁺; HPLC: 98.78%, rt: 4.76 min. |
| 74 | H-12 | 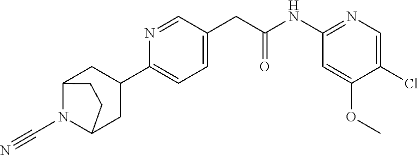 [SMILES: COc1cc(NC(=O)Cc2ccc(C3CC4CCC(C3)N4C#N)nc2)ncc1Cl] | ¹HNMR (DMSO-d₆, 400 MHz): δ 10.93 (s, 1H), 8.45 (s, 1H), 8.22 (s, 1H), 7.91 (s, 1H), 7.67-7.66 (m, 1H), 7.27-7.25 (m, 1H), 4.01 (s, 2H), 3.88 (s, 3H), 3.74 (s, 2H), 3.23 (m, 1H), 2.084-1.99 (m, 4H), 1.89-1.87(m, 2H), 1.75-1.71(m, 2H); LCMS m/z: 412.00 (M + H)⁺; HPLC: 98.55%, rt: 3.81 min. |
| 75 | E-53 | 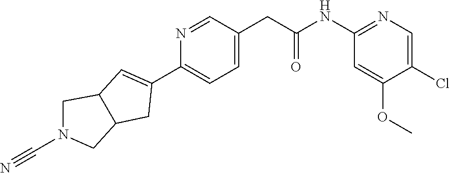 [SMILES: COc1cc(NC(=O)Cc2ccc(C3=CC4CN(C#N)CC4C3)nc2)ncc1Cl] | ¹HNMR (DMSO-d₆, 400 MHz): δ 10.93 (s, 1H), 8.47 (d, 1H), 8.22 (s, 1H), 7.90 (s, 1H), 7.72-7.70 (m, 1H), 7.53-7.51 (m, 1H), 6.42 (d, 1H), 3.88 (s, 3H), 3.78 ( s, 2H), 3.58-3.56 (m, 2H), 3.55-3.51 (m, 1H), 3.39-3.36 (m, 1H), 3.17-3.15 (m, 1H), 3.13-2.96 (m, 2H), 2.70- 2.66 (m, 1H); LCMS m/z: 410.10 (M + H)⁺; HPLC: 98.38%, rt: 5.67 min. |
| 76 | H-13 | 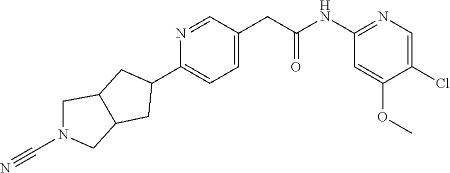 [SMILES: COc1cc(NC(=O)Cc2ccc(C3CC4CN(C#N)CC4C3)nc2)ncc1Cl] | ¹HNMR (DMSO-d₆, 400 MHz): δ 10.97 (s, 1H), 8.45 (s, 1H), 8.22 (s, 1H), 7.91 (s, 1H), 7.64 (d, 1H), 7.22 (d, 1H), 3.88 (s, 3H), 3.73 (s, 2H), 3.33-3.30 (m, 2H), 3.27-3.25 (m, 2H), 3.19-3.12 (m, 1H), 2.75- 2.70 (m, 2H), 2.22-2.18 (m, 2H), 1.62-1.57 (m, 2H); LCMS m/z: 412.10 (M + H)⁺; HPLC: 99.04%, rt: 6.03 min. |
| 77 | E-54 | 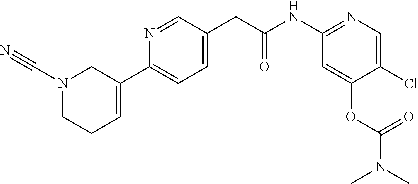 [SMILES: CN(C)C(=O)Oc1cc(NC(=O)Cc2ccc(C3=CCCN(C#N)C3)nc2)ncc1Cl] | ¹HNMR (DMSO-d₆, 400 MHz): δ 11.11 (s, 1H), 8.47-8.45 (m, 1H), 8.03 (s, 1H), 8.75 (d, 1H), 7.62 (d, 2H), 6.80-6.77(m, 1H), 4.21 (d, 2H), 3.78 (s, 2H), 3.36-3.35 (m, 2H), 3.06 (s, 3H), 2.92 (s, 3H), 2.42-2.41 (m, 2H); LCMS m/z: 441.10 (M + H)⁺; HPLC: 93.88%, rt: 6.59 min. |
| 78 | H-7 | 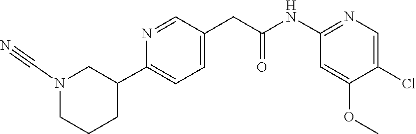 [SMILES: COc1cc(NC(=O)Cc2ccc(C3CCCN(C#N)C3)nc2)ncc1Cl] | ¹HNMR (DMSO-d₆, 400 MHz): δ 10.93 (s, 1H), 8.44 (s, 1H), 8.22 (s, 1H), 7.91 (s, 1H), 7.70-7.67 (m, 1H), 7.32-7.30 (m, 1H), 3.88 (s, 3H), 3.75 (s, 2H), 3.55-3.45 (m, 1H), 3.37-3.36 (m, 1H), 3.23-3.20 (m, 2H), 3.11-3.00 (m, 1H), 2.00- 1.90 (m, 1H), 1.74-1.66 (m, 3H); LCMS m/z: 386.10 (M + H)⁺; |
| | | | HPLC: 99.47%, rt: 6.21 min. |
| 79 | Chiral | Isomer-1 of compound-78 | ¹HNMR (DMSO-d₆, 400 MHz): δ |
| | separation | | 10.92 (s, 1H), 8.44 (s, 1H), 8.22 (s, |
| | of Cpd-78 | | 1H), 7.91 (s, 1H), 7.70-7.67 (m, |
| | | | 1H), 7.32-7.30 (m, 1H), 3.88 (s, |
| | | | 3H), 3.75 (s, 2H), 3.55-3.45 (m, |
| | | | 1H), 3.37-3.36 (m, 1H), 3.23-3.20 |
| | | | (m, 2H), 3.11-3.00 (m, 1H),2.00- |
| | | | 1.90 (m, 1H), 1.74-1.66 (m, 3H); |
| | | | LCMS m/z: 386.10 (M + H)⁺; |
| | | | HPLC: 99.65%, rt: 6.76 min, |
| | | | Chiral HPLC: 99.18%, rt: 19.67 |
| | | | min. |
| 80 | Chiral | Isomer-2 of compound-78 | ¹HNMR (DMSO-d₆, 400 MHz): δ |
| | separation | | 10.93 (s, 1H), 8.43 (s, 1H), 8.22 (s, |
| | of Cpd-78 | | 1H), 7.91 (s, 1H), 7.70-7.67 (m, |
| | | | 1H), 7.32-7.30 (m, 1H), 3.88 (s, |
| | | | 3H), 3.75 (s, 2H), 3.55-3.45 (m, |
| | | | 1H), 3.37-3.36 (m, 1H), 3.23-3.20 |
| | | | (m, 2H), 3.11-3.00 (m, 1H),2.00- |
| | | | 1.90 (m, 1H), 1.74-1.66 (m, 3H); |
| | | | LCMS m/z: 386.10 (M + H)⁺; |
| | | | HPLC: 99.65%, rt: 6.76 min, |
| | | | Chiral HPLC: 98.81%, rt: 16.02 |
| | | | min. |
| 81 | E-58 | 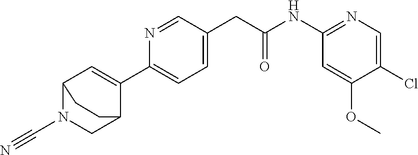 [SMILES: COc1cc(NC(=O)Cc2ccc(C3=CC4CCC3CN4C#N)nc2)ncc1Cl] | ¹HNMR (DMSO-d₆, 400 MHz): δ 10.94 (s, 1H), 8.51 (s, 1H), 8.22 (s, 1H), 7.90 (s, 1H), 7.74 (d, 2H), 7.18-7.21 (m, 1H), 4.30-4.20 (m, 1H), 3.87 (s, 3H), 3.82-3.79 (m, 3H), 3.50-3.48 (m, 1H), 3.90-3.80 (m, 1H), 2.05-2.10 (m, 1H), 1.80- 1.70 (m, 1H), 1.45-1.20 (m, 2H); LCMS m/z: 410.00 (M + H)⁺; HPLC: 97.79%, rt: 6.60 min. |
| 82 | H-8 | 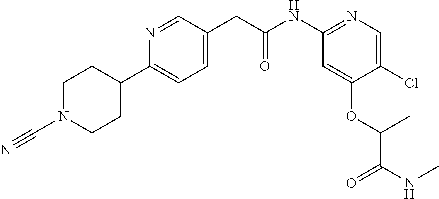 [SMILES: CNC(=O)C(C)Oc1cc(NC(=O)Cc2ccc(C3CCN(C#N)CC3)nc2)ncc1Cl] | ¹HNMR (DMSO-d₆, 400 MHz): δ 10.88 (s, 1H), 8.43 (s, 1H), 8.23 (s, 1H), 8.13-8.12 (m, 1H), 7.77 (s, 1H), 7.66-7.65 (m, 1H), 7.26 (d, 1H), 4.77-4.75 (m, 1H), 3.73 (s, 2H), 3.47-3.44 (m, 2H), 3.19-3.12 (m, 2H), 2.82-2.81 (m, 1H), 2.58- 2.57 (d, 2H), 1.84-1.75 (d, 3H), 1.45 (d, 3H); LCMS m/z: 457.10 (M + H)⁺; HPLC: 93.81%, rt: 5.63 min. |
| 83 | Chiral | Isomer-1 of compound-82 | ¹HNMR (DMSO-d₆, 400 MHz): δ |
| | separation | | 10.88 (s, 1H), 8.43 (s, 1H), 8.23 (s, |
| | of Cpd-82 | | 1H), 8.13-8.12 (m, 1H), 7.77 (s, |
| | | | 1H), 7.66-7.65 (m, 1H), 7.26 (d, |
| | | | 1H), 4.77-4.75 (m, 1H), 3.73 (s, |
| | | | 2H), 3.47-3.44 (m, 2H), 3.19-3.12 |
| | | | (m, 2H), 2.82-2.75 (m, 1H), 2.58 |
| | | | (d, 2H), 1.84-1.75 (d, 3H), 1.45 (d, |
| | | | 3H); LCMS m/z: 457.10 (M + H)⁺; |
| | | | HPLC: 96.38%, rt: 5.67 min, |
| | | | Chiral HPLC: 98.66%, rt: 9.87 |
| | | | min. |
| 84 | Chiral | Isomer-2 of compound-82 | ¹HNMR (DMSO-d₆, 400 MHz): δ |
| | separation | | 10.86 (s, 1H), 8.43 (s, 1H), 8.23 (s, |
| | of Cpd-82 | | 1H), 8.13-8.12 (m, 1H), 7.77 (s, |
| | | | 1H), 7.66-7.65 (m, 1H), 7.26 (d, |
| | | | 1H), 4.77-4.75 (m, 1H), 3.73 (s, |
| | | | 2H), 3.47-3.44 (m, 2H), 3.19-3.12 |
| | | | (m, 2H), 2.87-2.81 (m, 1H), 2.58 |
| | | | (d, 2H), 1.84-1.75 (d, 3H), 1.45 (d, |
| | | | 3H); LCMS m/z: 457.10 (M + H)⁺; |
| | | | HPLC: 96.38%, rt: 5.67 min, |
| | | | Chiral HPLC: 98.77%, rt: 12.13 |
| | | | min. |
| 85 | H-14 | 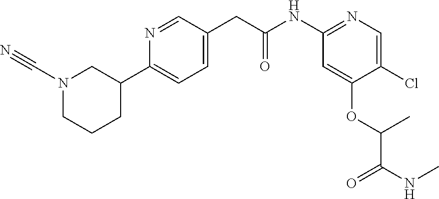 [SMILES: CNC(=O)C(C)Oc1cc(NC(=O)Cc2ccc(C3CCCN(C#N)C3)nc2)ncc1Cl] | 1HNMR (DMSO-d₆, 400 MHz): δ 10.93 (s, 1H), 8.45 (s, 1H), 8.22 (s, 1H), 8.17-8.15 (m, 1H), 7.76 (s, 1H), 7.67-7.66 (m, 1H), 7.27-7.25 (m, 1H), 4.75-4.67 (m, 1H), 3.73 (s, 2H), 3.55-3.47 (m, 1H), 3.35- 3.29 (m, 1H), 3.27-3.19 (m, 1H), 3.10-2.95 (m, 2H), 2.58-2.57 (m, 3H), 1.91-1..82 (m, 1H), 1.75-1.63 (m, 3H), 1.46-1.44 (m, 3H); LCMS m/z: 457.20 (M + H)⁺; HPLC: 95.68%, rt: 6.26 min. |
| 86 | Chiral | Diastereomer-1 of | 1HNMR (DMSO-d₆, 400 MHz): δ |
| | separation | compound-85 | 10.93 (s, 1H), 8.45 (s, 1H), 8.22 (s, |
| | of Cpd-85 | | 1H), 8.17-8.15 (m, 1H), 7.76 (s, |
| | | | 1H), 7.67-7.66 (m, 1H), 7.27-7.25 |
| | | | (m, 1H), 4.75-4.67 (m, 1H), 3.73 |
| | | | (s, 2H), 3.55-3.47 (m, 1H), 3.35- |
| | | | 3.29 (m, 1H), 3.27-3.19 (m, 1H), |
| | | | 3.10-2.95 (m, 2H), 2.58-2.57 (m, |
| | | | 3H), 1.91-1..82 (m, 1H), 1.75-1.63 |
| | | | (m, 3H), 1.46-1.44 (m, 3H); LCMS |
| | | | m/z: 457.10 (M + H)⁺; HPLC: |
| | | | 98.215%, rt: 5.411 min. |
| 87 | Chiral | Enantiomer-1 of compound-86 | 1HNMR (DMSO-d₆, 400 MHz): δ |
| | separation | | 10.93 (s, 1H), 8.45 (s, 1H), 8.22 (s, |
| | of Cpd-86 | | 1H), 8.17-8.15 (m, 1H), 7.76 (s, |
| | | | 1H), 7.67-7.66 (m, 1H), 7.27-7.25 |
| | | | (m, 1H), 4.75-4.67 (m, 1H), 3.73 |
| | | | (s, 2H), 3.55-3.47 (m, 1H), 3.35- |
| | | | 3.29 (m, 1H), 3.27-3.19 (m, 1H), |
| | | | 3.10-2.95 (m, 2H), 2.58-2.57 (m, |
| | | | 3H), 1.91-1..82 (m, 1H), 1.75-1.63 |
| | | | (m, 3H), 1.46-1.44 (m, 3H); LCMS |
| | | | m/z: 457.20 (M + H)⁺; HPLC: |
| | | | 98.07%, rt: 5.43 min. CHIRAL |
| | | | HPLC: 97.56%, rt: 9.75 min. |
| 88 | Chiral | Enantiomer-2 of compound-86 | 1HNMR (DMSO-d₆, 400 MHz): δ |
| | separation | | 10.93 (s, 1H), 8.45 (s, 1H), 8.22 (s, |
| | of Cpd-86 | | 1H), 8.17-8.15 (m, 1H), 7.76 (s, |
| | | | 1H), 7.67-7.66 (m, 1H), 7.27-7.25 |
| | | | (m, 1H), 4.75-4.67 (m, 1H), 3.73 |
| | | | (s, 2H), 3.55-3.47 (m, 1H), 3.35- |
| | | | 3.29 (m, 1H), 3.27-3.19 (m, 1H), |
| | | | 3.10-2.95 (m, 2H), 2.58-2.57 (m, |
| | | | 3H), 1.91-1..82 (m, 1H), 1.75-1.63 |
| | | | (m, 3H), 1.46-1.44 (m, 3H); LCMS |
| | | | m/z: 457.10 (M + H)⁺; HPLC: |
| | | | 95.00%, rt: 5.43 min. CHIRAL |
| | | | HPLC: 98.49%, rt: 10.833 min. |
| 89 | Chiral | Diastereomer-2 of | 1HNMR (DMSO-d₆, 400 MHz): δ |
| | separation | compound-85 | 10.93 (s, 1H), 8.45 (s, 1H), 8.22 (s, |
| | of Cpd-85 | | 1H), 8.17-8.15 (m, 1H), 7.76 (s, |
| | | | 1H), 7.67-7.66 (m, 1H), 7.27-7.25 |
| | | | (m, 1H), 4.75-4.67 (m, 1H), 3.73 |
| | | | (s, 2H), 3.55-3.47 (m, 1H), 3.35- |
| | | | 3.29 (m, 1H), 3.27-3.19 (m, 1H), |
| | | | 3.10-2.95 (m, 2H), 2.58-2.57 (m, |
| | | | 3H), 1.91-1..82 (m, 1H), 1.75-1.63 |
| | | | (m, 3H), 1.46-1.44 (m, 3H); LCMS |
| | | | m/z: 457.10 (M + H)⁺; HPLC: |
| | | | 94.151%, rt: 5.156 min. |
| 90 | Chiral | Enantiomer-1 of compound-89 | ¹HNMR (DMSO-d₆, 400 MHz): δ |
| | separation | | 10.93 (s, 1H), 8.45 (s, 1H), 8.22 (s, |
| | of Cpd-89 | | 1H), 8.17-8.15 (m, 1H), 7.76 (s, |
| | | | 1H), 7.67-7.66 (m, 1H), 7.27-7.25 |
| | | | (m, 1H), 4.75-4.67 (m, 1H), 3.73 |
| | | | (s, 2H), 3.55-3.47 (m, 1H), 3.35- |
| | | | 3.29 (m, 1H), 3.27-3.19 (m, 1H), |
| | | | 3.10-2.95 (m, 2H), 2.58-2.57 (m, |
| | | | 3H), 1.91-1 .. 82 (m, 1H), 1.75-1.63 |
| | | | (m, 3H), 1.46-1.44 (m, 3H); LCMS |
| | | | m/z: 458.00 (M + H)⁺; HPLC: |
| | | | 97.96%, rt: 5.72 min, Chiral |
| | | | HPLC: 97.94%, rt: 12.50 min. |
| 91 | Chiral | Enantiomer-2 of compound-89 | ¹HNMR (DMSO-d₆, 400 MHz): δ |
| | separation | | 10.93 (s, 1H), 8.45 (s, 1H), 8.22 (s, |
| | of Cpd-89 | | 1H), 8.17-8.15 (m, 1H), 7.76 (s, |
| | | | 1H), 7.67-7.66 (m, 1H), 7.27-7.25 |
| | | | (m, 1H), 4.75-4.67 (m, 1H), 3.73 |
| | | | (s, 2H), 3.55-3.47 (m, 1H), 3.35- |
| | | | 3.29 (m, 1H), 3.27-3.19 (m, 1H), |
| | | | 3.10-2.95 (m, 2H), 2.58-2.57 (m, |
| | | | 3H), 1.91-1..82 (m, 1H), 1.75-1.63 |
| | | | (m, 3H), 1.46-1.44 (m, 3H); LCMS |
| | | | m/z: 458.05 (M + H)⁺; HPLC: |
| | | | 98.49%, rt: 5.73 min, Chiral |
| | | | HPLC: 95.61%, rt: 13.81 min. |
| 92 | H-15 | 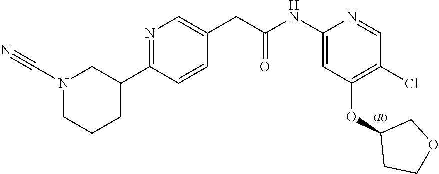 | ¹HNMR (DMSO-d₆, 400 MHz): δ 10.95 (s, 1H), 8.436 (s, 1H), 8.24 (s, 1H), 7.88 (s, 1H), 7.99 (m, 1H), 7.32-7.30 (m, 1H), 5.11 (s, 1H), 3.89-3.71 (m, 5H), 3.50-3.36 (m, 2H), 3.327-3.20 (m, 1H), 3.08-2.95 (m, 3H), 2.32-2.23 (m, 1H), 2.01- 1.95 (m, 2H), 1.74-1.64 (m, 3H),; LCMS m/z: 442.10 (M + H)⁺; HPLC: 97.23%, rt: 6.241 min. |
| 93 | Chiral | Isomer-1 of compound-92 | ¹HNMR (DMSO-d₆, 400 MHz): |
| | separation | | δ; 10.96 (s, 1H), 8.43 (s, 1H), 8.24 |
| | of Cpd-92 | | (s, 1H), 7.87 (s, 1H), 7.68-7.66 (d, |
| | | | 1H), 7.28-7.26 (d, 1H), 5.12-5.10 |
| | | | (m, 1H), 3.88-3.83 (m, 3H), 3.76 |
| | | | (s, 2H), 3.52-3.49 (m, 1H), 3.40- |
| | | | 3.38 (m, 1H), 3.26-3.18 (m, 2H), |
| | | | 3.11-2.97 (m, 3H), 2.28-2.21 (m, |
| | | | 1H), 2.00-1.94 (m, 2H), 1.71-1.64 |
| | | | (m, 2H). LCMS m/z: (M + H)⁺ |
| | | | 442.10; HPLC: 97.02%, rt: 5.52 |
| | | | min |
| 94 | Chiral | Isomer-2 of compound-92 | ¹HNMR (DMSO-d₆, 400 MHz): δ; |
| | separation | | 10.95 (s, 1H), 8.43 (s, 1H), 8.24 (s, |
| | of Cpd-92 | | 1H), 7.86 (s, 1H), 7.67-7.65 (d, |
| | | | 1H), 7.29-7.27 (d, 1H), 5.12-5.10 |
| | | | (m, 1H), 3.87-3.81 (m, 3H), 3.78- |
| | | | 3.72 (m, 3H), 3.52-3.47 (m, 1H), |
| | | | 3.41-3.36 (m, 1H), 3.28-3.18 (m, |
| | | | 1H), 3.10-2.97 (m, 3H), 2.28-2.21 |
| | | | (m, 1H),2.00-1.94 (m, 2H), 1.72- |
| | | | 1.63 (m, 2H). LCMS m/z: (M + |
| | | | H)⁺ 442.10; HPLC: 96.56%, rt: |
| | | | 6.75 min |
| 95 | H-16 | 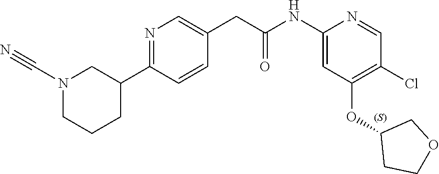 | ¹HNMR (DMSO-d₆, 400 MHz): δ 10.95 (s, 1H), 8.43-8.42 (s, 1H), 8.24 (s, 1H), 7.88 (s, 1H), 7.70- 7.67 (m, 1H), 7.32-7.30 (m, 1H), 5.11 (s, 1H), 3.86-3.73 (m, 6H), 3.50-3.46 (m, 1H), 3.39-3.33 (m, 1H), 3.26-3.20 (m, 1H), 3.058-2.50 (m, 2H), 2.27-2.23 (m, 1H), 1.99- 1.95 (m, 2H), 1.74-1.64 (m, 3H); LCMS m/z: 442.10 (M + H)⁺; HPLC: 96.414%, rt: 6.235 min. |
| 96 | Chiral | Isomer-1 of compound-95 | ¹HNMR (DMSO-d₆, 400 MHz): δ; |
| | separation | | 10.94 (s, 1H), 8.42 (s, 1H), 8.21 (s, |
| | of Cpd-95 | | 1H), 7.89 (s, 1H), 7.67-7.64 (d, |
| | | | 1H), 7.27-7.24 (d, 1H), 5.13-5.11 |
| | | | (m, 1H), 3.89-3.84 (m, 3H), 3.77- |
| | | | 3.73 (m, 3H), 3.52-3.48 (m, 1H), |
| | | | 3.41-3.38 (m, 1H), 3.27-3.18 (m, |
| | | | 1H), 3.10-2.98 (m, 2H), 2.28-2.20 |
| | | | (m, 1H), 2.01-1.9 5 (m, 2H), 1.70- |
| | | | 1.62 (m, 3H). LCMS m/z: (M + H)⁺ |
| | | | 442.10; HPLC: 98.31%, rt: 5.51 |
| | | | min |
| 97 | Chiral | Isomer-2 of compound-95 | ¹HNMR (DMSO-d₆, 400 MHz): δ |
| | separation | | 10.97 (s, 1H), 8.45 (s, 1H), 8.34 (s, |
| | of Cpd-95 | | 1H), 7.98 (s, 1H), 7.10-7.12 (m, |
| | | | 1H), 7.35-7.37 (m, 1H), 5.1 (m, |
| | | | 1H), 3.89-3.91 (m, 3H), 3.76-3.78 |
| | | | (m, 3H), 3.55-3.58 (m, 1H), 3.41- |
| | | | 3.43 (m, 1H), 3.21-3.23 (s, 1H), |
| | | | 2.96-2.98 (m, 2H), 2.25-2.27 (m, |
| | | | 1H), 1.98-2.01 (m, 2H), 1.68-1.70 |
| | | | (m, 3H), LCMS m/z: 442.10 |
| | | | (M + H)⁺; HPLC: 97.80%, rt: 3.95 |
| | | | min. |
| 98 | H-9 | 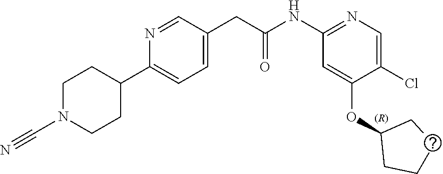 | ¹HNMR (DMSO-d₆, 400 MHz): δ 10.98 (s, 1H), 8.44 (s, 1H), 8.24 (s, 1H), 7.90 (s, 1H), 7.67 (d, 1H), 7.22 (d, 1H), 5.15-5.12 (m, 1H), 3.88-3.82 (m, 3H), 3.78-3.72 (m, 3H), 3.50-3.43 (m, 2H), 3.23-3.11 (m, 2H), 2.85-2.77 (m, 1H), 2.29- 2.22 (m, 1H), 2.02-1.96 (m, 1H), 1.85-1.80 (m, 4H); LCMS m/z: 442.00 (M + H)⁺; HPLC: 99.20%, rt: 6.41 min. |
| 99 | H-10 | 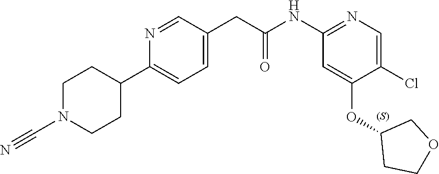 | ¹HNMR (DMSO-d₆, 400 MHz): δ 10.95 (s, 1H), 8.43 (s, 1H), 8.24 (s, 1H), 7.88 (s, 1H), 7.67 (d, 1H), 7.24 (d, 1H), 5.14-5.12 (m, 1H), 3.84-3.81 (m, 3H), 3.75-3.74 (m, 3H), 3.48-3.45 (m, 2H), 3.15-3.12 (m, 2H), 2.81-2.79 (m, 1H), 2.27- 2.24 (m, 1H), 2.03-1.98 (m, 1H), 1.89-1.75 (m, 4H); LCMS m/z: 442.10 (M + H)⁺; HPLC: 97.56.%, rt: 5.67 min. |
| 100 | E-55 | 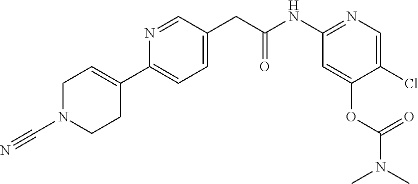 [SMILES: CN(C)C(=O)Oc1cc(NC(=O)Cc2ccc(C3=CCN(C#N)CC3)nc2)ncc1Cl] | ¹HNMR (DMSO-d₆, 400 MHz): δ 11.20 (s, 1H), 8.45 (s, 2H), 8.15 (s, 1H), 7.85 (d, 1H), 7.45 (d, 1H), 6.82 (s, 1H), 4.0 (s, 2H), 3.80 (s, 2H), 3.49-3.4 (m, 2H), 3.06 (s, 3H), 2.92 (m, 3H), 2.70 (s, 2H); LCMS m/z: 441.15 (M + H)⁺; HPLC: 97.82, rt: 6.64 min. |
| 101 | H-11 | 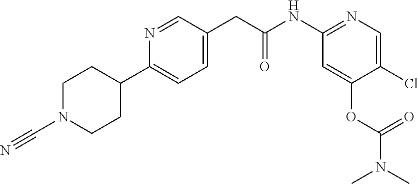 [SMILES: CN(C)C(=O)Oc1cc(NC(=O)Cc2ccc(C3CCN(C#N)CC3)nc2)ncc1Cl] | ¹HNMR (DMSO-d₆, 400 MHz): δ 11.2 (s, 1H), 8.50 (s, 1H), 8.41 (s, 2H), 8.02 (d, 1H), 7.56 (d, 1H), 3.75 (s, 2H), 3.45 (d, 2H), 3.25- 3.15 (m, 2H), 3.05 (s, 3H), 2.92 (s, 3H), 2.95-2.82 (m, 1H), 1.85-1.72 (m, 4H); LCMS m/z: 443.15 (M + H)⁺; HPLC: 99.42 rt: 3.40 min. |
| 102 | E-59 | 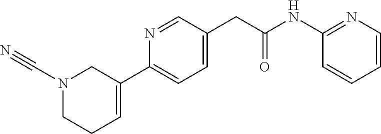 [SMILES: N#CN1CCC=C(c2ccc(CC(=O)Nc3ccccn3)cn2)C1] | ¹HNMR (DMSO-d₆, 400 MHz): δ 10.57 (s, 1H), 8.442 (s, 1H), 8.31- 8.29 (m, 1H), 8.02-7.99 (m, 1H), 7.76-7.7 (m, 2H), 7.60-7.57 (m, 1H), 7.09-7.05 (m, 1H), 6.78-6.75 (m, 1H), 4.199-4.193 (m, 2H), 3.75(s, 2H), 3.34-3.31 (m, 2H), 2.49-2.47 (m, 2H); |
| | | | LCMS m/z: 320.10 (M + H)⁺; |
| | | | HPLC: 99.1503%, rt: 6.006 min. |
| 103 | E-60 | 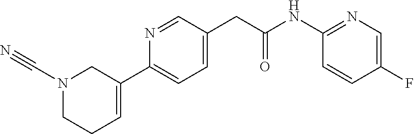 [SMILES: N#CN1CCC=C(c2ccc(CC(=O)Nc3ccc(F)cn3)cn2)C1] | ¹HNMR (DMSO-d₆, 400 MHz): δ 10.9 (s, 1H), 8.45 (s, 1H), 8.39 (m, 1H), 8.1 (m, 1H), 7.78 (m, 2H), 7.61 (m, 1H), 6.8 (m, 1H), 4.23 (m, 2H), 3.76 (s, 2H), 3.34 (m, 2H), 2.5 (m, 2H); LCMS m/z: 338.15(M + H)⁺; HPLC: 98.20%, rt: 5.751 min. |
| 104 | E-61 | 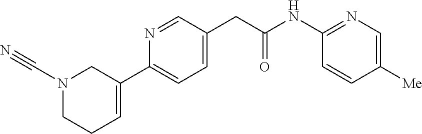 [SMILES: Cc1ccc(NC(=O)Cc2ccc(C3=CCCN(C#N)C3)nc2)nc1] | ¹HNMR (DMSO-d₆, 400 MHz): δ 10.66 (s, 1H), 8.44 (d, 1H), 8.13 (s, 1H), 7.92-7.89 (m, 1H), 7.72-7.70 (m, 1H), 7.60-7.54 (m, 2H), 6.78 (s, 1H), 4.19 (d, 2H), 3.72 (s, 2H), 3.35-3.3 (m, 2H), 2.40-2.35 (m, 2H), 2.21 (s, 3H). LCMS m/z: 334.10 (M + H)⁺; HPLC: 96.78%. |
| 105 | H-1 | 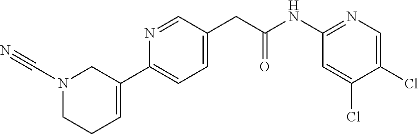 [SMILES: N#CN1CCC=C(c2ccc(CC(=O)Nc3cc(Cl)c(Cl)cn3)cn2)C1] | ¹HNMR (DMSO-d₆, 400 MHz): δ 11.20 (s, 1H), 8.53 (d, 1H), 8.43 (s, 1H), 8.27 (s, 1H), 7.69-7.58 (m, 2H), 6.85 (s, 1H), 4.19 (d, 2H), 3.78 (s, 2H), 3.35-3.3 (m, 2H), 2.40-2.35 (m, 2H). LCMS m/z: 388.10 (M + H)⁺; HPLC: 99.50%. |
| 106 | E-62 | 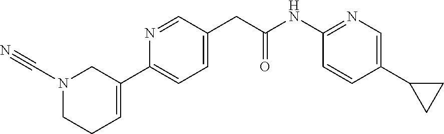 [SMILES: N#CN1CCC=C(c2ccc(CC(=O)Nc3ccc(C4CC4)cn3)cn2)C1] | ¹HNMR (DMSO-d₆, 400 MHz): δ 10.7 (s, 1H), 8.45 (d, 1H), 8.15 (s, 1H), 7.97 (d, 1H), 7.75-7.25 (m, 3H), 6.81 (s, 1H), 4.21 (d, 2H), 3.73 (s, 2H), 3.30 9 (s, 2H), 2.45 (s, 2H), 1.90-1.80 (m, 1H), 0.95- 0.85(m, 2H), 0.75-0.65 (m, 2H). LCMS m/z: 360.20 (M + H)⁺; HPLC: 98.41%. |
| 107 | E-63 | 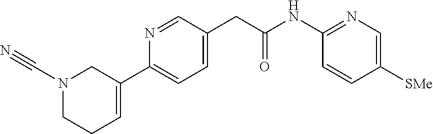 [SMILES: CSc1ccc(NC(=O)Cc2ccc(C3=CCCN(C#N)C3)nc2)nc1] | ¹HNMR (DMSO-d₆, 400 MHz): δ 10.81 (s, 1H), 8.45 (d, 1H), 8.25 (s, 1H), 7.98 (d, 1H), 7.75-7.59 (m, 3H), 6.78 (s, 1H), 4.21 (d, 2H), 3.76 (s, 2H), 3.36-3.32 (m, 2H), 2.50 (s, 3H), 2.41 (d, 2H). LCMS m/z: 366.15 (M + H)⁺; HPLC: 95.83%. |
| 108 | E-64 | 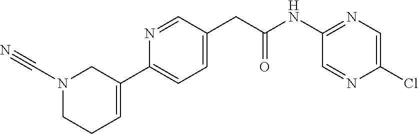 [SMILES: N#CN1CCC=C(c2ccc(CC(=O)Nc3cnc(Cl)cn3)cn2)C1] | ¹HNMR (DMSO-d₆, 400 MHz): δ 11.30 (s, 1H), 9.10 (s, 1H), 8.57 (d, 1H), 8.45 (d, 1H), 7.7-7.63 (m, 2H), 6.80 (s, 1H), 4.21 (d, 2H), 3.83 (s, 2H), 3.35 (s, 2H), 2.45 (s, 2H). LCMS m/z: 355.10 (M + H)⁺; HPLC: 98.63%. |
| 109 | E-65 | 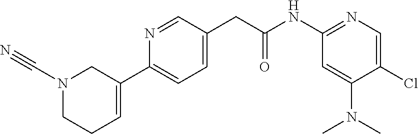 [SMILES: CN(C)c1cc(NC(=O)Cc2ccc(C3=CCCN(C#N)C3)nc2)ncc1Cl] | ¹HNMR (DMSO-d₆, 400 MHz): δ 10.73 (s, 1H), 8.43 (s, 1H), 8.07 (s, 1H), 7.75-7.73 (m, 2H), 7.62-7.64 (m, 1H), 6.79 (m, 1H), 4.21-4.19 (m, 2H), 3.77-3.75 (m, 2H), 3.34- 3.33 (m, 2H), 2.87-2.85 (s, 6H), 2.41 (s, 2H). LCMS m/z: 397.10 (M + H)⁺; HPLC: 97.22%, rt: 6.55 min. |
| 110 | E-66 | 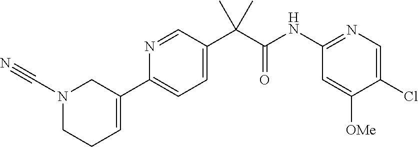 [SMILES: COc1cc(NC(=O)C(C)(C)c2ccc(C3=CCCN(C#N)C3)nc2)ncc1Cl] | ¹HNMR (DMSO-d₆, 400 MHz): δ 9.92 (s, 1H), 8.51 (d, 1H), 8.16 (s, 1H), 7.94 (s, 1H), 7.77-7.61 (m, 2H), 6.80 (s, 1H), 4.21 (d, 2H), 3.93 (s, 3H), 3.36-3.32 (m, 2H), 2.41 (s, 2H), 1.61 (s, 6H). LCMS m/z: 412.10 (M + H)⁺; HPLC: 99.34%. |
| 111 | E-67 | 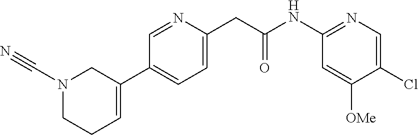 [SMILES: COc1cc(NC(=O)Cc2ccc(C3=CCCN(C#N)C3)cn2)ncc1Cl] | ¹HNMR (DMSO-d₆, 400 MHz): δ 10.90 (s, 1H), 8.56 (d, 1H), 8.22 (s, 1H), 7.93 (s, 1H), 7.82-7.80 (m, 1H), 7.37 (d, 1H), 6.38 (s, 1H), 4.18 (d, 2H), 3.94 (s, 2H), 3.88 (s, 3H), 3.34-3.3 (m, 2H), 2.37-2.35 (m, 2H). LCMS m/z: 383.60 (M + H)⁺; HPLC: 98.86%. |
| 112 | E-68 | 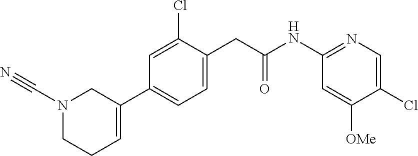 [SMILES: COc1cc(NC(=O)Cc2ccc(C3=CCCN(C#N)C3)cc2Cl)ncc1Cl] | ¹HNMR (DMSO-d₆, 400 MHz): δ 10.90 (s, 1H), 8.22 (d, 1H), 7.9 (s, 1H), 7.37-7.2 (m, 3H), 6.37 (s, 1H), 4.12 (d, 2H), 3.87 (s, 3H), 3.80 (s, 2H), 3.34-3.3 (m, 2H), 2.37-2.35 (m, 2H). LCMS m/z: 417.00 (M + H)⁺; HPLC: 99.21%. |
| 113 | E-69 | 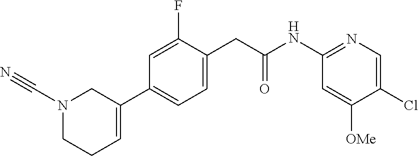 [SMILES: COc1cc(NC(=O)Cc2ccc(C3=CCCN(C#N)C3)cc2F)ncc1Cl] | ¹HNMR (DMSO-d₆, 400 MHz): δ 10.90 (s, 1H), 8.22 (d, 1H), 7.9 (s, 1H), 7.37-7.2 (m, 3H), 6.37 (s, 1H), 4.12 (d, 2H), 3.87 (s, 3H), 3.80 (s, 2H), 3.34-3.3 (m, 2H), 2.37-2.35 (m, 2H). LCMS m/z: 401.00 (M + H)⁺; HPLC: 99.51%. |
| 114 | E-70 | 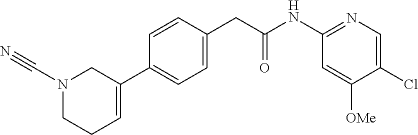 [SMILES: COc1cc(NC(=O)Cc2ccc(C3=CCCN(C#N)C3)cc2)ncc1Cl] | ¹HNMR (DMSO-d₆, 400 MHz): δ 10.97 (s, 1H), 8.21 (s, 1H), 7.91 (s, 1H), 7.29-7.31 (m, 4H), 6.38 (m, 1H), 4.31-4.33 (m, 2H), 3.88 (s, 3H), 3.58 (s, 2H), 3.32-3.34 (m, 2H), 2.45-2.47 (m, 2H), LCMS m/z: 383.20 (M + H)⁺; HPLC: 98.83%, rt: 7.42 min. |
| 115 | E-71 | 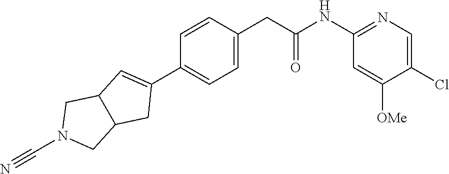 [SMILES: COc1cc(NC(=O)Cc2ccc(C3=CC4CN(C#N)CC4C3)cc2)ncc1Cl] | ¹HNMR (DMSO-d₆, 400 MHz): δ 10.86 (s, 1H), 8.21 (s, 1H), 7.91 (s, 1H), 7.42-7.40 (m, 2H), 7.31-7.29 (m, 2H), 6.09 (m, 1H), 3.88 (s, 3H), 3.71 (s, 2H), 3.55-3.53 (m, 2H), 3.16-3.14 (m, 1H), 3.02-2.91 (m, 2H), 2.66 (m, 1H), LCMS m/z: 409.20 (M + H)⁺; HPLC: 98.21%, rt: 6.95 min |
| 116 | E-72 | 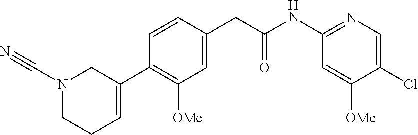 [SMILES: COc1cc(NC(=O)Cc2ccc(C3=CCCN(C#N)C3)c(OC)c2)ncc1Cl] | ¹HNMR (DMSO-d₆, 400 MHz): δ 10.81 (s, 1H), 8.22 (d, 1H), 7.92 (d, 1H), 7.1 (d, 1H), 6.89-6.81 (m, 2H), 5.64 (s, 1H), 3.88 (s, 3H), 3.82 (s, 2H), 3.7 (s, 2H), 3.73 (s, 3H), 3.26 (t, 2H), 2.2 (s, 2H). LCMS m/z: 413.00 (M + H)⁺; HPLC: 99.08%. |
| 117 | E-73 | 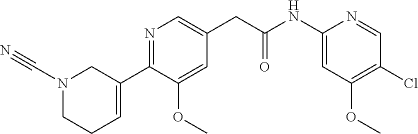 [SMILES: COc1cc(NC(=O)Cc2cnc(C3=CCCN(C#N)C3)c(OC)c2)ncc1Cl] | ¹HNMR (DMSO-d₆, 400 MHz): δ; 10.93 (s, 1H), 8.23 (s, 1H), 8.07 (s, 1H), 7.91 (s, 1H), 7.46(s, 1H), 6.83-6.82 (m, 1H), 4.15-4.14(d, 2H), 3.88 (s, 3H), 3.82 (s, 3H), 3.78 (s, 2H), 3.35-3.26 (m, 2H), 2.41-2.40 (m, 2H); LCMS m/z: (M + H)⁺ 414.10; HPLC: 97.52%, rt: 4.73 min |
| 118 | H-17 | 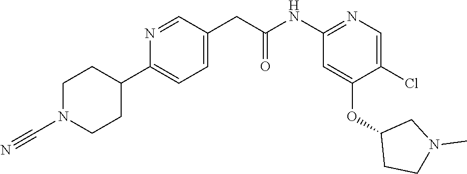 [SMILES: CN1CC[C@H](Oc2cc(NC(=O)Cc3ccc(C4CCN(C#N)CC4)nc3)ncc2Cl)C1] | ¹HNMR (DMSO-d₆, 400 MHz): δ 10.92 (s, 1H), 8.43 (s, 1H), 8.22 (s, 1H), 7.84 (s, 1H), 7.68-7.65 (m, 1H), 7.27-7.25 (m, 1H), 4.91 (s, 1H), 3.74 (s, 2H), 3.47-3.44 (m, 2H), 3.19-3.13 (m, 2H), 2.82-2.67 (m, 4H), 2.32-2.24 (m, 5H), 1.82- 1.75 (m, 5H); LCMS m/z: 455.20 (M + H)⁺; HPLC: 96.48%. |
| 119 | H-18 | 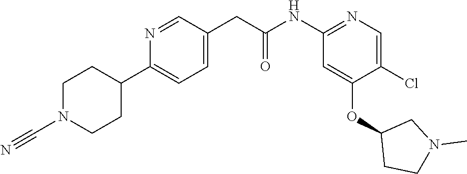 [SMILES: CN1CC[C@@H](Oc2cc(NC(=O)Cc3ccc(C4CCN(C#N)CC4)nc3)ncc2Cl)C1] | ¹HNMR (DMSO-d₆, 400 MHz): δ; 10.97 (s, 1H), 8.44 (s, 1H), 8.26 (s, 1H), 7.86 (s, 1H), 7.67-7.65 (d, 1H), 7.29-7.27 (d, 1H), 5.06 (s, 1H), 3.75 (s, 3H), 3.47-3.44 (m, 2H), 3.20-3.09 (m, 3H), 2.83-2.74 (m, 2H), 2.41-2.32 (m, 2H), 1.97- 1.73 (m, 4 H), 1.26-1.23 (m, 4H). LCMS m/z: (M + H)⁺ 455.20; HPLC: 96.56%, rt: 6.75 min |
| 120 | E-74 | 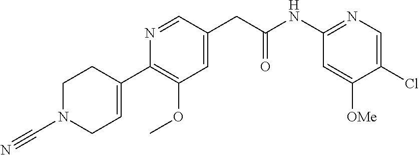 [SMILES: COc1cc(NC(=O)Cc2cnc(C3=CCN(C#N)CC3)c(OC)c2)ncc1Cl] | ¹HNMR (DMSO-d₆, 400 MHz): δ 10.92 (s, 1H), 8.21 (s, 1H), 8.06 (s, 1H), 7.89 (s, 1H), 7.42 (s, 1H), 6.50 (s, 1H), 4.94(s, 2H), 3.86 (s, 3H), 3.80 (s, 3H), 3.76 (s, 2H), 3.39- 3.37 (m, 2H), 2.62-2.61 (m, 2H); LCMS: 414.60; HPLC: 95.11%, rt: 5.42 min |
| 121 | H-20 | 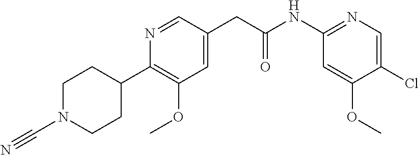 [SMILES: COc1cc(NC(=O)Cc2cnc(C3CCN(C#N)CC3)c(OC)c2)ncc1Cl] | ¹HNMR (DMSO-d₆, 400 MHz): δ 10.92 (s, 1H), 8.29 (s, 1H), 8.04 (s, 1H), 7.91 (s, 1H), 7.34 (s, 1H), 3.88 (s, 3H), 3.82-3.78 (m, 3H), 3.75(s, 2H), 3.45-3.42 (m, 2H), 3.20-3.14 (m, 3H), 1.83-1.79 (m, 2H), 1.70-1.67 (m, 2H); LCMS: 416.20; HPLC: 93.61%, rt: 6.81 min |
| 122 | E-75 | 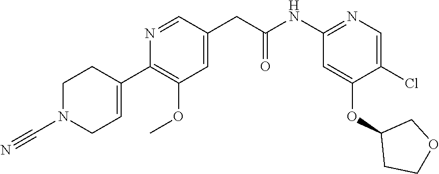 [SMILES: COc1cc(CC(=O)Nc2cc(O[C@@H]3CCOC3)c(Cl)cn2)cnc1C1=CCN(C#N)CC1] | ¹HNMR (DMSO-d₆, 400 MHz): δ 10.96 (s, 1H), 8.25 (s, 1H), 8.08 (s, 1H), 7.88 (s, 1H), 7.44 (s, 1H), 6.52 (s, 1H), 5.12 (s, 1H), 3.96 (s, 2H), 3.85-3.79 (m, 2H), 3.75-3.74 (m, 3H), 3.73 (s, 2H), 3.41-3.34 (m, 2H), 2.64-2.57 (m, 2H), 2.29- 2.23 (m, 1H), 2.00-1.96 (m, 1H), 1.26-1.23 (m, 2H). LCMS: 470.30 (M + H)⁺; HPLC: 99.61%, rt: 5.7 min |
| 123 | H-21 | 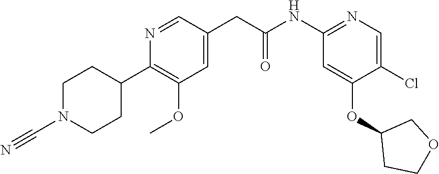 [SMILES: COc1cc(CC(=O)Nc2cc(O[C@@H]3CCOC3)c(Cl)cn2)cnc1C1CCN(C#N)CC1] | ¹HNMR (DMSO-d₆, 400 MHz): δ 10.93 (s, 1H), 8.24 (s, 1H), 8.04 (s, 1H), 7.88 (s, 1H), 7.34 (s, 1H), 5.12 (s, 1H), 3.87-3.85 (m, 4H), 3.75 (s, 3H), 3.42-3.41 (m, 2H), 3.17-3.16 (m, 3H), 2.33-2.32 (m, 2H), 2.10-2.00 (m, 2H), 1.80-1.70 (m, 4H); LCMS: 472.30, HPLC: 98.18%, rt: 6.8 min. |
| 124 | H-19 | 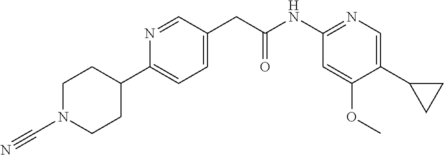 [SMILES: COc1cc(NC(=O)Cc2ccc(C3CCN(C#N)CC3)nc2)ncc1C1CC1] | ¹HNMR (DMSO-d₆, 400 MHz): δ 10.68 (s, 1H), 8.43 (s, 1H), 7.75 (s, 1H), 7.72 (s, 1H), 7.68-7.66 (m, 1H), 7.26-7.24 (m, 1H), 3.81 (s, 3H), 3.71 (s, 2H), 3.47-3.44 (m, 2H), 3.16-3.13 (m, 2H), 2.84-2.81 (m, 1H), 1.85-1.84 (m, 2H), 1.82- 1.79 (m, 3H), 0.84-0.82 (m, 2H), 0.65-0.64 (m, 2H), LCMS m/z: 392.3 (M + H)⁺; HPLC: 97.01%, rt: |
| | | | 5.37 min. |
| 125 | H-22 | 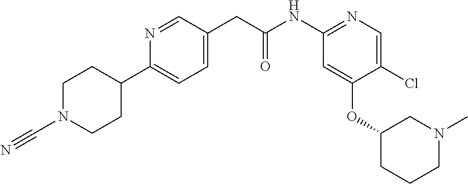 [SMILES: CN1CCC[C@H](Oc2cc(NC(=O)Cc3ccc(C4CCN(C#N)CC4)nc3)ncc2Cl)C1] | ¹HNMR (DMSO-d₆, 400 MHz): δ 10.90 (s, 1H), 8.43 (s, 1H), 8.23 (s, 1H), 7.91 (s, 1H), 7.68-7.66 (m, 1H), 7.27-7.24 (m, 1H), 4.48 (m, 1H), 3.74 (m, 2H), 3.47-3.44 (m, 2H), 3.1-3.16 (m, 2H), 2.82-2.80 (m, 2H), 2.15 (s, 3H), 2.08-2.06 (m, 3H), 1.85-1.82 (m, 1H), 7.48- 7.46 (m, 1H), 7.42 (s, 4H), 1.52- 1.50 (m, 2H), LCMS m/z: 469.3 (M + H)⁺; HPLC: 99.72%, rt: 4.15 min. |
| 126 | H-23 | 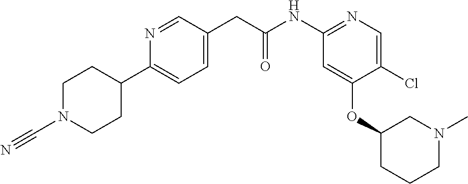 [SMILES: CN1CCC[C@@H](Oc2cc(NC(=O)Cc3ccc(C4CCN(C#N)CC4)nc3)ncc2Cl)C1] | ¹HNMR (DMSO-d₆, 400 MHz): δ 10.90 (s, 1H), 8.43 (s, 1H), 8.23 (s, 1H), 7.91 (s, 1H), 7.68-7.66 (m, 1H), 7.27-7.24 (m, 1H), 4.48 (m, 1H), 3.74 (m, 2H), 3.47-3.44 (m, 2H), 3.1-3.16 (m, 2H), 2.82-2.80 (m, 2H), 2.15 (s, 3H), 2.08-2.06 (m, 3H), 1.85-1.82 (m, 1H), 7.48- 7.46 (m, 1H), 7.42 (s, 4H), 1.52- 1.50 (m, 2H), LCMS m/z: 469.30 (M + H)⁺; HPLC: 98.44%, rt: 5.01 min. |
| 127 | E-76 | 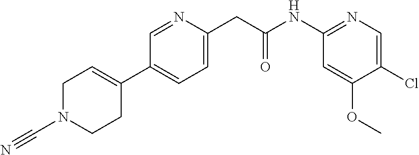 [SMILES: COc1cc(NC(=O)Cc2ccc(C3=CCN(C#N)CC3)cn2)ncc1Cl] | ¹HNMR (DMSO-d₆, 400 MHz): δ; 10.90 (s, 1H), 8.59 (s, 1H), 8.23 (s, 1H), 7.93 (s, 1H), 7.84-7.80 (d, 1H), 7.39-7.37 (d, 1H), 6.25-6.22 (m, 1H), 3.94 (s, 4H), 3.88 (s, 3H), 3.46-3.44 (m, 2H), 2.61-2.58 (m, 2H). LCMS m/z: 384.3 (M + H)⁺; HPLC: 98.25%, rt: 5.72 min |
| 128 | H-26 | 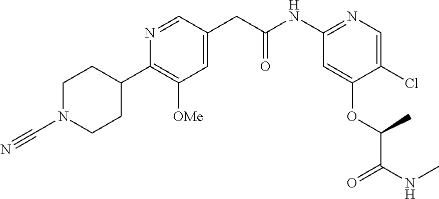 [SMILES: CNC(=O)[C@H](C)Oc1cc(NC(=O)Cc2cnc(C3CCN(C#N)CC3)c(OC)c2)ncc1Cl] | ¹HNMR (DMSO-d₆, 400 MHz): δ 10.87 (s, 1H), 8.24 (s, 1H), 8.19 (s, 1H), 8.03 (s, 1H), 7.77 (s, 1H), 7.34 (s, 1H), 4.77-4.75(m, 1H), 3.80 (s, 2H), 3.72 (s, 3H), 3.45- 3.42 (d, 2H), 3.19-3.14 (m, 3H), 2.58-2.57 (m, 3H), 1.79-1.70 (m, 4H), 1.46-1.45 (m, 3H); LCMS = 487.60 (M + 1)⁺; HPLC: 95.46%, rt: 5.72 min |
| 129 | H-24 | 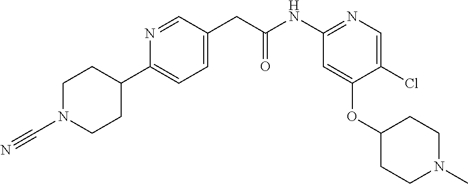 [SMILES: CN1CCC(Oc2cc(NC(=O)Cc3ccc(C4CCN(C#N)CC4)nc3)ncc2Cl)CC1] | ¹HNMR (DMSO-d₆, 400 MHz): δ; 10.90 (s, 1H), 8.42 (s, 1H), 8.23 (s, 1H), 7.90 (s, 1H), 7.67-7.65 (d, 1H), 7.26-7.24 (d, 1H), 4.52-4.50 (m, 1H), 3.74 (s, 2H), 3.50-3.44 (d, 2H), 3.32-3.13 (m, 2H), 2.84-2.78 (m, 2H), 2.22-2.21 (m, 5H), 2.20- 2.94 (m, 3H), 1.94-1.69 (m, 6H). LCMS m/z: 469.3 (M + H)⁺; HPLC: 99.41%, rt: 4.51 min |
| 130 | E-77 | 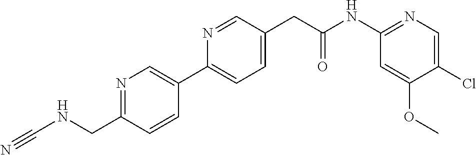 [SMILES: COc1cc(NC(=O)Cc2ccc(-c3ccc(CNC#N)nc3)nc2)ncc1Cl] | ¹HNMR (DMSO-d₆, 400 MHz): δ 11.00 (s, 1H), 8.84 (d, 1H), 8.62 (d, 1H), 8.25 (s, 1H), 8.01 (s, 1H), 7.92 (d, 1H), 7.90 (d, 1H), 7.48- 7.47 (m, 2H), 7.32 (s, 1H), 3.89 (s, 3H), 3.63-3.59 (m, 2H), 3.16-3.12 (m, 2H). LCMS: m/z = 409.1 (M + H)⁺; HPLC: 97.19%, rt: 5.17 min. |
| 131 | E-78 | 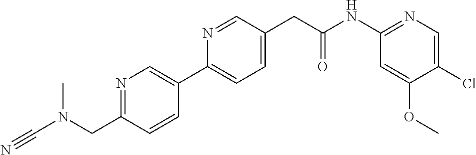 [SMILES: COc1cc(NC(=O)Cc2ccc(-c3ccc(CN(C)C#N)nc3)nc2)ncc1Cl] | ¹HNMR (DMSO-d₆, 400 MHz): δ 11.01 (s, 1H), 9.01 (s, 1H), 8.62- 8.60 (m, 3H), 8.24 (s, 1H), 7.96 (s, 1H), 7.92-7.90 (m, 2H), 7.53-7.52 (s, 2H). 3.88 (s, 3H), 3.87 (s, 2H), 3.74 (s, 3H); LCMS m/z: 423.20 (M + H)⁺; HPLC: 97.01%, rt: 5.46 min. |
| 132 | E-79 | 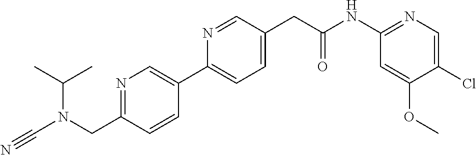 [SMILES: COc1cc(NC(=O)Cc2ccc(-c3ccc(CN(C#N)C(C)C)nc3)nc2)ncc1Cl] | ¹HNMR (DMSO-d₆, 400 MHz): δ 11.01 (s, 1H), 8.98 (s, 1H), 8.62 (s, 1H), 8.57 (m, 2H), 8.24 (s, 1H), 7.95-7.93 (m, 1H), 7.92-7.91 (m, 1H), 7.67 (s, 1H), 4.71 (s, 2H), 3.88 (s, 3H), 3.87 (s, 2H), 3.33 (m, 1H), 1.47-1.45 (s, 6H); LCMS m/z: 451.30 (M + H)⁺; HPLC: 98.85%, rt: 4.93 min. |

### Example-2: Synthesis of tert-butyl 6-(5-(2-((5-chloro-4-methoxypyridin-2-yl)amino)-2-oxoethyl)pyridin-2-yl)-3-azabicyclo[4.1.0]heptane-3-carboxylate (Compound 133)

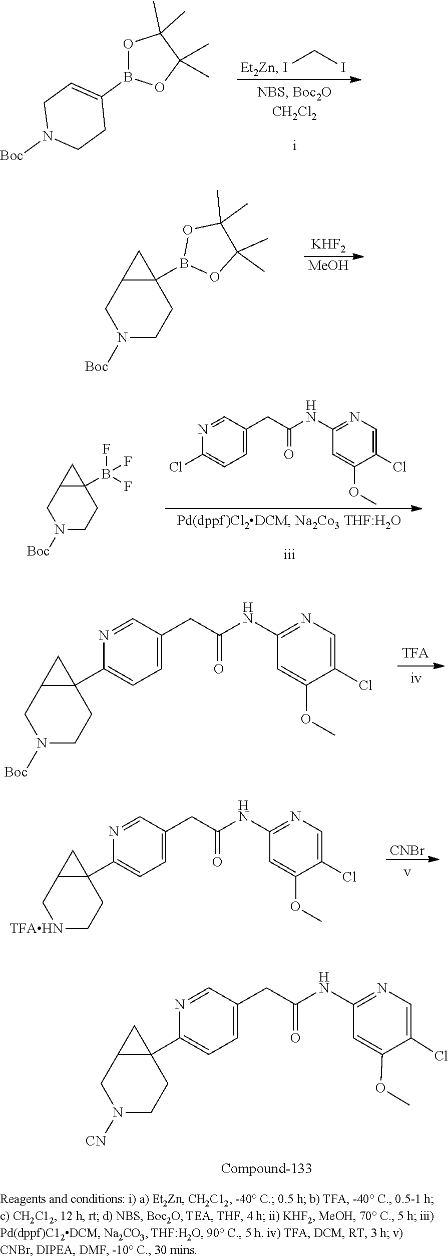

### Step-i: Synthesis of tert-butyl 6-(4,4,5,5-tetramethyl-1,3,2-dioxaborolan-2-yl)-3-azabicyclo[4.1.0]heptane-3-carboxylate

To a stirred solution of diethylzinc (9.13, 73.99 mmol) in dry DCM (100 mL) was added diiodomethane (39.63 g, 147.98 mmol) dropwise at −40° C., and the resulting mixture was stirred for 30 min. TFA (1.2 g, 110.4 mmol) in DCM (25 mL) was slowly added by addition funnel over 30 min. The reaction was then allowed to stir at −40° C. for 1 h before tert-butyl 4-(4,4,5,5-tetramethyl-1,3,2-dioxaborolan-2-yl)-3,6-dihydropyridine-1(2H)-carboxylate (2.86 g, 9.24 mmol) in DCM (25 mL) was added dropwise over 15 min. The reaction mixture was allowed to stir at rt overnight. The resulting reaction mass was concentrated to half of its volume, diluted with THF (100 mL), and treated with NBS (1.91 g, 10.72 mmol), TEA (9.34 g, 92.49 mmol), and Boc anhydride (10.08 g, 46.24 mmol). After addition of all reagents, the mixture was stirred for 4 h. The reaction mixture was diluted with EtOAc, washed with brine solution, dried over sodium sulphate, and concentrated under vacuum. The crude product was purified by flash chromatography using 20% ethyl acetate in hexane as an eluent (0.35 g, 11.71%). LCMS m/z: 324.00 (M+H)⁺

### Step-ii: Synthesis of tert-butyl 6-(trifluoro-λ⁴-boraneyl)-3-azabicyclo[4.1.0]heptane-3-carboxylate

To a stirred solution of tert-butyl 6-(4,4,5,5-tetramethyl-1,3,2-dioxaborolan-2-yl)-3-azabicyclo[4.1.0]heptane-3-carboxylate (2 g, 6.18 mmol) in MeOH (30 mL) was added KHF₂ (3.38 g, 43.30 mmol) at RT, and the reaction mixture was stirred at 70° C. for 5 h. The vessel was cooled to RT and agitated overnight. Then, the mixture was concentrated, and the crude residue was triturated with diethyl ether to obtain a semisolid compound which was taken up in ACN (10 mL) and heated at 50° C. for 30 min. The mixture was filtered through a celite pad. The filtrate was concentrated and taken to the next step without further purification (1.8 g, crude). LCMS m/z: 264.80 (M+H)⁺

### Step-iii: Synthesis of tert-butyl 6-(5-(2-((5-chloro-4-methoxypyridin-2-yl)amino)-2-oxoethyl)pyridin-2-yl)-3-azabicyclo[4.1.0]heptane-3-carboxylate

To a degassed solution of N-(5-chloro-4-methoxypyridin-2-yl)-2-(6-chloropyridin-3-yl)acetamide (D-2) (0.5 g, 1.60 mmol) in THF (10 mL), were added tert-butyl 6-(trifluoro-λ⁴-boraneyl)-3-azabicyclo[4.1.0]heptane-3-carboxylate (0.42 g, 1.60 mmol) and Na₂CO₃ (0.34 g, 3.20 mmol) in water (2 mL), and the resultant solution was further degassed for 10 min. Pd(dppf)Cl₂·DCM (0.13 g, 0.16 mmol) was added, and the mixture was heated at 90° C. for 5 h. The reaction mass was filtered through a celite bed, washed with ethyl acetate (50 mL), and the organic layer was separated, washed with brine, and dried over anhydrous sodium sulphate to yield the crude title compound. The crude product was further purified using flash chromatography using an ethyl acetate:hexane (1:1) mixture as eluent to afford title compound (0.2 g, 26.40%). LCMS m/z: 473.70 (M+H)⁺

### Step-iv and step-v: Synthesis of N-(5-chloro-4-methoxypyridin-2-yl)-2-(6-(3-isocyano-3-azabicyclo[4.1.0]heptan-6-yl)pyridin-3-yl)acetamide

N-(5-chloro-4-methoxypyridin-2-yl)-2-(6-(3-isocyano-3-azabicyclo[4.1.0]heptan-6-yl)pyridin-3-yl)acetamide was prepared by reacting the Boc-protected amine intermediate with TFA followed by cyanogen bromide using a procedure similar to the one described in Example 1 to give pure titled product; ¹HNMR (DMSO-d₆, 400 MHz): δ 10.91 (s, 1H), 8.39 (d, 1H), 8.22 (s, 1H), 7.90 (s, 1H), 7.72-7.70 (m, 1H), 7.31 (d, 1H), 3.87 (s, 3H), 3.73 (s, 2H), 3.18-3.17 (m, 2H), 2.61-2.50 (m, 2H), 2.10-2.08 (m, 2H), 1.29-1.28 (m, 1H), 1.10-1.00 (m, 1H), 0.87-0.85 (m, 1H); LCMS m/z: 398.10 (M+H)⁺; HPLC: 94.01%, rt: 3.49 min.

### Example-3: Synthesis of N-(5-chloro-4-methoxypyridin-2-yl)-2-(6-(4-cyanopiperazin-1-yl)pyridin-3-yl)acetamide (Compound 134)

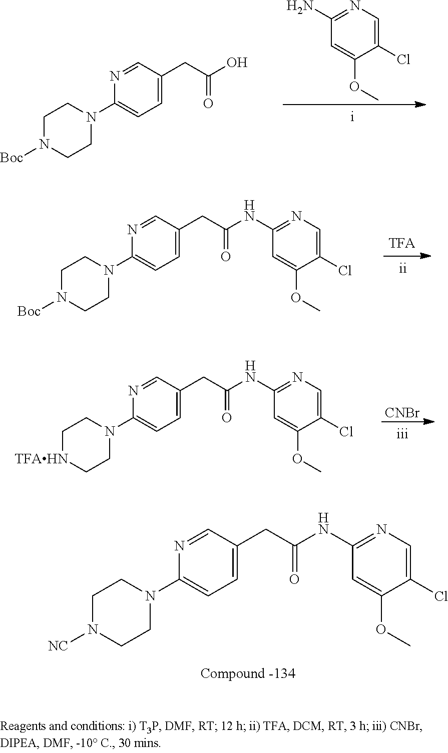

### Step-i: Synthesis of tert-butyl 4-(5-(2-((5-chloro-4-methoxypyridin-2-yl)amino)-2-oxoethyl)pyridin-2-yl)piperazine-1-carboxylate

To a solution of 2-(6-(4-(tert-butoxycarbonyl)piperazin-1-yl)pyridin-3-yl)acetic acid (1 g, 3.11 mmol) (synthesis carried out as described in reference WO2018/138356, 2018, A1) in DMF (10 mL) were added pyridine (0.48 g, 6.23 mmol) and a solution of T₃P (50% in EtOAc) (3.82 g, 7.77 mmol) at 0° C. over a period of 10 min. The reaction mass was stirred for 10 min at the same temperature, followed by lot wise addition of 5-chloro-4-methoxypyridin-2-amine (0.59 g, 3.73 mmol) over a period of 5 min. After the addition, the reaction mixture was warmed to room temperature and stirred for 12 h. The reaction mass was quenched with ice water, and the organic layer was isolated. The aqueous layer was further extracted with ethyl acetate, and the combined organic layers were dried over anhydrous sodium sulphate and concentrated under reduced pressure to afford crude product. The crude material was further purified by Combiflash using an ethyl acetate:hexane (20-30%) mixture as mobile phase to afford pure titled compound (0.8 g, 56%); LCMS: m/z=462.10 (M+H)

### Step-ii and iii: Synthesis of N-(5-chloro-4-methoxypyridin-2-yl)-2-(6-(4-cyanopiperazin-1-yl)pyridin-3-yl)acetamide

N-(5-chloro-4-methoxypyridin-2-yl)-2-(6-(4-cyanopiperazin-1-yl)pyridin-3-yl)acetamide was prepared by reacting the Boc-protected amine intermediate with TFA followed by cyanogen bromide using a procedure similar to the one described in Example-1 to give pure titled product; ¹HNMR (DMSO-d₆, 400 MHz): δ 10.93 (s, 1H), 8.22 (s, 1H), 8.14 (s, 1H), 7.91 (s, 1H), 7.55-7.50 (m, 1H), 7.84-7.82 (m, 1H), 3.88 (s, 3H), 3.60 (s, 2H), 3.56-3.53 (m, 4H), 3.28-3.27 (m, 4H); LCMS m/z: 387.10 (M+H)⁺; HPLC: 98.25%, rt: 5.79 min.

### Example-4: Synthesis of tert-butyl 5-(2-((5-chloro-4-(dimethylphosphoryl)pyridin-2-yl)amino)-2-oxoethyl)-5′,6′-dihydro-[2,3′-bipyridine]-1′(2′H)-carboxylate (Compound 135)

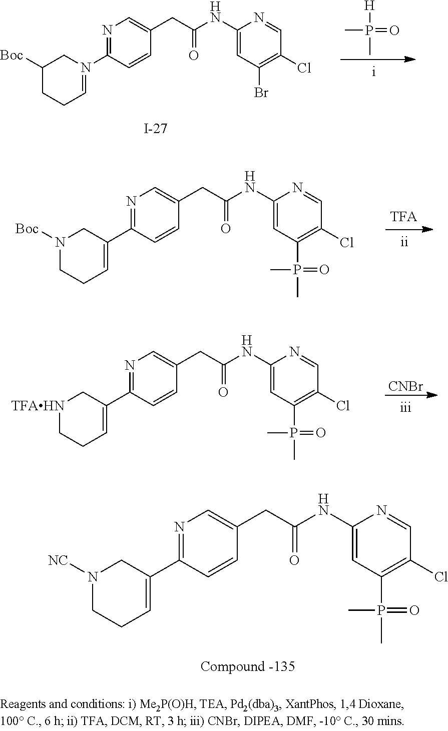

### Step-i: Synthesis of tert-butyl 5-(2-((5-chloro-4-(dimethylphosphoryl) pyridin-2-yl)amino)-2-oxoethyl)-5′,6′-dihydro-[2,3′-bipyridine]-1′(2′H)-carboxylate

To a degassed solution of tert-butyl 5-(2-((4-bromo-5-chloropyridin-2-yl)amino)-2-oxoethyl)-5′,6′-dihydro-[2,3′-bipyridine]-1′(2′H)-carboxylate (I-27) (0.2 g, 0.394 mmol) in 1,4-dioxane (8 mL) were added dimethylphosphine oxide (0.0 39 g, 0.51 mmol) and TEA (0.080 g, 0.78 mmol), and the resultant solution was further degassed for 10 min. Pd₂(dba)₃ (0.036 g. 0.030) and XantPhos (0.046 g, 0.070 mmol) were added, and the mixture was heated at 100° C. for 6 h. The reaction mass was filtered through a celite bed, washed with ethyl acetate (20 mL). The organic layer was separated, washed with brine, and dried over anhydrous sodium sulphate to yield the crude title compound. The crude material was further purified using flash chromatography using a 40% ethyl acetate in hexane mixture as eluent to afford title compound (0.15 g, 75.40%). LCMS m/z: 505.10 (M+H)⁺.

### Step-ii and iii: Synthesis of N-(5-chloro-4-(dimethylphosphoryl) pyridin-2-yl)-2-(1′-cyano-1′,2′,5′,6′-tetrahydro-[2,3′-bipyridin]-5-yl)acetamide

N-(5-chloro-4-(dimethylphosphoryl)pyridin-2-yl)-2-(1′-cyano-1′,2′,5′,6′-tetrahydro-[2,3′-bipyridin]-5-yl)acetamide was prepared by reacting the Boc-protected amine intermediate with TFA followed by cyanogen bromide using a procedure similar to the one described in Example 1 to give pure titled product; ¹HNMR (DMSO-d₆, 400 MHz): δ 11.21 (s, 1H), 8.61-8.63 (m, 1H), 8.55-8.43 (m, 2H), 7.79-7.75 (d, 1H), 7.65-7.61 (d, 1H), 6.89 (s, 1H), 4.23 (s, 2H), 3.82 (s, 2H), 3.49-3.41 (m, 2H), 2.42 (s, 2H), 1.83-1.77 (m, 6H); LCMS=429.60; HPLC: 98.92%, rt: 5.79 min.

### Example-5: Synthesis of N-(5-chloro-4-(piperidin-4-yloxy)pyridin-2-yl)-2-(1′-cyano-1′,2′,3′,6′-tetrahydro-[2,4′-bipyridin]-5-yl)acetamide (Compound 136)

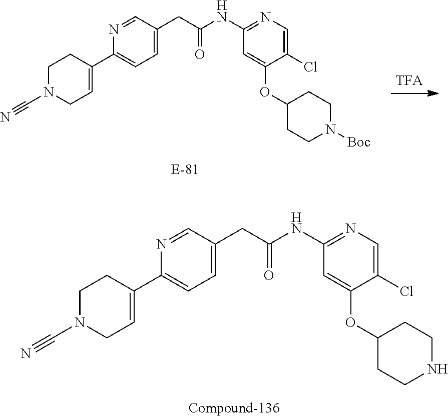 [SMILES: CC(C)(C)OC(=O)N1CCC(Oc2cc(NC(=O)Cc3ccc(C4=CCN(C#N)CC4)nc3)ncc2Cl)CC1 ; "E-81" ; N#CN1CC=C(c2ccc(CC(=O)Nc3cc(OC4CCNCC4)c(Cl)cn3)cn2)CC1 ; "Compound-136"]

To a stirred solution of tert-butyl 4-((5-chloro-2-(2-(1′-cyano-1′,2′,3′,6′-tetrahydro-[2,4′-bipyridin]-5-yl)acetamido)pyridin-4-yl)oxy)piperidine-1-carboxylate (E-81) (0.5 g, 0.91 mmol) in DCM (10 mL) was added TFA (0.16 g, 1.40 mmol) at 0° C. Then the reaction was stirred for 3 h at RT. Solvent was evaporated under vacuum, and the residue was diluted with dichloromethane. The resulting mixture was washed with aqueous sodium bicarbonate and brine and dried over anhydrous sodium sulfate before being concentrated under vacuum to give the desired product (0.3 g, 75%); ¹HNMR (DMSO-d₆, 400 MHz): δ 10.97 (s, 1H), 8.45 (m, 2H), 8.28 (s, 1H), 7.92 (s, 1H), 7.70-7.68 (m, 1H), 7.54-7.52 (m, 1H), 7.10 (s, 2H) 6.65 (s, 1H), 6.01 (m, 2H), 4.83 (s, 1H), 4.01 (s, 1H), 3.77 (m, 1H), 3.51-3.49 (m, 2H), 3.16 (m, 3H), 2.11-2.09 (m, 2H), 1.90-1.88 (m, 2H), 1.27-1.25 (1H), LCMS m/z: 471.10 (M+H)⁺; HPLC: 98.37%, rt: 4.35 min.

The compounds listed in below Table J were using a procedure similar to that described in Example 5 by reacting an appropriate Boc-protected amine intermediate as with TFA to give the deprotected product.

TABLE J

| | Inter- | | |
|---|---|---|---|
| Compd. | mediate | | |
| No. | used | Compound structure | Characterization Data |
| 137 | E-80 | 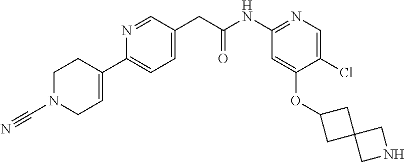 [SMILES: N#CN1CC=C(c2ccc(CC(=O)Nc3cc(OC4CC5(CNC5)C4)c(Cl)cn3)cn2)CC1] | ¹HNMR (DMSO-d₆, 400 MHz): δ 10.97 (s, 1H), 8.51-8.47 (m, 2H), 8.24 (s, 1H), 7.76- 7.73 (m, 1H), 7.58-7.54 (m, 1H), 6.68 (s, 2H), 6.03 (s, 1H), 4.77-4.71 (m, 2H), 4.01- 4.00 (m, 4H), 3.95-3.92 (m, 2H), 3.50-3.49 (m, 2H), 2.77-2.66 (m, 4H), 2.40-2.32 (m, 2H); LCMS m/z: 466 (M + H)⁺; HPLC: 90.47%, rt: 4.65 min. |
| 138 | E-82 | 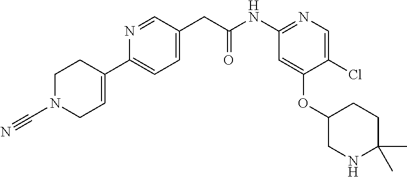 [SMILES: CC1(C)CCC(Oc2cc(NC(=O)Cc3ccc(C4=CCN(C#N)CC4)nc3)ncc2Cl)CN1] | ¹HNMR (DMSO-d₆, 400 MHz): δ 10.97 (s, 1H), 8.46 (d, 1H), 8.27 (d, 1H), 7.92 (s, 1H), 7.7-7.68 (m, 1H), 7.54-7.52, 6.65 (s, 1H), 6.05 (s, 2H), 4.65-4.6 (m, 1H), 4.11- 4.09 (m, 2H), 4.01 (d, 2H), 3.77 (s, 2H), 3.49 (t, 2H), 3.16 (d, 3H), 1.98-1.96 (m, 2H), 1.85-1.5 (m, 4H), 1.21 (d, 3H). LCMS m/z: 481.30 (M + H)⁺; HPLC: 96.97%. |
| 139 | E-1 | 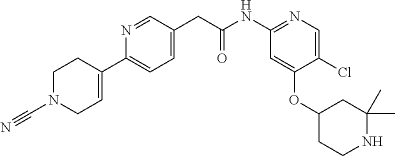 [SMILES: CC1(C)CC(Oc2cc(NC(=O)Cc3ccc(C4=CCN(C#N)CC4)nc3)ncc2Cl)CCN1] | ¹HNMR (DMSO-d₆, 300 MHz): δ 10.97 (s, 1H), 8.46 (s, 1H), 8.27 (s, 1H), 7.94 (s, 1H), 7.71-7.69 (d, 1H), 7.55-7.52 (d, 1H), 6.66 (m, 1H), 6.02 (s, 2H), 4.86 (m, 1H), 4.01 (d, 2H), 3.77 (m, 2H), 3.51-3.47 (m, 2H), 3.32-3.23 (m, 2H), 2.15-1.98 (m, 2H), 1.82-1.62 (m, 2H), 1.32-1.29, (m, 6H), 1.23 (m, 1H). LCMS: m/z = 481.30 (M + H)⁺; HPLC: 99.06%, rt: 4.22 min. |

### Example-6: Synthesis of N-(5-chloro-4-methoxypyridin-2-yl)-2-(6′-cyano-[2,3′-bipyridin]-5-yl)acetamide (Compound 140)

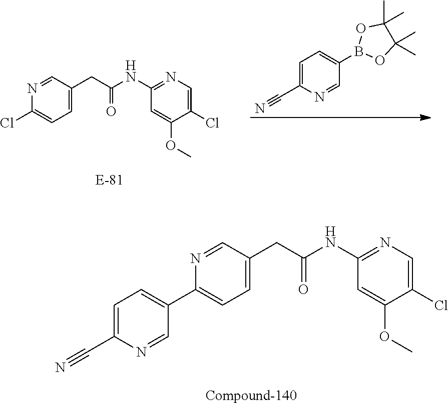 [SMILES: COc1cc(NC(=O)Cc2ccc(Cl)nc2)ncc1Cl ; CC1(C)OB(c2ccc(C#N)nc2)OC1(C)C ; "E-81" ; COc1cc(NC(=O)Cc2ccc(-c3ccc(C#N)nc3)nc2)ncc1Cl ; "Compound-140"]

To a degassed solution of N-(5-chloro-4-methoxypyridin-2-yl)-2-(6-chloropyridin-3-yl)acetamide (0.5 g, 1.770 mmol) in 1,4-dioxane (14 mL) were added Na₂CO₃ (0.75 g, 3.45 mmol) dissolved in water (2.0 M solution) and 5-(4,4,5,5-tetramethyl-1,3,2-dioxaborolan-2-yl)picolinonitrile (0.55 g, 1.77 mmol). The resultant solution was degassed for 10 min. before Pd(dppf)Cl₂·DCM (0.13 g, 10 mmol) was added, and the mixture was heated at 90° C. for 5 h. The reaction mass was filtered through a celite bed, and the organic layer was separated, washed with brine, and dried over anhydrous sodium sulphate and concentrated to yield the crude title compound. The crude material was further purified using flash chromatography using an ethyl acetate:hexane (1:1) mixture as eluent to afford title compound (0.5 g, 65.86%). ¹HNMR (DMSO-d₆, 400 MHz): δ 11.00 (s, 11H), 9.43 (d, 1 MHz), 8.70-8.66 (m, 2H), 8.24 (s, 11H), 8.18-8.16 (d, 2H), 7.95-7.92 (m, 2H), 3.89 (s, 2H), 3.88 (s, 3H). LCMS m/z: 380.0 (M+H)³⁰; HPLC: 95.64.3.

The compounds listed in below Table K were prepared by reacting an amide intermediate with respective boronate esters and suitable catalyst with a procedure similar to the one described in the synthesis of Example 6 with appropriate variations. The characterization data of the intermediates are summarized herein the below table

TABLE K

| Compd. | Amide | Boronate | Catalyst | | Characterization |
|---|---|---|---|---|---|
| No. | used | ester used | used | Compound structure | data |
| 141 | D-2 | 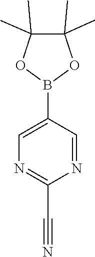 | Pd(dppf) Cl₂•DCM | 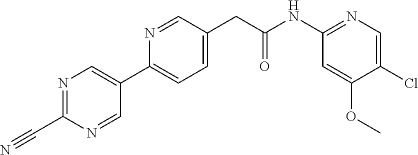 [SMILES: COc1cc(NC(=O)Cc2ccc(-c3cnc(C#N)nc3)nc2)ncc1Cl] | ¹H NMR (DMSO-d₆, 400 MHz): δ 11.002 (s, 1H), 9.63 (s, 2H), 8.73 (s, 1H), 8.24- 8.22 (m, 2H), 7.99- 7.96 (m, 1H), 7.91 (s, 1H), 3.91 (s, 2H), 3.88 (s, 3H); LCMS m/z: 381.10 (M + H)⁺; HPLC: 96.43%, rt: 6.20 min. |
| 142 | D-2 | 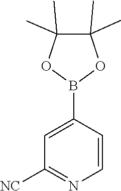 | Pd(dppf) Cl₂•DCM | 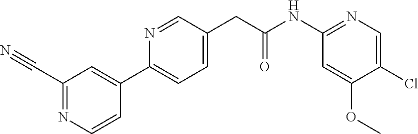 [SMILES: COc1cc(NC(=O)Cc2ccc(-c3ccnc(C#N)c3)nc2)ncc1Cl] | ¹HNMR (DMSO-d₆, 400 MHz): δ 10.97 (s, 1H), 8.83-8.82 (m, 1H), 8.67 (s, 1H), 8.63 (s, 1H), 8.36- 8.35 (m, 1H), 8.19- 8.17 (m, 2H), 7.93- 7.91 (m, 1H), 7.88 (s, 1H), 3.87 (s, 3H), 3.84 (s, 2H), LCMS m/z: 380.10 (M + H)⁺: HPLC: 98.02%, |
| | | | | | rt: 6.58 min. |
| 143 | D-22 | 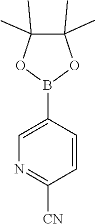 | Pd(PPh₃)₄ | 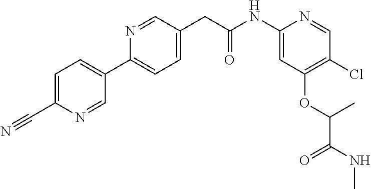 [SMILES: CNC(=O)C(C)Oc1cc(NC(=O)Cc2ccc(-c3ccc(C#N)nc3)nc2)ncc1Cl] | ¹HNMR (DMSO-d₆, 400 MHz): δ 10.96 (s, 1H), 9.42 (s, 1H), 8.69-8.65 (m, 2H), 8.25 (s, 1H), 8.17- 8.13 (m, 3H), 7.93- 7.91 (m, 1H) 7.77 (s, 1H), 4.77-4.75 (m, 1H), 3.87 (s, 2H), 2.58-2.57 (m, 3H), 1.46-1.44 (m, 3H); LCMS m/z: 451.20 (M + H)⁺; HPLC: 98.47%, rt: 6.71 min. |
| 144 | D-22 | 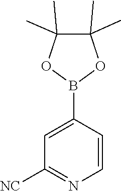 [SMILES: CC1(C)OB(c2ccnc(C#N)c2)OC1(C)C] | Pd(PPh₃)₄ | 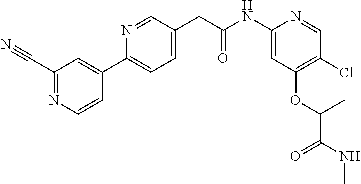 [SMILES: CNC(=O)C(C)Oc1cc(NC(=O)Cc2ccc(-c3ccnc(C#N)c3)nc2)ncc1Cl] | ¹HNMR (DMSO-d₆, 400 MHz): δ 10.96 (s, 1H), 8.87-8.85 (m, 1H), 8.70-8.67 (m, 2H), 8.40-8.39 (m, 1H), 8.25-8.21 (m, 2H), 8.14-8.13 (m, 1H), 7.95-7.93 (m, 1H) 7.77 (s, 1H), 4.77-4.75 (m, 1H), 3.89 (s, 2H), 2.57- 2.56 (m, 3H), 1.46- |
| | | | | | 1.44 (m, 3H); LCMS |
| | | | | | m/z: 451.1 (M + H)⁺; |
| | | | | | HPLC: 96.92%, rt: |
| | | | | | 5.96 min. |
| 145 | D-23 | 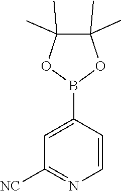 [SMILES: CC1(C)OB(c2ccnc(C#N)c2)OC1(C)C] | Pd(PPh₃)₄ | 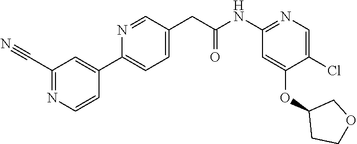 [SMILES: N#Cc1cc(-c2ccc(CC(=O)Nc3cc(O[C@@H]4CCOC4)c(Cl)cn3)cn2)ccn1] | ¹HNMR (DMSO-d₆, 400 MHz): δ 11.02 (s, 1H), 8.87-8.85 (m, 1H), 8.71-8.70 (m, 1H), 8.66 (s, 1H), 8.40 (s, 1H), 8.25 (s, 1H), 7.88 (s, 1H), 7.64-7.62 (m, 1H), 7.61-7.59 (m, 1H), 5.11 (m, 1H), 3.91 (s, 2H), 3.86- 3.85 (m, 2H), 3.82 (s, |
| | | | | | 1H), 3.76 (s, 1H), |
| | | | | | 2.26 (m, 1H), 1.99 |
| | | | | | (m, 1H), LCMS m/z: |
| | | | | | 436.20 (M + H)⁺: |
| | | | | | HPLC: 96.23%, rt: |
| | | | | | 6.53 min. |
| 146 | D-23 | 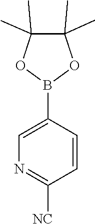 | Pd(dppf) Cl₂•DCM | 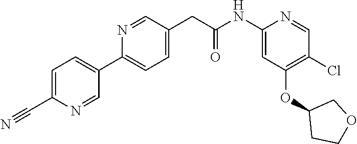 [SMILES: N#Cc1ccc(-c2ccc(CC(=O)Nc3cc(O[C@@H]4CCOC4)c(Cl)cn3)cn2)cn1] | ¹H NMR (DMSO-d₆, 400 MHz): δ 11.01 (s, 1H), 9.43 (s, 1H), 8.71-8.65 (m, 2H), 8.25 (s, 1H), 8.17 (d, 2H), 7.94-7.89 (m, 2H), 5.12 (s, 1H), 3.89-3.8 (m, 4H), 3.75-3.73 (m, 1H), 2.65-2.60 (m, 1H), 2.26-2.24 (m, 1H), 2.01-1.98 (s, 1H). LCMS m/z: 436.1 (M + H)⁺: HPLC: 97.69%, rt: |
| | | | | | 7.20 min. |
| 147 | D-22 | 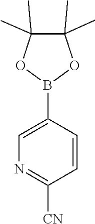 | Pd(PPh₃)₄ | 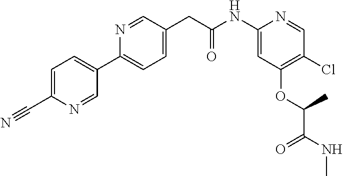 [SMILES: CNC(=O)[C@H](C)Oc1cc(NC(=O)Cc2ccc(-c3ccc(C#N)nc3)nc2)ncc1Cl] | ¹HNMR (DMSO-d₆, 400 MHz): δ 10.95 (s, 1H), 9.42 (s, 1H), 8.68-8.64 (m, 2H), 8.24 (s, 1H), 8.16- 8.12 (m, 3H), 7.92- 7.90 (m, 1H) 7.76 (s, 1H), 4.77-4.75 (m, 1H), 3.87 (s, 2H), 2.58-2.57 (m, 3H), 1.46-1.44 (m, 3H),; LCMS m/z: 451.20 (M + H)⁺; HPLC: 97.77%, rt: 9.39 |
| | | | | | min, CHIRAL |
| | | | | | HPLC: 100%, rt: |
| | | | | | 13.41 min. |
| 148 | D-22 | 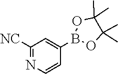 [SMILES: CC1(C)OB(c2ccnc(C#N)c2)OC1(C)C] | Pd(PPh₃)₄ | 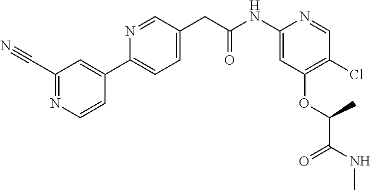 [SMILES: CNC(=O)[C@H](C)Oc1cc(NC(=O)Cc2ccc(-c3ccnc(C#N)c3)nc2)ncc1Cl] | ¹HNMR (DMSO-d₆, 400 MHz): δ 10.95 (s, 1H), 8.86-8.85 (m, 1H), 8.69-8.66 (m, 2H), 8.39-8.37 (m, 1H), 8.24-8.20 (m, 2H), 8.13-8.12 (m, 1H), 7.94-7.92 (m, 1H) 7.76 (s, 1H), 4.76-4.74 (m, 1H), 3.87 (s, 2H), 2.56- 2.55 (m, 3H), 1.45- |
| | | | | | 1.43 (m, 3H). LCMS |
| | | | | | m/z: 451.1 (M + H)⁺; |
| | | | | | HPLC: 100%, rt: |
| | | | | | 10.79 min. |
| 149 | D-2 | 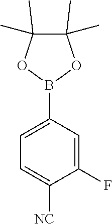 | Pd(dppf) Cl₂•DCM | 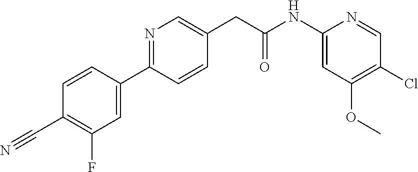 [SMILES: COc1cc(NC(=O)Cc2ccc(-c3ccc(C#N)c(F)c3)nc2)ncc1Cl] | ¹HNMR (DMSO-d₆, 400 MHz): δ 10.99 (s, 1H), 8.67 (s, 1H), 8.23-8.20 (d, 1H), 8.18 (s, 1H), 8.15- 8.11 (t, 2H), 8.06- 8.02 (t, 1H), 7.91 (s, 2H), 3.88 (s, 5H); LCMS m/z: 396.90 (M + H)⁺; HPLC: 96.19%, rt: 7.04 min. |
| 150 | D-23 | 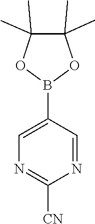 | Pd(dppf) Cl₂•DCM | 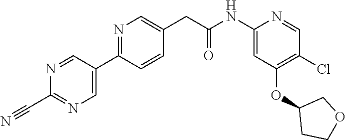 [SMILES: N#Cc1ncc(-c2ccc(CC(=O)Nc3cc(O[C@@H]4CCOC4)c(Cl)cn3)cn2)cn1] | ¹HNMR (DMSO-d₆, 400 MHz): δ 11.02 (s, 1H), 9.63 (s, 2H), 8.73 (s, 1H), 8.26- 8.25 (d, 1H), 8.23 (s, 1H), 7.99-7.96 (q, 1H), 7.88 (s, 1H), 5.11 (s, 1H), 3.91 (s, 2H), 3.86-3.82 (m, 2H), 3.74-3.73 (m, 1H), 2.3-2.2 (m, 2H), 2.0-1.94 (m, 1H); LCMS m/z: 437.20 (M + H)⁺; HPLC: |
| | | | | | 97.41%, rt: 7.26 min. |
| 151 | D-22 | 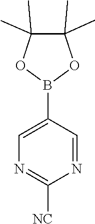 | Pd (amphos) Cl₂ | 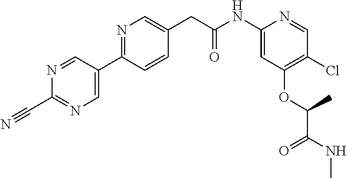 [SMILES: CNC(=O)[C@H](C)Oc1cc(NC(=O)Cc2ccc(-c3cnc(C#N)nc3)nc2)ncc1Cl] | ¹HNMR (DMSO-d₆, 400 MHz): δ 10.97 (s, 1H), 9.63 (s, 2H), 8.72 (d, 1H), 8.25- 8.23 (m, 2H), 8.2-8.1 (m, 1H), 7.98-7.95 (m, 1H), 7.77 (s, 1H), 4.77-4.76 (m, 1H), 3.90 (s, 1H), 3.16 (d, 1H), 2.56 (d, 3H), 1.44 (d, 3H); LCMS m/z: 452.20 (M + H)⁺; HPLC: 97.75%. |
| 152 | D-47 | I-21 | Pd(dppf) Cl₂•DCM | 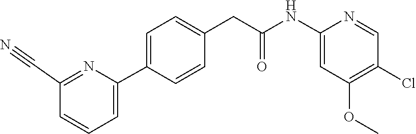 [SMILES: COc1cc(NC(=O)Cc2ccc(-c3cccc(C#N)n3)cc2)ncc1Cl] | ¹HNMR (DMSO-d₆, 400 MHz): δ 10.93 (s, 1H), 8.31 (m, 1H), 8.23 (s, 1H), 8.15- 8.12 (m, 1H), 8.07 (m, 2H), 8.0 (m, 1H), 7.9 (s, 1H), 7.5 (m, 2H), 3.89 (s, 3H), 3.83 (s, 2H); LCMS |
| | | | | | m/z: 378.81 (M + |
| | | | | | H)⁺; HPLC: 98.62%, |
| | | | | | rt: 6.6 min. |
| 153 | D-54 | I-21 | Pd(dppf) Cl₂•DCM | 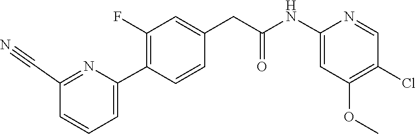 [SMILES: COc1cc(NC(=O)Cc2ccc(-c3cccc(C#N)n3)c(F)c2)ncc1Cl] | ¹HNMR (DMSO-d₆, 400 MHz): δ 10.93 (s, 1H), 8.25 (m, 1H), 8.21-8.17 (m, 1H), 8.13-8.11 (m, 1H), 8.08-8.06 (m, 1H), 7.95 (s, 1H), 7.92- 7.88 (m, 1H), 7.4- 7.35 (m, 2H), 3.91 (s, |
| | | | | | 3H), 3.87 (s, 2H); |
| | | | | | LCMS m/z: 396.90 |
| | | | | | (M + H)⁺; HPLC: |
| | | | | | 99.64%, rt: 7.17 min. |
| 154 | D-23 | I-21 | Pd(PPh₃)₄ | 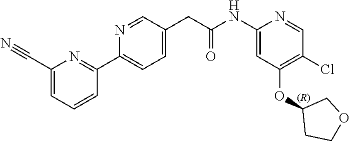 | ¹HNMR (DMSO-d₆, 400 MHz): δ 11.0 (s, 1H), 8.64-8.63 (m, 1H), 8.61 (s, 1H), 8.31-8.29 (m, 1H), 8.23 (s, 1H), 8.19- 8.15 (m, 1H), 8.076- 8.074 (m, 1H), 7.93- 7.90 (m, 1H), 7.86 |
| | | | | | (s, 1H), 5.10-5.09 |
| | | | | | (m, 1H), 3.87 (s, 2H), |
| | | | | | 3.85-3.82 (m, 3H), |
| | | | | | 3.77-3.30 (m, 1H), |
| | | | | | 2.23-2.20 (m, 1H) |
| | | | | | 1.96-1.92 (m, 1H); |
| | | | | | LCMS m/z: (M + |
| | | | | | H)⁺; 436.2 HPLC: |
| | | | | | 99.33%, rt: 7.594 |
| | | | | | min. |
| 155 | D-22 | I-21 | Pd(PPh₃)₄ | 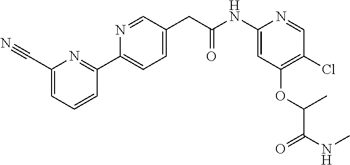 [SMILES: CNC(=O)C(C)Oc1cc(NC(=O)Cc2ccc(-c3cccc(C#N)n3)nc2)ncc1Cl] | ¹HNMR (DMSO-d₆, 400 MHz): δ 10.97 (s, 1H) 8.66-8.64 (d, 1H), 8.34-8.32 (d, 1H), 8.25 (s, 1H), 8.22-8.18 (t, 1H), 8.15-8.14 (d, 1H), 8.10-8.08 (d, 1H), 7.94-7.92 (q, 1H), 7.78 (s, 1H), 4.78- 4.76 (q, 1H), 3.88 (s, |
| | | | | | 1H), 2.58-2.57 (d, |
| | | | | | 3H), 1.46-1.44 (d, |
| | | | | | 3H) 1.25-1.23 (d, |
| | | | | | 2H); LCMS m/z: |
| | | | | | 451.2 (M + H)⁺; |
| | | | | | HPLC: 98.98%, rt: |
| | | | | | 6.04 min, |
| 188 | D-2 | 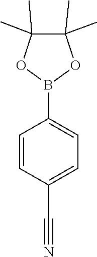 | Pd(dppf) Cl₂•DCM | 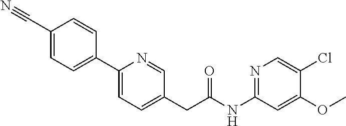 [SMILES: COc1cc(NC(=O)Cc2ccc(-c3ccc(C#N)cc3)nc2)ncc1Cl] | ¹HNMR (DMSO-d₆, 400 MHz): δ 11.0 (s, 1H), 8.65 (s, 1H), ), 8.28-8.23 (m, 3H), 8.08 (s, 1H), 7.97-7.88 (m, 4H), 3.89 (s, 3H), 3.87 (s, 2H), LCMS m/z: (M + H)⁺; 379.10 HPLC: 98.37%. |
| 189 | D-2 | 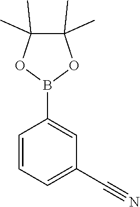 | Pd(dppf) Cl₂•DCM | 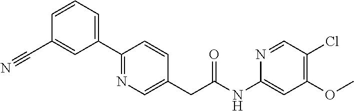 [SMILES: COc1cc(NC(=O)Cc2ccc(-c3cccc(C#N)c3)nc2)ncc1Cl] | ¹HNMR (DMSO-d₆, 400 MHz): δ 10.99 (s, 1H), 8.65 (s, 1H), ), 8.56-8.31 (m, 2H), 8.23 (s, 1H), 8.06 (s, 1H), 7.92- 7.88 (m, 3H), 7.71 (s, 1H), 3.89 (s, 3H), 3.87 (s, 2H). LCMS m/z: (M + H)⁺; 379.0 HPLC: 97.22%. |

### Example-7: Synthesis of N-(5-chloro-4-methoxypyridin-2-yl)-2-(6′-cyano-[2,2′-bipyridin]-5-yl)acetamide (Compound-156)

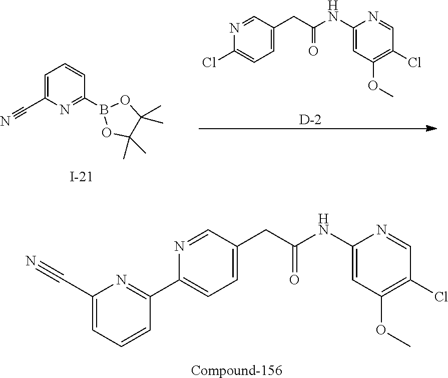

A stirred solution of N-(5-chloro-4-methoxypyridin-2-yl)-2-(6-chloropyridin-3-yl) acetamide (10 g, 32.03 mmol) and 6-(4,4,5,5-tetramethyl-1,3,2-dioxaborolan-2-yl)picolinonitrile (14.74 g, 64.07 mmol), was taken up in DMF (50 mL). The reaction mixture was degassed with Argon for 10 min, then Pd(PPh₃)₄ (3.7 g, 3.2 mmol) was added, followed by K₂CO₃ (4.42 g, 32.02 mmol). The mixture was heated to 100° C. for 14 h. The reaction mixture was quenched with ice cold water, and the precipitated solid was filtered and washed with excess water. The filtered solid was re-dissolved in 10% MeOH/DCM and washed with brine solution. The organic layer was dried over anhydrous Na₂SO₄ Activated charcoal (2 g) was added to the organic layer and the mixture was and refluxed for 0.5 h. The mixture was filtered through a celite bed and washed with 10% MeOH/DCM. The filtrate was evaporated under vacuo and redissolved in 5 volumes of acetonitrile at reflux conditions and cooled slowly to RT. The precipitated solid was filtered and dried to obtain titled product as white coloured solid (6.2 g, 51%); ¹HNMR (DMSO-d₆, 400 MHz): δ 11.02 (s, 1H), 8.66-8.65 (m, 1H), 8.63 (s, 1H), 8.33-8.31 (m, 1H), 8.23 (m, 1H), 8.19-8.17 (m, 1H), 8.09 (m, 1H), 7.99 (m, 1H), 7.91 (s, 1H), 3.87 (s, 3H), 3.84 (s, 2H), LCMS m/z: 380.0 (M+H)⁺; HPLC: 99.84%, rt: 7.54 min.

### Example 8: Synthesis of N-(5-chloro-4-methoxypyridin-2-yl)-2-(6-(6-cyanopyrazin-2-yl)pyridin-3-yl)acetamide (Compound 157)

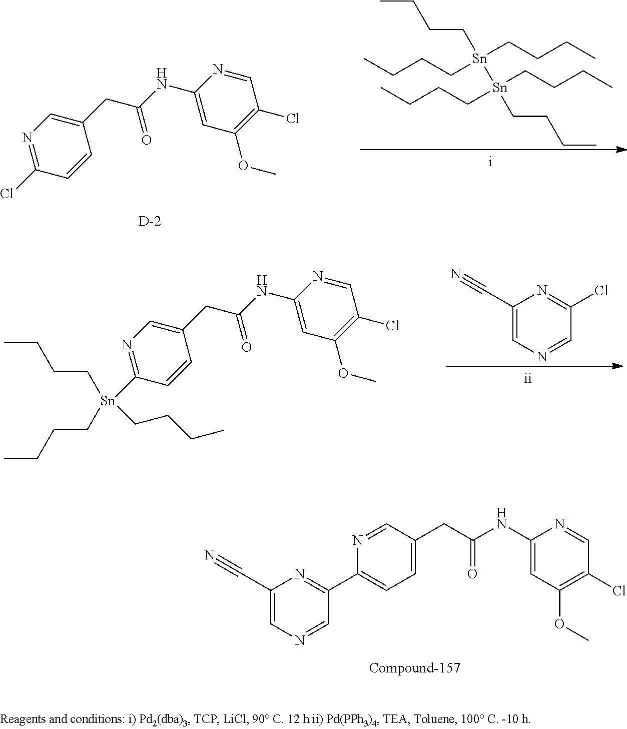

### Step-i: Synthesis of N-(5-chloro-4-methoxypyridin-2-yl)-2-(6-(tributylstannyl)pyridin-3-yl)acetamide

To a degassed solution of N-(5-chloro-4-methoxypyridin-2-yl)-2-(6-chloropyridin-3-yl)acetamide (2 g, 6.40 mmol) and 1,1,1,2,2,2-hexabutyldistannane (3.71 g, 6.40 mmol) in 1,4 Dioxane (25 mL) were added Pd₂(dba)₃ (0.58 g, 0.64 mmol) and tricyclohexylphosphine (0.35 g, 1.28 mmol). The mixture was stirred for 10 min at RT and degassed further for 10 min before LiCl (0.54 g, 12.81 mmol) was added. The reaction mass was stirred in a sealed tube at 90° C. for 12 h. The reaction mixture was concentrated and diluted with water and ethyl acetate. The organic layer was separated, and the aqueous layer was extracted again with ethyl acetate. The combined organic layer was dried over anhydrous sodium sulphate and concentrated under vacuum. The crude compound was purified by silica gel column chromatography by eluting with 0-50% ethyl acetate-hexane to afford the title compound (1.1 g, 30.29%). LCMS: m/z=568.30 (M+H)⁺.

### Step-ii: Synthesis of N-(5-chloro-4-methoxypyridin-2-yl)-2-(6-(6-cyanopyrazin-2-yl)pyridin-3-yl)acetamide

TEA (0.22 g, 1.76 mmol) was added to a degassed solution of N-(5-chloro-4-methoxypyridin-2-yl)-2-(6-(tributylstannyl)pyridin-3-yl)acetamide (0.5 g, 0.88 mmol) and 6-chloropyrazine-2-carbonitrile (0.16 g, 1.14 mmol) in Toluene (5 mL). The mixture was stirred for 10 min at RT and degassed further for 10 min, before Pd(PPh₃)₄ (0.102 g, 0.08 mmol) was added. The reaction mass was stirred at 100° C. for 10 h. The reaction mixture was concentrated and diluted with water and ethyl acetate. The organic layer was separated, and the aqueous layer was extracted again with ethyl acetate. The combined organic layer was dried over anhydrous sodium sulphate and concentrated under vacuum. The crude compound was purified by silica gel column chromatography by eluting with 15% ethyl acetate-hexane to afford the title compound (0.1 g, 29.77%). ¹HNMR (DMSO-d₆, 400 MHz): δ 11 (s, 1H), 9.73 (s, 1H), 9.24 (s, 1H), 8.7 (d, 1H), 8.3-8.28 (d, 1H), 8.21 (s, 1H), 7.99-7.95 (q, 1H), 7.89 (s, 1H), 3.90 (s, 2H), 3.85 (s, 3H); LCMS m/z: 381.1 (M+H)⁺; HPLC: 96.83%, rt: 6.93 min.

The compounds listed in below Table L were prepared by reacting an amide intermediate with 1,1,1,2,2,2-hexabutyldistannane, followed by Stille coupling with a chloropyridine using a suitable catalyst, following a procedure similar to the one described in the synthesis of Example 8 with appropriate variations. The characterization data of the intermediates are summarized herein the below table:

TABLE L

| Comp. | SM used | Catalyst | | |
|---|---|---|---|---|
| No. | for Step-ii | used | Compound structure | Characterization Data |
| 158 | 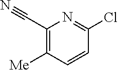 | XantPhos Pd G3 | 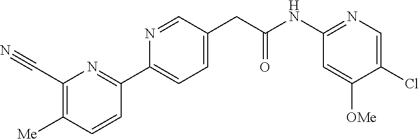 [SMILES: COc1cc(NC(=O)Cc2ccc(-c3ccc(C)c(C#N)n3)nc2)ncc1Cl] | ¹HNMR (DMSO-d₆, 400 MHz): δ 11.02 (s, 1H), 8.6 (s, 1H), 8.65 (m, 1H), 8.31 (m, 1H), 8.24 (s, 1H), 8.0 (m, 1H), 7.94 (s, 2H), 3.8 (s, 5H), 2.5 (s, 3H); LCMS m/z: 393.82 (M + H)⁺; HPLC: 99.02%, rt: 6.4 min. |
| 159 | 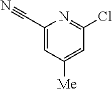 | XantPhos Pd G3 | 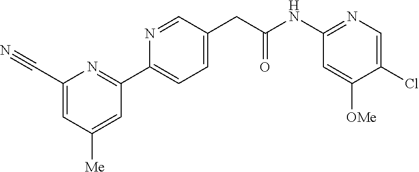 [SMILES: COc1cc(NC(=O)Cc2ccc(-c3cc(C)cc(C#N)n3)nc2)ncc1Cl] | ¹HNMR (DMSO-d₆, 400 MHz): δ 11.02 (s, 1H), 8.6 (s, 1H), 8.65 (m, 1H), 8.31 (m, 1H), 8.24 (s, 1H), 8.0 (m, 1H), 7.94 (s, 2H), 3.8 (s, 5H), 2.5 (s, 3H); LCMS m/z: 393.82 (M + H)⁺; HPLC: 98.09%, rt: 6.5 min. |
| 160 | 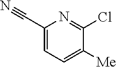 | Pd(PPh₃)₄ | 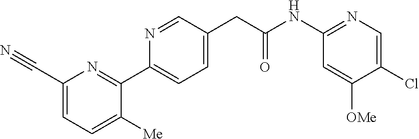 [SMILES: COc1cc(NC(=O)Cc2ccc(-c3nc(C#N)ccc3C)nc2)ncc1Cl] | ¹HNMR (DMSO-d₆, 400 MHz): δ 10.9 (s, 1H), 8.6 (s, 1H), 8.2 (s, 1H), 8.04 (m, 1H), 8.01 (m, 1H), 7.94 (m, 1H), 7.92 (m, 1H), 7.8 (s, 1H) 3.9 (s, 5H), 2.5 (s, 3H); LCMS m/z: 393.82 (M + H)⁺; HPLC: 98.93%, rt: 6.1 min. |
| 165 | I-28 | Pd(PPh₃)₄ | 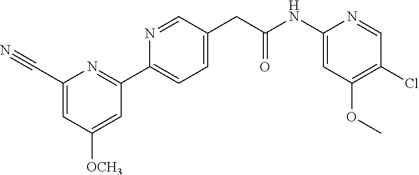 | ¹HNMR (DMSO-d₆, 400 MHz): δ 11.08 (s, 1H), 8.67 (s, 1H),), 8.32 (d, 1H) , 8.30 (s, 1H), 8.24 (s, 1H), 7.93-7.78 (m, 2H), 7.76 (s, 1H), 4.00 (s, 3H), 3.9 (s, 2H), 3.89 (s, 3H). LCMS m/z: (M + H)⁺; 413.1 HPLC: 97.85%. |
| 169 | I-29 | Pd(PPh₃)₄ | 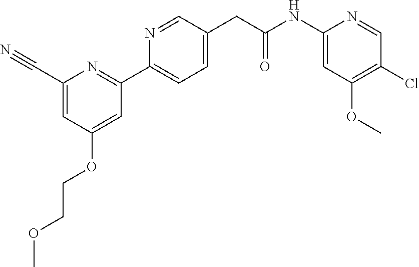 [SMILES: COCCOc1cc(C#N)nc(-c2ccc(CC(=O)Nc3cc(OC)c(Cl)cn3)cn2)c1] | ¹HNMR (DMSO-d₆, 400 MHz): δ 11 (s, 1H), 8.666-8.663 (d, 1H), 8.33-8.31 (q, 1H), 8.24 (s, 1H), 8.14-8.13 (d, 1H), 7.95-7.93 (d, 2H), 7.79-7.78 (d, 1H), 4.40-4.38 (m, 2H), 3.90-3.89 (d, 5H), 3.73- 3.71 (m, 2H), 3.327-3.322 (d, 3H); LCMS m/z: 454.2 (M + H)⁺; HPLC: 99.68%, rt: 6.83 min. |
| 170 | P-14 | Pd(PPh₃)₄ | 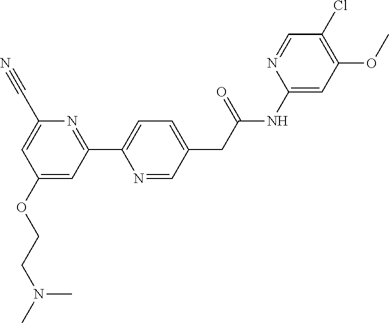 [SMILES: COc1cc(NC(=O)Cc2ccc(-c3cc(OCCN(C)C)cc(C#N)n3)nc2)ncc1Cl] | ¹HNMR (DMSO-d₆, 400 MHz): δ 11.0 (s,1H), 8.66 (s, 1H), 8.33-8.30 (m, 1H), 8.24 (s, 1H), 8.13-8.12 (m, 1H), 7.95-7.93 (m, 2H), 7.78 (m, 1H), 4.34-4.31 (t, 2H), 3.90-3.89 (m, 5H), 2.69-2.67 (m, 2H), 2.2 (s, 6H); LCMS m/z: 467.3 (M + H)⁺; HPLC: 98.40%, rt: 5.293m |
| 171 | P-1 | Pd(PPh₃)₄ | 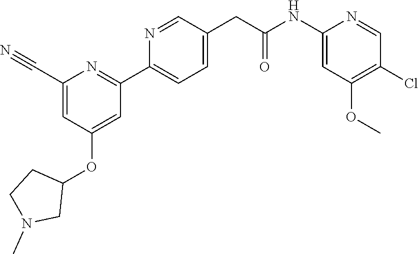 [SMILES: COc1cc(NC(=O)Cc2ccc(-c3cc(OC4CCN(C)C4)cc(C#N)n3)nc2)ncc1Cl] | ¹HNMR (DMSO-d₆, 400 MHz): δ 11.03 (s, 1H), 8.65-8.65 (s, 1H), 8.31-8.29 (m, 1H), 8.23 (m, 1H), 8.04-8.04 (m, 1H), 7.94-7.93 (m, 1H), 7.92 (s, 1H), 7.71-7.70 (m, 1H), 5.17-5.15 (m, 1H), 3.91-3.89 (s, 2H), 3.88 (s, 3H), 2.79-2.77 (m, 3H), 2.40-2.39 (m, 2H), 2.37- 2.36 (s, 3H), 1.86-1.84 (m, 1H); LCMS m/z: 479.3 (M + H)⁺; HPLC: 98.41%, rt: 5.57 min. |
| 173 | P-15 | Pd(PPh₃)₄ | 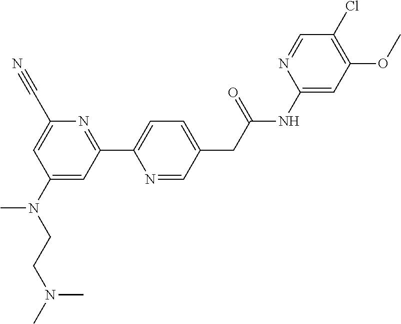 | ¹HNMR (DMSO-d₆, 400 MHz): δ 10.99 (s,1H), 8.62-8.26 (s, 1H), 8.27-8.24 (m, 2H), 7.93-7.87 (m, 2H), 7.8-7.79 (s, 1H), 7.32-7.31 (s, 1H), 3.89-3.87 (m, 5H), 3.61-3.58 (m, 2H), 3.06 (s, 3H), 2.45-2.42 (m, 2H), 2.2 (s, 6H); LCMS m/z: 480.2 (M + H)⁺; HPLC: 98.70%, rt: 5.305 min. |
| 176 | I-31 | Pd(PPh₃)₄ | 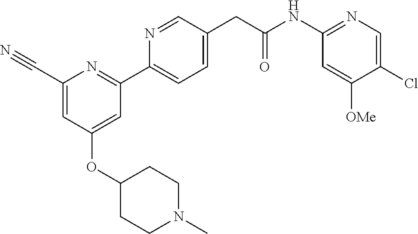 [SMILES: COc1cc(NC(=O)Cc2ccc(-c3cc(OC4CCN(C)CC4)cc(C#N)n3)nc2)ncc1Cl] | ¹HNMR (DMSO-d₆, 400 MHz): δ 11.00 (s, 1H), 8.65-8.64 (m, 1H), 8.30-8.28 (m, 1H), 8.23 (s, 1H), 8.08-8.07 (m, 1H), 7.94-7.93 (m, 1H), 7.92 (s, 1H). 7.81-7.80 (m, 1H), 4.76-4.75 (m, 1H), 3.89 (s, 2H) 3.88 (s, 3H), 2.59-2.58 (m, 2H), 2.26-2.24 (m, 2H), 2.21-2.19 (s, 3H), 2.00-1.97 (m, 2H), 1.71- 1.70 (m, 2H); LCMS m/z: 493.3 (M + H)⁺; HPLC: 96.54%, rt: 4.98 min. |
| 181 | P-2 | Pd(PPh₃)₄ | 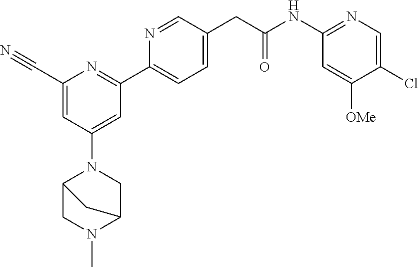 [SMILES: COc1cc(NC(=O)Cc2ccc(-c3cc(N4CC5CC4CN5C)cc(C#N)n3)nc2)ncc1Cl] | ¹HNMR (DMSO-d₆, 400 MHz): δ 11.0 (s,1H), 8.62-8.619 (s, 1H), 8.269 (s, 1H), 8.247-8.245 (s,1H), 7.930 (s, 1H), 7.90-7.879 (m, 1H), 7.598 (s, 1H), 7.39 (m, 1H), 4.634 (s, 1H), 3.89 (s, 3H), 3.88 (s, 2H), 3.536 (s, 1H), 3.422-3.40 (m, 3H) 2.95-2.955 (m, 1H), 2.339- 2.30 (s, 3H), 1.95-1.93 (m, 1H), 1.806-1.781 (m, 1H); LCMS m/z: (M + H)⁺; 490.2 HPLC: 99.26%, rt: 6.65 min. |
| 182 | P-13 | Pd(PPh₃)₄ | 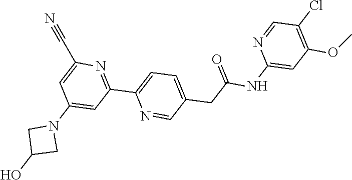 [SMILES: COc1cc(NC(=O)Cc2ccc(-c3cc(N4CC(O)C4)cc(C#N)n3)nc2)ncc1Cl] | ¹HNMR (DMSO-d₆, 400 MHz): δ 10.99 (s, 1H), 8.62-8.61 (s, 1H), 8.27-8.24 (m, 2H), 7.92-7.88 (m, 2H), 7.47 (s, 1H), 7.06 (s, 1H), 2.85-5.83 (d, 1H), 4.65-4.62 (m, 1H), 4.31-4.27 (m, 2H), 3.89-3.88 (m, 5H), 3.82-3.79 (m, 2H); LCMS m/z: 451.1 (M + H)⁺; HPLC: 98.64%, rt: 5.830m |
| 184 | P-10 | Pd(PPh₃)₄ | 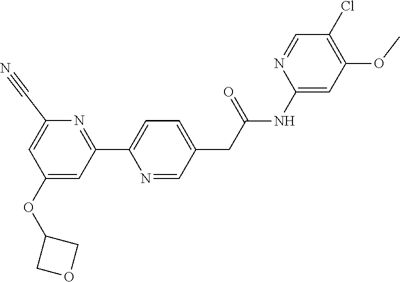 [SMILES: COc1cc(NC(=O)Cc2ccc(-c3cc(OC4COC4)cc(C#N)n3)nc2)ncc1Cl] | ¹HNMR (DMSO-d₆, 400 MHz): δ 11.007 (s, 1H), 8.669-8.665 (d, 1H), 8.322-8.300 (d, 1H), 8.244 (s, 1H), 7.970-7.928 (m, 3H), 7.679-7.673 (d, 1H), 5.610-5.581 (m, 1H), 5.028-4.993 (t, 2H), 4.636-4.604 (q, 2H), 3.907-3.893 (d, 5H); LCMS m/z: 452.2 (M + H)⁺; HPLC: 98.27%, rt: 6.374 min. |
| 191 | P-3 | Pd(PPh₃)₄ | 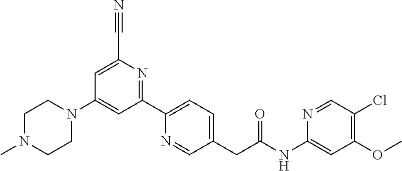 | ¹HNMR (DMSO-d₆, 400 MHz): δ 10.99 (s, 1H), 8.61 (s, 1H), 8.27- 8.25 (s, 1H), 8.23 (s, 1H), 7.97-7.96 (m, 1H), 7.92 (s, 1H), 7.90-7.88 (s, 1H), 7.57-7.56 (m, 1H), 3.88 (s, 2H), 3.87 (s, 3H), 3.50-3.48 (m, 4H), 2.44-2.42 (m, 4H), 2.22 (s, 3H); LCMS m/z : 478.3 (M + H)⁺; HPLC: 95.09%, rt: 4.86 min. |
| 192 | P-4 | Pd(PPh₃)₄ | 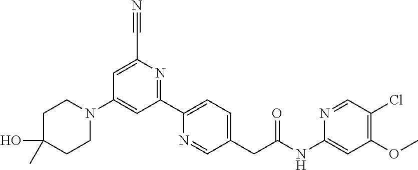 [SMILES: COc1cc(NC(=O)Cc2ccc(-c3cc(N4CCC(C)(O)CC4)cc(C#N)n3)nc2)ncc1Cl] | ¹HNMR (DMSO-d₆, 400 MHz): δ 10.99 (s, 1H), 8.62 (s, 1H), 8.27- 8.25 (m, 1H), 8.24 (s, 1H), 7.92- 7.90 (m, 3H), 7.54 (s, 1H), 4.47 (s, 1H). 3.89 (s, 3H), 3.88 (s, 2H), 3.76-3.73 (m, 2H), 3.38-3.36 (m, 2H), 1.54-1.52 (m, 4H), 1.16 (s, 3H),; LCMS m/z: 493.2 (M + H)⁺; HPLC: 99.28%, rt: 6.09 min |
| 193 | P-5 | Pd(PPh₃)₄ | 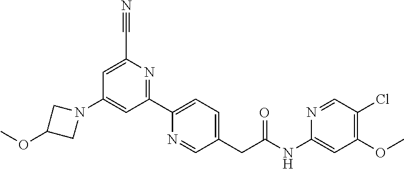 | ¹HNMR (DMSO-d₆, 400 MHz): δ 10.99 (s, 1H), 8.62 (s, 1H), 8.27- 8.25 (m, 1H), 8.24 (m, 1H), 7.92 (s, 1H), 7.90-7.88 (m, 1H), 7.49 (s, 1H). 7.06 (s, 1H), 4.40-4.39 (m, 1H), 4.29-4.27 (m, 2H), 3.92 (s, 3H), 3.90 (s, 2H), 3.88 (m, 2H), 3.32 (s, 3H),; LCMS m/z: 465.2 (M + H)⁺; HPLC: 99.16 %, rt: 6.36 min. |
| 194 | P-6 | Pd(PPh₃)₄ | 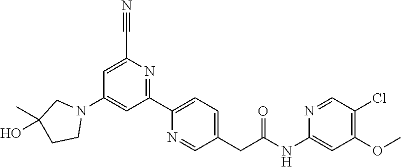 | ¹HNMR (DMSO-d₆, 400 MHz): δ 11.04 (s, 1H), 8.62 (s, 1H), 8.28- 8.26 (m, 1H), 8.24 (s, 1H), 7.93 (s, 1H), 7.90-7.88 (m, 1H), 7.61 (s, 1H). 7.13 (s, 1H), 4.93 (s, 1H), 3.89 (s, 3H), 3.88 (s, 1H), 3.52 (m, 2H), 1.97-1.95 (m, 2H), 1.38 (s, 3H), 1.24 (m, 2H),; LCMS m/z: 479.2 (M + H)⁺; HPLC: 99.38%, rt: 6.00 min. |
| 195 | P-7 | Pd(PPh₃)₄ | 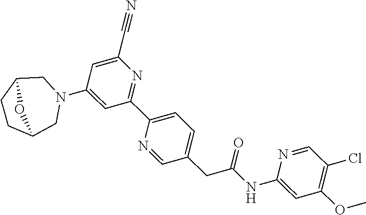 [SMILES: COc1cc(NC(=O)Cc2ccc(-c3cc(N4C[C@H]5CC[C@@H](C4)O5)cc(C#N)n3)nc2)ncc1Cl] | ¹HNMR (DMSO-d₆, 400 MHz): δ 11.00 (s, 1H), 8.66 (s, 1H), 8.32- 8.30 (m, 1H), 8.24 (s, 1H), 8.09 (m, 1H), 7.95-7.94 (m, 1H), 7.93 (s, 1H). 7.80 (m, 1H), 4.78 (s, 1H), 3.90 (s, 3H), 3.89 (s, 2H), 2.83 (m, 1H), 2.51-2.49 (m, 1H), 2.23-2.19 (m, 4H), 2.12 (m, 1H), 1.97-1.96 (m, 1H), 1.74-1.73 (m, 1H), 1.60- 1.58 (m, 1H), 1.48 (m, 1H); LCMS m/z: 491.3 (M + H)⁺; HPLC: 98.84%, rt: 5.02 |
| 196 | P-8 | Pd(PPh₃)₄ | 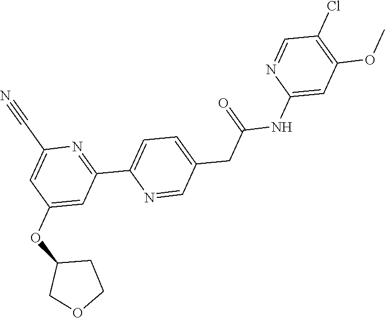 [SMILES: COc1cc(NC(=O)Cc2ccc(-c3cc(O[C@H]4CCOC4)cc(C#N)n3)nc2)ncc1Cl] | ¹HNMR (DMSO-d₆, 400 MHz): δ 10.977 (s, 1H), 8.625-8.621 (d, 1H), 8.291-8.270 (d, 1H), 8.207 (s, 1H), 8.061-8.055 (d, 1H), 7.961-7.910 (d, 1H), 7.891 (s, 1H), 7.750-7.745 (d, 1H), 5.328-5.325 (s, 1H), 3.914-3.807 (m, 8H), 3.779-3.725 (m, 1H), 2.324-2.274 (m, 1H), 2.037- 1.975 (m, 1H); LCMS m/z: 466.2 (M + H)⁺; HPLC: 98.70%, rt: 5.193 min. |
| 197 | P-9 | Pd(PPh₃)₄ | 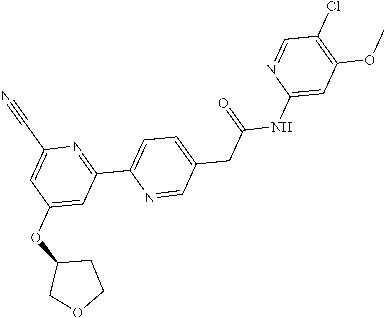 [SMILES: COc1cc(NC(=O)Cc2ccc(-c3cc(O[C@H]4CCOC4)cc(C#N)n3)nc2)ncc1Cl] | ¹HNMR (DMSO-d₆, 400 MHz): δ 10.977 (s, 1H), 8.625-8.621 (d, 1H), 8.291-8.271 (d, 1H), 8.207 (s, 1H), 8.062-8.055 (d, 1H), 7.961-7.910 (d, 1H), 7.891 (s,1H), 7.751-7.745 (d, 1H), 5.325 (s, 1H), 3.914-3.807 (m, 7H), 3.779-3.736 (m, 1H), 2.324-2.275 (m, 2H), 2.051-1.989 (m, 1H); LCMS m/z: 466.1 (M + H)⁺; HPLC: 98.12%, rt: 5.169 min. |
| 198 | P-11 | Pd(PPh₃)₄ | 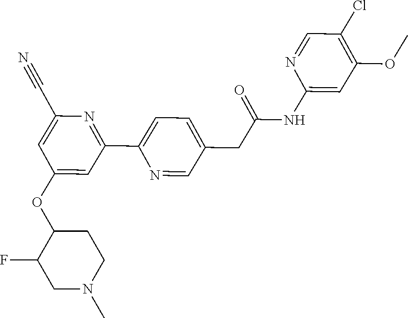 [SMILES: COc1cc(NC(=O)Cc2ccc(-c3cc(OC4CCN(C)CC4F)cc(C#N)n3)nc2)ncc1Cl] | ¹HNMR (DMSO-d₆, 400 MHz): δ 11.008 (s,1H), 8.666-8.661 (d, 1H), 8.321-8.300 (d, 1H), 8.246 (s, 1H), 8.174-8.150 (q, 1H), 7.954-7.930 (m, 2H), 7.909-7.897 (t, 1H), 5.05- 4.90 (d, 1H), 3.906-3.894 (d, 4H), 2.68 (t, 1H), 2.33 (t, 1H), 2.27 (s, 3H), 2.17 (m, 1H), 2.01-1.95 (t, 1H), 1.241 (s, 3H), 0.876 (m, 1H); LCMS m/z: 511.3 (M + H)⁺; HPLC: 99.50%, rt: 5.103 min. |
| 199 | P-12 | Pd(PPh₃)₄ | 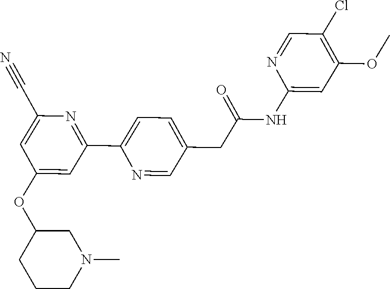 [SMILES: COc1cc(NC(=O)Cc2ccc(-c3cc(OC4CCCN(C)C4)cc(C#N)n3)nc2)ncc1Cl] | ¹HNMR (DMSO-d₆, 400 MHz): δ 11.00 (s, 1H), 8.66 (s, 1H), 8.32- 8.30 (m, 1H), 8.24 (s, 1H), 8.09 (m, 1H), 7.95-7.94 (m, 1H), 7.93 (s, 1H). 7.80 (m, 1H), 4.78 (s, 1H), 3.90 (s, 3H), 3.89 (s, 2H), 2.83 (m, 1H), 2.51-2.49 (m, 1H), 2.23-2.19 (m, 4H), 2.12 (m, 1H), 1.97-1.96 (m, 1H), 1.74-1.73 (m, 1H), 1.60- 1.58 (m, 1H), 1.48 (m, 1H); LCMS m/z: 493.3 (M + H)⁺; HPLC: 98.84%, rt: 5.02 min |

### Example-9: N-(5-chloro-4-methoxypyridin-2-yl)-2-(6′-cyano-4′-(2-hydroxypropan-2-yl)-[2,2′-bipyridin]-5-yl)acetamide (Compound 175)

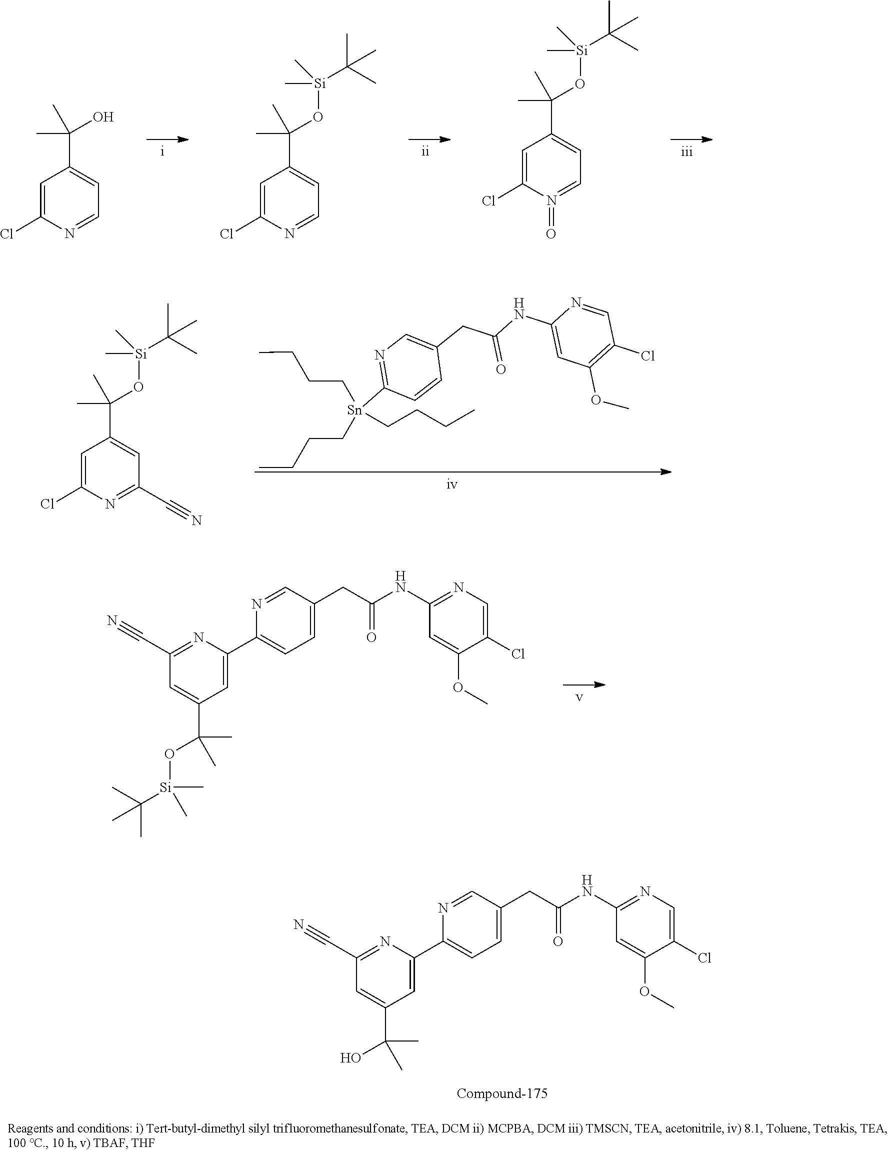

### Step-i: Synthesis of 4-(2-((tert-butyldimethylsilyl) oxy) propan-2-yl)-2-chloropyridine

To a stirred solution of 2-(2-chloropyridin-4-yl) propan-2-ol (0.8 g, 4.66 mmol) in dry DCM (10 mL), was added TEA (0.9 g, 9.32 mmol) at 0° C. After addition, the reaction mixture was stirred at same temperature for 30 min, then added Tert-butyl-dimethyl silyl trifluoromethanesulfonate (1.84 gm, 7 mmole) and stirred for 12 hr. The reaction mixture was diluted with DCM and filtered through celite. The filtrate was concentrated to get crude. The crude residue was purified by using 12 g combi flash column chromatography to afford the title compound (1.20 g, 90.06%); LCMS m/z: 286.2 (M+H)+

### Step-ii: Synthesis of 4-(2-((tert-butyldimethylsilyl) oxy) propan-2-yl)-2-chloropyridine 1-oxide

To a stirred solution of 4-(2-((tert-butyldimethylsilyl) oxy) propan-2-yl)-2-chloropyridine (1 g, 3.94 mmol) in DCM (50 mL) was added mCPBA (1.2 g, 6.99 mmol) at 0° C. for 10 min. After addition stirred the reaction mixture at RT for 12 h. The reaction mixture was diluted with saturated NaHCO₃, separated the organic layer and aqueous layer. The organic layer was dried over anhydrous sodium sulphate and concentrated under vacuum. The crude compound was purified by silica gel column chromatography by eluting with 0-50% ethyl acetate-hexane to afford the title compound (0.9 g 85.23%), LCMS: m/z=302.1 (M+H)⁺.

### Step-iii: Synthesis of 4-(2-((tert-butyldimethylsilyl) oxy) propan-2-yl)-6-chloropicolinonitrile

To a stirred solution of 4-(2-((tert-butyldimethylsilyl) oxy) propan-2-yl)-2-chloropyridine 1-oxide (0.7 g, 2.38 mmol) in acetonitrile (30 mL) was added TEA (0.46 g, 4.63 mmol), TMSCN (0.9 g, 9.27 mmol) at 0° C. The reaction mixture was heated at 80° C. for 24 h. The reaction mixture was diluted with ice water, extracted with EtOAc, The organic layer was dried over anhydrous sodium sulphate and concentrated under vacuum. The crude compound was purified by silica gel column chromatography by eluting with 0-50% EtOAc/Hexane to afford the title compound (0.6 g 83.22%), LCMS: m/z=311.2 (M+H)⁺.

### Step-iv: Synthesis of 2-(4′-(2-((tert-butyldimethylsilyl) oxy) propan-2-yl)-6′-cyano-[2,2′-bipyridin]-5-yl)-N-(5-chloro-4-methoxypyridin-2-yl) acetamide

TEA (0.1 g, 0.99 mmol) was added to a degassed solution of N-(5-chloro-4-methoxypyridin-2-yl)-2-(6-(tributylstannyl) pyridin-3-yl) acetamide (Reference: Example-8 step-1)(0.32 g, 1.05 mmol) and 4-(2-((tert-butyldimethylsilyl) oxy) propan-2-yl)-6-chloropicolinonitrile (0.5 g, 0.88 mmol) in Toluene (10 mL). The mixture was degassed for 10 min, before Pd (PPh₃)₄ (0.102 g, 0.08 mmol) was added. The reaction mass was stirred at 100° C. for 10 h. The reaction mixture was concentrated and diluted with water and ethyl acetate. The organic layer was separated, and the aqueous layer was extracted again with ethyl acetate. The combined organic layer was dried over anhydrous sodium sulphate and concentrated under vacuum. The crude compound was purified by silica gel column chromatography by eluting with 15% ethyl acetate-hexane to afford the title compound (0.4 g, 82.12%). LCMS: m/z=552.3 (M+H)⁺.

### Step-v: Synthesis of N-(5-chloro-4-methoxypyridin-2-yl)-2-(6′-cyano-4′-(2-hydroxypropan-2-yl)-[2,2′-bipyridin]-5-yl) acetamide

To a stirred solution of 2-(4′-(2-(tert-butyldimethylsilyl) oxy) propan-2-yl)-6′-cyano-[2,2′-bipyridin]-5-yl)-N-(5-chloro-4-methoxypyridin-2-yl)acetamide (0.4 g, 0.724 mmol) in THF (10 mL) was added TBAF (0.56 g, 2.17 mmol) at 0° C. The reaction mixture was stirred at room temperature for 12 h. After the completion of reaction, the reaction mixture was diluted with EtOAc, washed with water, brine and the layers were separated. The organic layer was dried over sodium sulphate, filtered, and concentrated. The crude was purified by combi flash column chromatography to afford the title compound (0.1 g, 31.54%).

¹HNMR (DMSO-d₆, 400 MHz): δ 11.01 (s, 1H), 8.777 (d, 1H), 8.67 (d, 1H), 8.34-8.31 (m, 1H), 8.24 (s, 1H), 8.13 (d, 1H), 7.95-7.92 (m, 2H), 5.6 (s, 1H), 3.9 (s, 2H), 3.88 (s, 3H), 1.48 (s, 6H). LCMS m/z: 438.1 (M+H)⁺; HPLC: 99.89%, rt: 6.47 min.

### Example-10: Chiral separation of racemic compound 2-((5-chloro-2-(2-(6′-cyano-[2,2′-bipyridin]-5-yl)acetamido)pyridin-4-yl)oxy)-N-methylpropanamide

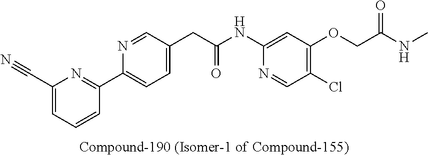 [SMILES: CNC(=O)COc1cc(NC(=O)Cc2ccc(-c3cccc(C#N)n3)nc2)ncc1Cl]

Above enantiomer was isolated from racemic 2-((5-chloro-2-(2-(6′-cyano-[2,2′-bipyridin]-5-yl)acetamido)pyridin-4-yl)oxy)-N-methylpropanamide (Compound-155) by using chiral preparative HPLC column (Method: Column-Regis (s,s) Whelk-ol (250×21.2 mm) 5), Elution:isocratic (45:55), A=Hexane, B=0.1% DEA in EtOH, Flow: 15 mL/min). ¹HNMR (DMSO-d₆, 400 MHz): δ 10.97 (s, 1H) 8.67-8.64 (d, 1H), 8.33-8.32 (d, 1H), 8.24 (s, 1H), 8.21-8.18 (t, 1H), 8.15-8.14 (d, 1H), 8.10-8.07 (d, 1H), 7.93-7.92 (q, 1H), 7.78 (s, 1H), 4.77-4.76 (q, 1H), 3.87 (s, 1H), 2.58-2.57 (d, 3H), 1.46-1.44 (d, 3H) 1.25-1.23 (d, 2H); LCMS m/z: 451.2 (M+H)⁺; HPLC: 98.08%, rt: 6.04 min, Chiral HPLC: 99.21%, rt: 5.44 min.

Second enantiomer was separated but not characterized as it was obtained as a mixture along with the other enantiomer.

Although the present application has been illustrated by certain of the preceding examples, it is not to be construed as being limited thereby; but rather, the present application encompasses the generic area as hereinbefore disclosed. For example, the compounds in the below table-M which can be prepared by following similar procedure as described in above Schemes/Examples with suitable modifications known to the one ordinary skilled in the art are also included in the scope of the present application:

TABLE M

| Compd. | |
|---|---|
| No. | IUPAC Name |
| 161 | N-(5-chloro-4-methoxypyridin-2-yl)-2-(6-(4-cyanopyrimidin-2-yl)pyridin-3- |
| | yl)acetamide; |
| 162 | N-(5-chloro-4-methoxypyridin-2-yl)-2-(6-(2-cyanopyrimidin-4-yl)pyridin-3- |
| | yl)acetamide; |
| 163 | N-(5-chloro-4-methoxypyridin-2-yl)-2-(6′-cyano-5′-fluoro-[2,2′-bipyridin]-5- |
| | yl)acetamide; |
| 164 | N-(5-chloro-4-methoxypyridin-2-yl)-2-(6′-cyano-3′-fluoro-[2,2′-bipyridin]-5- |
| | yl)acetamide; |
| 166 | N-(5-chloro-4-methoxypyridin-2-yl)-2-(6′-cyano-4′-(trifluoromethoxy)-[2,2′-bipyridin]- |
| | 5-yl)acetamide; |
| 167 | N-(5-chloro-4-methoxypyridin-2-yl)-2-(6′-cyano-4′-(trifluoromethyl)-[2,2′-bipyridin]-5- |
| | yl)acetamide; |
| 168 | N-(5-chloro-4-methoxypyridin-2-yl)-2-(6′-cyano-4′-(difluoromethyl)-[2,2′-bipyridin]-5- |
| | yl)acetamide; |
| 172 | N-(5-chloro-4-methoxypyridin-2-yl)-2-(6′-cyano-4′-(methyl(1-methylpyrrolidin-3- |
| | yl)amino)-[2,2′-bipyridin]-5-yl)acetamide; |
| 174 | N-(5-chloro-4-methoxypyridin-2-yl)-2-(6′-cyano-4′-(1-hydroxyethyl)-[2,2′-bipyridin]-5- |
| | yl)acetamide; |
| 177 | N-(5-chloro-4-methoxypyridin-2-yl)-2-(6′-cyano-4′-(1-methylpiperidin-4-yl)-[2,2′- |
| | bipyridin]-5-yl)acetamide; |
| 178 | N-(5-chloro-4-methoxypyridin-2-yl)-2-(6′-cyano-4′-(1-methylpyrrolidin-3-yl)-[2,2′- |
| | bipyridin]-5-yl)acetamide; |
| 179 | N-(5-chloro-4-methoxypyridin-2-yl)-2-(6′-cyano-4′-(1-hydroxycyclopropyl)-[2,2′- |
| | bipyridin]-5-yl)acetamide; |
| 180 | N-(5-chloro-4-methoxypyridin-2-yl)-2-(6′-cyano-4′-(1-methylpiperidin-3-yl)-[2,2′- |
| | bipyridin]-5-yl)acetamide; |
| 183 | N-(5-chloro-4-methoxypyridin-2-yl)-2-(6′-cyano-4′-(8-methyl-3,8- |
| | diazabicyclo[3.2.1]octan-3-yl)-[2,2′-bipyridin]-5-yl)acetamide; |
| 185 | N-(5-chloro-4-methoxypyridin-2-yl)-2-(6′-cyano-4′-((1-methylazetidin-3-yl)oxy)-[2,2′- |
| | bipyridin]-5-yl)acetamide; |
| 186 | N-(5-chloro-4-methoxypyridin-2-yl)-2-(6′-cyano-4′-((tetrahydrofuran-3-yl)oxy)-[2,2′- |
| | bipyridin]-5-yl)acetamide; |
| 187 | N-(5-chloro-4-methoxypyridin-2-yl)-2-(6′-cyano-4′-((2-methyl-2-azaspiro[3.3]heptan-6- |
| | yl)oxy)-[2,2′-bipyridin]-5-yl)acetamide. |

### Biochemical Assay for CDK12 (IC₅₀ Determination):

The inhibitory activity of the test compounds was assessed by the LANCE TR-FRET assay, which detects the ATP-dependent phosphorylation of an ULight-4E-BP1 (Thr37/Thr46) substrate peptide (100 nM) by CDK12 (30 nM). Briefly, the enzyme reaction was run in reaction buffer (25 mM HEPES (pH 7.5), 10 mM MgCl₂, 0.0100 BSA, 0.01% Triton x, 1 mM DTT). The assay was performed in 384-well plate format. The end concentration of the ATP substrate was 100 M, and that of the ULight-4E-BP1 (Thr37/Thr46) substrate peptide was 100 nM, and of CDK12 was 30 nM. Pre-incubation of the compound and enzyme was performed for 60 min at room temperature. After 60 min incubation at room temperature, the reaction was terminated by the addition of 40 mM EDTA and 0.5 nM Eu-labeled anti-phospho-eIF4E-binding protein (Thr37/46) antibody in LANCE detection buffer. Time-resolved fluorescence (excitation, 320 nm; emission donor, 615 nm; emission acceptor, 665 nm) was monitored by using 2030 multilabel reader Victor5 (PerkinElmer). The Fluorescence emission of the samples were measured and the ratio was plotted against the compound concentration to generate the dose response curve. The IC₅₀ values were derived by fitting a sigmoidal dose-response curve to a plot of assay readout over inhibitor concentration. All fits were computed with the program Prism 5.03 (Graph Pad Software, San Diego, CA). The % inhibition values at 10 M were calculated with respect to enzyme activity.

Exemplary compounds of the present application were screened by the above mentioned assay and the results were tabulated; the CDK12% Inhibition at 10 M and IC₅₀ values (in range) of the selected compounds are set forth below in table-N wherein “A” refers to a IC₅₀ value less than 0.01 M, “B” refers to a IC₅₀ value in range of 0.01 M to 0.1 M (both inclusive) and “C” refers to a IC₅₀ value above 0.1 M.

TABLE N
CDK12 % Inhibition and IC₅₀ values (in range)

| Compd. | CDK12 % | IC₅₀ μM |
|---|---|---|
| No. | Inhibition @ 10 μM | (in range) |
| 1 | 92 | A |
| 2 | 90 | A |
| 3 | 84 | A |
| 4 | 75 | B |
| 5 | 75 | B |
| 6 | 78 | A |
| 7 | 88 | B |
| 8 | 82 | A |
| 9 | 64 | -ND- |
| 10 | 72 | A |
| 11 | 89 | B |
| 12 | 84 | A |
| 13 | 75 | B |
| 14 | 96 | A |
| 15 | 96 | A |
| 16 | 89 | B |
| 17 | 92 | A |
| 18 | 87 | B |
| 19 | 91 | A |
| 20 | 109 | A |
| 21 | 94 | A |
| 22 | 91 | A |
| 23 | 96 | A |
| 24 | 94 | A |
| 25 | 90 | A |
| 26 | 99 | A |
| 27 | 93 | A |
| 28 | 95 | A |
| 29 | 86 | B |
| 30 | 82 | B |
| 31 | 89 | A |
| 32 | 84 | A |
| 33 | 88 | A |
| 34 | 91 | A |
| 35 | 95 | A |
| 36 | 95 | A |
| 37 | 94 | A |
| 38 | 88 | A |
| 39 | 98 | B |
| 40 | 91 | A |
| 41 | 96 | A |
| 42 | 116 | C |
| 43 | 87 | A |
| 44 | 89 | A |
| 45 | 92 | A |
| 46 | 88 | A |
| 47 | 85 | B |
| 48 | 90 | A |
| 49 | 89 | A |
| 50 | 72 | C |
| 51 | 102 | A |
| 52 | 102 | A |
| 53 | 90 | B |
| 54 | 77 | C |
| 55 | 86 | C |
| 56 | 100 | A |
| 57 | 78 | A |
| 58 | 98 | A |
| 59 | 89 | A |
| 60 | 87 | B |
| 61 | 89 | A |
| 62 | 96 | A |
| 63 | 92 | A |
| 64 | 92 | A |
| 65 | 87 | A |
| 66 | 88 | B |
| 67 | 86 | B |
| 68 | 87 | A |
| 69 | 93 | A |
| 70 | 94 | A |
| 71 | 93 | A |
| 72 | 92 | A |
| 73 | 87 | A |
| 74 | 100 | A |
| 75 | 96 | A |
| 76 | 90 | A |
| 77 | 79 | A |
| 78 | 87 | B |
| 79 | 79 | C |
| 80 | 87 | B |
| 81 | 87 | A |
| 82 | 87 | B |
| 83 | 84 | B |
| 84 | 84 | A |
| 85 | 92 | B |
| 86 | 84 | C |
| 87 | 85 | C |
| 88 | 79 | C |
| 89 | 90 | B |
| 90 | 93 | B |
| 91 | 95 | B |
| 92 | 90 | B |
| 93 | 73 | B |
| 94 | 77 | B |
| 95 | 90 | B |
| 96 | 91 | C |
| 97 | 96 | B |
| 98 | 87 | B |
| 99 | 95 | B |
| 100 | 77 | B |
| 101 | 86 | C |
| 102 | 87 | C |
| 103 | 98 | B |
| 104 | 79 | B |
| 105 | 100 | A |
| 106 | 90 | A |
| 107 | 95 | B |
| 108 | 85 | B |
| 109 | 89 | B |
| 110 | 57 | -ND- |
| 111 | 94 | B |
| 112 | 90 | B |
| 113 | 92 | B |
| 114 | 91 | A |
| 115 | 98 | B |
| 116 | 23 | -ND- |
| 117 | 88 | A |
| 118 | 81 | C |
| 119 | 78 | C |
| 120 | 89 | A |
| 121 | 82 | A |
| 122 | 90 | A |
| 123 | 86 | A |
| 124 | 86 | C |
| 125 | 72 | C |
| 126 | 52 | C |
| 127 | 86 | B |
| 128 | 83 | A |
| 129 | 94 | B |
| 130 | 95 | B |
| 131 | 93 | B |
| 132 | 93 | C |
| 133 | 94 | A |
| 134 | 82 | B |
| 135 | 78 | C |
| 136 | 93 | C |
| 137 | 89 | B |
| 138 | 80 | C |
| 139 | 95 | B |
| 140 | 83 | C |
| 141 | 92 | B |
| 142 | -NA- | B |
| 143 | 92 | B |
| 144 | 92 | B |
| 145 | 79 | B |
| 146 | 90 | B |
| 147 | 89 | B |
| 148 | 89 | B |
| 149 | 54 | -ND- |
| 150 | 83 | B |
| 151 | 79 | B |
| 152 | -NA- | C |
| 153 | 34 | -ND- |
| 154 | 77 | A |
| 155 | 97 | B |
| 156 | 80 | A |
| 157 | 84 | A |
| 158 | 70% @ 2 μM | C |
| 159 | 90% @ 2 μM | B |
| 160 | 76 | C |
| 165 | 72 | B |
| 169 | 95 | B |
| 170 | 90 | B |
| 171 | 83 | A |
| 173 | 90 | B |
| 175 | 97 | B |
| 176 | 96 | B |
| 181 | 92 | B |
| 182 | 75 | C |
| 184 | 80 | B |
| 188 | 65 | C |
| 189 | 82 | C |
| 190 | 87 | A |
| 191 | 94 | B |
| 192 | 88 | B |
| 193 | 68 | C |
| 194 | 94 | C |
| 195 | 84 | B |
| 196 | 89 | B |
| 197 | 87 | B |
| 198 | 100 | B |
| 199 | 88 | A |

NA—Not Applicable
ND—Not Determined

1: A compound of formula (I): 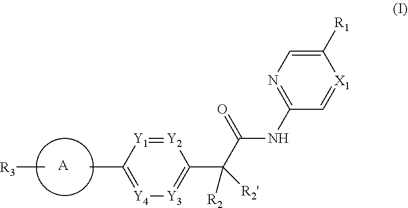
or a pharmaceutically acceptable salt, an N-oxide or a stereoisomer thereof;
wherein,
X₁ is CR₅ or N;
each of Y₁, Y₂, Y₃ and Y₄ is independently CR₆ or N, wherein 0-2 of Y₁, Y₂, Y₃ and Y₄ are N;
ring A is 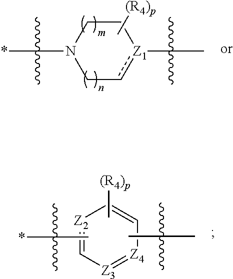
wherein * is the point of attachment with R₃;
‘-----’ is an optional bond;
Z₁ is C or N;
Z₂, Z₃ and Z₄ are each independently C or N;
R₁ is hydrogen, halogen, alkyl, cycloalkyl or alkylthio;
each of R₂ and R₂′ independently is hydrogen or alkyl;
R₃ is —CN or 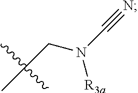
wherein wavy bond indicates the point of attachment with ring A;
R₃ₐ is hydrogen or alkyl;
R₄ at each occurrence independently is halogen, alkyl, haloalkyl, hydroxyalkyl, —OR₄ₐ, —NR_{4b}R_{4c}, unsubstituted or substituted cycloalkyl, unsubstituted or substituted heterocycloalkyl; wherein the substituent is selected from one or more alkyl, halo, alkoxy, haloalkyl or hydroxy;
alternatively, two R₄ each on different carbon atoms form a bridging (C₁-C₃) alkylene or a bridging bond;
R₄ₐ is alkyl, haloalkyl, alkoxyalkyl, alkylaminoalkyl, unsubstituted or alkyl substituted heterocycloalkyl;
R_{4b} and R_{4c} are each independently hydrogen, alkyl, alkylaminoalkyl or unsubstituted or alkyl substituted heterocycloalkyl;
R₅ is:
i) hydrogen, halogen, hydroxyalkyl, alkyl, —PO(CH₃)₂, —OR₅ₐ or —NR_{5b}R_{5c}; or
ii) unsubstituted or substituted heterocycloalkyl, wherein, the substituents are 1 or 2 substituents independently selected from alkyl and hydroxy;
R₅ₐ is alkyl, unsubstituted or alkyl substituted heterocycloalkylalkyl, unsubstituted or alkyl substituted heterocycloalkyl, —CONR_{5d}R₅ₑ, -alkyl-CONR_{5d}R₅ₑ, cycloalkyl, alkoxyalkyl or alkylaminoalkyl;
R_{5b} and R_{5c} are each independently hydrogen or alkyl;
R_{5d} and R₅ₑ are each independently hydrogen or alkyl;
R₆ at each occurrence, is independently hydrogen, alkyl, alkoxy or halogen;
‘p’ is selected from 0 to 3; and
‘m’ and ‘n’ are each independently selected from 0 to 2.

2: The compound of claim 1, having a compound of formula (IA): 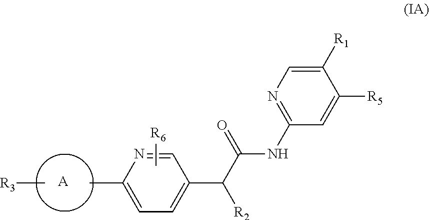
or a pharmaceutically acceptable salt, an N-oxide or a stereoisomer thereof.

3: The compound of claim 1, having a compound of formula:

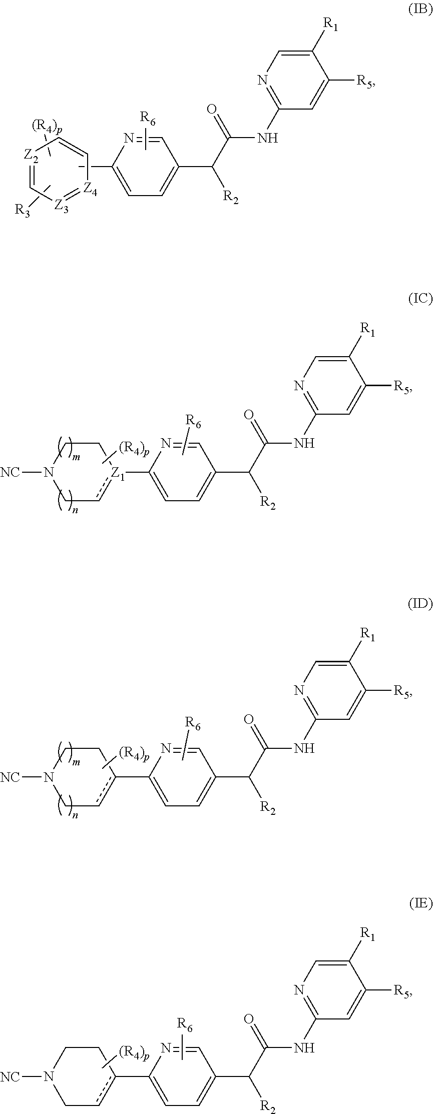

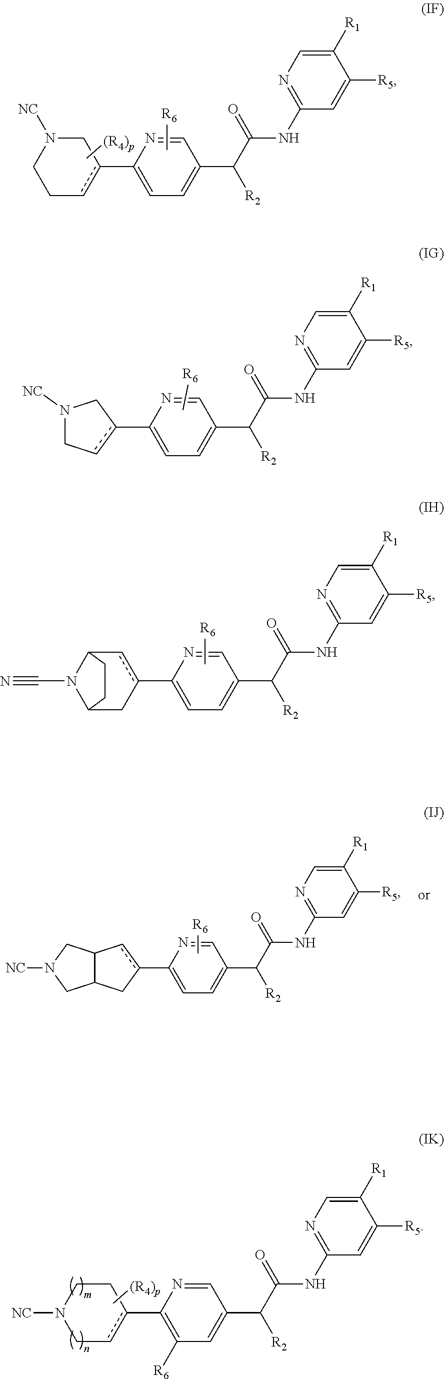

4-10. (canceled)

11: The compound of claim 1, wherein 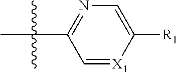
is 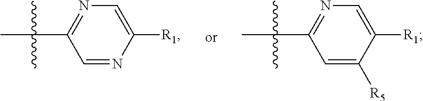
wherein the wavy line indicates the point of attachment to the rest of the molecule in formula (I).

12: The compound of claim 1, wherein 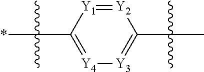
is 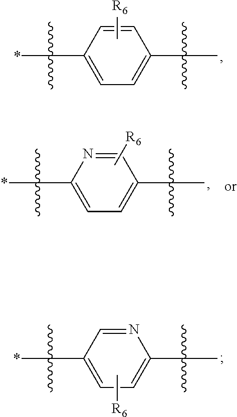
wherein the wavy line with the asterisk symbol indicates the point of attachment to ring A and the wavy line absent of asterisk symbol indicates the point of attachment to the carbon atom having R₂ and R₂′ substituents in formula (I).

13: The compound of claim 12, wherein 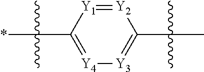
is 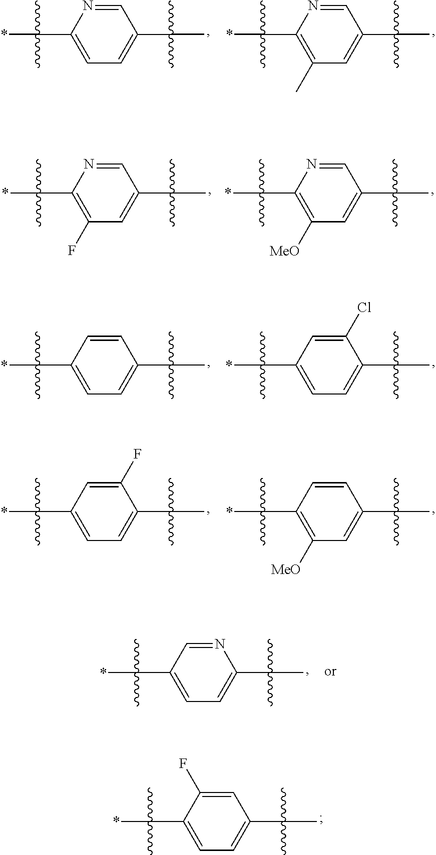
wherein the wavy line with the asterisk symbol indicates the point of attachment to ring A and the wavy line absent of asterisk symbol indicates the point of attachment to the carbon atom having R₂ and R₂′ substituents in formula (I).

14: The compound of claim 1, wherein, ring 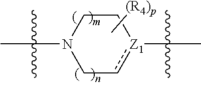
is 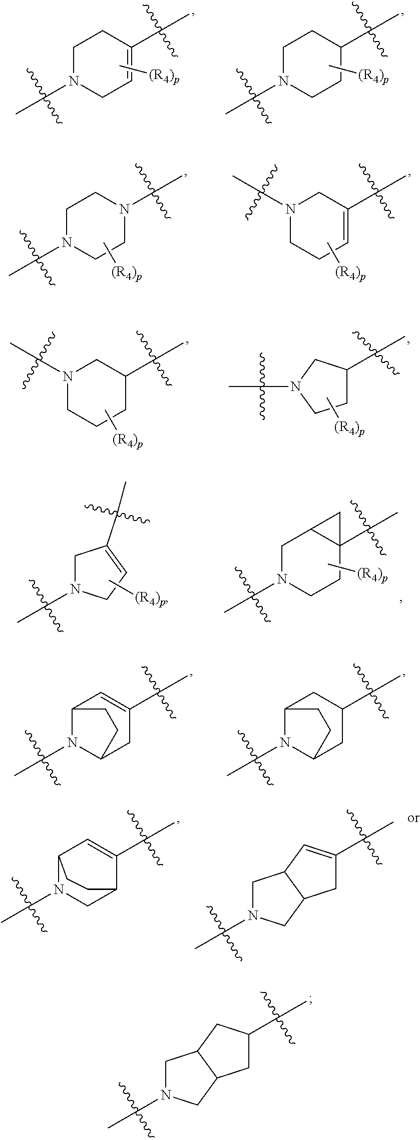
wherein the wavy line next to the nitrogen atom indicates the point of attachment to R₃ and the other wavy line indicates the point of attachment to the rest of molecule in formula (I).

15: The compound of claim 1, wherein, ring 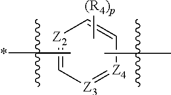
is 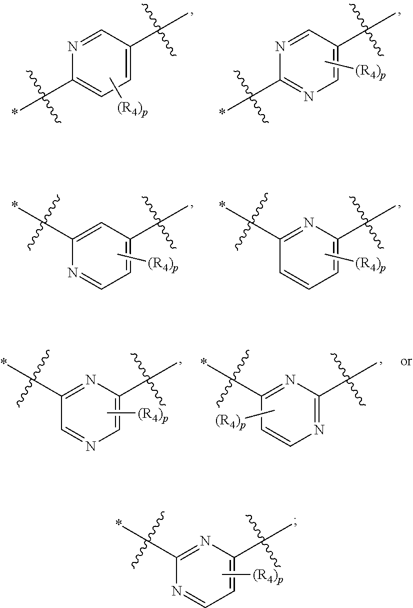
wherein the wavy line with asterisk symbol indicates the point of attachment to R₃ and the wavy line absent of asterisk symbol indicates the point of attachment to the rest of molecule in formula (I).

16: The compound of claim 1, wherein 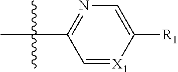
is 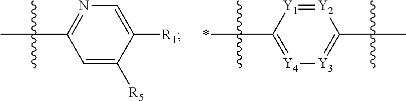
is 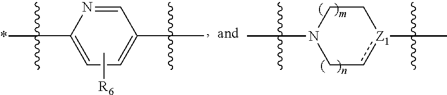
is

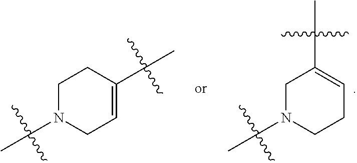 [SMILES: *C1=CCN(*)CC1 ; *C1=CCCN(*)C1]

17: The compound of claim 1, wherein R1 is chloro, fluoro, methyl, cyclopropyl or —SMe.

18: The compound of claim 1, wherein, each of R₂ and R₂′ is hydrogen.

19-20. (canceled)

21: The compound of claim 1, wherein, R₃ is —CN.

22. (canceled)

23: The compound of claim 1, wherein R₅ is:
i) hydrogen, hydroxyalkyl, alkyl or —OR₅ₐ; or
ii) substituted or unsubstituted heterocycloalkyl, wherein, the substituents on heterocycloalkyl are 1 or 2 substituents independently selected from alkyl and hydroxy.

24: The compound of claim 1, wherein, R₅ is hydrogen or —OR₅ₐ, wherein R₅ₐ is alkyl, unsubstituted or alkyl substituted heterocycloalkylalkyl, unsubstituted or alkyl substituted heterocycloalkyl, —CONR_{5d}R₅ₑ, -alkyl-CONR_{5d}R₅ₑ, cycloalkyl, alkoxyalkyl or alkylaminoalkyl, wherein R_{5d} and R₅ₑ are each independently hydrogen or alkyl.

25. (canceled)

26: A compound that is selected from:

| Compound | |
|---|---|
| No. | IUPAC Names |
| 1 | 2-((5-chloro-2-(2-(1′-cyano-1′,2′,3′,6′-tetrahydro-[2,4′-bipyridin]-5- |
| | yl)acetamido)pyridin-4-yl)oxy)-N-methylpropanamide; |
| 2 | 2-((5-chloro-2-(2-(1′-cyano-1′,2′,3′,6′-tetrahydro-[2,4′-bipyridin]-5- |
| | yl)acetamido)pyridin-4-yl)oxy)-N-methylpropanamide (Isomer-1 of Compound 1); |
| 3 | 2-((5-chloro-2-(2-(1′-cyano-1′,2′,3′,6′-tetrahydro-[2,4′-bipyridin]-5- |
| | yl)acetamido)pyridin-4-yl)oxy)-N-methylpropanamide (Isomer-2 of Compound 1); |
| 4 | N-(5-chloropyridin-2-yl)-2-(1′-cyano-1′,2′,3′,6′-tetrahydro-[2,4′-bipyridin]-5- |
| | yl)acetamide; |
| 5 | N-(5-chloropyridin-2-yl)-2-(1′-cyano-1′,2′,5′,6′-tetrahydro-[2,3′-bipyridin]-5- |
| | yl)acetamide; |
| 6 | N-(5-chloropyridin-2-yl)-2-(6-(1-cyano-2,5-dihydro-1H-pyrrol-3-yl)pyridin-3- |
| | yl)acetamide; |
| 7 | N-(5-chloro-4-morpholinopyridin-2-yl)-2-(1′-cyano-1′,2′,3′,6′-tetrahydro-[2,4′- |
| | bipyridin]-5-yl)acetamide; |
| 8 | N-(5-chloro-4-methoxypyridin-2-yl)-2-(1′-cyano-1′,2′,3′,6′-tetrahydro-[2,4′- |
| | bipyridin]-5-yl)acetamide; |
| 9 | N-(5-chloro-4-(4-methylpiperazin-1-yl)pyridin-2-yl)-2-(1′-cyano-1′,2′,3′,6′- |
| | tetrahydro-[2,4′-bipyridin]-5-yl)acetamide; |
| 10 | N-(5-chloro-4-((1-methylpyrrolidin-3-yl)oxy)pyridin-2-yl)-2-(1′-cyano-1′,2′,3′,6′- |
| | tetrahydro-[2,4′-bipyridin]-5-yl)acetamide; |
| 11 | N-(5-chloro-4-((1-methylpiperidin-4-yl)oxy)pyridin-2-yl)-2-(1′-cyano-1′,2′,3′,6′- |
| | tetrahydro-[2,4′-bipyridin]-5-yl)acetamide; |
| 12 | N-(5-chloro-4-((tetrahydrofuran-3-yl)oxy)pyridin-2-yl)-2-(1′-cyano-1′,2′,3′,6′- |
| | tetrahydro-[2,4′-bipyridin]-5-yl)acetamide |
| 13 | N-(5-chloropyridin-2-yl)-2-(1′-cyano-3-methyl-1′,2′,3′,6′-tetrahydro-[2,4′-bipyridin]- |
| | 5-yl)acetamide; |
| 14 | N-(5-chloro-4-((1-methoxypropan-2-yl)oxy)pyridin-2-yl)-2-(1′-cyano-1′,2′,3′,6′- |
| | tetrahydro-[2,4′-bipyridin]-5-yl)acetamide; |
| 15 | N-(5-chloro-4-(oxetan-3-yloxy)pyridin-2-yl)-2-(1′-cyano-1′,2′,3′,6′-tetrahydro-[2,4′- |
| | bipyridin]-5-yl)acetamide; |
| 16 | N-(5-chloropyridin-2-yl)-2-(6-(1-cyanopyrrolidin-3-yl)pyridin-3-yl)acetamide; |
| 17 | N-(5-chloro-4-(3-hydroxyoxetan-3-yl)pyridin-2-yl)-2-(1′-cyano-1′,2′,3′,6′- |
| | tetrahydro-[2,4′-bipyridin]-5-yl)acetamide; |
| 18 | N-(5-chloropyridin-2-yl)-2-(1′-cyano-3-fluoro-1′,2′,3′,6′-tetrahydro-[2,4′-bipyridin]- |
| | 5-yl)acetamide; |
| 19 | N-(5-chloro-4-((tetrahydro-2H-pyran-4-yl)oxy)pyridin-2-yl)-2-(1′-cyano-1′,2′,3′,6′- |
| | tetrahydro-[2,4′-bipyridin]-5-yl)acetamide; |
| 20 | N-(5-chloro-4-ethoxypyridin-2-yl)-2-(1′-cyano-1′,2′,3′,6′-tetrahydro-[2,4′-bipyridin]- |
| | 5-yl)acetamide; |
| 21 | N-(5-chloro-4-isopropoxypyridin-2-yl)-2-(1′-cyano-1′,2′,3′,6′-tetrahydro-[2,4′- |
| | bipyridin]-5-yl)acetamide; |
| 22 | N-(5-chloro-4-(2-methoxyethoxy)pyridin-2-yl)-2-(1′-cyano-1′,2′,3′,6′-tetrahydro- |
| | [2,4′-bipyridin]-5-yl)acetamide; |
| 23 | N-(5-chloro-4-(2-hydroxypropan-2-yl)pyridin-2-yl)-2-(1′-cyano-1′,2′,3′,6′- |
| | tetrahydro-[2,4′-bipyridin]-5-yl)acetamide; |
| 24 | (R)-N-(5-chloro-4-(1-hydroxy-2-methoxyethoxy)pyridin-2-yl)-2-(1′-cyano- |
| | 1′,2′,3′,6′-tetrahydro-[2,4′-bipyridin]-5-yl)acetamide; |
| 25 | (S)-N-(5-chloro-4-(1-hydroxy-2-methoxyethoxy)pyridin-2-yl)-2-(1′-cyano-1′,2′,3′,6′- |
| | tetrahydro-[2,4′-bipyridin]-5-yl)acetamide; |
| 26 | (R)-N-(5-chloro-4-((tetrahydrofuran-3-yl)oxy)pyridin-2-yl)-2-(1′-cyano-1′,2′,3′,6′- |
| | tetrahydro-[2,4′-bipyridin]-5-yl)acetamide; |
| 27 | (S)-N-(5-chloro-4-((tetrahydrofuran-3-yl)oxy)pyridin-2-yl)-2-(1′-cyano-1′,2′,3′,6′- |
| | tetrahydro-[2,4′-bipyridin]-5-yl)acetamide; |
| 28 | N-(5-chloro-4-cyclobutoxypyridin-2-yl)-2-(1′-cyano-1′,2′,3′,6′-tetrahydro-[2,4′- |
| | bipyridin]-5-yl)acetamide; |
| 29 | (R)-N-(5-chloro-4-((1-methylpyrrolidin-3-yl)oxy)pyridin-2-yl)-2-(1′-cyano- |
| | 1′,2′,3′,6′-tetrahydro-[2,4′-bipyridin]-5-yl)acetamide; |
| 30 | (S)-N-(5-chloro-4-((1-methylpyrrolidin-3-yl)oxy)pyridin-2-yl)-2-(1′-cyano- |
| | 1′,2′,3′,6′-tetrahydro-[2,4′-bipyridin]-5-yl)acetamide; |
| 31 | N-(5-chloro-4-((1-(dimethylamino)propan-2-yl)oxy)pyridin-2-yl)-2-(1′-cyano- |
| | 1′,2′,3′,6′-tetrahydro-[2,4′-bipyridin]-5-yl)acetamide; |
| 32 | (R)-N-(5-chloro-4-((1-methylpiperidin-3-yl)oxy)pyridin-2-yl)-2-(1′-cyano-1′,2′,3′,6′- |
| | tetrahydro-[2,4′-bipyridin]-5-yl)acetamide; |
| 33 | (S)-N-(5-chloro-4-((1-methylpiperidin-3-yl)oxy)pyridin-2-yl)-2-(1′-cyano-1′,2′,3′,6′- |
| | tetrahydro-[2,4′-bipyridin]-5-yl)acetamide; |
| 34 | N-(5-chloro-4-methoxypyridin-2-yl)-2-(1′-cyano-1′,2′,5′,6′-tetrahydro-[2,3′- |
| | bipyridin]-5-yl)acetamide; |
| 35 | (S)-N-(5-chloro-4-((1-methoxypropan-2-yl)oxy)pyridin-2-yl)-2-(1′-cyano-1′,2′,5′,6′- |
| | tetrahydro-[2,3′-bipyridin]-5-yl)acetamide; |
| 36 | (R)-N-(5-chloro-4-((1-methoxypropan-2-yl)oxy)pyridin-2-yl)-2-(1′-cyano-1′,2′,5′,6′- |
| | tetrahydro-[2,3′-bipyridin]-5-yl)acetamide; |
| 37 | N-(5-chloro-4-((1-methylpiperidin-4-yl)oxy)pyridin-2-yl)-2-(1′-cyano-1′,2′,5′,6′- |
| | tetrahydro-[2,3′-bipyridin]-5-yl)acetamide; |
| 38 | (R)-N-(5-chloro-4-((1-methylpyrrolidin-3-yl)oxy)pyridin-2-yl)-2-(1′-cyano- |
| | 1′,2′,5′,6′-tetrahydro-[2,3′-bipyridin]-5-yl)acetamide; |
| 39 | (S)-N-(5-chloro-4-((1-methylpyrrolidin-3-yl)oxy)pyridin-2-yl)-2-(1′-cyano- |
| | 1′,2′,5′,6′-tetrahydro-[2,3′-bipyridin]-5-yl)acetamide; |
| 40 | (R)-N-(5-chloro-4-((tetrahydrofuran-3-yl)oxy)pyridin-2-yl)-2-(1′-cyano-1′,2′,5′,6′- |
| | tetrahydro-[2,3′-bipyridin]-5-yl)acetamide; |
| 41 | (S)-N-(5-chloro-4-((tetrahydrofuran-3-yl)oxy)pyridin-2-yl)-2-(1′-cyano-1′,2′,5′,6′- |
| | tetrahydro-[2,3′-bipyridin]-5-yl)acetamide; |
| 42 | N-(5-chloro-4-((1-(dimethylamino)propan-2-yl)oxy)pyridin-2-yl)-2-(1′-cyano- |
| | 1′,2′,5′,6′-tetrahydro-[2,3′-bipyridin]-5-yl)acetamide; |
| 43 | N-(5-chloro-4-((tetrahydro-2H-pyran-3-yl)oxy)pyridin-2-yl)-2-(1′-cyano-1′,2′,5′,6′- |
| | tetrahydro-[2,3′-bipyridin]-5-yl)acetamide; |
| 44 | N-(5-chloro-4-((tetrahydro-2H-pyran-3-yl)oxy)pyridin-2-yl)-2-(1′-cyano-1′,2′,5′,6′- |
| | tetrahydro-[2,3′-bipyridin]-5-yl)acetamide (Isomer-1 of compound-43); |
| 45 | N-(5-chloro-4-((tetrahydro-2H-pyran-3-yl)oxy)pyridin-2-yl)-2-(1′-cyano-1′,2′,5′,6′- |
| | tetrahydro-[2,3′-bipyridin]-5-yl)acetamide (Isomer-2 of compound-43); |
| 46 | (R)-N-(5-chloro-4-((1-methylpiperidin-3-yl)oxy)pyridin-2-yl)-2-(1′-cyano-1′,2′,5′,6′- |
| | tetrahydro-[2,3′-bipyridin]-5-yl)acetamide; |
| 47 | (S)-N-(5-chloro-4-((1-methylpiperidin-3-yl)oxy)pyridin-2-yl)-2-(1′-cyano-1′,2′,5′,6′- |
| | tetrahydro-[2,3′-bipyridin]-5-yl)acetamide; |
| 48 | N-(5-chloro-4-methoxypyridin-2-yl)-2-(1′-cyano-1′,2′,5′,6′-tetrahydro-[2,3′- |
| | bipyridin]-5-yl)propanamide; |
| 49 | N-(5-chloro-4-methoxypyridin-2-yl)-2-(1′-cyano-1′,2′,5′,6′-tetrahydro-[2,3′- |
| | bipyridin]-5-yl)propenamide (Isomer-1 of compound-48); |
| 50 | N-(5-chloro-4-methoxypyridin-2-yl)-2-(1′-cyano-1′,2′,5′,6′-tetrahydro-[2,3′- |
| | bipyridin]-5-yl)propenamide (Isomer-2 of compound-48); |
| 51 | N-(5-chloro-4-methoxypyridin-2-yl)-2-(1′-cyano-1′,2′,3′,6′-tetrahydro-[2,4′- |
| | bipyridin]-5-yl)propanamide; |
| 52 | N-(5-chloro-4-methoxypyridin-2-yl)-2-(1′-cyano-1′,2′,3′,6′-tetrahydro-[2,4′- |
| | bipyridin]-5-yl)propenamide (Isomer-1 of compound-52); |
| 53 | N-(5-chloro-4-methoxypyridin-2-yl)-2-(1′-cyano-1′,2′,3′,6′-tetrahydro-[2,4′- |
| | bipyridin]-5-yl)propenamide (Isomer-2 of compound-52); |
| 54 | N-(5-chloro-4-((1-methylpyrrolidin-2-yl)methoxy)pyridin-2-yl)-2-(1′-cyano- |
| | 1′,2′,3′,6′-tetrahydro-[2,4′-bipyridin]-5-yl)acetamide; |
| 55 | N-(5-chloro-4-((1-methylpyrrolidin-2-yl)methoxy)pyridin-2-yl)-2-(1′-cyano- |
| | 1′,2′,5′,6′-tetrahydro-[2,3′-bipyridin]-5-yl)acetamide; |
| 56 | 2-((5-chloro-2-(2-(1′-cyano-1′,2′,5′,6′-tetrahydro-[2,3′-bipyridin]-5- |
| | yl)acetamido)pyridin-4-yl)oxy)-N-methylpropanamide; |
| 57 | 2-((5-chloro-2-(2-(1′-cyano-1′,2′,5′,6′-tetrahydro-[2,3′-bipyridin]-5- |
| | yl)acetamido)pyridin-4-yl)oxy)-N-methylpropanamide (Isomer-1 of compound-56); |
| 58 | 2-((5-chloro-2-(2-(1′-cyano-1′,2′,5′,6′-tetrahydro-[2,3′-bipyridin]-5- |
| | yl)acetamido)pyridin-4-yl)oxy)-N-methylpropanamide (Isomer-2 of compound-56); |
| 59 | N-(5-chloro-4-methoxypyridin-2-yl)-2-(6-(1-cyanopiperidin-4-yl)pyridin-3- |
| | yl)acetamide; |
| 60 | N-(5-chloro-4-methoxypyridin-2-yl)-2-(6-(1-cyano-3-methylpiperidin-4-yl)pyridin- |
| | 3-yl)acetamide; |
| 61 | N-(5-chloro-4-((tetrahydro-2H-pyran-4-yl)oxy)pyridin-2-yl)-2-(1′-cyano-1′,2′,5′,6′- |
| | tetrahydro-[2,3′-bipyridin]-5-yl)acetamide; |
| 62 | N-(5-chloro-4-((8-methyl-8-azabicyclo[3.2.1]octan-3-yl)oxy)pyridin-2-yl)-2-(1′- |
| | cyano-1′,2′,5′,6′-tetrahydro-[2,3′-bipyridin]-5-yl)acetamide; |
| 63 | N-(5-chloro-4-((1,2-dimethylpiperidin-4-yl)oxy)pyridin-2-yl)-2-(1′-cyano-1′,2′,5′,6′- |
| | tetrahydro-[2,3′-bipyridin]-5-yl)acetamide; |
| 64 | N-(5-chloro-4-((1,2-dimethylpiperidin-4-yl)oxy)pyridin-2-yl)-2-(1′-cyano-1′,2′,5′,6′- |
| | tetrahydro-[2,3′-bipyridin]-5-yl)acetamide (Isomer-1 of compound-63); |
| 64A | N-(5-chloro-4-((1,2-dimethylpiperidin-4-yl)oxy)pyridin-2-yl)-2-(1′-cyano-1′,2′,5′,6′- |
| | tetrahydro-[2,3′-bipyridin]-5-yl)acetamide (Isomer-2 of compound-63); |
| 65 | N-(5-chloro-4-methoxypyridin-2-yl)-2-(1′-cyano-2′-methyl-1′,2′,3′,6′-tetrahydro- |
| | [2,4′-bipyridin]-5-yl)acetamide; |
| 66 | N-(5-chloro-4-methoxypyridin-2-yl)-2-(1′-cyano-2′-methyl-1′,2′,3′,6′-tetrahydro- |
| | [2,4′-bipyridin]-5-yl)acetamide (Isomer-1 of compound-65); |
| 67 | N-(5-chloro-4-methoxypyridin-2-yl)-2-(1′-cyano-2′-methyl-1′,2′,3′,6′-tetrahydro- |
| | [2,4′-bipyridin]-5-yl)acetamide (Isomer-2 of compound-65); |
| 68 | N-(5-chloro-4-methoxypyridin-2-yl)-2-(1′-cyano-2′-methyl-1′,2′,3′,6′-tetrahydro- |
| | [2,4′-bipyridin]-5-yl)acetamide; |
| 69 | N-(5-chloro-4-((2-methyl-2-azaspiro[3.3]heptan-6-yl)oxy)pyridin-2-yl)-2-(1′-cyano- |
| | 1′,2′,5′,6′-tetrahydro-[2,3′-bipyridin]-5-yl)acetamide; |
| 70 | N-(5-chloro-4-methoxypyridin-2-yl)-2-(1′-cyano-3′-methyl-1′,2′,3′,6′-tetrahydro- |
| | [2,4′-bipyridin]-5-yl)acetamide; |
| 71 | N-(5-chloro-4-methoxypyridin-2-yl)-2-(1′-cyano-3′-methyl-1′,2′,3′,6′-tetrahydro- |
| | [2,4′-bipyridin]-5-yl)acetamide (Isomer-1 of compound 70); |
| 72 | N-(5-chloro-4-methoxypyridin-2-yl)-2-(1′-cyano-3′-methyl-1′,2′,3′,6′-tetrahydro- |
| | [2,4′-bipyridin]-5-yl)acetamide (Isomer-2 of compound 70); |
| 73 | N-(5-chloro-4-methoxypyridin-2-yl)-2-(6-(8-cyano-8-azabicyclo[3.2.1]oct-2-en-3- |
| | yl)pyridin-3-yl)acetamide; |
| 74 | N-(5-chloro-4-methoxypyridin-2-yl)-2-(6-(8-cyano-8-azabicyclo[3.2.1]octan-3- |
| | yl)pyridin-3-yl)acetamide; |
| 75 | N-(5-chloro-4-methoxypyridin-2-yl)-2-(6-(2-cyano-1,2,3,3a,4,6a- |
| | hexahydrocyclopenta[c]pyrrol-5-yl)pyridin-3-yl)acetamide; |
| 76 | N-(5-chloro-4-methoxypyridin-2-yl)-2-(6-(2-cyanooctahydrocyclopenta[c]pyrrol-5- |
| | yl)pyridin-3-yl)acetamide; |
| 77 | 5-chloro-2-(2-(1′-cyano-1′,2′,5′,6′-tetrahydro-[2,3′-bipyridin]-5- |
| | yl)acetamido)pyridin-4-yl dimethylcarbamate; |
| 78 | N-(5-chloro-4-methoxypyridin-2-yl)-2-(6-(1-cyanopiperidin-3-yl)pyridin-3- |
| | yl)acetamide; |
| 79 | N-(5-chloro-4-methoxypyridin-2-yl)-2-(6-(1-cyanopiperidin-3-yl)pyridin-3- |
| | yl)acetamide (Isomer-1 of compound-78); |
| 80 | N-(5-chloro-4-methoxypyridin-2-yl)-2-(6-(1-cyanopiperidin-3-yl)pyridin-3- |
| | yl)acetamide (Isomer-2 of compound-78); |
| 81 | N-(5-chloro-4-methoxypyridin-2-yl)-2-(6-(2-cyano-2-azabicyclo[2.2.2]oct-5-en-5- |
| | yl)pyridin-3-yl)acetamide; |
| 82 | 2-((5-chloro-2-(2-(6-(1-cyanopiperidin-4-yl)pyridin-3-yl)acetamido)pyridin-4- |
| | yl)oxy)-N-methylpropanamide; |
| 83 | 2-((5-chloro-2-(2-(6-(1-cyanopiperidin-4-yl)pyridin-3-yl)acetamido)pyridin-4- |
| | yl)oxy)-N-methylpropanamide (Isomer-1 of compound-82); |
| 84 | 2-((5-chloro-2-(2-(6-(1-cyanopiperidin-4-yl)pyridin-3-yl)acetamido)pyridin-4- |
| | yl)oxy)-N-methylpropanamide (Isomer-2 of compound-82); |
| 85 | 2-((5-chloro-2-(2-(6-(1-cyanopiperidin-3-yl)pyridin-3-yl)acetamido)pyridin-4- |
| | yl)oxy)-N-methylpropanamide; |
| 86 | 2-((5-chloro-2-(2-(6-(1-cyanopiperidin-3-yl)pyridin-3-yl)acetamido)pyridin-4- |
| | yl)oxy)-N-methylpropanamide; |
| 87 | 2-((5-chloro-2-(2-(6-(1-cyanopiperidin-3-yl)pyridin-3-yl)acetamido)pyridin-4- |
| | yl)oxy)-N-methylpropanamide; |
| 88 | 2-((5-chloro-2-(2-(6-(1-cyanopiperidin-3-yl)pyridin-3-yl)acetamido)pyridin-4- |
| | yl)oxy)-N-methylpropanamide; |
| 89 | 2-((5-chloro-2-(2-(6-(1-cyanopiperidin-3-yl)pyridin-3-yl)acetamido)pyridin-4- |
| | yl)oxy)-N-methylpropanamide; |
| 90 | 2-((5-chloro-2-(2-(6-(1-cyanopiperidin-3-yl)pyridin-3-yl)acetamido)pyridin-4- |
| | yl)oxy)-N-methylpropanamide; |
| 91 | 2-((5-chloro-2-(2-(6-(1-cyanopiperidin-3-yl)pyridin-3-yl)acetamido)pyridin-4- |
| | yl)oxy)-N-methylpropanamide; |
| 92 | N-(5-chloro-4-(((R)-tetrahydrofuran-3-yl)oxy)pyridin-2-yl)-2-(6-(1-cyanopiperidin- |
| | 3-yl)pyridin-3-yl)acetamide; |
| 93 | N-(5-chloro-4-(((R)-tetrahydrofuran-3-yl)oxy)pyridin-2-yl)-2-(6-(1-cyanopiperidin- |
| | 3-yl)pyridin-3-yl)acetamide (Isomer-1 of Compound-92); |
| 94 | N-(5-chloro-4-(((R)-tetrahydrofuran-3-yl)oxy)pyridin-2-yl)-2-(6-(1-cyanopiperidin- |
| | 3-yl)pyridin-3-yl)acetamide (Isomer-2 of Compound-92); |
| 95 | N-(5-chloro-4-(((S)-tetrahydrofuran-3-yl)oxy)pyridin-2-yl)-2-(6-(1-cyanopiperidin- |
| | 3-yl)pyridin-3-yl)acetamide; |
| 96 | N-(5-chloro-4-(((S)-tetrahydrofuran-3-yl)oxy)pyridin-2-yl)-2-(6-(1-cyanopiperidin- |
| | 3-yl)pyridin-3-yl)acetamide (Isomer-1 of Compound-95); |
| 97 | N-(5-chloro-4-(((S)-tetrahydrofuran-3-yl)oxy)pyridin-2-yl)-2-(6-(1-cyanopiperidin- |
| | 3-yl)pyridin-3-yl)acetamide (Isomer-2 of Compound-95); |
| 98 | (R)-N-(5-chloro-4-((tetrahydrofuran-3-yl)oxy)pyridin-2-yl)-2-(6-(1-cyanopiperidin- |
| | 4-yl)pyridin-3-yl)acetamide; |
| 99 | (S)-N-(5-chloro-4-((tetrahydrofuran-3-yl)oxy)pyridin-2-yl)-2-(6-(1-cyanopiperidin- |
| | 4-yl)pyridin-3-yl)acetamide; |
| 100 | 5-chloro-2-(2-(1′-cyano-1′,2′,3′,6′-tetrahydro-[2,4′-bipyridin]-5- |
| | yl)acetamido)pyridin-4-yl dimethylcarbamate; |
| 101 | 5-chloro-2-(2-(6-(1-cyanopiperidin-4-yl)pyridin-3-yl)acetamido)pyridin-4-yl |
| | dimethylcarbamate; |
| 102 | 2-(1′-cyano-1′,2′,5′,6′-tetrahydro-[2,3′-bipyridin]-5-yl)-N-(pyridin-2-yl)acetamide; |
| 103 | 2-(1′-cyano-1′,2′,5′,6′-tetrahydro-[2,3′-bipyridin]-5-yl)-N-(5-fluoropyridin-2- |
| | yl)acetamide; |
| 104 | 2-(1′-cyano-1′,2′,5′,6′-tetrahydro-[2,3′-bipyridin]-5-yl)-N-(5-methylpyridin-2- |
| | yl)acetamide; |
| 105 | 2-(1′-cyano-1′,2′,5′,6′-tetrahydro-[2,3′-bipyridin]-5-yl)-N-(4,5-dichloropyridin-2- |
| | yl)acetamide; |
| 106 | 2-(1′-cyano-1′,2′,5′,6′-tetrahydro-[2,3′-bipyridin]-5-yl)-N-(5-cyclopropylpyridin-2- |
| | yl)acetamide; |
| 107 | 2-(1′-cyano-1′,2′,5′,6′-tetrahydro-[2,3′-bipyridin]-5-yl)-N-(5-(methylthio)pyridin-2- |
| | yl)acetamide; |
| 108 | N-(5-chloropyrazin-2-yl)-2-(1′-cyano-1′,2′,5′,6′-tetrahydro-[2,3′-bipyridin]-5- |
| | yl)acetamide; |
| 109 | N-(5-chloro-4-(dimethylamino)pyridin-2-yl)-2-(1′-cyano-1′,2′,5′,6′-tetrahydro-[2,3′- |
| | bipyridin]-5-yl)acetamide; |
| 110 | N-(5-chloro-4-methoxypyridin-2-yl)-2-(1′-cyano-1′,2′,5′,6′-tetrahydro-[2,3′- |
| | bipyridin]-5-yl)-2-methylpropanamide; |
| 111 | N-(5-chloro-4-methoxypyridin-2-yl)-2-(1′-cyano-1′,2′,5′,6′-tetrahydro-[3,3′- |
| | bipyridin]-6-yl)acetamide; |
| 112 | 2-(2-chloro-4-(1-cyano-1,2,5,6-tetrahydropyridin-3-yl)phenyl)-N-(5-chloro-4- |
| | methoxypyridin-2-yl)acetamide; |
| 113 | N-(5-chloro-4-methoxypyridin-2-yl)-2-(4-(1-cyano-1,2,5,6-tetrahydropyridin-3-yl)- |
| | 2-fluorophenyl)acetamide; |
| 114 | N-(5-chloro-4-methoxypyridin-2-yl)-2-(4-(1-cyano-1,2,5,6-tetrahydropyridin-3- |
| | yl)phenyl)acetamide; |
| 115 | N-(5-chloro-4-methoxypyridin-2-yl)-2-(4-(2-cyano-1,2,3,3a,4,6a- |
| | hexahydrocyclopenta[c]pyrrol-5-yl)phenyl)acetamide; |
| 116 | N-(5-chloro-4-methoxypyridin-2-yl)-2-(4-(1-cyano-1,2,5,6-tetrahydropyridin-3-yl)- |
| | 3-methoxyphenyl)acetamide; |
| 117 | N-(5-chloro-4-methoxypyridin-2-yl)-2-(1′-cyano-3-methoxy-1′,2′,5′,6′-tetrahydro- |
| | [2,3′-bipyridin]-5-yl)acetamide; |
| 118 | (S)-N-(5-chloro-4-((1-methylpyrrolidin-3-yl)oxy)pyridin-2-yl)-2-(6-(1- |
| | cyanopiperidin-4-yl)pyridin-3-yl)acetamide; |
| 119 | (R)-N-(5-chloro-4-((1-methylpyrrolidin-3-yl)oxy)pyridin-2-yl)-2-(6-(1- |
| | cyanopiperidin-4-yl)pyridin-3-yl)acetamide; |
| 120 | N-(5-chloro-4-methoxypyridin-2-yl)-2-(1′-cyano-3-methoxy-1′,2′,3′,6′-tetrahydro- |
| | [2,4′-bipyridin]-5-yl)acetamide; |
| 121 | N-(5-chloro-4-methoxypyridin-2-yl)-2-(6-(1-cyanopiperidin-4-yl)-5- |
| | methoxypyridin-3-yl)acetamide; |
| 122 | (R)-N-(5-chloro-4-((tetrahydrofuran-3-yl)oxy)pyridin-2-yl)-2-(1′-cyano-3-methoxy- |
| | 1′,2′,3′,6′-tetrahydro-[2,4′-bipyridin]-5-yl)acetamide; |
| 123 | (R)-N-(5-chloro-4-((tetrahydrofuran-3-yl)oxy)pyridin-2-yl)-2-(6-(1-cyanopiperidin- |
| | 4-yl)-5-methoxypyridin-3-yl)acetamide; |
| 124 | 2-(6-(1-cyanopiperidin-4-yl)pyridin-3-yl)-N-(5-cyclopropyl-4-methoxypyridin-2- |
| | yl)acetamide; |
| 125 | (S)-N-(5-chloro-4-((1-methylpiperidin-3-yl)oxy)pyridin-2-yl)-2-(6-(1- |
| | cyanopiperidin-4-yl)pyridin-3-yl)acetamide; |
| 126 | (R)-N-(5-chloro-4-((1-methylpiperidin-3-yl)oxy)pyridin-2-yl)-2-(6-(1- |
| | cyanopiperidin-4-yl)pyridin-3-yl)acetamide; |
| 127 | N-(5-chloro-4-methoxypyridin-2-yl)-2-(1′-cyano-1′,2′,3′,6′-tetrahydro-[3,4′- |
| | bipyridin]-6-yl)acetamide; |
| 128 | (S)-2-((5-chloro-2-(2-(6-(1-cyanopiperidin-4-yl)-5-methoxypyridin-3- |
| | yl)acetamido)pyridin-4-yl)oxy)-N-methylpropanamide; |
| 129 | N-(5-chloro-4-((1-methylpiperidin-4-yl)oxy)pyridin-2-yl)-2-(6-(1-cyanopiperidin-4- |
| | yl)pyridin-3-yl)acetamide; |
| 130 | N-(5-chloro-4-methoxypyridin-2-yl)-2-(6′-(cyanamidomethyl)-[2,3′-bipyridin]-5- |
| | yl)acetamide; |
| 131 | N-(5-chloro-4-methoxypyridin-2-yl)-2-(6′-((N-methylcyanamido)methyl)-[2,3′- |
| | bipyridin]-5-yl)acetamide; |
| 132 | N-(5-chloro-4-methoxypyridin-2-yl)-2-(6′-((N-isopropylcyanamido)methyl)-[2,3′- |
| | bipyridin]-5-yl)acetamide; |
| 133 | N-(5-chloro-4-methoxypyridin-2-yl)-2-(6-(3-cyano-3-azabicyclo[4.1.0]heptan-6- |
| | yl)pyridin-3-yl)acetamide; |
| 134 | N-(5-chloro-4-methoxypyridin-2-yl)-2-(6-(4-cyanopiperazin-1-yl)pyridin-3- |
| | yl)acetamide; |
| 135 | N-(5-chloro-4-(dimethylphosphoryl)pyridin-2-yl)-2-(1′-cyano-1′,2′,5′,6′-tetrahydro- |
| | [2,3′-bipyridin]-5-yl)acetamide; |
| 136 | N-(5-chloro-4-(piperidin-4-yloxy)pyridin-2-yl)-2-(1′-cyano-1′,2′,3′,6′-tetrahydro- |
| | [2,4′-bipyridin]-5-yl)acetamide; |
| 137 | N-(4-((2-azaspiro[3.3]heptan-6-yl)oxy)-5-chloropyridin-2-yl)-2-(1′-cyano-1′,2′,3′,6′- |
| | tetrahydro-[2,4′-bipyridin]-5-yl)acetamide; |
| 138 | N-(5-chloro-4-((6,6-dimethylpiperidin-3-yl)oxy)pyridin-2-yl)-2-(1′-cyano-1′,2′,3′,6′- |
| | tetrahydro-[2,4′-bipyridin]-5-yl)acetamide; |
| 139 | N-(5-chloro-4-((2,2-dimethylpiperidin-4-yl)oxy)pyridin-2-yl)-2-(1′-cyano-1′,2′,3′,6′- |
| | tetrahydro-[2,4′-bipyridin]-5-yl)acetamide; |
| 140 | N-(5-chloro-4-methoxypyridin-2-yl)-2-(6′-cyano-[2,3′-bipyridin]-5-yl)acetamide;; |
| 141 | N-(5-chloro-4-methoxypyridin-2-yl)-2-(6-(2-cyanopyrimidin-5-yl)pyridin-3- |
| | yl)acetamide; |
| 142 | N-(5-chloro-4-methoxypyridin-2-yl)-2-(2′-cyano-[2,4′-bipyridin]-5-yl)acetamide; |
| 143 | 2-((5-chloro-2-(2-(6′-cyano-[2,3′-bipyridin]-5-yl)acetamido)pyridin-4-yl)oxy)-N- |
| | methylpropanamide; |
| 144 | 2-((5-chloro-2-(2-(2′-cyano-[2,4′-bipyridin]-5-yl)acetamido)pyridin-4-yl)oxy)-N- |
| | methylpropanamide; |
| 145 | (R)-N-(5-chloro-4-((tetrahydrofuran-3-yl)oxy)pyridin-2-yl)-2-(2′-cyano-[2,4′- |
| | bipyridin]-5-yl)acetamide; |
| 146 | (R)-N-(5-chloro-4-((tetrahydrofuran-3-yl)oxy)pyridin-2-yl)-2-(6′-cyano-[2,3′- |
| | bipyridin]-5-yl)acetamide; |
| 147 | (S)-2-((5-chloro-2-(2-(6′-cyano-[2,3′-bipyridin]-5-yl)acetamido)pyridin-4-yl)oxy)- |
| | N-methylpropanamide; |
| 148 | (S)-2-((5-chloro-2-(2-(2′-cyano-[2,4′-bipyridin]-5-yl)acetamido)pyridin-4-yl)oxy)- |
| | N-methylpropanamide; |
| 149 | N-(5-chloro-4-methoxypyridin-2-yl)-2-(6-(4-cyano-3-fluorophenyl)pyridin-3- |
| | yl)acetamide; |
| 150 | (R)-N-(5-chloro-4-((tetrahydrofuran-3-yl)oxy)pyridin-2-yl)-2-(6-(2-cyanopyrimidin- |
| | 5-yl)pyridin-3-yl)acetamide; |
| 151 | (S)-2-((5-chloro-2-(2-(6-(2-cyanopyrimidin-5-yl)pyridin-3-yl)acetamido)pyridin-4- |
| | yl)oxy)-N-methylpropanamide; |
| 152 | N-(5-chloro-4-methoxypyridin-2-yl)-2-(4-(6-cyanopyridin-2-yl)phenyl)acetamide; |
| 153 | N-(5-chloro-4-methoxypyridin-2-yl)-2-(4-(6-cyanopyridin-2-yl)-3- |
| | fluorophenyl)acetamide; |
| 154 | (R)-N-(5-chloro-4-((tetrahydrofuran-3-yl)oxy)pyridin-2-yl)-2-(6′-cyano-[2,2′- |
| | bipyridin]-5-yl)acetamide; |
| 155 | 2-((5-chloro-2-(2-(6′-cyano-[2,2′-bipyridin]-5-yl)acetamido)pyridin-4-yl)oxy)-N- |
| | methylpropanamide; |
| 156 | N-(5-chloro-4-methoxypyridin-2-yl)-2-(6′-cyano-[2,2′-bipyridin]-5-yl)acetamide; |
| 157 | N-(5-chloro-4-methoxypyridin-2-yl)-2-(6-(6-cyanopyrazin-2-yl)pyridin-3- |
| | yl)acetamide; |
| 158 | N-(5-chloro-4-methoxypyridin-2-yl)-2-(6′-cyano-5′-methyl-[2,2′-bipyridin]-5- |
| | yl)acetamide; |
| 159 | N-(5-chloro-4-methoxypyridin-2-yl)-2-(6′-cyano-4′-methyl-[2,2′-bipyridin]-5- |
| | yl)acetamide; |
| 160 | N-(5-chloro-4-methoxypyridin-2-yl)-2-(6′-cyano-3′-methyl-[2,2′-bipyridin]-5- |
| | yl)acetamide; |
| 161 | N-(5-chloro-4-methoxypyridin-2-yl)-2-(6-(4-cyanopyrimidin-2-yl)pyridin-3- |
| | yl)acetamide; |
| 162 | N-(5-chloro-4-methoxypyridin-2-yl)-2-(6-(2-cyanopyrimidin-4-yl)pyridin-3- |
| | yl)acetamide; |
| 163 | N-(5-chloro-4-methoxypyridin-2-yl)-2-(6′-cyano-5′-fluoro-[2,2′-bipyridin]-5- |
| | yl)acetamide; |
| 164 | N-(5-chloro-4-methoxypyridin-2-yl)-2-(6′-cyano-3′-fluoro-[2,2′-bipyridin]-5- |
| | yl)acetamide; |
| 165 | N-(5-chloro-4-methoxypyridin-2-yl)-2-(6′-cyano-4′-methoxy-[2,2′-bipyridin]-5- |
| | yl)acetamide; |
| 166 | N-(5-chloro-4-methoxypyridin-2-yl)-2-(6′-cyano-4′-(trifluoromethoxy)-[2,2′- |
| | bipyridin]-5-yl)acetamide; |
| 167 | N-(5-chloro-4-methoxypyridin-2-yl)-2-(6′-cyano-4′-(trifluoromethyl)-[2,2′- |
| | bipyridin]-5-yl)acetamide; |
| 168 | N-(5-chloro-4-methoxypyridin-2-yl)-2-(6′-cyano-4′-(difluoromethyl)-[2,2′- |
| | bipyridin]-5-yl)acetamide; |
| 169 | N-(5-chloro-4-methoxypyridin-2-yl)-2-(6′-cyano-4′-(2-methoxyethoxy)-[2,2′- |
| | bipyridin]-5-yl)acetamide; |
| 170 | N-(5-chloro-4-methoxypyridin-2-yl)-2-(6′-cyano-4′-(2-(dimethylamino)ethoxy)- |
| | [2,2′-bipyridin]-5-yl)acetamide; |
| 171 | N-(5-chloro-4-methoxypyridin-2-yl)-2-(6′-cyano-4′-((1-methylpyrrolidin-3-yl)oxy)- |
| | [2,2′-bipyridin]-5-yl)acetamide; |
| 172 | N-(5-chloro-4-methoxypyridin-2-yl)-2-(6′-cyano-4′-(methyl(1-methylpyrrolidin-3- |
| | yl)amino)-[2,2′-bipyridin]-5-yl)acetamide; |
| 173 | N-(5-chloro-4-methoxypyridin-2-yl)-2-(6′-cyano-4′-((2- |
| | (dimethylamino)ethyl)(methyl)amino)-[2,2′-bipyridin]-5-yl)acetamide; |
| 174 | N-(5-chloro-4-methoxypyridin-2-yl)-2-(6′-cyano-4′-(1-hydroxyethyl)-[2,2′- |
| | bipyridin]-5-yl)acetamide; |
| 175 | N-(5-chloro-4-methoxypyridin-2-yl)-2-(6′-cyano-4′-(2-hydroxypropan-2-yl)-[2,2′- |
| | bipyridin]-5-yl)acetamide; |
| 176 | N-(5-chloro-4-methoxypyridin-2-yl)-2-(6′-cyano-4′-((1-methylpiperidin-4-yl)oxy)- |
| | [2,2′-bipyridin]-5-yl)acetamide; |
| 177 | N-(5-chloro-4-methoxypyridin-2-yl)-2-(6′-cyano-4′-(1-methylpiperidin-4-yl)-[2,2′- |
| | bipyridin]-5-yl)acetamide; |
| 178 | N-(5-chloro-4-methoxypyridin-2-yl)-2-(6′-cyano-4′-(1-methylpyrrolidin-3-yl)-[2,2′- |
| | bipyridin]-5-yl)acetamide; |
| 179 | N-(5-chloro-4-methoxypyridin-2-yl)-2-(6′-cyano-4′-(1-hydroxycyclopropyl)-[2,2′- |
| | bipyridin]-5-yl)acetamide; |
| 180 | N-(5-chloro-4-methoxypyridin-2-yl)-2-(6′-cyano-4′-(1-methylpiperidin-3-yl)-[2,2′- |
| | bipyridin]-5-yl)acetamide; |
| 181 | N-(5-chloro-4-methoxypyridin-2-yl)-2-(6′-cyano-4′-(5-methyl-2,5- |
| | diazabicyclo[2.2.1]heptan-2-yl)-[2,2′-bipyridin]-5-yl)acetamide; |
| 182. | N-(5-chloro-4-methoxypyridin-2-yl)-2-(6′-cyano-4′-(3-hydroxyazetidin-1-yl)-[2,2′- |
| | bipyridin]-5-yl)acetamide; |
| 183 | N-(5-chloro-4-methoxypyridin-2-yl)-2-(6′-cyano-4′-(8-methyl-3,8- |
| | diazabicyclo[3.2.1]octan-3-yl)-[2,2′-bipyridin]-5-yl)acetamide; |
| 184 | N-(5-chloro-4-methoxypyridin-2-yl)-2-(6′-cyano-4′-(oxetan-3-yloxy)-[2,2′- |
| | bipyridin]-5-yl)acetamide; |
| 185 | N-(5-chloro-4-methoxypyridin-2-yl)-2-(6′-cyano-4′-((1-methylazetidin-3-yl)oxy)- |
| | [2,2′-bipyridin]-5-yl)acetamide; |
| 186 | N-(5-chloro-4-methoxypyridin-2-yl)-2-(6′-cyano-4′-((tetrahydrofuran-3-yl)oxy)- |
| | [2,2′-bipyridin]-5-yl)acetamide; |
| 187 | N-(5-chloro-4-methoxypyridin-2-yl)-2-(6′-cyano-4′-((2-methyl-2- |
| | azaspiro[3.3]heptan-6-yl)oxy)-[2,2′-bipyridin]-5-yl)acetamide; |
| 188 | N-(5-chloro-4-methoxypyridin-2-yl)-2-(6-(4-cyanophenyl)pyridin-3-yl)acetamide; |
| 189 | N-(5-chloro-4-methoxypyridin-2-yl)-2-(6-(3-cyanophenyl)pyridin-3-yl)acetamide; |
| 190 | (S)-2-((5-chloro-2-(2-(6′-cyano-[2,2′-bipyridin]-5-yl)acetamido)pyridin-4-yl)oxy)- |
| | N-methylpropanamide; |
| 191 | N-(5-chloro-4-methoxypyridin-2-yl)-2-(6′-cyano-4′-(4-methylpiperazin-1-yl)-[2,2′- |
| | bipyridin]-5-yl)acetamide; |
| 192. | N-(5-chloro-4-methoxypyridin-2-yl)-2-(6′-cyano-4′-(4-hydroxy-4-methylpiperidin- |
| | 1-yl)-[2,2′-bipyridin]-5-yl)acetamide; |
| 193. | N-(5-chloro-4-methoxypyridin-2-yl)-2-(6′-cyano-4′-(3-methoxyazetidin-1-yl)-[2,2′- |
| | bipyridin]-5-yl)acetamide; |
| 194. | N-(5-chloro-4-methoxypyridin-2-yl)-2-(6′-cyano-4′-(3-hydroxy-3-methylpyrrolidin- |
| | 1-yl)-[2,2′-bipyridin]-5-yl)acetamide; |
| 195. | 2-(4′-((1R,5S)-8-oxa-3-azabicyclo[3.2.1]octan-3-yl)-6′-cyano-[2,2′-bipyridin]-5-yl)- |
| | N-(5-chloro-4-methoxypyridin-2-yl)acetamide; |
| 196. | (S)-N-(5-chloro-4-methoxypyridin-2-yl)-2-(6′-cyano-4′-((tetrahydrofuran-3-yl)oxy)- |
| | [2,2′-bipyridin]-5-yl)acetamide; |
| 197. | (S)-N-(5-chloro-4-methoxypyridin-2-yl)-2-(6′-cyano-4′-((tetrahydrofuran-3-yl)oxy)- |
| | [2,2′-bipyridin]-5-yl)acetamide; |
| 198. | N-(5-chloro-4-methoxypyridin-2-yl)-2-(6′-cyano-4′-((3-fluoro-1-methylpiperidin-4- |
| | yl)oxy)-[2,2′-bipyridin]-5-yl)acetamide; and |
| 199. | N-(5-chloro-4-methoxypyridin-2-yl)-2-(6′-cyano-4′-((1-methylpiperidin-3-yl)oxy)- |
| | [2,2′-bipyridin]-5-yl)acetamide; |

or a pharmaceutically acceptable salt or a stereoisomer thereof.

27: A pharmaceutical composition comprising a compound of claim 1, or a pharmaceutically acceptable salt or a stereoisomer thereof and at least one pharmaceutically acceptable carrier or excipient.

28: The pharmaceutical composition of claim 27, for use in treating a subject suffering from a disease or condition associated with aberrant activity of CDK12/13.

29-34. (canceled)

35: A method of treating a disease and/or disorder or a condition mediated by CDK12/13 in a subject comprising administering a therapeutically effective amount of a compound according to claim 1; wherein the disease and/or disorder or the condition mediated by CDK12/13 is selected from a cancer, an inflammatory disorder, an auto-inflammatory disorder and an infectious disease, wherein the cancer is selected from a carcinoma, including that of the breast, liver, lung, colon, kidney, bladder, including small cell lung cancer, non-small cell lung cancer, head and neck, thyroid, esophagus, stomach, pancreas, ovary, gall bladder, cervix, prostate and skin, including squamous cell carcinoma; hematopoietic tumors of lymphoid lineage, including leukemia, acute lymphoblastic leukemia, acute lymphocytic leukemia, Hodgkin's lymphoma, non-Hodgkin's lymphoma, B-cell lymphoma, T-cell lymphoma, hairy cell lymphoma, myeloma, mantle cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; and other tumors, including seminoma, melanoma, osteosarcoma, teratocarcinoma, keratoacanthoma, xeroderma pigmentosum, thyroid follicular cancer and Kaposi's sarcoma.

36-37. (canceled)

38: The method of claim 35, wherein the disorder or condition mediated by CDK12/13 is Myotonic Dystrophy type 1, Myotonic Dystrophy type 2, Fragile X associated tremor/ataxia syndrome, amyotrophic lateral sclerosis (ALS) and frontotemporal dementia, Huntington's disease like 2, Huntington's disease, several types of Spinocerebellar Ataxia, Dentatorubral-pallidoluysian atrophy and Spinal and Bulbar Muscular Atrophy.

39: The method of claim 35, further comprising administering to the subject in need thereof one or more chemotherapeutic agents independently selected from anti-proliferative agents, anti-cancer agents, immunosuppressant agents and pain-relieving agents.

40. (canceled)
